(19) United States
(12) Patent Application Publication
(10) Pub. No.: US 2011/0293558 A1
(43) Pub. Date: Dec. 1, 2011

(54) MATERIAL PROPERTIES OF T CELLS AND RELATED METHODS AND COMPOSITIONS

(75) Inventors: Subra Suresh, Wellesley, MA (US); Jianzhu Chen, Lexington, MA (US); Irene Yin-Ting Chang, Kaohsiung City (CN)

(73) Assignee: Massachusetts Institute of Technology, Cambridge, MA (US)

(21) Appl. No.: 13/053,151

(22) Filed: Mar. 21, 2011

Related U.S. Application Data
(60) Provisional application No. 61/316,259, filed on Mar. 22, 2010.
(60) Provisional application No. 61/370,155, filed on Aug. 3, 2010.
(60) Provisional application No. 61/382,478, filed on Sep. 13, 2010.
(60) Provisional application No. 61/382,481, filed on Sep. 13, 2010.
(60) Provisional application No. 61/382,484, filed on Sep. 13, 2010.
(60) Provisional application No. 61/382,486, filed on Sep. 13, 2010.

(51) Int. Cl. A61K 38/20; C12Q 1/02; C40B 30/06; A61K 38/18; A61K 39/395; A61K 31/436; A61K 38/02; A61K 38/13; A61K 31/337; A61K 31/475; A61K 31/165; A61K 31/4184; A61K 31/135; A61K 31/427; A61P 37/06; A61P 31/00; G01N 33/566

(52) U.S. Cl. 424/85.2; 435/7.24; 435/29; 506/10; 514/7.6; 424/173.1; 514/291; 514/1.1; 514/20.5; 514/449; 514/283; 514/630; 514/388; 514/656; 514/369

(57) ABSTRACT

The invention in some aspects relates to methods, devices and compositions for evaluating material properties, such as mechanical and rheological properties of substances, particularly biological substances, such as cells, tissues, and biological fluids. In some aspects, the invention relates to methods, devices and compositions for evaluating material properties of deformable objects, such as cells. In further aspects, the invention relates to methods, devices and compositions for diagnosing and/or characterizing disease based on material properties of biological cells.

### RELATED APPLICATIONS

This application claims the benefit under 35 U.S.C. §119 of U.S. provisional applications 61/316,259, filed Mar. 22, 2010, 61/370,155, filed Aug. 3, 2010, 61/382,486, filed Sep. 13, 2010, 61/382,478, filed Sep. 13, 2010, 61/382,481, filed Sep. 13, 2010, and 61/382,484, filed Sep. 13, 2010, the entire contents of each of which are incorporated herein by reference.

### FEDERALLY SPONSORED RESEARCH

This invention was made with Government support under Grant Numbers HL094270 and GM076689 awarded by the National Institutes of Health. The Government has certain rights in the invention.

### FIELD OF THE INVENTION

The invention relates to methods, devices and compositions for evaluating material properties, such as mechanical and rheological properties of substances, particularly biological substances, such as cells, tissues, and biological fluids.

### BACKGROUND OF INVENTION

Cell deformability is pathologically altered in a variety of disease states, including inherited genetic disorders and both non-infectious (1) and infectious (2) diseases. Cell deformability has thus been used as a biomarker for certain disease states (3). Malaria, a disease threatening approximately 2.2 billion people globally, and causing about 250 million clinical episodes and 1 million deaths annually (4), is an example of an infectious disease process that involves decreased RBC deformability (5, 6).

While methods for studying cell biochemical characteristics (e.g. fluorescence-activated cell sorting (FACS)) of cells are common, there is a paucity of techniques for investigating mechanical properties of cells. Many existing methods for analyzing cell deformability fail to account for certain factors, e.g., cell population heterogeneity. Furthermore, certain methods are not readily translated into low-cost field diagnostic devices. Methods for studying red blood cell (RBC) deformability, for example, include filtration (9) and laser diffraction ellipsometry (10), both of which measure bulk properties of a cell population.

Examining cells individually is a strategy for characterizing inherently heterogeneous cell populations. Micropipette aspiration is one such method, and it has been applied to study infected RBC deformability (11-12) Other techniques include atomic force microscopy (13), optical stretching (14), and optical tweezers (15). Cell movement through microfabricated pores has also been evaluated (16-18). Still, these methods are labor-intensive, expensive, and time-consuming. Furthermore, the relevance of evaluating static mechanical responses of cells that function in the circulation of a living organism may be limited. There remains a need for improved methods for characterizing cell deformability.

### SUMMARY OF INVENTION

Aspects of the present invention relate to methods and devices for evaluating material properties (e.g., mechanical and rheological properties) of certain substances, particularly biological substances. In some aspects of the invention, devices and related methods are provided for evaluating material properties of deformable objects such as, for example, polymeric objects, biological cells, particles, viscoelastic objects, etc.

In some aspects, devices for evaluating material properties are provided that comprise a structure defining one or more microfluidic channels that contain one or more constrictions through which a deformable object (e.g., a biological cell) may pass. Devices and methods are provided, in some embodiments, for evaluating mechanics of deformation of such objects based, in part, on temporal and spatial parameters associated with passage through one or more constrictions.

In other aspects, the present invention features methods for analyzing, characterizing and/or predicting the deformability of biological cells, such as hematopoietic cells. In further aspects of the invention, methods and devices are provided for diagnosing, assessing, characterizing, evaluating, and/or predicting disease based on material properties of biological substances, such as cells and other deformable objects, e.g., lymphocytes, leukocytes, red blood cells, platelets, cancer cells, and tissues, e.g., blood.

According to some aspects of the invention, devices and methods are provided for modeling and predicting material properties (e.g., mechanical and rheological properties) of certain substances (e.g., biological cells and tissues (e.g., blood)). In some embodiments, the device and methods provided for modeling and predicting material properties are useful for evaluating, assessing, monitoring, and/or predicting disease status, disease prognosis, treatment course (e.g., therapeutic selection, dosing schedules, administration routes, etc.), response to treatment and/or treatment efficacy.

According to some aspects, any of the methods or devices provided herein can be used to assess the health of any of the subjects described herein, used to detect or determine the stage of any of the diseases or conditions described herein and can be used for determining the level of infectivity of cells as well as the number of diseased versus healthy cells.

Moreover, this invention relates to a method for characterizing deformability of one or more deformable objects, including: (a) perfusing a fluid containing one or more deformable objects through a microfluidic channel that includes a plurality of constrictions arranged in series such that a flow path through each constriction of the plurality is longitudinally aligned with a flow path through each other constriction of the plurality, such that the one or more deformable objects enters or passes through one or more constrictions of the plurality, the one or more deformable objects deforming as it enters or passes through a constriction; and (b) determining a transit characteristic as described herein of one or more deformable objects from a first position within the microfluidic channel that is upstream of a constriction to a second position within the microfluidic channel that is downstream of a constriction. Step (b) can be performed by acquiring a first photomicrographic image of the one or more deformable objects at the first position and acquiring a second photomicrographic image of the one or more deformable objects at the second position, and determining the duration between acquisition of the first photomicrographic image and acquisition of the second photomicrographic image, wherein the duration is the travel time.

Alternatively, a method for characterizing the deformability of a deformable object can be performed by (a) perfusing a first fluid comprising a first deformable object through a first microfluidic channel that comprises a first constriction, such that the first deformable object passes through the first constriction, the first deformable object deforming as it passes through the first constriction; (b) determining a first travel time of the first deformable object from a position within the first microfluidic channel that is upstream of the constriction to a position within the first microfluidic channel that is downstream of the first constriction; (c) perfusing a second fluid comprising a second deformable object through a second microfluidic channel that comprises a second constriction that is geometrically different from the first constriction, such that the second deformable object passes through the second constriction, the second deformable object deforming as it passes through the second constriction; and (d) determining a second travel time of the second deformable object from a position within the second microfluidic channel that is upstream of the second constriction to a position within the second microfluidic channel that is downstream of the second constriction. The first travel time and the second travel time together define a signature that characterizes the deformability of the deformable object.

In one example, the first travel time is determined under a first test condition (e.g., the first fluid being at a first predetermined temperature, perfused under a first pressure gradient, or containing a test agent) and the second travel time is determined under a second test condition (e.g., the second fluid being at a second predetermined temperature, perfused under a second pressure gradient, or not containing the test agent), which is different from the first test condition.

Also disclosed herein is a method for detecting a condition or disease in a subject, the method including (a) obtaining a test agent that is a cell (e.g., hematopoietic cell such as hematopoietic stem cell, leukocyte, red blood cell or reticulocyte, stem cell, or plasma cell), vesicle, biomolecular aggregate or platelet from the subject, the deformability of the test agent being indicative of the presence of the condition or disease; (b) perfusing a fluid containing the test agent through a microfluidic channel that comprises a constriction, such that the test agent passes through the constriction, the test agent deforming as it passes through the constriction; (c) determining a transit characteristic of the test agent as it moves through the microfluidic channel; (d) comparing the transit characteristic to an appropriate standard, the results of the comparison being indicative of whether the subject has the condition or disease; and optionally, (e) diagnosing the subject as having the condition or disease based on the results in (d). The appropriate standard can be a transit characteristic of a cell, vesicle, biomolecular aggregate or platelet obtained from a subject who is identified as not having the condition or disease or a transit characteristic of a cell, vesicle, biomolecular aggregate or platelet obtained from a subject who is identified as having the condition or disease.

The condition or disease to be detected can be a fetal cell condition, fetal chromosomal abnormality, HPV infection, or a hematological disorder, such as hematological cancer, anemia, infectious mononucleosis, HIV, malaria, leishmaniasis, sickle cell disease, babesiosis, spherocytosis, monoclonal gammopathy of undetermined significance or multiple myeloma. Examples of hematological cancer include, but are not limited to, Hodgkin's disease, Non-Hodgkin's lymphoma, Burkitt's lymphoma, anaplastic large cell lymphoma, splenic marginal zone lymphoma, hepatosplenic T-cell lymphoma, angioimmunoblastic T-cell lymphoma (AILT), multiple myeloma, Waldenström macroglobulinemia, plasmacytoma, acute lymphocytic leukemia (ALL), chronic lymphocytic leukemia (CLL), B cell CLL, acute myelogenous leukemia (AML), chronic myelogenous leukemia (CML), T-cell prolymphocytic leukemia (T-PLL), B-cell prolymphocytic leukemia (B-PLL), chronic neutrophilic leukemia (CNL), hairy cell leukemia (HCL), T-cell large granular lymphocyte leukemia (T-LGL) and aggressive NK-cell leukemia.

The following methods are also within the scope of this invention:

A method for characterizing the status of a fetus in a subject, the method including (a) separating a fetal cell or other deformable object from maternal cells or other deformable objects, the difference in deformability between the fetal cell or other deformable object from that of the mother being indicative of whether or not a cell or other deformable object is that of a fetus; and (b) performing a test on the fetal cell or other deformable object to determine the status of the fetus. In one embodiment, step (a) comprises (i) perfusing a fluid containing, or suspected of containing, a fetal cell or other deformable object, through a microfluidic channel that comprises a constriction, such that if present the fetal cell or other deformable object passes through the constriction and deforms as it passes through the constriction; (ii) determining a transit characteristic of a fetal cell or other deformable object, or one suspected of being a fetal cell or other deformable object, as it moves through the microfluidic channel; and (iii) comparing the transit characteristic to an appropriate standard, the results of the comparison being indicative of whether or not the cell or other deformable object is of the fetus. The appropriate standard can be a transit characteristic of a cell, vesicle, biomolecular aggregate or platelet obtained from a subject who has a fetus having a known status. In some embodiments, the status of the fetus is health, age, gender, presence or absence of a chromosomal abnormality, presence or absence of a genetic abnormality, etc. In some embodiments, the other deformable object is a vesicle, biomolecular aggregate or platelet obtained from maternal blood. In other embodiments, the cell or other deformable object is in maternal blood and the blood is perfused through the microfluidic channel.

A method for characterizing an immune cell or platelet, the method including (a) perfusing a fluid containing the immune cell or platelet through a microfluidic channel that comprises a constriction, such that the immune cell or platelet passes through the constriction, and such that the immune cell or platelet deforms as it passes through the constriction; (b) determining a transit characteristic of the immune cell or platelet as it moves through the microfluidic channel; and (c) comparing the transit characteristic to an appropriate standard, the results of the comparison being indicative of a characteristic of an immune cell or platelet. In one embodiment, the immune cell is a T cell or a B cell. The appropriate standard may be a transit characteristic of an immune cell or platelet having a known activation state. The appropriate standard may be a transit characteristic of an activated immune cell or platelet. The appropriate standard may be a transit characteristic of an immune cell or platelet that is not activated. In some embodiments, the platelet is obtained from a subject having, or suspected of having a platelet disorder, such as, for example, Bernard-Soulier syndrome, Glanzmann's thrombasthenia, Scott's syndrome, von Willebrand disease, Hermansky-Pudlak Syndrome, and Gray platelet syndrome.

A method for monitoring the effectiveness of a therapeutic agent for treating a condition or disease in a subject, including: (a) obtaining a test agent as described herein, the deformability of the test agent being indicative of the presence of the condition or disease; (b) perfusing a fluid comprising the test agent through a microfluidic channel that comprises a constriction, such that the test agent passes through the constriction; and (c) determining a transit characteristic of the test agent cell from a position within the microfluidic channel that is upstream of the constriction to a position within the microfluidic channel that is downstream of the constriction; (d) treating the subject with the therapeutic agent; and (e) repeating steps (a) through (c). A difference in the transit characteristic of the test agent determined prior to the treatment compared with the transit characteristic of the test agent determined after the treatment is indicative of the effectiveness of the therapeutic agent.

A method for identifying a candidate therapeutic agent for treating a condition or disease in a subject, including: (a) contacting a test agent as described herein with the candidate therapeutic agent, the deformability of the test agent being indicative of the condition or disease; (b) perfusing a fluid containing the test agent through a microfluidic channel that includes a constriction, such that the test agent passes through the constriction; (c) determining a transit characteristic of the test agent from a position within the microfluidic channel that is upstream of the constriction to a position within the microfluidic channel that is downstream of the constriction; and (d) comparing the transit characteristic to an appropriate standard as described herein. In some embodiments, the results of the comparison are indicative of whether the candidate therapeutic agent is useful for treating the condition or disease in the subject.

A method for detecting a condition or disease in a subject, the method including: (a) obtaining a sample from the subject, the sample including a deformable object having a mechanical property that is indicative of the presence of the condition or disease, e.g., stiffness, deformability, viscoelasticity, viscosity, adhesiveness, or a combination thereof; (b) analyzing the mechanical property using a non-microfluidic channel device, and (c) comparing the mechanical property to an appropriate standard. The results of the comparison are indicative of whether the subject has the condition or disease. Step (b) can be performed by determining a value for at least one mechanical property of the one or more deformable objects. The non-microfluidic channel device used in this step can be AFM, optical tweezers, micropipette, magnetic twisting cytometer, cytoindenter, microindenter, nanoindenter, microplate stretcher, microfabricated post array detector, micropipette aspirator, substrate stretcher, shear flow detector, diffraction phase microscope, or tomographic phase microscope.

A method including at least the steps of (a) perfusing a fluid (e.g., blood, urine, synovial fluid, or cerebrospinal fluid) comprising more than one type of deformable object through a flow test device, and (b) separating one type of deformable object from another type of deformable object based on the deformability of the deformable objects through the device. In one embodiment, the method further includes (c) collecting or removing one type of deformable object from the fluid. In another embodiment, a method including at least the steps of (a) perfusing a fluid (e.g., blood) comprising one or more red blood cells through a flow test device, and (b) separating the reticulocytes from mature red blood cells. In another embodiment, the method further comprises (c) collecting or removing the reticulocytes from the fluid.

A method including (a) perfusing a fluid as described herein comprising one or more red blood cells through a flow test device, (b) separating the reticulocytes from mature red blood cells, and (c) making a determination based on the results of the separation.

A method including (a) perfusing a fluid as described herein comprising cells or platelets through a flow test device, (b) separating a first type of cell (e.g., reticulotytes or white blood cells such as T or B cells) or platelets from another component of the fluid (e.g., mature red blood cells or non-red blood cells) based on a mechanical or rheological property, wherein the mechanical property is stiffness, deformability, viscoelasticity, viscosity and/or adhesiveness, and (c) collecting or removing the first type of cell or platelets from the fluid. The fluid can be obtained from a subject. In one embodiment the fluid comprises more than one type of deformable object. Either the first type of cell or platelets or the other component(s) collected can be returned to the same subject or administered to a different subject.

A method including perfusing a fluid comprising one or more red blood cells through a flow test device, and collecting or removing elite red blood cells from the fluid, as well as a composition containing the elite blood cells thus prepared.

A method including analyzing the deformability of one or more red blood cells from a subject, and determining the fitness of the subject.

A method for isolating a target cell (e.g., stem cell or fetal cell) from a fluid (e.g., a maternal blood sample), including perfusing a fluid having multiple cell types including the target cell through a microfluidic device; and separating the target cell from other cell types in the fluid based on the deformability of the cells.

A method for detecting a condition or disease (e.g., abnormal fetal condition or diabetes) in a subject, including at least the following steps: (a) obtaining a maternal blood sample from the subject, the sample containing a deformable object (e.g., a cell such as a fetal cell) having a mechanical property as described herein, which is indicative of the presence of a fetal cell associated with an abnormal fetal condition; (b) analyzing the mechanical property using a device; and (c) comparing the mechanical property to an appropriate standard. The results of the comparison are indicative of the condition/disease. In one example, the device is not a microfluidic channel.

A method of detecting drug use in a subject, including: (a) perfusing a fluid from the subject comprising a deformable object through a microfluidic device; (b) analyzing the transit of the deformable object through one or more constrictions of a microfluidic channel of the device; and (c) comparing the transit to an appropriate standard. The results of the comparison are indicative of whether the fluid is from a subject who has used a drug.

A method of detecting drug usage of a subject using a non-microfluidic channel device, including: AFM, optical tweezers, micropipette, magnetic twisting cytometer, cytoindenter, microindenter, nanoindenter, microplate stretcher, microfabricated post array detector, micropipette aspirator, substrate stretcher, shear flow detector, diffraction phase microscope, or tomographic phase microscope. Such a non-microfluidic channel device can be used to probe at least one mechanical or rheological property of a cell or other deformable object from a subject who has used a drug. In one embodiment, when the device is a non-microfluidic channel device the device is used to probe the cell or other deformable object once or multiple times in succession.

Any of the steps for assessing of a material property (e.g., the deformability) of a deformable object with a fluid test device or non-microfluidic device as provided herein can be used in any of the various methods of collecting, separating, testing, analyzing, detecting, diagnosing, etc. provided herein.

One aspect of the present invention features a device including a structure (e.g., two-dimensional or three-dimensional) defining one or more microfluidic channels. When the structure defines two or more microfluidic channels, each of the channels is at least partially fluidically isolated from the other(s).

Each of the microfluidic channels contains one or more of constrictions (e.g., two or three-dimensional), each including an inlet orifice and an outlet orifice. The inlet orifice of at least one of the constrictions is geometrically different from the outlet orifice of the same constriction. At least one inlet orifice or at least one outlet orifice can have a polygonal (e.g., triangular), curvilinear or circular shape. In one example, the shape of the at least one inlet/outlet orifice is two-dimensional. In another example, it is three-dimensional. In either case, one or more dimensions of the at least one inlet orifice is less than, greater than, or equal to a dimension of a deformable object. In some embodiments, the cross-sectional area of the at least one inlet orifice is less than, greater than, or equal to any select cross-sectional area of a deformable object.

The inlet orifice(s) in one or more of the constrictions can have a larger cross-sectional area than the outlet orifice(s) in the same constriction(s), e.g., 19 μm² to 23 μm² versus 10 μm² to 15 μm². Alternatively, the inlet orifice(s) has a smaller cross-sectional area than the outlet orifice(s) in the same constriction, e.g., 10 μm² to 15 μm² versus 19 μm² to 23 μm². The one or more constrictions can have a length in a range of 5 μm to 50 μm (e.g., 5 μm to 15 μm).

In one example, the one or more microfluidic channels in the device described herein each contain two constrictions: (a) a first constriction having a first inlet orifice and a first outlet orifice, and (b) a second constriction having a second inlet orifice and a second outlet orifice. (a) and (b) can be arranged in parallel such that a flow path through (a) is parallel with a flow path through (b). The first inlet orifice and the first outlet orifice can be geometrically equal to the second inlet orifice and the second outlet orifice, respectively. In another example, the one or more microfluidic channels in the device each contain a plurality of constrictions arranged in series, each constriction of the plurality being a non-uniform conduit.

The constrictions can be arranged in series such that a flow path through each of the constrictions is aligned, longitudinally or non-longitudinally, with a flow path through each other constriction(s). At least one of the constrictions is a convergent conduit or a divergent conduit. If desired, the constrictions can include both convergent and divergent conduits. When the device containing at least two microfluidic channels, the constrictions in one of the channels can be arranged in parallel with those in each other channel(s) such that a flow path through the former is parallel with a flow path through the latter.

The one or more microfluidic channels in the device described herein, when each containing at least two constrictions, can further contain a gap region between each successive constriction. In one example, this gap region is of a length that allows one or more deformable objects (e.g., cells, vesicles, biomolecular aggregates, platelets, or particles) to recover, at least partially, their shape after passing through the first constriction (e.g., equal to the length of one of the constrictions and/or the length of its successive constriction). In another example, the gap region is of a length that does not allow one or more deformable objects to recover their shape after passing through each constriction.

The one or more microfluidic channels can further contain a substantially planar transparent wall that defines a surface of at least one of the constrictions. This substantially planar transparent wall, which can be glass or plastic, permits observation into the microfluidic channel by microscopy so that at least one measurement of each deformable object that passes through one of the microfluidic channels can be obtained. Preferably, it contains binding agents. In one example, this wall has a thickness of 0.05 mm to 0.1 mm. The microfluidic channel(s) can have a height in a range of 1 μm to 10 μm (e.g., 3 μm to 5 μm or 0.5 μm to 3 μm).

The device described herein can further contain a reservoir fluidically connected with the one or more microfluidic channels, and a pump that perfuses fluid from the reservoir through the one or more microfluidic channels, and optionally, a microscope arranged to permit observation within the one or more microfluidic channels. The reservoir contains deformable objects suspended in a fluid. Preferably, the deformable objects are 0.1-100 μm in diameter (e.g., 1-30 μm, 1-20 μm, 1-10 μm, 2-5 μm, 7-15 μm, 5-20 μm, 10-30 μm, or 15-25 μm in diameter). It can further contain a filter.

In one example, the deformable objects are cells, e.g., red blood cells, white blood cells, stem cells, cancer cells, epithelial cells (e.g., epithelial cells of the cervix, pancreas, breast or bladder), B cells, T cells, or plasma cells. The red blood cells can be fetal red blood cells, red blood cells infected with a parasite, red blood cells from an athlete, or a subject having or is suspected of having a disease (e.g., diabetes, infection with a virus such as HIV, anemia, a hematological cancer such as leukemia, a spleen disease, multiple myeloma, monoclonal gammopathy of undetermined significance, sickle cell disease, or spherocytosis).

Alternatively or additionally, the device described herein further contains a heating or heat transfer element, which can maintain the fluid at a predetermined temperature (e.g., a physiologically relevant temperature such as 30° C. to 45° C., preferably 37° C., 40° C. or 41° C.).

Another aspect of the invention features a method including (a) perfusing a fluid containing one or more deformable objects through any of the devices described herein; and (b) analyzing the transit of the one or more deformable objects through the device. This method can further include (c) comparing the transit characteristic to an appropriate standard. The results obtained from step (c) are indicative of whether a subject, from whom the first fluid is obtained, has a disease or condition, and/or the stage of the disease or condition in the subject. The appropriate standard can be the transit characteristic of one or more deformable objects obtained from a subject who is identified as not having the disease or condition (e.g., compromised hemostasis). Alternatively, it can be the transit characteristic of one or more deformable objects obtained from a subject who is identified as having the disease or condition and/or having the disease or condition at a particular stage.

In some embodiments, the methods disclosed herein comprise evaluating a material property of the deformable object using a non-microfluidic device. In some embodiments, the non-microfluidic device is AFM, optical tweezers, micropipette, magnetic twisting cytometer, cytoindenter, microindenter, nanoindenter, microplate stretcher, microfabricated post array detector, micropipette aspirator, substrate stretcher, shear flow detector, diffraction phase microscope, or tomographic phase microscope or as otherwise provided herein.

In one example, step (b) mentioned herein is performed by determining a transit characteristic of one or more deformable objects through one or more constrictions of a microfluidic channel or through a device. The transit through each constriction, which characterizes one or more material properties (e.g., one or more mechanical properties) of the deformable object (e.g., stiffness, deformability, viscoelasticity, viscosity, or adhesiveness), can be assessed based on a measurement taken at a first position upstream of one of the constrictions and a measurement taken at a second position that is downstream of the same constriction. Alternatively, it can be assessed from a measurement taken between two constrictions. In another example, step (b) is performed by determining the pressure needed for one or more deformable objects to travel a certain distance or by a certain time through one or more constrictions of the microfluidic channel or through the device. In yet another example, this step is performed by determining the distance traveled by one or more deformable objects and/or the time to travel a certain distance through one or more constrictions of the microfluidic channel or through the device at a certain pressure.

In still another aspect, the invention features a method including perfusing a fluid comprising one or more deformable objects through any of the devices described herein; and (b) collecting the deformable objects that flow through the device at a predetermined time or at a predetermined velocity.

In yet another aspect, the invention features a method for monitoring the effectiveness of a therapeutic agent for treating a disease or condition in a subject. This method includes (a) perfusing a fluid comprising one or more deformable objects from the subject through any of the devices described herein, (b) determining a transit characteristic of the one or more deformable objects through the device; (c) treating the subject with the therapeutic agent; and (d) repeating steps (a) and (b). A difference in the transit characteristic of the one or more deformable objects is indicative of the effectiveness of the therapeutic agent.

The present invention also features a method for identifying a candidate therapeutic agent for a treating a disease or condition in a subject, including (a) perfusing a fluid comprising one or more deformable objects that has been or is contacted with the candidate therapeutic agent through any of the devices described herein, (b) determining a transit characteristic of the one or more deformable objects through the device; and (c) comparing the transit characteristic to an appropriate standard. The results of the comparison are indicative of whether the candidate therapeutic agent is useful for treating the disease or condition in the subject. In this method, the appropriate standard can be the transit characteristic of one or more deformable objects obtained from a subject who is identified as not having the disease or condition. Alternatively, it can be the transit characteristic of one or more deformable objects that exhibit at least one certain material (e.g., mechanical) property.

In addition, the invention features a method including (a) perfusing a fluid comprising one or more red blood cells from a subject through any of the devices described herein, and (b) separating one or more types of red blood cells from the fluid.

In any of the methods described herein, the fluid can be perfused, for example, through one or more microfluidic channels in a device of this invention at a predetermined pressure gradient, e.g., ranging from about 0.20 Pa/μm to about 0.40 Pa/μm. Alternatively or additionally, the fluid is perfused at a predetermined temperature, e.g., a physiologically relevant temperature.

Also within the scope of this invention is a method for characterizing the deformability of one or more deformable objects. This method includes: (a) perfusing a fluid comprising one or more deformable objects through a microfluidic channel that comprises a constriction, such that the one or more deformable objects passes through the constriction, the one or more deformable objects deforming as it enters or passes through the constriction; and (b) determining a transit characteristic of the one or more deformable objects from a first position within the microfluidic channel that is upstream of the constriction to a second position within the microfluidic channel that is downstream of the constriction. The transit characteristic, characterizing the deformability of the one or more deformable objects, can be travel distance, travel time (e.g., the time to travel a certain distance or the time traveled at a certain pressure), velocity, or a combination thereof.

The microfluidic channel used in any of the methods described herein can be a channel within a three-dimensional network of channels or within a two-dimensional network of channels.

The constriction(s) in the microfluidic channel can define a non-uniform conduit, which can be either a convergent conduit or a divergent conduit. The non-uniform conduit contains an inlet orifice having an area in a range of 19 μm² to 23 μm² and an outlet orifice having an area in a range of 10 μm² to 15 μm². Alternatively, it contains an inlet orifice having an area in a range of 10 μm² to 15 μm² and an outlet orifice having an area in a range of 19 μm² to 23 μm². Either the inlet orifice or the outlet orifice can have a polygonal, curvilinear or circular shape. Preferably, the constriction has a conduit length in a range of 5 μm to 50 μm (e.g., 5 μm to 15 μm) or a height in a range of 1 μm to 10 μm (e.g., 3 μm to 5 μm or 0.5 μm to 3 μm).

In one aspect, the present invention features a method including at least two steps: (a) obtaining data from at least one flow test performed on a fluid that contains more than one type of deformable object, and (b) comparing the data with one or more predicted values calculated with at least one closed-form equation that correlates flow behavior to at least one material property (e.g., mechanical or rheological property (e.g., velocity, shear modulus, shear rate, shear stress, strain rate, yield stress, or hematocrit)). Optionally, this method further includes one or more of step (c), i.e., calculating the predicted values with the at least one closed-form equation, step (d), i.e., assessing the health of a subject from which the fluid is derived, and step (e), i.e., sorting and/or collecting one type of deformable object from another based on the comparison.

In step (a), the at least one flow test performed on a fluid can be carried out at a specific pressure gradient (or pressure differential). In one example, the flow test is performed by passing the fluid through one or more microfluidic channels, which can contain one or more constrictions or form part of a microfluidic device (e.g., any of the microfluidic devices described in this application). In another example, the flow test is performed by passing the fluid through a microbead suspension, a flow cytometer, or a suspended microchannel resonator.

The fluid can contain more than one type of cell (e.g., a mixture of both healthy and diseased cells), vesicle, biomolecular aggregate, platelet or particle, or a combination thereof. In one example, it contains red blood cells, white blood cells, epithelial cells, or a mixture thereof. In another example, it contains cancer cells. In yet another example, the fluid (e.g., whole blood) contains T cells, B cells, platelets, reticulocytes, mature red blood cells, or a combination thereof.

Epithelial cells can be those of the cervix, pancreas, breast or bladder. Red blood cells can be fetal red blood cells, red blood cells infected with a parasite, red blood cells from a subject having or is suspected of having a disease, such as diabetes, HIV infection, anemia, cancer (e.g., a hematological cancer such as leukemia), multiple myeloma, monoclonal gammopathy of undetermined significance, or a disease that affects the spleen.

The data obtained in this step can include a value for a transit characteristic, e.g., the velocity for one of the deformable objects or the average velocity for a population of the deformable objects, the distance traveled by one of the deformable objects, the time for one of the deformable objects to travel a certain distance, the average distance traveled by a population of the deformable objects, or the average time for a population of the deformable objects to travel a certain distance.

Step (b) can be performed with at least one processor. The at least one closed-form equation employed in this step can be developed from one or more simulations of flow of a fluid in combination with experimental data. The one or more stimulations can be performed using dissipative particle dynamics model or a stochastic bond formation/dissociation model. The experimental data preferably is from an assay that measures membrane shear modulus, membrane bending rigidity, membrane viscosity, interior/exterior fluid viscosities, or a combination thereof, on a deformable object.

Step (d) can be performed by determining the presence or absence of a disease or condition in the subject or determining the stage of a disease or condition.

In another aspect, the invention features a method including (a) obtaining data for one or more material properties (e.g., mechanical properties) of a deformable object, and (b) determining one or more predicted values of flow behavior. The one or more predicted values are determined with at least one closed-form equation as described herein that correlates flow behavior of any of the fluids described herein or elsewhere in this application to the one or more properties.

In still another aspect, this invention features an apparatus for performing at least one of the methods described herein. This apparatus contains (i) a device for performing a flow test on a fluid, both being described herein or elsewhere in this application, (ii) a computer system for obtaining data from the flow test and comparing the data with one or more predicted values also described herein. Alternatively, this apparatus contains (i) a device for obtaining data for one or more material properties (e.g., mechanical properties) of a deformable object, and (ii) a computer system for obtaining the data and determining one or more predicted values. The predicted value(s) can be calculated with at least one closed-form equation that correlates flow behavior of the deformable object-containing fluid described herein to the one or more material properties (e.g., mechanical properties).

Also within the scope of this invention is a method for manufacturing a diagnostic test apparatus that contains (i) a device either for performing a flow test or for determining one or more material properties (e.g., mechanical properties) of a deformable object; and (ii) a computing device that predicts at least one material property (e.g., mechanical or rheological property) of a sample (e.g., any of the deformable object-containing fluids described herein) based on flow behavior measured on the sample passing through the device, compares a value for a measurement of a sample as it passes through the device, or calculates one or more predicted values for flow behavior of the fluid described herein.

In one example, this method includes (a) generating, with at least one processor and a model of deformable objects within a fluid, a closed-form equation relating at least one parameter of flow of the fluid through the device to at least one material property (e.g., mechanical or rheological property); and (b) encoding the closed-form equation in software configured for execution on the computing device.

In another example, this method includes (a) comparing, with at least one processor, the value with one or more predicted values calculated with a closed-form equation relating at least one parameter of flow of the fluid to at least one material property (e.g., mechanical or rheological property); and (b) encoding the one or more predicted values in software configured for execution on the computing device.

In yet another example, the manufacturing method includes (a) calculating, with at least one processor, one or more predicted values with the one or more material properties (e.g., mechanical properties), the one or more predicted values being calculated with a closed-form equation relating at least one parameter of flow of the fluid to the one or more properties; and (ii) encoding the one or more predicted values in software configured for execution on the computing device.

In addition, the present invention features a method including an inputting step and a calculating or comparing step. The inputting step can be performed by inputting a value for a measurement of any of the deformable object-containing fluids described herein as it passes through a flow test device. Alternatively, it is performed by inputting a value for one or more mechanical properties of a deformable object. The calculating step can be performed by calculating at least one material property (e.g., mechanical or rheological property) with a closed-form equation and the inputted value, the equation relating at least one parameter of flow of the fluid through the device to the at least one material property (e.g., mechanical or rheological property), or by calculating one or more predicted values for flow behavior of any of the fluids described herein, the one or more predicted values being calculated with a closed-form equation relating at least one parameter of flow of the fluid the one or more properties. When the just-described method includes a comparing step, it is performed by comparing the value with a predicted value from a calculation with at least one closed-form equation that correlates flow behavior to at least one material property (e.g., mechanical or rheological property). Any of the methods described in this paragraph can further include step (c), i.e., calculating the predicted value with the closed-form equation.

Moreover, this invention features at least one non-transitory computer-readable storage medium encoded with computer-executable instructions that, when executed by a processor, perform one of the methods described in the preceding paragraph.

In yet another aspect, this invention relates to a method including: (a) obtaining a value for one or more material properties (e.g., mechanical or rheological properties) of a deformable object, (b) determining a material (mechanical or rheological property (e.g., velocity)) of the fluid described herein comprising the deformable object using a closed-form equation that correlates the properties, and optionally, (c) making a prediction about the health of a subject (e.g., a subject having malaria or diabetes) based on the determination. The one or more properties (e.g., mechanical) can be measured by, e.g., AFM, optical tweezers, micropipette, magnetic twisting cytometer, cytoindenter, microindenter, nanoindenter, microplate stretcher, microfabricated post array detector, micropipette aspirator, substrate stretcher, shear flow detector, diffraction phase microscope, or tomographic phase microscope. The prediction can include an assessment of the aggregation of the deformable objects in the fluid.

Still, this invention relates to a method including performing one or more assays on one or more deformable objects to obtain a measurement of one or more material properties; simulating, with at least one processor, flow of a fluid comprising more than one type of deformable object; and obtaining a closed-form equation with data from the simulation in combination with the measurement.

In one aspect, the present invention features a method including at least two steps: (i) analyzing the deformability or elasticity of a T cell, and (ii) making a determination about the state of the T cell based on the analysis. In one embodiment, the state of the T cells is its activation state, a function or a disease state, examples of which include the stimulation of the T cells by a chemokine or chemotaxis.

In another aspect, the invention features a method including at least: (a) determining the deformability or elasticity of a T cell, (b) contacting the T cell with a compound, and (c) analyzing the deformability or elasticity of the T cell after (b). In some embodiments, the compound is a chemokine.

In yet another aspect, the invention features a method including a step of contacting a T cell with a compound that affects the deformability of the T cell. Examples of the compound include, but are not limited to, small molecules and proteins. In one embodiment, the small molecule is a cytochalasin, latrunculin A and B, nocodazole, colchicine, vincristine, colcemid, or paclitaxel. In another embodiment, the small molecule affects (stimulates or inhibits) T cell function, either through inhibition of kinases and phosphatases, such as SB203580 (inhibitor of p38 kinase), SP600125 (inhibitor of JNK), U0126 (inhibitor of ERK), cyclosporin A and FK506 (calcineurin), or through inhibition of transcription factors. In another embodiment, the protein is a cytokine, growth factor or antibody. In yet another embodiment, the cytokine is IL-2, -4, -7, -15, or -21. In still another embodiment, the antibody is specific for a T-cell surface protein. In one embodiment, the T-cell surface protein is CD3, CTLA4, CD28 or IL-7R.

In yet another aspect, the invention features a method including a step of contacting a T cell with a compound that affects the elasticity of the T cell. In one embodiment, the compound is a chemokine.

The contacting step can be performed by administering the compound to a subject, e.g., a subject in need of an improved or inhibited T cell response. In one example, this subject has or is suspected to have a disease or condition against which an improved or inhibited T cell response is beneficial. In one embodiment, the subject has cancer, an infection or an infectious disease.

In yet another aspect, the invention features a method including a step of contacting a T cell with a compound that affects the deformability or elasticity of the T cell, by administering the compound to a subject in need of a reduced T cell response. In one example, this subject has or is suspected to have a disease or condition for which a reduced or inhibited T cell response is beneficial. In one embodiment, the subject has cancer, an autoimmune disease, an infection or an infectious disease.

Also within the scope of this invention are (a) a pharmaceutical composition for use in eliciting or inhibiting a T cell response, the composition containing a compound that affects the deformability or elasticity of a T cell, and (b) the use of the just-described pharmaceutical composition in manufacturing a medicament for eliciting or inhibiting a T cell response.

One aspect of the present invention features a method including: (i) attaching a first type of cell (or vesicle or platelet) to a first surface by, e.g., growing the first type of cell (or vesicle or platelet) on the first surface, (ii) attaching a second type of cell (or vesicle or platelet) to a first surface and then attaching the second type of cell (or vesicle or platelet) to a second surface by, e.g., initially stabilizing the second type of cell (or vesicle or platelet) through light adhesion to the first surface and subsequently transferring it to the second surface through mediation with a stronger adhesive molecule, (iii) contacting the two types of cells (or vesicles or platelets) and then separating the second type of cell (or vesicle or platelet) from the first type of cell (or vesicle or platelet), and (iv) determining the adhesion force between the first type of cell (or vesicle or platelet) and the second type of cell (or vesicle or platelet) with atomic force microscopy (AFM). In step (iv), the force of binding satisfies the following relationship:

f_{A2}>f_{A1},f_{A3},

in which f_{A1} is the force of binding of the second type of cell (or vesicle or platelet) to the first surface, f_{A2} is the force of binding of the second type of cell (or vesicle or platelet) to the second surface, and f_{A3} is the force of binding of the second type of cell (or vesicle or platelet) to the first type of cell (or vesicle or platelet). In one embodiment, the second surface is a surface of a tipless cantilever. When necessary, the tipless cantilever is functionalized with a molecule that binds the second type of cell (or vesicle or platelet). In one embodiment, the first surface to which the first type of cell (or vesicle or platelet) is attached and the first surface to which the second type of cell (or vesicle or platelet) is attached is the same. In another embodiment, they are different.

The first type of cell can be a cell (or vesicle or platelet), e.g., a CHO cell, that expresses a receptor. The second type of cell (or vesicle or platelet) can express a ligand that binds to the first type of cell (or vesicle or platelet) via, e.g., interaction with the receptor expressed thereon. In one example, the second type of cell (or vesicle or platelet) is infected or is thought to be infected with, e.g., a microbe or parasite. In another example, it is diseased or is thought to be diseased, e.g., a cancer cell. In yet another example, the second type of cell (or vesicle or platelet) is a blood cell or the like, such as a red blood cell, T cell (activated or inactivated), or a B cell.

In the method described above, steps (ii) and (iii) can be performed repeatedly and step (iv) is based on the results of the repeated steps. In one example, this method further includes a step of assessing the health of a subject or selecting a therapeutic agent based on the determination of the adhesion force. In another example, the method further includes treating the first type of cell (or vesicle or platelet) or the second type of cell (or vesicle or platelet) with a candidate therapeutic agent. If desired, this method can further include, after the treating step, contacting the first type of cell (or vesicle or platelet) and the second type of cell (or vesicle or platelet), subsequently separating the two types of cells (or vesicles or platelets), determining the adhesion force between the first type of cell (or vesicle or platelet) and the second type of cell (or vesicle or platelet), and optionally, comparing the adhesion force before and after treatment with the candidate therapeutic agent.

Another aspect of the present invention features a method of detecting a diseased cell, which can be a blood cell or the like as described above. This method includes at least the following steps: (a) determining the force of adhesion between a cell or the like that is or is suspected to be diseased (e.g., being infected or suspected to be infected with a microbe or parasite) and another cell or the like, and (b) assessing whether or not the cell or the like is diseased by comparing the force of adhesion with an appropriate standard, which can either be the force of adhesion of a healthy cell or the like with the other cell or the like or the force of adhesion of a diseased cell or the like with the other cell or the like. The force of adhesion between the cell or the like that is or is suspected to be diseased and the other cell or the like is determined with an assay (e.g., AFM) such that the relationship is satisfied:

f_{A2}>f_{A1},f_{A3},

in which f_{A1} is the force of binding of the cell or the like that is or is suspected to be diseased to a first surface, f_{A2} is the force of binding of the cell or the like that is or is suspected to be diseased to a second surface, and f_{A3} is the force of binding of the of the cell or the like that is or is suspected to be diseased to the other cell or the like. In one embodiment, the first surface to which the first type of cell or the like is attached and the first surface to which the second type of cell or the like is attached are the same. In another embodiment, they are different.

Also within the scope of this invention is a method including at least: (a) determining the force of adhesion between a diseased cell that is or has been contacted with a candidate agent and another cell, and (b) comparing the force of adhesion with an appropriate standard, wherein the appropriate standard is the force of adhesion of either a diseased cell or a healthy cell with the other cell. The force of adhesion between the diseased, candidate agent-treated cell and the other cell is determined with an assay such that the f_{A2}>f_{A1},f_{A3} relationship described above is satisfied.

In any of the methods described above, adhesion force determination can be performed at a physiologically relevant temperature, e.g., 37° C., 40° C. or 41° C.

Any of the methods described herein, if applicable, can be used to assess the health of any of the subjects described in this application, to detect any of the diseased cells also described herein, and to determine the level of infectivity of cells as well as the number of diseased versus healthy cells as described herein.

### BRIEF DESCRIPTION OF DRAWINGS

The patent or application file contains at least one drawing executed in color.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1A illustrates an exemplary microfluidic device design.

FIG. 1B shows exemplary images of ring-stage P. falciparum-infected (dark arrows) and uninfected (light arrows) RBCs in the channels at a pressure gradient of 0.24 Pa/μm. The small fluorescent dot inside the infected cell is the GFP-transfected parasite. At 8.3 s, the uninfected cell moved about twice as far as each infected cell.

FIG. 1C depicts an exemplary computational RBC model that consists of 5000 particles connected with links. The P. falciparum parasite is modeled as a rigid sphere inside the cell.

FIG. 1D depicts exemplary DPD simulation images of P. falciparum-infected RBCs traveling in channels of converging (left) and diverging (right) pore geometry at 0.48 Pa/μm.

FIG. 2A depicts a velocity vs. pressure gradient in converging pore geometry for late ring-stage P. falciparum-infected RBCs. In this experiment, approximately 1,000 RBCs were tracked for each geometry over a distance of 200 μm (10 constrictions). The symbol ** indicates a P-value<0.005 and the symbol * indicates a P-value<0.05. Mean velocities are indicated by horizontal lines. The experiment was run simultaneously with the experiment associated with FIG. 2B.

FIG. 2B depicts a velocity vs. pressure gradient in diverging pore geometry for late ring-stage P. falciparum-infected RBCs. In this experiment, approximately 1,000 RBCs were tracked for at each pressure gradient over a distance of 200 μm (10 constrictions). The symbol ** Indicates a P-value<0.005 and the symbol * indicates a P-value<0.05. Mean velocities are indicated by horizontal lines. The experiment was run simultaneously with the experiment associated with FIG. 2A.

FIG. 2C depicts a FACS-like plot of velocity vs. intensity for ring-stage P. falciparum infected RBCs at a pressure gradient of 0.24 Pa/μm travelling in the converging geometry. Points to the right of the vertical line represent velocities of infected RBCs, while points to the left represent velocities of uninfected RBCs. The velocities of 381 RBCs were tracked.

FIG. 2D depicts a plot of velocity vs. infection state for RBCs infected with late ring-stage parasites at a pressure gradient of 0.24 Pa/μm. For each infected cell that was tracked, the next uninfected cell was tracked. Twenty cells were tracked for each measurement.

FIG. 3A depicts the results of a dissipative particle dynamics (DPD) simulation evaluating the effects of RBC size variation on transit time at a pressure gradient of 0.24 Pa/μm. Cells with surface area of 125, 135 and 145 μm² are modeled with corresponding volumes of 85, 94 and 103 μm³.

FIG. 3B depicts the results of a DPD simulation evaluating the effects of membrane viscosity variation on RBC transit time at a pressure gradient of 0.24 Pa/μm. The membrane viscosity is normalized by the healthy cell membrane viscosity value.

FIG. 3C depicts the results of a DPD simulation evaluating the effects of RBC transit time vs. membrane shear modulus at 0.24 Pa/μm.

FIG. 4A depicts the results of an experiment evaluating the velocity of individual 200-nm-diameter beads at a pressure difference of 0.49 Pa/μm. In this experiment, there is no statistically significant difference in the velocity of beads travelling through the converging and diverging geometries. The beads travelling through the channel with rectangular obstacles moved slower on average in this experiment.

FIG. 4B depicts the results of an experiment evaluating the velocity of individual 200-nm-diameter at different pressure gradients for different obstacle geometries.

FIG. 5A depicts the results of an experiment evaluating the velocity at different pressure gradients for RBCs moving through the two pore geometries. Error bars indicate standard deviation for each measurement.

FIG. 5B depicts the results of an experiment evaluating the velocity at different glutaraldehyde concentrations of RBCs moving through the two pore geometries. RBCs were treated with the indicated concentrations of glutaraldehyde for 30 minutes in PBS and then washed 3 times. The pressure difference/length was approximately 0.61 Pa/μm.

FIG. 6 depicts the results of an experiment evaluating the velocity of RBCs at different cell maturation states for two pore geometries. Experiments were run simultaneously, at a pressure gradient of 0.24 Pa/μm. Whole blood RBCs were stained for nucleic acid content with thiazole orange. Cells homogeneously fluorescesing under the GFP filter set were identified as reticulocytes. For every reticulocyte that was identified and tracked for 200 μm, the next cell appearing in the field of view was also tracked.

FIG. 7A depicts an exemplary relationship between velocity and pressure gradient for healthy and ring-stage-infected RBCs in diverging pore geometry. A comparison of simulation and experimental results are shown. For experimental data, mean values are shown. The error bars correspond to one standard deviation.

FIG. 7B depicts an exemplary relationship between velocity and pressure gradient for healthy and ring-stage-infected RBCs in converging pore geometry. A comparison of simulation and experimental results are shown. For experimental data, mean values are shown. The error bars correspond to one standard deviation.

FIG. 7C depicts the effect of intracellular parasite presence on the velocity of ring-stage infected cells. The parasite is modeled in simulations as a rigid sphere, 2 microns in diameter, placed inside the cell. A comparison of simulation and experimental results are shown. For experimental data, mean values are shown. The error bars correspond to one standard deviation.

FIG. 8 depicts ring-stage malaria-infected cells at different temperatures. The experiment was conducted under constant pressure operation, whereby the same pressure drop was maintained at all conditions.

FIG. 9 depicts ring-stage malaria-infected cells at different temperatures. The experiment was conducted under constant local velocity, whereby pressures were changed to maintain the constant local flow velocity at the device.

FIG. 10 depicts a schematic view of a pressure-control flow system and channels used in flow experiments. A combination of pneumatic regulators and relative height adjustments are used to set the desired pressure difference.

FIG. 11 depicts shape characteristics of RBC traversal across microfluidic channels. FIG. 11A depicts experimental (left) and simulated (right) images of erythrocyte traversal across a 4 μm wide, 30 μm long, 2.7 μm high channel at 22° C. and an applied pressure difference of 0.085 kPa. FIG. 11B depicts local area expansion contours for an RBC traversing a 3 μm and 6 μm. wide (h=2.7 μm) channel under ΔP=0.085 kPa. FIG. 11C depicts measured and simulated cell lengths at the center of the microfluidic channel for varying channel widths. FIG. 11D depicts estimated maximum stretch ratios of RBC spectrin network. FIG. 11E depicts asphericity indices of cells passing through different channel widths under ΔP=0.085 kPa. In FIG. 2D all channel heights are 2.7 μm. In FIG. 11E, channel height and width dimensions are indicated. Vertical dashed lines in FIGS. 11D and 11E indicate locations of channel entrance and exit. Horizontal dashed line in FIG. 11E indicates the stress-free, resting asphericity of a normal RBC (α=0.15).

FIG. 12 depicts quantitive flow behaviors of RBC traversal of microfluidic channels. FIG. 12A depicts a comparison of DPD simulation results (open markers) with experimentally measured mean velocities (filled markers) of RBC traversal as a function of measured local pressure differences for 3, 4, 5 and 6 μm channel widths (height=2.7 μm, length=30 μm). Error bars on experimental data points represent an average +/− one standard deviation of a minimum of 18 cells. Error bars on modeling data points indicate minimum and maximum variations resulting from a case study exploring the sensitivity of the RBC traversal to channel geometry and cell volume. FIG. 12B depicts experimentally measured and modeled total transit time broken into entrance, channel and exit components for RBC traversal across varying channel widths under ΔP=0.085 kPa. (*) Modeling results with 2× domain size to examine the role of fluid inertia and periodic boundary conditions

FIG. 13 depicts temperature dependent RBC flow behaviors. FIG. 13A illustrates a comparison of DPD simulation results with experimentally measured effects of temperature on ratio of local pressure difference and mean velocity of erythrocyte traversal in a 4 μm and 6 μm wide (h=2.7 μm, L=30 μm) microfluidic channel. Data points represent an average of a minimum of 18 cells (all p<0.05 in experimental data) (13). Independent effects of external fluid viscosity, membrane viscosity and internal fluid viscosity on the modeled flow characteristics of RBCs in 4 μm channels subjected to a pressure difference of 0.14 kPa.

FIG. 14 illustrates case studies using the DPD model to evaluate the sensitivity of RBC flow in a 4 μm wide×2.7 μm high channel subjected to a pressure difference of 0.14 kPa with respect to variations in initial RBC position (B: Off-centerline initial position). channel geometry (C: Non-rectangular, beveled corner cross-section with the same cross-sectional area), and cell volume (D,E,F: 0.8, 1.1, and 1.25 times the standard cell volume of 100 μm³, respectively).

FIG. 15 depicts a coarse-grained RBC, represented by a collection of points connected by links. The model takes into account the effects of membrane viscosity, in-plane shear energy, bending energy, constraints of fixed surface area and enclosed volume.

FIG. 16 illustrates a relationship between average velocities of 1 μm diameter beads and local pressure difference at room, body and febrile temperatures (22° C., 37° C., 40° C. and 41° C., respectively) for 2.7 μm high, 30 μm long channels of varying width.

FIG. 17 illustrates a comparison of analytical solutions and CFD results for fluid and bead velocities at various positions along the width of the channel. (Inset: Pressure-velocity relationship for beads and fluid along channel center-line)

FIG. 18 provides an illustrative sketch of microfluidic device. The height of the device is 4 microns.

FIG. 19 depicts the average velocity of healthy and malaria infected RBCs as a function of pressure gradient, comparing simulation with experimental results. Results for converging and diverging geometries are shown on left and right, respectively.

FIG. 20 depicts the effect of variation of RBC properties on the traversal of a cell through the micropores. FIG. 20A depicts the effect of presence of the malaria parasite inside the cell on average traverse velocity as a function of applied pressure gradient. FIG. 20B depicts the effect of RBC size on traversal time at pressure gradient of 0.24 Pa/μm. FIG. 20C depicts the effect of membrane viscosity variation on traversal time with a pressure gradient of 0.24 Pa/μm. FIG. 20D depicts the effect of membrane shear modulus variation on traversal time at pressure gradient of 0.24 Pa/μm.

FIG. 21 depicts RBC velocity with different values of membrane shear modulus as a function of pressure. Symbols—simulation results. Lines—fitting function V.

FIG. 22 illustrates an enrichment of CD8+ T cells after negative selection confirmed by FACS. FIG. 1A and FIG. 1B show results before enrichment, and FIG. 22C and FIG. 22D show results after enrichment.

FIG. 23A depicts a microwell array for confining T cells. Shown in this figure are 16-μm microwells that were used to trap activated Balb/c T cells. The AFM probe (dark triangle) points to a cell that was later indented. FIG. 23B illustrates fitting a Hertz model to the approach curve obtained while indenting a naïve Balb/c T cell. Despite the simplicity of the model, the fit is very good. FIG. 23C illustrates changes in the apparent Young's modulus of T cells with indentation depth. At the beginning of indentation, the modulus fluctuates significantly, but eventually settles such that the modulus stabilizes to a near constant value.

FIG. 24 provides examples of AFM cell indentation of T cells. FIG. 24A illustrates that the apparent Young's modulus increased with indentation speed for both naïve and activated Balb/c T cells. The shape of the curves is similar. FIG. 24B depicts the apparent Young's modulus of naïve WASp cells as it increases with indentation speed. The curve however is shifted to the left—toward low indentation speeds—compared to that of Balb/c T cells.

FIG. 25 illustrates changes in the apparent Young's modulus of Balb/c and WASp T-cells as a result of activation. Student's T test was conducted to determine the significance of the data to a 95% (p=0.05) confidence level.

FIG. 26 provides an exemplary illustration of force spectroscopy experiments. As depicted, a glass slide is provided that is precoated with PDL and that presents a CHO monolayer culture. Erythrocytes are poured onto the slide and allowed to stand and bind lightly to the substrate (step A); a tipless cantilever previously functionalized with ConA is engaged on an iRBC at late trophozoite stage (step B); the iRBC attached to the tipless cantilever is used as a single-cell probe (steps D, E, and F). Setup optimization required regulation of ConA and PDL adhesive strength, so that f_{iRBC/substrate}<f_{iRBC/cantilever}>f_{iRBC/CHO}.

FIG. 27 depicts photomicrographs of cells subjected to a cytoadherence test.

FIG. 28 depicts an illustrative cytoadherence test configuration.

FIG. 29 depicts illustrative cytoadherence results for an RBC isolated from a subject with a normal temperature.

FIG. 30 depicts illustrative cytoadherence results for an RBC isolated from a subject with a normal temperature.

FIG. 31 depicts illustrative cytoadherence results for an RBC isolated from a subject with a febrile temperature.

FIG. 32 depicts illustrative cytoadherence results for an RBC isolated from a subject with a febrile temperature.

FIG. 33 depicts illustrative cytoadherence results for an RBC isolated from a subject with a febrile temperature.

FIG. 34 depicts a table of illustrative results from temperature dependent cytoadherence tests.

FIG. 35 depicts illustrative results from temperature dependent cytoadherence tests.

FIG. 36 depicts illustrative control assays for cytoadherence tests.

FIG. 37. Increased stiffening of Pf-RBCs: Simulated stretching of healthy and Pf-RBCs at different malaria stages compared with optical tweezer experiments [24]. DA and DT refer to the axial and transverse diameters.

FIG. 38. Depicts an analysis of Flow Resistance. Panels A and B: An example of a CFL edge (left) and CFL thickness distribution (right) for Ht=0.45 and D=20 μm. Panel C: Relative apparent viscosity in comparison with experimental data [31] for various Ht values and tube diameters. Inset plot is a snapshot of RBCs in Poiseuille flow in a tube of a diameter D=20 μm at Ht=0.45.

FIG. 39. Flow resistance in malaria: Healthy (red) and Pf-RBCs (blue) in Poiseuille flow in a tube of diameter D=20 μm. Ht=0.45, parasitemia level 25%. Plotted is the relative apparent viscosity of blood in malaria for various parasitemia levels and tube diameters. Symbol “x” corresponds to the schizont stage with a near-spherical shape. Experimental data from the empirical fit by Pries et al. [31].

FIG. 40. Adhesive dynamics. Panel A: Top and side views of successive snapshots of a single flipping of an infected RBC. Panel B: Top and side views of several snapshots of a rolling RBC with a parasite body inside the cell (drawn in green). Panel C: Average rolling velocity of infected RBCs depending on the shear stress compared with the experiments of cell rolling on purified ICAM-1 [8]. Experimental data include mean values and curves that correspond to the 10th, 25th, 75th, and 90th percentiles. Panel D: Velocities of Pf-RBCs with/without parasitic body, and for the case of complete detachment.

FIG. 41. Validation of simulation results for whole blood and Ringer ES. (a) Plot of non-Newtonian viscosity relative to solvent viscosity as a function of shear rate at H=45% and 37° C.: simulated curves of this work, as indicated, and experimental points: Whole blood: greencrosses—Merril et al. (1963); black circles—Chien et al. (1966), black squares—Skalak et al. (1981). Ringer ES: red circles—Chien et al. (1966); red squares—Skalak et al. (1981). (b) Plot of relative viscosity as a function of hematocrit (H) at shear rates 0.052 (black) and 5.2 (red) s-1: simulated (LD-RBC points), and Chien's (1966) experimental fits for whole blood (solid lines) and Ringer ES (dashed lines).

FIG. 42. Visualization of aggregation. Simulated reversible rouleaux are formed by LD-RBC models (upper row) and MS-RBC models (lower row). The left column corresponds to low shear rates, middle column to moderate share rates, and right column to high shear rates as indicated on the plots. See also on-line videos.

FIG. 43. Correlation of aggregation with yield stress. (a) Casson plots showing the extrapolated intercept T_{y} for simulated MS-RBC and LD-RBC suspensions with, dashed lines, and without aggregation, solid lines, at H=45%. (b) Yield stress as a function of hematocrit H for simulated suspensions with aggregation compared with experimental values derived from viscosity measurements: blue stars—Merril et al. (1963); green triangles—Chien et al. (1966); open circles—Picart et al. (1998).

FIG. 44. Non-Newtonian characteristics of human blood. (a) Normal-stress differences N₁=T_{yy}−Tₓₓ and N₂=Tₓₓ−T_{zz} derived from simulations of this work as functions of shear rate. (b) Effect of aggregation on the mean relaxation time

$${λ}_{0} = \frac{{N}_{1}}{2 {τ}_{xy} \dot{γ}} .$$

FIG. 45A depicts a sketch of RBC adhesion with receptors and ligands shown.

FIG. 45B depicts a sketch of a modeled WBC with receptors and ligands shown.

FIG. 46 depicts center-of-mass displacements (x_{c}) and velocities (v_{c}) for various adhesion states of a WBC. A—firm adhesion, B—stop-and-go rolling, C—stable rolling, and D—free motion.

FIG. 47 shows an on-off state diagram of WBC adhesion dynamics states: firm adhesion (squares), stop-and-go rolling (triangles), stable rolling (circles), and free motion (crosses). The letters “A-D” mark simulations shown in FIG. 46. Dashed lines were drawn for the eye to identify regions corresponding to different states.

FIG. 48 shows a contour plot of the on-off diagram of the average WBC velocity (left) and the average pause time (right). Dashed lines indicate regions of different states of leukocyte adhesive dynamics shown in FIG. 48.

FIG. 49 depicts a contour plot of the on-off diagram of the WBC contact area (left) and the deformation index (right). Dashed lines indicate states of leukocyte adhesive dynamics shown in FIG. 47.

FIG. 50 depicts a MS-RBC membrane model.

FIG. 51 depicts aggregation interactions for the MS-RBC model.

FIG. 52 depicts a sketch of the low-dimensional closed-torus like RBC model.

FIG. 53 depicts LD-RBC shape evolution at different Nc (number of particles in LD-RBC model) and stretching forces.

FIG. 54 depicts a schematic of the aggregation algorithm. The two neighbor RBCs (1 and 2) are decided to aggregate or not according to that the angles, θ1 and θ2, are smaller or greater than π/4.

FIG. 55A depicts an exemplary microfluidic device.

FIG. 55B provides experimental images of iRBCs (white arrows) and uRBCs (blue arrows) in 3 μm channels. Driven by a constant pressure gradient of 0.36 Pa/μm, the cell motion was tracked at three different temperatures: 30° C., 37° C., and 40° C. While an uninfected cell appeared as a dark shadow, the GFP-transfected parasite inside an infected cell was observed as a small fluorescent dot. The red and black arrows indicate the distance moved by iRBCs and uRBCs, respectively. With one second time interval, the lengths of the arrows reveal the mobility of cells. The images on the top right corner illustrate how a cell passes through the pores.

FIG. 56A provides a cell mobility vs. temperature plot for infected cells passing though 3 μm channels at a constant pressure gradient of 0.36 Pa/μm.

FIG. 56B depicts results from a fluid velocity calibration experiment. It was noted that the viscosities of buffer solution and red blood cells decrease with increasing temperature resulting in an overall increased local fluid velocity at elevated temperatures. A control experiment was conducted to achieve equalized fluid velocity of 226 μm/s in 4 μm channels at all temperatures. Local fluid velocity is calibrated by 200 nm fluorescent microspheres.

FIG. 56C provides a cell mobility vs. temperature plot for infected cells passing through 4 μm channels. Data were obtained at a constant local fluid velocity of 226 μm/s as calibrated by beads. Translated to pressure gradients, the gradient applied was 0.36 Pa/μm at 30° C., 0.312 Pa/μm at 37° C., and 0.288 Pa/μm at 40° C. The mobility of iRBCs was fairly constant from 30° C. to 37° C. and a significant drop in iRBC mobility was observed at 40° C.

FIG. 57A provides a cell mobility vs. temperature plot for parasite co-cultured but uninfected RBCs passing through 3 μm channels. Approximately 600 cells were tracked at a constant pressure gradient of 0.36 Pa/μm.

FIG. 57B provides a cell mobility vs. temperature plot indicating that normalized cell mobility was fairly constant from 30.0° C. to 37.0° C. This indicates that the increase in cell mobility in constant pressure gradient experiments was influenced by a viscosity change in buffer solution as well as in the cells. An apparent drop in cell mobility beyond 37° C. was detected in constant fluid velocity experiments.

FIG. 57C provides a cell mobility vs. temperature plot for healthy RBCs passing through 3 μm channels. Approximately 1000 cells were tracked at a constant pressure gradient of 0.36 Pa/μm. From 27.5° C. to 37.5° C., the RBC mobility increased linearly with temperature and was maximized around body temperature. From 37.5° C. to 40.0° C., the cells exhibit gradual stiffening with increasing temperature as indicated by the subtle drop in cell mobility.

FIG. 57D provides results from a control experiment conducted at constant fluid velocity in 4 um channels at all temperatures. From 30.0° C. to 37.0° C., the normalized cell mobility was fairly constant. This indicates that the increase in cell mobility in constant pressure gradient experiments is influenced by the viscosity change in buffer solution as well as in the cells. An apparent drop in cell mobility beyond 37° C. was detected in constant fluid velocity experiments. The fluid velocity calibration by microspheres is illustrated in FIG. 56B.

FIG. 58A provides a cell mobility plot for both infected and co-cultured uninfected cells passing though 3 μm channels at 30° C.

FIG. 58B provides a cell mobility plot for both infected and co-cultured uninfected cells passing though 3 μm channels at 37° C.

FIG. 58C provides a cell mobility plot for both infected and co-cultured uninfected cells passing though 3 μm channels at 40° C.

FIG. 59A provides a cell mobility histogram with normal fit for both infected (red curve) and co-cultured uninfected (black curve) RBCs at 37° C. For the uninfected RBCs, their mean mobility and standard deviation were (52.02, 9.41). For the infected RBCs, their mean mobility and standard deviation were (33.04, 11.61).

FIG. 59B provides a cell mobility histogram with normal fit for both infected (red curve) and co-cultured uninfected (black curve) RBCs at 40° C. For the uninfected RBCs, their mean mobility and standard deviation were (44.82, 8.19). For the infected RBCs, their mean mobility and standard deviation were (21.29, 5.87). The normal fit graphs at 37° C. and 40° C. were overlaid in FIG. 58C.

FIG. 59C overlays the normal fit graphs at 37° C. (FIG. 59A) and at 40° C. (FIG. 59B). At two standard deviations away from the mean uRBC mobility, a hypothetical line was drawn to represent the threshold value of splenic filtration.

FIG. 60 provides cell mobility plots for both infected cells (shown by diamond symbols) and co-cultured uninfected cells (shown by circle symbols) with and without Artesunate drug treatment. The drug effect was traced at 2, 4 and 6 hours after drug treatment. Measurements were done at physiologically relevant dosage from 0.01 to 0.1 μg/ml. The significant decrease in cell mobility due to drug treatment is expected to effectively promote spleen clearance of infected RBCs.

FIG. 61A depicts cell mobility vs. pressure for hRBCs at 37° C.

FIG. 61B compares RBC mobility at 37° C. vs 40° C. at low pressure gradients of 0.072 Pa/μm and 0.12 pa/μm.

FIG. 61C compares cell mobility for hRBCs and uRBCs at 37° C.

FIG. 61D compares hRBC mobility with or without TO staining.

FIG. 62 depicts results of a FACS analysis of the expression of the cell activation marker, CD25, before (left) and after (right) four days of activation of WT (in this case Balb/c) T cells. The shift in the peak of the PE fluorescence signal indicates successful activation.

FIG. 63 depicts results of an analysis of average apparent Young's modulus of WT T cells before (naive) and after (activated) cell activation as determined by the micropipette aspiration method. The modulus is 290+/−102.

FIG. 64A depicts a representative approach curve from an AFM cell indentation experiment fitted with the linear elastic Hertz model. Overlap of the Hertzian fit (red line) and the experimental data indicates and accurate efit of the model.

FIG. 64B provides a plot showing variation of the apparent Young's modulus of a T cell with cell indentation depth.

FIG. 65 provides a plot depicting variation of the apparent Young's modulus of naïve and activated WT T cells with AFM indentation speed. Cells were tested at 200 nm/sec, 1 μm/sec, 10 μm/sec, 20 μm/sec, and 50 μm/sec. The data points shown are the averages of the modulus values obtained at the indicated testing speeds.

FIG. 66A provides a graph showing the average apparent Young's modulus of WAS−/− T cells before (naive) and after (activated) cell activation, as determined by the micropipette aspiration method. The modulus is 190+/−69 Pa (mean+/−SD) and 121+/−41 Pa for naïve and activated WAS−/− T cells, respectively.

FIG. 66B provides a graph showing the average moduli for WT T cells before and after activation are shown side-by-side for comparison. The error bars designate the standard deviation.

FIG. 67 shows the average apparent Young's modulus of WT and WAS−/− T cells under different treatment conditions determined by the micropipette aspiration method. The modulus is 128+/−33 Pa (mean+/−SD) and 152+/−102 Pa for CCL19-stimulated WT and WAS−/− T cells, respectively. The average moduli for WT and WAS−/− T cells before and after activation are described elsewhere herein and are shown side-by-side here for comparison. The error bars designate the standard deviation.

FIG. 68 depicts results of a FACS analysis of phenotype of naïve WT T cells induced to migrate by the chemokine CCL19. T cells were first gated on PI to exclude dead cells. The cell samples tested were naïve T cells not exposed to CCL 19 (A, D), cells that remained in the insert after CCL19 exposure (100 ng/mL) for seven hours (B, E), and cells that migrated across the insert membrane after the same chemokine treatment (C, F). Gating on Thy1 (A-C), one marker that identifies T cells, revealed that more than 99% of the cells in all three samples were most likely T cells. Gating simultaneously on CD62L and CD44 (D-F) showed that a similar percentage of cells stained CD62L high and CD44 low, indicative of the naïve T cell phenotype.

### DETAILED DESCRIPTION OF INVENTION

Aspects of the present invention relate to methods and devices for evaluating, characterizing, and assessing material properties (e.g., mechanical and rheological properties) of certain substances, particularly biological substances. Any of a variety of material properties may be evaluated, depending on the method, device, and/or substance. Illustrative examples of such material properties include deformability, compressive strength, Poisson's ratio, shear modulus, shear strength, softness, specific modulus, specific weight, tensile strength, yield strength, Young's modulus, apparent Young's modulus, viscosity, apparent viscosity, time-dependent viscosity, oscillatory shear, and extensional flow.

In some aspects, a material property includes the viscoelasticity of a biological substance such as a cell or a cell-containing fluid. In some embodiments, methods are provided for disjoining viscosity from the elastic properties of a substance, such as a cell or fluid. Viscoelasticity can be linear or non-linear. In some embodiments, methods are provided for measuring the rigidity (e.g., deformability) of a biological substance with or without regard to the viscosity of the biological substance and/or its surrounding fluid. Provided herein are methods and devices useful for dissociating the rigidity of a biological substance from the adjacent viscosity. In some embodiments, the time taken by a substance (e.g., a red blood cell) to return to a normal shape and/or associated relaxation characteristics following deformation provide a measure of the viscoelasticity of the substance.

Methods and devices provided herein for evaluating, characterizing, and/or assessing material properties may be applied to a variety of different substances, including solids and fluids. The substance may be, for example, an elastic substance or a viscoelastic substance. The substance may be a Newtonian fluid or a non-Newtonian fluid. Substances may be natural or synthetic. Substances may be pure or may be a mixture. Substances that are mixtures may be homogeneous mixtures or heterogeneous mixtures. Substances may possess material properties of a solid, a fluid or a combination thereof. Substances may possess material properties that are linear or non-linear. The substance may be a biological substance, including, for example, a cell, a tissue, or biological fluid. Biological fluids include, for example, spinal fluid, lymphatic fluid, mucus, semen, sputnum and blood. Blood includes whole-blood, plasma, plasma components, serum, bone marrow, and components thereof.

In some cases, the substance is a deformable object. As used herein the term “deformable object” refers to an object that is capable of altering its shape in response to an applied force. A deformable object may or may not return to its original shape after deforming in response to an applied force. A deformable object that returns to its original shape after deforming in response to an applied force may return to its original shape essentially instantaneously after removal of the applied force, or after a certain period of time has elapsed after removal of the applied force. Often, a deformable object is an object that exhibits viscoelastic properties.

A deformable object may be a synthetic object such as, for example, a polymeric bead (e.g., a polyethylene bead), a micelle, a liposome, a particle, etc. A deformable object may be a microparticle or nanoparticle. The term “microparticle,” as used herein, refers to a particle having an average diameter on the order of micrometers (between about 1 micrometer and about 1 mm), while the term “nanoparticle” refers to a particle having an average diameter on the order of nanometers (between about 1 nm and about 1 micrometer). The particles may also have any shape or size. For instance, the particles may have an average diameter of less than about 5 mm or 2 mm, or less than about 1 mm, or less than about 500 μm, less than about 200 μm, less than about 100 μm, less than about 60 μm, less than about 50 μm, less than about 40 μm, less than about 30 μm, less than about 25 μm, less than about 10 μm, less than about 3 μm, less than about 1 μm, less than about 300 nm, less than about 100 nm, less than about 30 nm, or less than about 10 nm. The particles may be spherical or non-spherical. The average diameter of a non-spherical particle is the diameter of a perfect sphere having the same volume as the non-spherical particle.

A deformable object may be a biological object such as, for example, a vesicle, a eukaryotic cell, a prokaryotic cell, an organelle, a cell fragment (e.g., a platelet), a virus, a biomolecular aggregate, etc. Eukaryotic cells may be primary cells isolated from any tissue or organ (e.g., connective, nervous, muscle, fat or epithelial tissue). The cells may be mesenchymal, ectodermal, or endodermal. The cells may be nucleated or non-nucleated.

In one example, the deformable objects are cells, e.g., red blood cells, white blood cells, stem cells, cancer cells, epithelial cells (e.g., epithelial cells of the cervix, pancreas, breast or bladder), B cells, T cells, or plasma cells. The red blood cells can be fetal red blood cells, red blood cells infected with a parasite, red blood cells from an athlete, or a subject having or is suspected of having a disease (e.g., diabetes, infection with a virus such as HIV, anemia, a hematological cancer such as leukemia, a spleen disease, multiple myeloma, monoclonal gammopathy of undetermined significance, sickle cell disease, or spherocytosis). The cells may be infected with a pathogen. The pathogen may be, for example, a virus, bacterium, fungus or parasite. The parasite may be, for example, Plasmodium, Toxoplasma gondii, Leishmania, or Babesia.

Cells may be derived from, or contained in, isolated connective, nervous, muscle, fat or epithelial tissue. The connective tissue may be, for example, blood, bone, ligament, cartilage, tendon, or adipose tissue. The muscle tissue may be vascular smooth muscle, heart smooth muscle, or skeletal muscle, for example. The epithelial tissue may be of the blood vessels, ducts of submandibular glands, attached gingiva, dorsum of tongue, hard palate, esophagus, pancreas, adrenal glands, pituitary glands, prostate, liver, thyroid, stomach, small intestine, large intestine, rectum, anus, gallbladder, thyroid follicles, ependyma, lymph vessel, skin, sweat gland ducts, mesothelium of body cavities, ovaries, fallopian tubes, uterus, endometrium, cervix (endocervix), cervix (ectocervix), vagina, labia majora, tubuli recti, rete testis, ductuli efferentes, epididymis, vas deferens, ejaculatory duct, bulbourethral glands, seminal vesicle, oropharynx, larynx, vocal cords, trachea, respiratory bronchioles, cornea, nose, proximal convoluted tubule of kidney, ascending thin limb of kidney, distal convoluted tubule of kidney, collecting duct of kidney, renal pelvis, ureter, urinary bladder, prostatic urethra, membranous urethra, penile urethra, or external urethral orifice, for example.

The cells may be any mammalian cells. The cells may be any human cells. The cells may be selected from the group consisting of lymphocytes, B cells, T cells, cytotoxic T cells, natural killer T cells, regulatory T cells, T helper cells, myeloid cells, granulocytes, basophil granulocytes, eosinophil granulocytes, neutrophil granulocytes, hypersegmented neutrophils, monocytes, macrophages, reticulocytes, platelets, mast cells, thrombocytes, megakaryocytes, dendritic cells, thyroid cells, thyroid epithelial cells, parafollicular cells, parathyroid cells, parathyroid chief cells, oxyphil cells, adrenal cells, chromaffin cells, pineal cells, pinealocytes, glial cells, glioblasts, astrocytes, oligodendrocytes, microglial cells, magnocellular neurosecretory cells, stellate cells, boettcher cells; pituitary cells, gonadotropes, corticotropes, thyrotropes, somatotrope, lactotrophs, pneumocyte, type I pneumocytes, type II pneumocytes, Clara cells; goblet cells, alveolar macrophages, myocardiocytes, pericytes, gastric cells, gastric chief cells, parietal cells, goblet cells, paneth cells, G cells, D cells, ECL cells, I cells, K cells, S cells, enteroendocrine cells, enterochromaffin cells, APUD cell, liver cells, hepatocytes, Kupffer cells, bone cells, osteoblasts, osteocytes, osteoclast, odontoblasts, cementoblasts, ameloblasts, cartilage cells, chondroblasts, chondrocytes, skin cells, hair cells, trichocytes, keratinocytes, melanocytes, nevus cells, muscle cells, myocytes, myoblasts, myotubes, adipocyte, fibroblasts, tendon cells, podocytes, juxtaglomerular cells, intraglomerular mesangial cells, extraglomerular mesangial cells, kidney cells, kidney cells, macula densa cells, spermatozoa, sertoli cells, leydig cells, oocytes, and mixtures thereof.

The cells may also be isolated from a healthy tissue or a diseased tissue, e.g., a cancer. Accordingly, the cells may be cancer cells. For example, the cells may be isolated or derived from any of the following types of cancers: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia, e.g., B Cell CLL; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Cancer cells may be cells derived from any stage of cancer progression including, for example, precancerous cells, cancerous cells, and metastatic cells. Cancer cells also include cells from a primary tumor, secondary tumor or metastasis.

The cells may be selected from the group consisting of cord-blood cells, stem cells, embryonic stem cells, adult stem cells, cancer stem cells, progenitor cells, autologous cells, isograft cells, allograft cells, xenograft cells, and genetically engineered cells. The cells may be induced progenitor cells. The cells may be cells isolated from a subject, e.g., a donor subject, which have been transfected with a stem cell associated gene to induce pluripotency in the cells. The stem cell-associated genes may be selected from the group consisting of Oct3, Oct4, Sox1, Sox2, Sox3, Sox15, Klf1, Klf2, Klf4, Klf5, Nanog, Lin28, C-Myc, L-Myc, and N-Myc. The cells may be cells which have been isolated from a subject, transfected with a stem cell associated gene to induce pluripotency, and differentiated along a predetermined cell lineage.

In one example, the deformable objects are prokaryotic cells. Prokaryotic cells may be from any phyla, including Aquificae, Bacteroids, Chlorobia, Chrysogenetes, Cyanobacteria, Fibrobacter, Firmicutes, Flavobacteria, Fusobacteria, Proteobacteria, Sphingobacteria, Spirochaetes, Thermomicrobia, and/or Xenobacteria, among others. Such bacteria may be gram-negative, gram-positive, harmful, beneficial, and/or pathogenic. Exemplary prokaryotic cells may include E. coli, S. typhimurium, B subtilis, S. aureus, C. perfiingens, V. parahaemolyticus, and/or B. anthracis, among others.

In another example, the deformable objects are viruses (or cells infected therewith) including, for example, any DNA, RNA, and/or protein containing particle that infects and/or replicates in cells. The term virus encompasses DNA viruses, RNA viruses, retroviruses, virions, viroids, prions, etc. Exemplary viruses may include HIV, RSV, rabies, hepatitis virus, Epstein-Barr virus, rhinoviruses, bacteriophages, and diseases causing prions.

In another example, the deformable objects are organelles. The term, “organelle” as used herein refers to any component of a cell. Organelles may include, for example, nuclei, Golgi apparatus, lysosomes, endosomes, mitochondria, peroxisomes, endoplasmic reticulum, phagosomes, vacuoles, chloroplasts, etc.

The foregoing examples of deformable objects are not intended to be limiting. It should thus be appreciated that devices and methods disclosed herein may be used with any appropriate deformable object.

### Methods

Methods are provided herein for evaluating, characterizing, and/or assessing material properties of deformable objects. In particular, methods are provided for measuring, evaluating and characterizing dynamic mechanical responses of biological cells, e.g., red blood cells, white blood cells, reticulocytes, platelets, etc. The methods typically involve obtaining measurements of cell deformability. Measurements of cell deformability often involve an assessment of the transit time of one or more deformable objects through one or more constrictions within a fluid channel of a microfluidic device, or an assessment of another parameter indicative of a resistance to deformation. In some cases, the methods may be carried out in a high throughput manner. In further aspects, methods are provided that are useful for diagnosing, assessing, characterizing, evaluating, and/or predicting disease based on transit characteristics of cells, e.g., red blood cells, platelets, cancer cells, and tissues, e.g., blood in microfluidic devices.

In some cases, the methods involve acquiring microscopic measurements, e.g., fluorescence measurements, on deformable objects passing through one or more constrictions of a microfluidic device. In cases, where the deformable objects are, for example, cells, a combination of acquired microfluidic data (e.g., flow, pressure, transit time, constriction geometry, flow length, etc.) and microscopic data (e.g., presence or absence of a cell surface markers), enables a population-based correlation between cellular and/or biochemical properties and dynamic mechanical deformability.

### Characterizing Deformable Objects

Method for characterizing deformability of one or more deformable objects are provided herein. The methods typically involve perfusing a fluid containing one or more deformable objects through a microfluidic channel that includes at least one constriction and determining a transit characteristic of the one or more deformable objects. The transit characteristic may be for example the transit time for the one or more deformable object to travel from a first position within the microfluidic channel that is upstream of a constriction to a second position within the microfluidic channel that is downstream of a constriction. The transit characteristic may be, for example, the average velocity of the one or more deformable objects between a first position within the microfluidic channel that is upstream of a constriction and a second position within the microfluidic channel that is downstream of a constriction.

The transit characteristic may be determined in any of a variety of ways. Typically, the transit characteristic determination involves performing microscopy to acquire photomicrographic images of the deformable object as it passes through the channel. The object can be tracked manually, e.g., by examining the images by eye, or automatically, by implementing an image processing and/or image object tracking algorithm. For example, the transit characteristic may be determined by acquiring a first photomicrographic image of the one or more deformable objects at the first position and acquiring a second photomicrographic image of the one or more deformable objects at the second position, and determining the duration between acquisition of the first photomicrographic image and acquisition the second photomicrographic image. The duration, in this example, is the transit time. The average velocity can be readily determined, in this example, by computing the ratio of the transit time to the transit distance.

The constriction typically has an inlet orifice, outlet orifice and/or conduit that has a geometry that causes the object to deform as it passes through the constriction. Thus, the size and/or shape of the constriction may be configured so as to require that the object deform in order to pass through the constriction. For example, the constriction may have an inlet orifice, outlet orifice, and/or conduit having a dimension (e.g., diameter), perpendicular to the flow path, that is smaller in length than the diameter of the object, such that the object must deform in order to pass through the constriction.

In some cases, the methods involve perfusing a fluid containing one or more deformable objects through a microfluidic channel that includes a plurality of constrictions arranged in series. The plurality of constrictions are typically arranged in series such that a flow path through each constriction of the plurality is longitudinally aligned with a flow path through each other constriction of the plurality. In this configuration, the one or more deformable objects can be tracked, e.g., by microscopy, as it enters or passes through each constriction of the plurality. However, the methods and devices are not so limited and configurations are envisioned where the plurality of constrictions are arranged sequentially such that a flow path through each constriction of the plurality is not longitudinally aligned with a flow path through each other constriction of the plurality.

The deformability of an object may be characterized, in some cases, by evaluating the effects of constriction geometries on the transit of a deformable object through a microfluidic channel. For example, the transit times of a deformable object through two or more different constrictions (e.g., constrictions having different geometries, e.g., different inlet orifice, outlet orifice, and/or conduit geometries) may be used to define a signature that characterizes the deformability of the deformable object.

### Diagnosis

Also disclosed herein are methods for detecting a condition or disease in a subject. “Subject,” as used herein, refers to any animal. Typically a subject is a mammal, particularly a domesticated mammal (e.g., dogs, cats, etc.), primate, human or laboratory animal. In certain embodiments, the subject is a human. In certain embodiments, the subject is a laboratory animal such as a mouse or rat. A subject under the care of a physician or other health care provider may be referred to as a “patient.” In the context of diagnosis, typically the subject has or is suspected of having a disease. The diagnostic methods disclosed herein may be used in combination with one or more known diagnostic approaches in order to diagnose a subject as having a disease.

The methods typically involve obtaining a biological sample from the subject. As used herein, the phrase “obtaining a biological sample” refers to any process for directly or indirectly acquiring a biological sample from a subject. For example, a biological sample may be obtained (e.g., at a point-of-care facility, e.g., a physician's office, a hospital, laboratory facility) by procuring a tissue or fluid sample (e.g., blood draw, marrow sample, spinal tap) from a subject. Alternatively, a biological sample may be obtained by receiving the biological sample (e.g., at a laboratory facility) from one or more persons who procured the sample directly from the subject. The biological sample may be, for example, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

The biological sample typically serves as a test agent for a deformability assay. The results of the deformability assay of the test agent are often indicative of the disease status of the subject. For example, in some cases, deformability of the test agent, e.g., a cell, is indicative of the presence of the condition or disease in the subject. In some cases, the deformability assay involves perfusing a fluid containing a test agent through a microfluidic channel that comprises a constriction, such that the test agent passes through the constriction, and deforms as it passes through the constriction. The assay further involves determining a transit characteristic of the test agent as it moves through the microfluidic channel and comparing the transit characteristic to an appropriate standard. The results of the comparison are typically indicative of whether the subject has the condition or disease. Thus, the subject may be diagnosed as having the condition or disease based on the results of the deformability assay, in some cases.

Any appropriate condition or disease of a subject may be evaluated using the methods herein, typically provided that a test agent may be obtained from the subject that has a material property (e.g., deformability, shear modulus, viscosity, Young's modulus, etc.) that is indicative of the condition or disease. The condition or disease to be detected may be, for example, a fetal cell condition, HPV infection, or a hematological disorder, such as hematological cancer, anemia, infectious mononucleosis, HIV, malaria, leishmaniasis, sickle cell disease, babesiosis, spherocytosis, monoclonal gammopathy of undetermined significance or multiple myeloma. Examples of hematological cancer include, but are not limited to, Hodgkin's disease, Non-Hodgkin's lymphoma, Burkitt's lymphoma, anaplastic large cell lymphoma, splenic marginal zone lymphoma, hepatosplenic T-cell lymphoma, angioimmunoblastic T-cell lymphoma (AILT), multiple myeloma, Waldenström macroglobulinemia, plasmacytoma, acute lymphocytic leukemia (ALL), chronic lymphocytic leukemia (CLL), B cell CLL, acute myelogenous leukemia (AML), chronic myelogenous leukemia (CML), T-cell prolymphocytic leukemia (T-PLL), B-cell prolymphocytic leukemia (B-PLL), chronic neutrophilic leukemia (CNL), hairy cell leukemia (HCL), T-cell large granular lymphocyte leukemia (T-LGL) and aggressive NK-cell leukemia. The foregoing diseases or conditions are not intended to be limiting. It should thus be appreciated that other appropriate diseases or conditions may be evaluated using the methods disclosed herein.

Methods are also provided for detecting and/or characterizing a condition or disease such as diabetes characterized by substantial glycosylation of cell surface membranes. In particular, a plurality of cell-surface associated carbohydrates detectably alters the deformability of the coated cell, providing a prognostic indicator of cell function and disease progression, in some examples. Such prognostic indicators are useful, in some cases, in other diseases characterized by abnormal levels of circulating factors, such as cholesterol.

Methods are also provided for detecting and characterizing a leukocyte-mediated condition or disease. For example, methods are provided for detecting and characterizing a leukocyte-mediated condition or disease associated with the lungs of a subject being highly susceptible to injury, possibly due to activated leukocytes with altered deformability, having altered ability to circulate through the pulmonary capillary bed. Methods such as these, and others disclosed herein, can also be applied to detect and/or characterize septic shock (sepsis) that is associated with both rigid and activated neutrophils. Such neutrophils can, in some cases, occlude capillaries and damage organs where changes in neutrophil cytoskeleton are induced by molecular signals leading to decreased deformability.

Further, certain methods of the invention provide for measurement of cytoadhesive properties of a cell population, in combination with or separate from measurement of the deformability of the cell population. The combination of determining cytoadhesive properties and the deformative properties of a cell population, particularly a cell population containing a plurality of different cell types (e.g., red blood cells and white blood cells), may be used to generate a “Health Signature” that comprises an array of properties that can be tracked in a subject over a period of time. Such a Health Signature may facilitate effective monitoring of a subject's health over time. Such monitoring may lead to an early detection of potential acute or chronic infection, or other disease, disorder, fitness, or condition. In some cases, further, knowledge of the overall rheology of a material, along with either the deformative or cytoadhesive property of a cell, allows the determination of the other property.

A method for detecting a condition or disease (e.g., abnormal fetal condition, fetal health, fetal gender, fetal age or diabetes) in a subject may, in some cases, include at least the following steps: (a) obtaining a maternal blood sample from the subject, the sample containing a deformable object (e.g., a cell such as a fetal cell) (b) analyzing a mechanical property of the blood sample using a device; and (c) comparing the mechanical property to an appropriate standard. The results of the comparison are typically indicative of the status of the condition or disease in the subject or the identity of a fetal cell. In embodiments, the method can further comprise performing a test on the fetal cell. In one example, the device is a microfluidic channel. In another example, the device is not a microfluidic channel. The deformable object, in this example, typically has a mechanical property, the value of which is indicative of the presence of an abnormal fetal condition. In one example, the method is used to distinguish between fetal red blood cells and maternal red blood cells based on differences in mechanical properties. In another example, the method is used to separate fetal cells from maternal cells (e.g., maternal red blood cells) based on differences in mechanical properties. In such methods, the methods can also comprise a step of performing a test on the separated fetal cells.

A method for detecting a condition or disease in a subject may, in some cases, include at least the following steps: (a) obtaining a sample from the subject, the sample including a deformable object having a mechanical property that is indicative of the presence of the condition or disease, e.g., stiffness, deformability, viscoelasticity, viscosity, adhesiveness, or a combination thereof; (b) analyzing the mechanical property using a non-microfluidic channel device, and (c) comparing the mechanical property to an appropriate standard. The results of the comparison are indicative of whether the subject has the condition or disease. Step (b) of this example can be performed by determining a value for at least one mechanical property of the one or more deformable objects. The non-microfluidic channel device used in this step can be AFM, optical tweezers, micropipette, magnetic twisting cytometer, cytoindenter, microindenter, nanoindenter, microplate stretcher, microfabricated post array detector, micropipette aspirator, substrate stretcher, shear flow detector, diffraction phase microscope, or tomographic phase microscope.

An “appropriate standard” is a parameter, value or level indicative of a known outcome, status or result (e.g., a known disease or condition status). An appropriate standard can be determined (e.g., determined in parallel with a test measurement) or can be pre-existing (e.g., a historical value, etc.). The parameter, value or level may be, for example, a transit characteristic (e.g., transit time), a value representative of a mechanical property, a value representative of a rheological property, etc. For example, an appropriate standard may be the transit characteristic of a test agent obtained from a subject known to have a disease, or a subject identified as being disease-free. In the former case, a lack of a difference between the transit characteristic and the appropriate standard may be indicative of a subject having a disease or condition. Whereas in the latter case, the presence of a difference between the transit characteristic and the appropriate standard may be indicative of a subject having a disease or condition. The appropriate standard can be a mechanical property or rheological property of a cell obtained from a subject who is identified as not having the condition or disease or can be a mechanical property or rheological property of a cell obtained from a subject who is identified as having the condition or disease.

The magnitude of a difference between a parameter, level or value and an appropriate standard that is indicative of known outcome, status or result may vary. For example, a significant difference that indicates a known outcome, status or result may be detected when the level of a parameter, level or value is at least 1%, at least 5%, at least 10%, at least 25%, at least 50%, at least 100%, at least 250%, at least 500%, or at least 1000% higher, or lower, than the appropriate standard. Similarly, a significant difference may be detected when a parameter, level or value is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the level of the appropriate standard. Significant differences may be identified by using an appropriate statistical test. Tests for statistical significance are well known in the art and are exemplified in Applied Statistics for Engineers and Scientists by Petruccelli, Chen and Nandram Reprint Ed. Prentice Hall (1999).

### Identifying Candidate Therapeutic Agents and Monitoring Efficacy of Therapeutic Agents

Methods are also provided for identifying candidate therapeutic agents for treating a condition or disease in a subject. The methods typically involve: (a) contacting a test agent with the candidate therapeutic agent, the deformability of the test agent being indicative of the condition or disease; (b) perfusing a fluid containing the test agent through a microfluidic channel that includes a constriction; (c) determining a transit characteristic of the test agent from a position within the microfluidic channel that is upstream of the constriction to a position within the microfluidic channel that is downstream of the constriction; and (d) comparing the transit characteristic to an appropriate standard as described herein. The results of the comparison are typically indicative of whether the candidate therapeutic agent can be used for treating the condition or disease in the subject. The test agent may be contacted with the candidate therapeutic agent before, during and/or throughout step (b), in this example. In some embodiments, the appropriate standard is the value of a transit characteristic for a test agent that has been contacted with a control therapeutic agent (e.g., artesunate). Typically, a control therapeutic agent is a molecule that has a known effect on deformability of a test agent and that is effective for treating the condition or disease. Thus, comparing the transit characteristic of a candidate therapeutic agent with that of a control therapeutic agent provides a basis for identifying candidate therapeutic agents that are likely to be useful for treating the disease or condition. For example, a candidate therapeutic agent that results in the same or a similar value for a particular transit characteristic as that of a control therapeutic agent that is known to be effective for treating the disease or condition is likely to be an agent that is also effective for treating the disease or condition.

By example, this method may be used to identify candidate therapeutic agents that improve blood flow in subjects with circulation problems such as leg ulcers, pain from diabetic neuropathy, eye and ear disorders, and altitude sickness. Similarly for subjects with aggregation or clotting disorders of cells or insufficient delivery of essential chemicals such as oxygen to the brain in subjects with strokes from blood clots.

Methods are also provided for monitoring the effectiveness of a therapeutic agent for a treating a condition or disease in a subject. The methods typically include: (a) obtaining a test agent, having a deformability that is indicative of the presence of the condition or disease; (b) perfusing a fluid comprising the test agent through a microfluidic channel that comprises a constriction, such that the test agent passes through the constriction; and (c) determining a transit characteristic of the test agent from a position within the microfluidic channel that is upstream of the constriction to a position within the microfluidic channel that is downstream of the constriction; (d) treating the subject with the therapeutic agent; and (e) repeating steps (a) through (c) one or more times. A difference in the transit characteristic of the test agent determined prior to the treatment compared with the transit characteristic of the test agent determined after the treatment is typically indicative of the effectiveness of the therapeutic agent.

Typically the therapeutic agent or candidate therapeutic agent is a small molecule or pharmaceutical agent. “Small molecule” refers to organic compounds, whether naturally-occurring or artificially created (e.g., via chemical synthesis) that have relatively low molecular weight and that are not proteins, polypeptides, or nucleic acids. Small molecules are typically not polymers with repeating units. In certain embodiments, a small molecule has a molecular weight of less than about 1500 g/mol. In certain embodiments, the molecular weight of the polymer is less than about 1000 g/mol. Also, small molecules typically have multiple carbon-carbon bonds and may have multiple stereocenters and functional groups.

“Pharmaceutical agent,” also referred to as a “drug,” is used herein to refer to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (USP), Goodman and Gilman's The Pharmacological Basis of Therapeutics, 10^{th} Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17^{th} ed. (1999), or the 18^{th} ed (2006) following its publication, Mark H. Beers and Robert Berkow (eds.), Merck Publishing Group, or, in the case of animals, The Merck Veterinary Manual, 9^{th} ed., Kahn, C. A. (ed.), Merck Publishing Group, 2005.

In some cases, the therapeutic agent or candidate therapeutic agent is a polynucleotide, protein or polysaccharide. The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least two nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, 2γ-methoxyribose, 2γ-aminoribose, ribose, 2′-deoxyribose, arabinose, and hexose), or modified phosphate groups (e.g., phosphorothioates and 5′-N phosphoramidite linkages). Enantiomers of natural or modified nucleosides may also be used. Nucleic acids also include nucleic acid-based therapeutic agents, for example, nucleic acid ligands, siRNA, short hairpin RNA, antisense oligonucleotides, ribozymes, aptamers, and SPIEGELMERS™, oligonucleotide ligands described in Wlotzka, et al., Proc. Natl. Acad. Sci. USA, 2002, 99(13):8898, the entire contents of which are incorporated herein by reference.

A “polypeptide”, “peptide”, or “protein” comprises a string of at least three amino acids linked together by peptide bonds. The terms “polypeptide”, “peptide”, and “protein”, may be used interchangeably. Peptide may refer to an individual peptide or a collection of peptides. Peptides may contain only natural amino acids, although non natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In one embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

The terms “polysaccharide” and “carbohydrate” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, galactose, mannose, ribose, arabinose, xylose, and fructose. Disaccharides are two joined monosaccharides. Exemplary disaccharides include sucrose, maltose, cell obiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose. (e.g., 2′-fluororibose, deoxyribose, and hexose). Carbohydrates may exist in many different forms, for example, conformers, cyclic forms, acyclic forms, stereoisomers, tautomers, anomers, and isomers.

### Isolating Target Cells

Methods of isolating target cells are also provided herein. The methods may be implemented using any of the devices disclosed herein. The methods may generally be used to separate any two populations of cells that differ with respect to one or more mechanical properties, e.g., deformability. The methods may therefore be applied to any of a variety of different cell populations. For example, reticulocytes may be separated from mature red blood cells, activated T-Cells may be separated from naïve T-Cells, cancer cells may be separated from normal cells, stem cells may be separated from differentiated cells, and so on.

In a typical example, a method is provided for isolating a target cell (e.g., stem cell or fetal cell) from a fluid (e.g., a maternal blood sample). The method typically involves perfusing a fluid having multiple cell types including the target cell through a microfluidic device and separating the target cell from other cell types in the fluid based on the deformability of the cells.

The methods may include, in some cases, at least the steps of (a) perfusing a fluid comprising one or more red blood cells through a flow test device, (b) separating the reticulocytes from mature red blood cells, and (c) collecting or removing the reticulocytes from the fluid. In other cases, the methods involve (a) perfusing a fluid comprising cells or platelets through a flow test device, (b) separating a first type of cell (e.g., reticulotytes or white blood cells such as T or B cells) or platelets from another component of the fluid (e.g., mature red blood cells or non-red blood cells) based on a mechanical property, wherein the mechanical property is stiffness, deformability, viscoelasticity, viscosity and/or adhesiveness, and (c) collecting or removing the first type of cell or platelets from the fluid. The fluid can be obtained from a subject. Either the first type of cell or platelets or the other component(s) collected can be returned to the same subject or administered to a different subject.

These methods may be used, for example, to identify red blood cells with biomechanical properties indicative of better oxygen-carrying capacity than other red blood cells such as to better treat anemia by red blood cell transfusion. Methods can be employed on stored red blood cells throughout the time of storage to monitor cell quality such as with packed red blood cells that are administered as therapy.

Using methods disclosed herein, elite blood cells may be separated from a sample. For example, a method may involve perfusing a fluid comprising one or more red blood cells through a flow test device, and collecting or removing elite red blood cells from the fluid. By applying this method to a blood sample taking from a subject, and determining the quantity of elite blood cells in the sample, the fitness of the subject may be determined, in some cases. As used herein, the term “elite blood cell” is meant to include red blood cells that have a greater oxygen-carrying capacity than an average red blood cell (i.e., the oxygen-carrying capacity that is expected for a “normal” or “average” red blood cell). In some embodiments, the elite blood cells are the red blood cells from a marathon runner or are those with an oxygen-carrying capacity of a red blood cell of a marathon runner. In other embodiments, elite red blood cells exhibit a deformability that would be expected of a red blood cell that is up to 80 days old. In still other embodiments, the elite red blood cell is one that is up to 80 days old. In some embodiments, the age of a blood cell is measured from the time of acquisition of a blood cell phenotype.

### Detecting Drug Use in a Subject

With wide spread use of controlled substances or narcotics such as morphine, cocaine, amphetamines, tranquilizers, synthetic analgesics, steroids, growth hormones, etc., it has become desirable to institute drug testing in certain circumstances. For example, drug testing is routinely performed on professional athletes, individuals working in both the private and public sectors, and others. Accordingly, in some aspects, methods of detecting drug use in a subject are provided herein. The methods are based, in part, on evaluating deformability characteristics of a biological sample, or component thereof, obtained from a subject. The methods typically include: (a) perfusing a fluid from the subject comprising a deformable object through a microfluidic device; (b) analyzing the transit of the deformable object through one or more constrictions of a microfluidic channel of the device; and (c) comparing one or more characteristics of the transit to an appropriate standard. The results of the comparison are indicative of whether the subject has used a drug. In some embodiments, the method comprises evaluating a material property of the deformable object using a non-microfluidic device. In some embodiments, the non-microfluidic device is AFM, optical tweezers, micropipette, magnetic twisting cytometer, cytoindenter, microindenter, nanoindenter, microplate stretcher, microfabricated post array detector, micropipette aspirator, substrate stretcher, shear flow detector, diffraction phase microscope, or tomographic phase microscope.

### Devices

Devices are provided herein for evaluating, characterizing, and assessing material properties of deformable objects. In particular, devices are provided for measuring, evaluating and characterizing dynamic mechanical responses of biological cells, e.g., red blood cells, reticulocytes, platelets, etc. The devices are typically designed and configured to permit measurements of cell deformability in a high throughput manner.

In some cases, the devices are designed and configured to permit microscopic measurements, e.g., fluorescence measurements, on deformable objects passing through the device. The devices, in some examples, are designed and configured to create low Reynolds number fluid regimes. Such fluid regimes are useful for evaluating the effects of constriction entrance architecture (e.g., inlet orifice size and/or shape) on the sensitivity of cell deformability measurements.

The devices typically include a structure defining one or more microfluidic channels through which a fluid that comprises one or more deformable objects may pass. When the structure defines two or more microfluidic channels, typically each of the channels is at least partially fluidically isolated from the other(s).

Each of the one or more microfluidic channels typically contains one or more of constrictions (e.g., two or three-dimensional). As used herein, the term “constriction” refers to a relatively narrow portion of a fluid passage, having an inlet orifice and an outlet orifice. As used herein, the term “inlet orifice” refers to an opening that defines an entrance into a narrow portion of a fluid passage and the term “outlet orifice” refers to an opening that defines an exit from a narrow portion of a fluid passage. Between an inlet orifice and outlet orifice, the constriction comprises a “conduit” through which a fluid and/or object may pass.

The inlet orifices and outlet orifices can have any of variety of shapes, including, for example, polygonal (e.g., triangular, rectangular), curvilinear or circular shape. In one example, the shape of the at least one inlet/outlet orifice is two-dimensional. In another example, it is three-dimensional. In either case, one or more dimensions of the at least one inlet orifice is less than, greater than, or equal to a dimension of a deformable object.

An inlet orifice may have a cross-sectional area of up to 0.1 μm², 0.5 μm², 1 μm², 2 μm², 3 μm², 4 μm², 5 μm², 6 μm², 7 μm², 8 μm², 9 μm², 10 μm², 11 μm², 12 μm², 13 μm², 14 μm², 15 μm², 16 μm², 17 μm², 18 μm², 19 μm², 20 μm², 21 μm², 22 μm², 23 μm², 24 μm², 25 μm², 26 μm², 27 μm², 28 μm², 29 μm², 30 μm², 31 μm², 32 μm², 33 μm², 34 μm², 35 μm², 36 μm², 37 μm², 38 μm², 39 μm², 40 μm², 41 μm², 42 μm², 43 μm², 44 μm², 45 μm², 46 μm², 47 μm², 48 μm², 49 μm², 50 μm², 55 μm², 60 μm², 65 μm², 70 μm², 75 μm², 80 μm², 85 μm², 90 μm², 95 μm², 100 μm², 150 μm², 200 μm², 250 μm², or more.

An inlet orifice may have a cross-sectional area in a range of 0.1 μm² to 1 μm², 1 μm² to 2 μm², 1 μm² to 10 μm², 2 μm² to 5 μm², 5 μm² to 10 μm², 5 μm² to 50 μm², 10 μm² to 15 μm², 15 μm² to 20 μm², 20 μm² to 30 μm², 30 μm² to 40 μm², 40 μm² to 50 μm², 50 μm² to 100 μm², or 100 μm² to 200 μm², for example.

An outlet orifice may have a cross-sectional area of up to 0.1 μm², 0.5 μm², 1 μm², 2 μm², 3 μm², 4 μm², 5 μm², 6 μm², 7 μm², 8 μm², 9 μm², 10 μm², 11 μm², 12 μm², 13 μm², 14 μm², 15 μm², 16 μm², 17 μm², 18 μm², 19 μm², 20 μm², 21 μm², 22 μm², 23 μm², 24 μm², 25 μm², 26 μm², 27 μm², 28 μm², 29 μm², 30 μm², 31 μm², 32 μm², 33 μm², 34 μm², 35 μm², 36 μm², 37 μm², 38 μm², 39 μm², 40 μm², 41 μm², 42 μm², 43 μm², 44 μm², 45 μm², 46 μm², 47 μm², 48 μm², 49 μm², 50 μm², 55 μm², 60 μm², 65 μm², 70 μm², 75 μm², 80 μm², 85 μm², 90 μm², 95 μm², 100 μm², 150 μm², 200 μm², 250 μm², or more.

An outlet orifice may have a cross-sectional area in a range of 0.1 μm² to 1 μm², 1 μm² to 2 μm², 1 μm² to 10 μm², 2 μm² to 5 μm², 5 μm² to 10 μm², 5 μm² to 50 μm², 10 μm² to 15 μm², 15 μm² to 20 μm², 20 μm² to 30 μm², 30 μm² to 40 μm², 40 μm² to 50 μm², 50 μm² to 100 μm², or 100 μm² to 200 μm², for example.

The geometry, e.g., size and shape, of the inlet and outlet orifices may or may not be the same. In some cases, the inlet orifice of at least one of the constrictions is geometrically different from the outlet orifice of the same constriction. As used herein, the term “geometrically different” means different in size and/or shape. For example, the inlet orifice(s) in one or more of the constrictions can have a larger cross-sectional area than the outlet orifice(s) in the same constriction(s), e.g., 19 μm² to 23 μm² versus 10 μm² to 15 μm². Alternatively, the inlet orifice(s) has a smaller cross-sectional area than the outlet orifice(s) in the same constriction, e.g., 10 μm² to 15 μm² versus 19 μm² to 23 μm².

The difference between the cross-sectional area of an inlet orifice and the cross-sectional area of an outlet orifice may be up to 0.1 μm², 0.5 μm², 1 μm², 2 μm², 3 μm², 4 μm², 5 μm², 6 μm², 7 μm², 8 μm², 9 μm², 10 μm², 11 μm², 12 μm², 13 μm², 14 μm², 15 μm², 16 μm², 17 μm², 18 μm², 19 μm², 20 μm², 21 μm², 22 μm², 23 μm², 24 μm², 25 μm², 26 μm², 27 μm², 28 μm², 29 μm², 30 μm², 31 μm², 32 μm², 33 μm², 34 μm², 35 μm², 36 μm², 37 μm², 38 μm², 39 μm², 40 μm², 41 μm², 42 μm², 43 μm², 44 μm², 45 μm², 46 μm², 47 μm², 48 μm², 49 μm², 50 μm², 55 μm², 60 μm², 65 μm², 70 μm², 75 μm², 80 μm², 85 μm², 90 μm², 95 μm², 100 μm², or more.

The difference between the cross-sectional area of an inlet orifice and the cross-sectional area of an outlet orifice may be in a range of 0.1 μm² to 1 μm², 1 μm² to 2 μm², 1 μm² to 10 μm², 2 μm² to 5 μm², 5 μm² to 10 μm², 5 μm² to 50 μm², 10 μm² to 15 μm², 15 μm² to 20 μm², 20 μm² to 30 μm², 30 μm² to 40 μm², 40 μm² to 50 μm², or 50 μm² to 100 μm², for example.

The one or more constrictions can have a conduit length (distance between inlet orifice and outlet orifice) of up to 0.1 μm, 0.5 μm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 1 mm or more.

The one or more constrictions can have a conduit length (distance between inlet orifice and outlet orifice) in a range of 0.1 μm to 1 μm, 1 μm to 10 μm, 5 μm to 50 μm, 25 μm to 100 μm, 50 μm to 200 μm, 150 μm to 500 μm, or 500 μm to 1 mm.

The one or more constrictions may have an average cross-sectional area, perpendicular to the flow direction through its conduit, of up to 0.1 μm², 0.5 μm², 1 μm², 2 μm², 3 μm², 4 μm², 5 μm², 6 μm², 7 μm², 8 μm², 9 μm², 10 μm², 11 μm², 12 μm², 13 μm², 14 μm², 15 μm², 16 μm², 17 μm², 18 μm², 19 μm², 20 μm², 21 μm², 22 μm², 23 μm², 24 μm², 25 μm², 26 μm², 27 μm², 28 μm², 29 μm², 30 μm², 31 μm², 32 μm², 33 μm², 34 μm², 35 μm², 36 μm², 37 μm², 38 μm², 39 μm², 40 μm², 41 μm², 42 μm², 43 μm², 44 μm², 45 μm², 46 μm², 47 μm², 48 μm², 49 μm², 50 μm², 55 μm², 60 μm², 65 μm², 70 μm², 75 μm², 80 μm², 85 μm², 90 μm², 95 μm², 100 μm², 150 μm², 200 μm², 250 μm², or more.

The one or more constrictions may have an average cross-sectional area, perpendicular to the flow direction through its conduit, in a range of 0.1 μm² to 1 μm², 1 μm² to 2 μm², 1 μm² to 10 μm², 2 μm² to 5 μm², 5 μm² to 10 μm², 5 μm² to 50 μm², 10 μm² to 15 μm², 15 μm² to 20 μm², 20 μm² to 30 μm², 30 μm² to 40 μm², 40 μm² to 50 μm², 50 μm² to 100 μm², or 100 μm² to 200 μm², for example.

The one or more constrictions may define a convergent conduit. The one or more constrictions may define a conduit having a cross-sectional area, perpendicular to the flow direction through the conduit, that converges (narrows) at a rate of 0.001 μm²/μm, 0.01 μm²/μm, 0.05 μm²/μm, 0.1 μm²/μm, 0.2 μm²/μm, 0.3 μm²/μm, 0.4 μm²/μm, 0.5 μm²/μm, 0.6 μm²/μm, 0.7 μm²/μm, 0.8 μm²/μm, 0.9 μm²/μm, 1 μm²/μm, 2 μm²/μm, 5 μm²/μm, 10 μm²/μm, or more.

The one or more constrictions may define a conduit having a cross-sectional area, perpendicular to the flow direction through the conduit, that converges at a rate in a range of 0.001 μm²/μm to 0.01 μm²/μm, 0.01 μm²/μm to 0.1 μm²/μm, 0.1 μm²/μm to 0.5 μm²/μm, 0.1 μm²/μm to 1 μm²/μm, or 1 μm²/μm to 10 μm²/μm, or more.

The one or more constrictions may define a divergent conduit. The one or more constrictions may define a conduit having a cross-sectional area, perpendicular to the flow direction through the conduit, that diverges (widens) at a rate of 0.001 μm²/μm, 0.01 μm²/μm, 0.05 μm²/μm, 0.1 μm²/μm, 0.2 μm²/μm, 0.3 μm²/μm, 0.4 μm²/μm, 0.5 μm²/μm, 0.6 μm²/μm, 0.7 μm²/μm, 0.8 μm²/μm, 0.9 μm²/μm, 1 μm²/μm, 2 μm²/μm, 5 μm²/μm, 10 μm²/μm, or more.

The one or more constrictions may define a conduit having a cross-sectional area, perpendicular to the flow direction through the conduit, that diverges at a rate in a range of 0.001 μm²/μm to 0.01 μm²/μm, 0.01 μm²/μm to 0.1 μm²/μm, 0.1 μm²/μm to 0.5 μm²/μm, 0.1 μm²/μm to 1 μm²/μm, or 1 μm²/μm to 10 μm²/μm, or more.

Other non-uniform conduit geometries are envisioned. For example, a constriction may have a conduit with an undulating, wavy, jagged, irregular or randomly altering cross-sectional area along its length.

The one or more microfluidic channels in the device described herein, when each contains at least two constrictions, can further contain a gap region between each successive constriction. In one example, this gap region is of a length that allows one or more deformable objects (e.g., cells, vesicles, biomolecular aggregates, platelets or particles) to recover, at least partially, their shape after passing through the first constriction (e.g., equal to the length of one of the constrictions and/or the length of its successive constriction). In another example, the gap region is of a length that does not allow one or more deformable objects to recover their shape after passing through each constriction.

The gap region may have a length (e.g., distance between outlet orifice of a first constriction and an inlet orifice of a second constriction, aligned in series) of up to 0.1 μm, 0.5 μm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 1 mm or more.

The gap region may have a length in a range of 0.1 μm to 1 μm, 1 μm to 10 μm, 5 μm to 50 μm, 25 μm to 100 μm, 50 μm to 200 μm, 150 μm to 500 μm, or 500 μm to 1 mm.

In one example, the one or more microfluidic channels each comprise at least two constrictions: (a) a first constriction having a first inlet orifice and a first outlet orifice, and (b) a second constriction having a second inlet orifice and a second outlet orifice. The first constriction and the second constrictions can be arranged in parallel such that a flow path through one constriction is parallel with a flow path through the other constriction. The first constriction and the second constriction can be arranged in series such that a flow path through one constriction is parallel with a flow path through the other constriction. The first constriction and the second constriction can be arranged in series such that a flow path through one constriction is parallel with a flow path through the other constriction. In these examples, the first inlet orifice and the first outlet orifice may be geometrically equal to or geometrically different than the second inlet orifice and the second outlet orifice, respectively.

In another example, the one or more microfluidic channels in the device each contain a plurality of constrictions arranged in series, each constriction of the plurality being a non-uniform conduit. In both examples described above, the constrictions can be arranged in series such that a flow path through each of the constrictions is aligned, longitudinally or non-longitudinally, with a flow path through each other constriction(s). Moreover, one, more than one, or all of the constrictions in the series may be a non-uniform conduit, e.g., a convergent conduit or a divergent conduit.

When a device contains at least two microfluidic channels, the constrictions in one of the channels can be arranged in parallel with those in each other channel(s) such that a flow path through the former is parallel with a flow path through the latter. Devices containing at least two microfluidic channels, may be designed and constructed such that the resistance to flow through each channel is different. Alternatively, devices containing at least two microfluidic channels, may be designed and constructed such that the resistance to flow through each channel is essentially same.

Furthermore, when a device contains at least two microfluidic channels, the fluidics associated the channels can be arranged such that flow through each channel(s) travels in the same direction, or in opposite directions. When a device contains at least two microfluidic channels and the fluidics associated the channels are arranged such that flow through each channel(s) travels in the same direction, the channels are typically either partially fluidically isolated or fluidically isolated. When a device contains at least two microfluidic channels and the fluidics associated the channels are arranged such that flow through each channel(s) travels in opposite directions, the channels are typically fluidically isolated. Channels that are “fluidically isolated” are configured and designed such that there is no fluid exchanged directly between the channels. Channels that are “partially fluidically isolated” are configured and designed such that there is partial (e.g., incidental) fluid exchanged directly between the channels.

Devices containing one or more microfluidic channels can further contain a substantially planar transparent wall that defines a surface of at least one of the constrictions. This substantially planar transparent wall, which can be, for example, glass or plastic, permits observation into the microfluidic channel by microscopy so that at least one measurement of each deformable object that passes through one of the microfluidic channels can be obtained. In one example, the transparent wall has a thickness of 0.05 mm to 1 mm. In some cases, the transparent wall may be a microscope cover slip, or similar component. Microscope coverslips are widely available in several standard thicknesses that are identified by numbers, as follows: No. 0-0.085 to 0.13 mm thick, No. 1-0.13 to 0.16 mm thick, No. 1.5-0.16 to 0.19 mm thick, No. 2-0.19 to 0.23 mm thick, No. 3-0.25 to 0.35 mm thick, No. 4-0.43 to 0.64 mm thick, any one of which may be used as a transparent wall, depending on the device, microscope, deformable object size, and deformable object detection strategy.

The transparent wall, or any wall of the microfluidic channel contains binding agents. Exemplary binding agents include antibodies, aptamers, or other suitable affinity capture reagents for binding to a target of interest, e.g., an deformable object, e.g., a cell, etc.

The microfluidic channel(s) may have a height in a range of 0.5 μm to 100 μm, 0.1 μm to 100 μm, 1 μm to 50 μm, 1 μm to 50 μm, 10 μm to 40 μm, 5 μm to 15 μm, 0.1 μm to 5 μm, or 2 μm to 5 μm. The microfluidic channel(s) may have a height of up to 0.5 μm, 1 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 20 μm, 30 μm, 40 μm, 50 μm, 75 μm, 100 μm, or more.

The microfluidic channel(s) may, in some cases, comprise 2, 3, 4, 5, 6, 7, 8, 9, 10, 15, 20, 25, 30, 35, 40, 45, 50, 75, 100, 200, or more constrictions, arranged in series. The microfluidic channel(s) may comprise 2 to 5, 2 to 10, 2 to 20, 2 to 50, 10 to 50, 10 to 100, or 50 to 200 constrictions, arranged in series, for example.

The microfluidic channel(s) may, in some cases, comprise 2, 3, 4, 5, 6, 7, 8, 9, 10, 15, 20, 25, 30, 35, 40, 45, 50, 75, 100, 200, or more constrictions, arranged in parallel. The microfluidic channel(s) may comprise 2 to 5, 2 to 10, 2 to 20, 2 to 50, 10 to 50, 10 to 100, or 50 to 200 constrictions, arranged in parallel, for example.

The device described above can further contain a reservoir fluidically connected with the one or more microfluidic channels, and a pump that perfuses fluid from the reservoir through the one or more microfluidic channels, and optionally, a microscope arranged to permit observation within the one or more microfluidic channels. The reservoir may contain deformable objects suspended in a fluid. The fluidics connecting the reservoir to the microfluidic channel(s) may include one or more filters to prevent the passage of unwanted or undesirable components into the microfluidic channels.

The device may be designed and configured to create a pressure gradient from the channel inlet to the channel outlet of 0.05 Pa/μm, 0.1 Pa/μm, 0.15 Pa/μm, 0.2 Pa/μm, 0.25 Pa/μm, 0.3 Pa/μm, 0.35 Pa/μm, 0.4 Pa/μm, 0.45 Pa/μm, 0.5 Pa/μm, 0.55 Pa/μm, 0.6 Pa/μm, 0.65 Pa/μm, 0.7 Pa/μm, 0.75 Pa/μm, 0.8 Pa/μm, 0.85 Pa/μm, 0.9 Pa/μm, 0.95 Pa/μm, 1 Pa/μm, 2 Pa/μm, 3 Pa/μm, 4 Pa/μm, 5 Pa/μm, 10 Pa/μm, or more.

The device may be designed and configured to create a pressure gradient from the channel inlet to the channel outlet in a range of 0.05 Pa/μm to 0.1 Pa/μm, 0.1 Pa/μm to 0.3 Pa/μm, 0.1 Pa/μm to 0.5 Pa/μm, 0.1 Pa/μm to 0.8 Pa/μm, 0.5 Pa/μm to 1 Pa/μm, 1 Pa/μm to 10 Pa/μm, for example. The pressure gradient may be linear or non-linear.

The device may be designed and configured to create a pressure (gauge pressure) in the channel of up to 50 Pa, 100 Pa, 200 Pa, 300 Pa, 400 Pa, 500 Pa, 600 Pa, 700 Pa, 800 Pa, 900 Pa, 1 kPa, 2 kPa, 5 kPa, 10 kPa or more. The device may be designed and configured to create a pressure (gauge pressure) in the channel in a range of 50 Pa to 200 Pa, 100 Pa to 500 Pa, 100 Pa to 800 Pa, 100 Pa to 1 kPa, 500 Pa to 5 kPa, or 500 Pa to 10 kPa.

The device may be designed and configured to create an average fluid velocity within the channel of up to 1 μm/s, 2 μm/s, 5 μm/s, 10 μm/s, 20 μm/s, 50 μm/s, 100 μm/s, or more.

The device may be designed and configured to create an average fluid velocity within the channel in a range of 1 μm/s to 5 μm/s, 1 μm/s to 10 μm/s, 1 μm/s to 20 μm/s, 1 μm/s to 50 μm/s, 10 μm/s to 100 μm/s, or 10 μm/s to 200 μm/s, for example.

The device may be designed and configured to have a channel cross-sectional area, perpendicular to the flow direction, of 1 μm², 10 μm², 20 μm², 30 μm², 40 μm², 50 μm², 60 μm², 70 μm², 80 μm², 90 μm², 100 μm², 150 μm², 200 μm², 300 μm², 400 μm², 500 μm², 600 μm², 700 μm², 800 μm², 900 μm², 1000 μm², or more.

The device may be designed and configured to have a channel cross-sectional area, perpendicular to the flow direction, in a range of 1 μm² to 10 μm², 10 μm² to 50 μm², 50 μm² to 100 μm², 100 μm² to 500 μm², 500 μm² to 1500 μm², for example.

The device may be designed and configured to produce any of a variety of different shear rates (e.g., up to 1000 s⁻¹). For example, the device may be designed and configured to produce a shear rate in a range of 10 s⁻¹ to 50 s⁻¹, 10 s⁻¹ to 100 s⁻¹, 50 s⁻¹ to 200 s⁻¹, 100 s⁻¹ to 200 s⁻¹, 100 s⁻¹ to 500 s⁻¹, 50 s⁻¹ to 500 s⁻¹, or 50 s⁻¹ or 1000 s⁻¹.

Alternatively or additionally, the device described herein further contains a heat transfer element, which can maintain the fluid at a predetermined temperature (e.g., a physiologically relevant temperature (e.g., a temperature that would be found in vivo in a healthy or diseased subject or one with a particular condition as provided herein), such as 30° C. to 45° C., preferably 37° C., 40° C. or 41° C.).

In some embodiments, non-microfluidic devices are provided. In some embodiments, the non-microfluidic device is AFM, optical tweezers, micropipette, magnetic twisting cytometer, cytoindenter, microindenter, nanoindenter, microplate stretcher, microfabricated post array detector, micropipette aspirator, substrate stretcher, shear flow detector, diffraction phase microscope, or tomographic phase microscope.

### Computational Methods, Systems and Devices

A computational framework is provided in some aspects that quantitatively predicts mechanical properties of deformable objection. The computational framework uses as inputs, in some cases, information (e.g., transit characteristics) about the passage of a deformable object through the microfluidic devices disclosed herein. For example, a computational framework is provided in some aspects that quantitatively predicts mechanical properties of healthy and infected red blood cells (RBCs) given the information about the passage of RBCs through micropores.

A computational approach for modeling deformable objects by means of a Dissipative Particle Dynamics (DPD) model, or other appropriate model, provides a unique means to assess the influence of a variety of different properties on the deformation of a deformable object. Depending on the deformable object, the properties may include size, shape, membrane shear modulus, membrane viscosity, bending modulus, viscosity of internal fluid and suspending medium. In some aspects, each of these properties can be varied independently of each other in model simulations.

In some aspects, computational models provided herein have led to the development of numerical closed form functions that can predict mechanical properties of deformable objects based on flow characteristics through a microfluidics device. Often the input parameters for the closed-form function include characteristics specific to the flow device used in the development of the model, and of the deformable object under investigation. For example, input parameters may include, dimensions of the constriction (micropore), applied pressure differential driving the flow, transit time of the object, and transit velocity of the object. The output of the closed-form function is typically a quantitative estimate of the value of a deformable object property, such as shear modulus or membrane viscosity. The approach can be generalized to constrictions of various dimensions, as disclosed herein, and any of the cells disclosed herein.

In some cases, methods are provided that involve performing one or more assays on one or more deformable objects to obtain a measurement of one or more mechanical properties; simulating, with at least one processor, flow of a fluid comprising more than one type of deformable object; and obtaining a closed-form equation with data from the simulation in combination with the measurement.

An illustrative example of the methods include at least obtaining data from at least one flow test performed on a fluid that contains more than one type of deformable object, and comparing the data with one or more predicted values calculated with at least one closed-form equation that correlates flow behavior to at least one material property (e.g., velocity, shear modulus, shear rate, shear stress, strain rate, yield stress, or hematocrit). Optionally, this method further includes one or more of: calculating the predicted values with the at least one closed-form equation, assessing the health of a subject from which the fluid is derived, and sorting and/or collecting one type of deformable object from another based on the comparison.

The flow test may be performed on a fluid under a predetermined set of microfluidic conditions, e.g., at a specific pressure, pressure gradient, velocity, etc. In one example, the flow test is performed by passing the fluid through one or more microfluidic channels, which can contain one or more constrictions or form part of a microfludic device (e.g., any of the microfludic devices described herein). In another example, the flow test is performed by passing the fluid through a microbead suspension, a flow cytometer, or a suspended microchannel resonator. A combination of different flow tests and/or mechanical or rheological assessments may be used in some cases.

The fluid can contain more than one type of cell (e.g., a mixture of both healthy and diseased cells), vesicles, biomolecular aggregates, platelet or particle, or a combination thereof. In one example, the fluid contains red blood cells, white blood cells, epithelial cells, or a mixture thereof. In another example, it contains cancer cells. In yet another example, the fluid (e.g., whole blood) contains T cells, B cells, platelets, reticulocytes, mature red blood cells, or a combination thereof. In some case, the fluid is substantially pure. The fluid may be whole-blood, serum, or plasma.

Any of the cells disclosed herein may be used in the methods. For example, epithelial cells of the cervix, pancreas, breast or bladder may be used. Red blood cells may be used, including, for example, fetal red blood cells, red blood cells infected with a parasite, red blood cells from a subject having or is suspected of having a disease, such as diabetes, HIV infection, anemia, cancer (e.g., a hematological cancer such as leukemia), multiple myeloma, monoclonal gammopathy of undetermined significance, or a disease that affects the spleen.

Flow test data can include a value for a transit characteristic, e.g., the velocity for one of the deformable objects, the average velocity for a population of the deformable objects, the distance traveled by one of the deformable objects, the time for one of the deformable objects to travel a certain distance, the average distance traveled by a population of the deformable objects or the average time for a population of the deformable objects to travel a certain distance.

A further illustrative method involves obtaining a value for one or more mechanical properties of a deformable object, determining a rheologic property (e.g., velocity) of the fluid described herein comprising the deformable object using a closed-form equation that correlates the mechanical property with the rheologic property, and optionally, making a prediction about the health of a subject (e.g., a subject having malaria or diabetes) based on the determination of the rheologic property. The one or more mechanical properties can be measured by, e.g., AFM, optical tweezers, micropipette, magnetic twisting cytometer, cytoindenter, microindenter, nanoindenter, microplate stretcher, microfabricated post array detector, micropipette aspirator, substrate stretcher, shear flow detector, diffraction phase microscope, or tomographic phase microscope. The prediction can include an assessment of the aggregation of the deformable objects in the fluid.

Data comparison can be performed using at least one processor. The at least one close-form equation employed in this step can be developed from one or more simulations of flow of a fluid in combination with experimental data. The one or more stimulations can be performed using dissipative particle dynamics model, a stochastic bond formation/dissociation model, or other appropriate model. The experimental data preferably is from an assay that measures membrane shear modulus, membrane bending rigidity, membrane viscosity, interior/exterior fluid viscosities, or a combination thereof, on a deformable object. However, any of a variety of experimental inputs may be used.

The step of assessing the health of a subject from which a fluid or cell is derived can be performed by determining the presence or absence of a disease or condition in the subject or determining the stage of a disease or condition.

An further illustrative example of the methods include obtaining data for one or more mechanical properties of a deformable object, and determining one or more predicted values of flow behavior. The one or more predicted values are determined with at least one closed-form equation that correlates flow behavior of any of the fluids or cells described herein to the one or more material properties (e.g., mechanical and/or rheological properties) of the fluid or a component thereof. For example, one or more predicted values may determined with at least one closed-form equation that correlates flow behavior of blood to the one or more rheological properties of the blood. Information regarding the rheological properties of the blood may be used to evaluate the likelihood of a clinical condition, e.g., aggregate formation, capillary occlusion in the brain, heart or other tissue, etc. in a subject. Thus, the closed form equation together with information regarding the flow behavior of a biological fluid obtained from a subject may be used in some case to diagnosis or evaluate a disease or condition in the subject.

Apparatus are provided in some aspects for performing at least one of the methods described herein. An illustrative example of such an apparatus contains a device for performing a flow test on a fluid, a computer system for obtaining data from the flow test and comparing the data with one or more predicted values. Alternatively, the apparatus contains a device for obtaining data for one or more mechanical properties of a deformable object, and a computer system for obtaining the data and determining one or more predicted values. The predicted value(s) can be calculated with at least one closed-form equation that correlates flow behavior of the deformable object-containing fluid described herein to the one or more mechanical properties.

Also provided are methods for manufacturing a diagnostic test apparatus that contains a device either for performing a flow test or for determining one or more mechanical properties of a deformable object; and a computing device that predicts at least one rheologic property of a sample (e.g., any of the deformable object-containing fluids described herein) based on flow behavior measured on the sample passing through the device, compares a value for a measurement of a sample as it passes through the device, or calculates one or more predicted values for flow behavior of the fluid described herein. Further methods may include generating, with at least one processor and a model of deformable objects within a fluid, a closed-form equation relating at least one parameter of flow of the fluid through the device to the at least one rheologic property; and encoding the closed-form equation in software configured for execution on the computing device. In another example, this method includes comparing, with at least one processor, the value with one or more predicted values calculated with a closed-form equation relating at least one parameter of flow of the fluid to at least one rheologic property; and encoding the one or more predicted values in software configured for execution on the computing device.

In some embodiments, the apparatus comprises a non-microfluidic device. In some embodiments, the non-microfluidic device is AFM, optical tweezers, micropipette, magnetic twisting cytometer, cytoindenter, microindenter, nanoindenter, microplate stretcher, microfabricated post array detector, micropipette aspirator, substrate stretcher, shear flow detector, diffraction phase microscope, or tomographic phase microscope.

Manufacturing methods include calculating, with at least one processor, one or more predicted values with the one or more mechanical properties, the one or more predicted values being calculated with a closed-form equation relating at least one parameter of flow of the fluid to the one or more mechanical properties; and encoding the one or more predicted values in software configured for execution on the computing device.

In addition, the present invention features a method including an inputting step and a calculating or comparing step. The inputting step can be performed by inputting a value for a measurement of any of the deformable object-containing fluids described herein as it passes through a flow test device. Alternatively, it is performed by inputting a value for one or more mechanical properties of a deformable object. The calculating step can be performed by calculating at least one mechanical or rheological property with a closed-form equation and the inputted value, the equation relating at least one parameter of flow of the fluid through the device to the at least one mechanical or rheological property, or by calculating one or more predicted values for flow behavior of any of the fluids described herein, the one or more predicted values being calculated with a closed-form equation relating at least one parameter of flow of the fluid the one or more mechanical properties. The comparing step may involve comparing the value with a predicted value from a calculation with at least one closed-form equation that correlates flow behavior to at least one mechanical or rheological property. Any of the methods described in this paragraph can further involve calculating the predicted value with the closed-form equation.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above. As used herein, the term “non-transitory computer-readable storage medium” encompasses only a computer-readable medium that can be considered to be a manufacture (i.e., article of manufacture) or a machine.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

### Assessment of T-Cells

Aspects of the invention are based on the recognition that changes in the mechanical properties (e.g., apparent Young's (elastic) modulus) of T cells occur as a result of the T cell activation process. It has been discovered, for example, that the apparent Young's modulus of T cells decreases upon activation. It has been further discovered, in some aspects, that certain T cells obtained from subjects having a T-cell related disease (e.g., T lymphocytes from subjects having Wiskott-Aldrich Syndrome (WAS)) exhibit differences, compared with normal T-cells, with respect to the extent to which changes in mechanical properties (e.g., Young's Modulus) occur during T-cell activation. Accordingly, methods are provided for analyzing material properties and activation states of T-cell. The methods typically involve analyzing the deformability of a T cell, and determining the activation state of the T cell based on the analysis.

Methods are provided for identifying candidate therapeutic agents that modulate T-cell activation. In some embodiments, the candidate therapeutic agents enhance T-cell activation. In some embodiments, the candidate therapeutic agents inhibit T-cell activation. The methods typically involve assessing mechanical properties of T-cells during activation in the presence or absence of a therapeutic agent. Any of the therapeutic agents or candidate therapeutic agents disclosed herein may be used. In some cases, the methods involve determining the deformability of a T cell, contacting the T cell with a compound, and analyzing the deformability of the T cell after contact with the compound.

A further illustrative method involves contacting a T cell with a compound or protein that affects the deformability of the T cell. Examples of such compound include, but are not limited to, cytochalasins, latrunculin A and B, nocodazole, colchicine, vincristine, colcemid, or paclitaxel. In some embodiments, the compound is attached to a solid surface. In some embodiments, the protein may be a cytokine, growth factor or antibody. The cytokine may be, for example, IL-2, IL-4, IL-7, IL-15, or IL-21. The antibody may be, for example, an antibody, or antibody fragment, that is specific for a T-cell surface protein such as, for example, CD3, CTLA4, CD28 or IL-7R. The contacting step can be performed by administering the compound to a subject, e.g., a subject in need of an improved or reduced or inhibited T cell response. In one example, the subject has or is suspected to have a disease or condition for which an improved T cell response is beneficial. In another embodiment, the subject has or is suspected to have a disease or condition for which a reduced or inhibited T cell response is beneficial. In one example, the subject has or is suspected to have a disease or condition for which a T cell response is detrimental. In some embodiments, the subject has cancer, an autoimmune disease, an infection or an infectious disease.

Pharmaceutical compositions for use in eliciting or inhibiting a T cell response are provided in some aspects. Compositions are provided that comprise a compound that affects deformability of a T cell, as is the use of the composition in manufacturing a medicament for eliciting a T cell response.

Cytoadherence Methods

Methods for evaluating cell adhesion properties of cells are provided in some aspects. The methods may involve the use of a device, such as an atomic force microscope (AFM), to probe cell adhesion. An illustrative method includes attaching a first type of cell to a first surface, attaching a second type of cell to a first surface, attaching the second type of cell to a second surface, contacting the two types of cells and then separating the second type of cell from the first type of cell, and determining the adhesion force between the first type of cell and the second type of cell. According to the method, the force of binding satisfies the following relationship:

f_{A2}>f_{A1},f_{A3},

and wherein f_{A1} is the force of binding of the second type of cell to the first surface, f_{A2} is the force of binding of the second type of cell to a second surface, and f_{A3} is the force of binding of the second type of cell to the first type of cell. In some embodiments, the cell is a nucleated cell. In other embodiments, the cell is a non-nucleated cell.

A further illustrative method includes attaching a first type of cell to a first surface by, e.g., growing the first type of cell on the first surface, attaching a second type of cell to a second surface by initially stabilizing the second type of cell through light adhesion to the first surface and subsequently transferring it to the second surface through mediation with a stronger adhesive molecule, contacting the two types of cells and then separating the second type of cell from the first type of cell, and determining the adhesion force between the first type of cell and the second type of cell with an atomic force microscope (AFM). The second surface can be the surface of a tipless cantilever. When necessary, the first surface is functionalized with a molecule that lightly binds to the second type of cell and the tipless cantilever is functionalized with a molecule that strongly binds to the second type of cell. The first surface may be functionalized for example with a polypeptide and the tipless cantilever may be functionalized for example, with a lectin protein.

Any of the cells disclosed herein may be used with any of the methods for evaluating cytoadherence. The first type of cell can be a cell that expresses a human receptor, e.g., CHO cells. The second type of cell can express a ligand that binds to the first type of cell via, e.g., interaction with the receptor expressed thereon. In one example, the second type of cell is infected or is suspected of being infected with, e.g., a microbe or parasite. In another example, the cell is diseased or is suspected of being diseased, e.g., a cancer cell. In yet another example, the second type of cell is a blood cell or the like, such as a T cell (activated or inactivated), a B cell, a vesicle, or a platelet. In one embodiment, the cell is infected or is thought to be infected with a microbe or a parasite.

In one example, the methods further involve assessing the health of a subject or selecting a therapeutic agent based on the determination of the adhesion force. In another example, the method further involves treating the first type of cell or the second type of cell with a candidate therapeutic agent. If desired, this method can further include, after the treating step, contacting the first type of cell and the second type of cell, subsequently separating the two types of cells, determining the adhesion force between the first type of cell and the second type of cell, and optionally, comparing the adhesion force before and after treatment with the candidate therapeutic agent.

Another illustrative method involves at least the following steps: determining the force of adhesion between a cell that is or is suspected to be diseased (e.g., being infected or suspected to be infected with a parasite) and another cell, and assessing whether or not the cell is diseased by comparing the force of adhesion with an appropriate standard, which can either be the force of adhesion of a healthy cell with the other cell or the force of adhesion of a diseased cell with the other cell. The force of adhesion between the cell that is or is suspected to be diseased and the other cell is determined with an assay (e.g., by using an AFM) such that the force relationship described above is satisfied.

Another illustrative method involves at least the following steps: force of adhesion between a diseased cell treated with a candidate agent and another cell, and comparing the force of adhesion with an appropriate standard, wherein the appropriate standard is the force of adhesion of either a diseased cell or a healthy cell with the other cell. The force of adhesion between the diseased, candidate agent-treated cell and another cell is determined with an assay such that the force relationship described above is satisfied.

In any of the methods described above, adhesion force determination can be performed at a physiologically relevant temperature, e.g., 37° C., 40° C. or 41° C.

All references described herein are incorporated by reference for the purposes described herein.

Exemplary embodiments of the invention will be described in more detail by the following examples. These embodiments are exemplary of the invention, which one skilled in the art will recognize is not limited to the exemplary embodiments.

### EXAMPLES

### Example 1

### An Automated Deformability Cytometer

An automated, microfabricated ‘deformability cytometer’ that measures dynamic mechanical responses of approximately 10³-10⁴ individual RBCs in a population has been developed. The device provides a novel method relying on low Reynolds number fluid mechanics to evaluate the effect of entrance architecture on the sensitivity of cell deformability measurements. The device can be used with many different cell types and used in field diagnostic applications. In some embodiments, optimized pore geometries have been identified using the device, which are suited for “deformability selection” of cells.

An algorithm was developed using commercially available software to automate video processing and facilitate the analysis of thousands of RBCs. In some embodiments, this high throughput device enabled the measurement of statistically significant differences in deformability between two cell populations. Fluorescence measurements on each RBC were simultaneously acquired with cell deformation measurements, resulting in a population-based correlation between biochemical properties (e.g. cell surface markers) and dynamic mechanical deformability.

The device design includes periodically spaced, triangle-shaped pillars and the gaps between these pillars result in well-controlled constrictions for RBCs to pass. The height of the device was set to 4.2 μm. RBCs were forced to assume a flat orientation before entering each constriction. This height, in addition to filters at the reservoirs, prevented white blood cells from entering the device, and permitted diluted whole blood to be used directly.

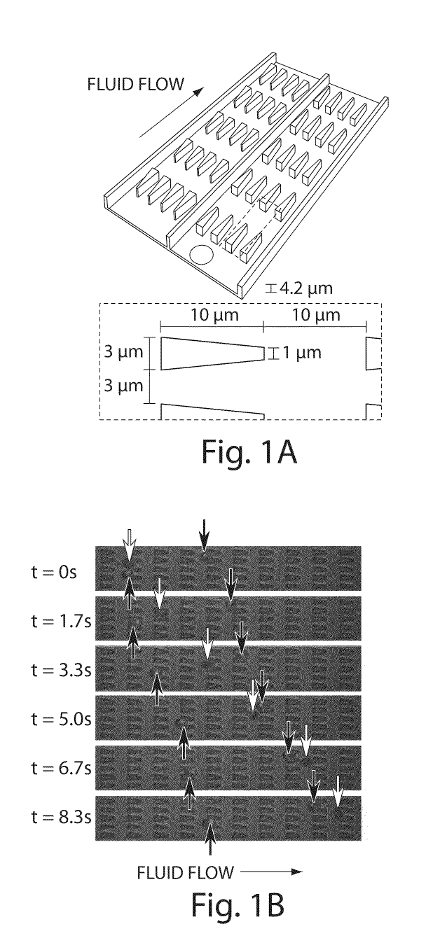

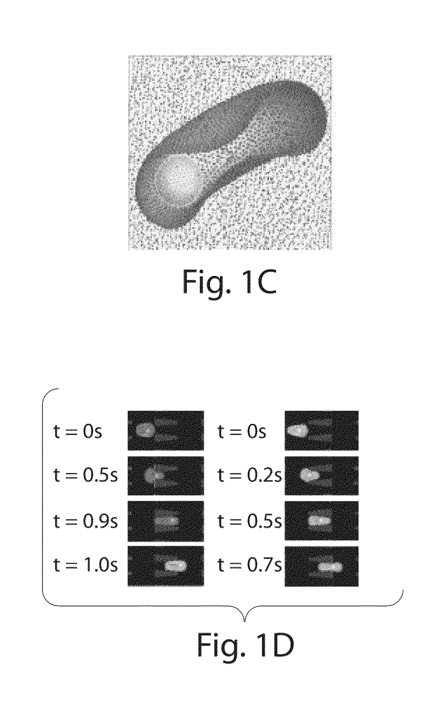

The concentration of RBCs was sufficiently low such that there was minimal interaction between cells and such that transit times were independent. Constrictions in parallel across the width of the channel provided high throughput, and constrictions in series along the length of the channel enabled repeated measurements of the same cell, which provided increased precision. FIGS. 1A and B illustrate the device design and depict infected and uninfected RBCs moving at different velocities across the channel.

### Materials and Methods

### Device Fabrication

A mold of the device was made on a silicon wafer using photolithography and reactive-ion etching techniques. A 5× reduction step-and-repeat projection stepper (Nikon NSR2005i9, Nikon Precision) was used for patterning. The spacing between pillars was 3 μm, and the depth of the device was 4.2 μm. Details regarding the device structure are presented in FIG. 1A. The device was made using standard PDMS casting protocols and bonded to a glass slide.

### Parasite Culture

P. falciparum was cultured in leukocyte-free human RBCs (Research Blood Components, Brighton, Mass.) under an atmosphere of 5% O₂, 5% CO₂ and 95% N₂, at 5% hematocrit in RPMI culture medium 1640 (Gibco Life Technologies) supplemented with 25 mM HEPES (Sigma), 200 mM hypoxanthyne (Sigma), 0.20% NaHCO₃ (Sigma) and 0.25% Albumax II (Gibco Life Technologies). Parasites were synchronized by treatment with 5% sorbitol at least 12 hours before sample collection. The strain FUP-GFP, expressing a GFPmut2-neo fusion protein, was constructed by transfecting P. falciparum strain FUP with the plasmid pFGNr (Malaria Research and Reference Reagent Resource Center). Parasites expressing GFPm2:neo were selected with 350 mg/L G-418. Transfection was performed by the spontaneous DNA uptake method (35).

### Experimental Protocol

PBS was mixed with 0.2% w/v Pluronic F-108 (BASF, Mount Olive, N.J.) and 1% w/v Bovine Serum Albumin (BSA) (Sigma-Aldrich, St. Louis, Mo.) as a stock solution to prevent RBC adhesion to the device walls. For the fluorescent bead experiments, 200 nm FluoSpheres europium luminescent microspheres (Molecular Probes, Eugene, Oreg.) diluted to a final concentration of 1.25×10⁻⁵ percent solids were used.

In experiments involving blood, 1 μl of whole blood (˜50% hematocrit) was diluted in 100 μl of the PBS-pluronic-BSA solution for all of the experiments. In experiments involving parasites that express GFP, no further treatment was performed. These cells appear as shadows with a small fluorescent circle inside, as shown in FIG. 1B. In experiments involving healthy RBCs, 1 μl of whole blood (Research Blood Components, Brighton, Mass.), 1 μl of 50 μg/ml of Cell Tracker Orange (Invitrogen, Carlsbad, Calif.), and 98 μl of PBS were mixed with the indicated concentration of glutaraldehyde and allowed to sit for 30 minutes. The sample was then washed 3 times with the PBS-Pluronic-BSA solution. In experiments involving reticulocytes, 1 μl of whole blood, 89 μl of the PBS-Pluronic-BSA solution, and 10 μl of 1×10⁻⁶M thiazole orange were mixed and allowed to sit for 20 minutes before starting experiments. Videos were obtained in which reticulocytes appear as uniformly fluorescent cells under the GFP filter set, while mature RBCs appear as shadows.

The PBS-Pluronic-BSA solution was pumped through the device for 30 minutes to coat the device walls with Pluronic and BSA. The RBC-PBS-Pluronic-BSA suspension was then injected into the device. Differences in pressure between the two reservoirs were generated hydrostatically by a difference in water column height. Liquid columns were connected to 60-ml plastic syringes lacking plungers to minimize surface tension effects. A Hamamatsu Model C4742-80-12AG CCD camera (Hamamatsu Photonics, Japan), connected to an inverted epi-fluorescent Olympus IX71 microscope (Olympus, Center Valley, Pa.) was used for imaging. IPLab (Scanalytics, Rockville, Md.) was used for video acquisition, resulting in an .avi file.

Data Analysis

A custom-written MATLAB program tracked the RBCs and generated data used for velocity histograms. This program first applies a high-pass filter to the video frames and then identifies RBCs based on areas of intensity above a certain threshold and within a preset size. After identifying the RBCs in a particular frame, the program first attempts to match the RBCs in the current frame to RBCs in the previous frame based on proximity. The program then takes the location and velocity of RBCs in the previous frame to confirm the match to RBCs in the current frame. The end result of this program is a video with RBCs identified by number and a spreadsheet of each RBC's velocity. The video was then checked for RBC identification accuracy.

Comparison of Fluid Velocities in Two Channels with Differing Constriction Geometry

The deformability cytometer device was used to analyze the effects of constriction geometry on cell traversal. Two otherwise identical, parallel microfluidic channels were designed such that only inlet geometries were characterized by different rates of constriction. Apart from this variable, the channels maintained according to laws of laminar flow, identical forward and backward flow velocities and resistances (19).

DPD simulations were used to confirm this assumption. The difference in fluid flow velocity between the two channels was less than 0.3%. This implies that bulk fluid resistance is independent of constriction geometry. A streamline study confirmed almost complete reversibility of the flow.

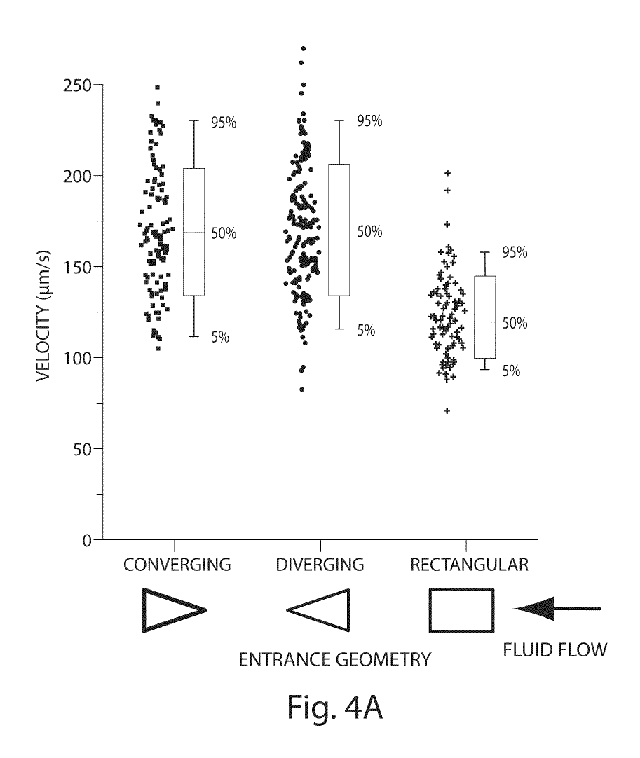

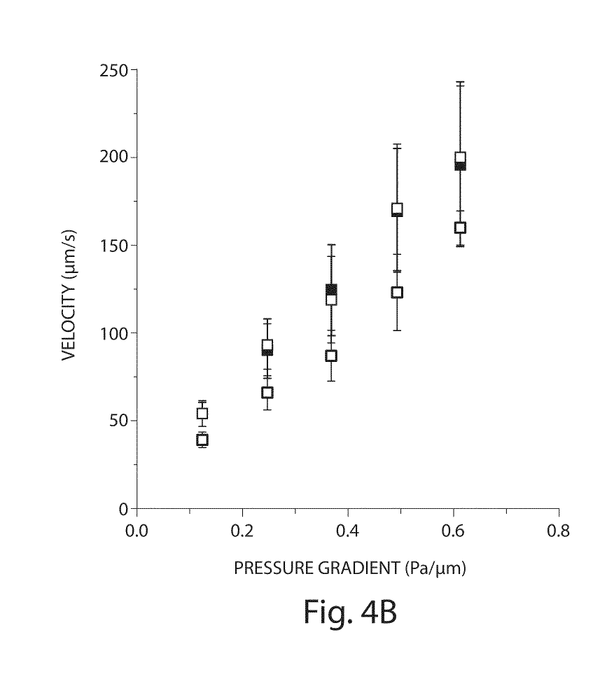

200 nm non-deformable polystyrene beads were introduced into the fluid in order to track fluid resistance and flow rate. Bead velocity through the channels showed no statistically significant differences when tested under experimental conditions (FIG. 4). Variation in bead velocities witnessed however may be attributed to viscous effects within the channels.

Experiments were performed with RBCs diluted to 1% hematocrit where cell-cell interactions were negligible and approximately 1000 cells could be analyzed in 10 minutes. The low concentration of cells enabled observation from a microscope.

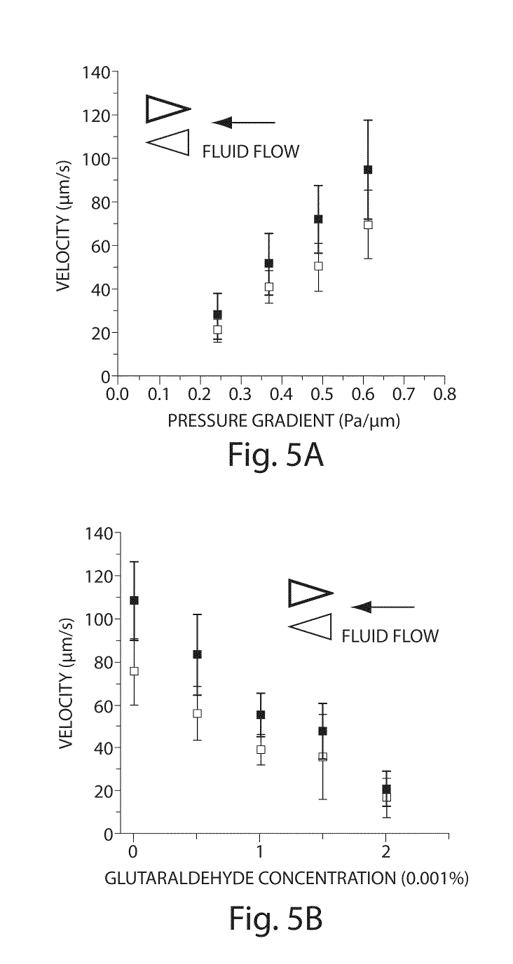

Different RBC velocities were obtained from flow through the two parallel channels. For given pressure gradients, RBCs exhibited faster velocity in the channel with converging entrance geometries (FIG. 5A). RBCs traveled 26% slower in channels with diverging geometries. Parameters such as temperature (15), cell age (20), buffer conditions (21), pressure, and device variability were held constant, thus indicating that constriction geometry plays a significant role in the effects of cell deformation.

### Effect of RBC Stiffness on Velocity Through Differing Constriction Geometry

RBCs treated with increasing concentration of glutaraldehyde for a given period of time results in increased cell stiffness (22). For concentrations of glutaraldehyde less than 0.002% and treatment for 30 minutes, more than 95% of the RBCs passed through the channels. As concentration increased, RBCs became progressively stiffer with decreasing velocity shown in FIG. 5B. For concentrations greater than 0.003%, most RBCs were held up at the entrance to the channel, unable to deform. Cell shape and size are preserved during glutaraldehyde treatment (22) and thus, these experiments demonstrated that reduced deformability alone leads to slower RBC travel through the given device.

Deformability of Late Ring-Stage P. falciparum-Infected RBCs

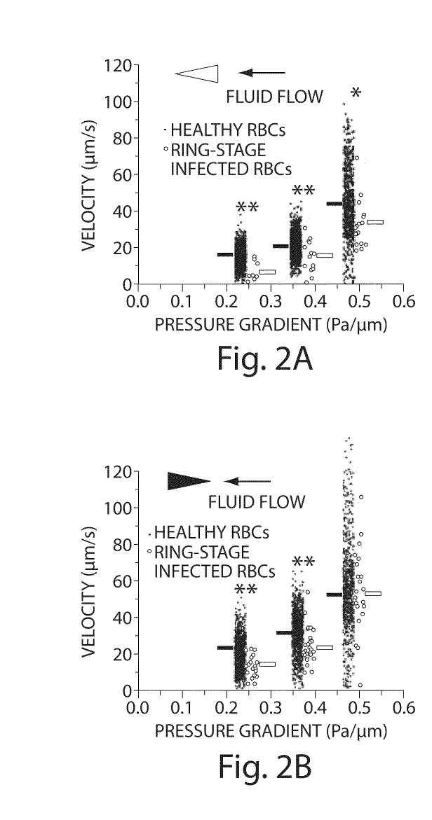

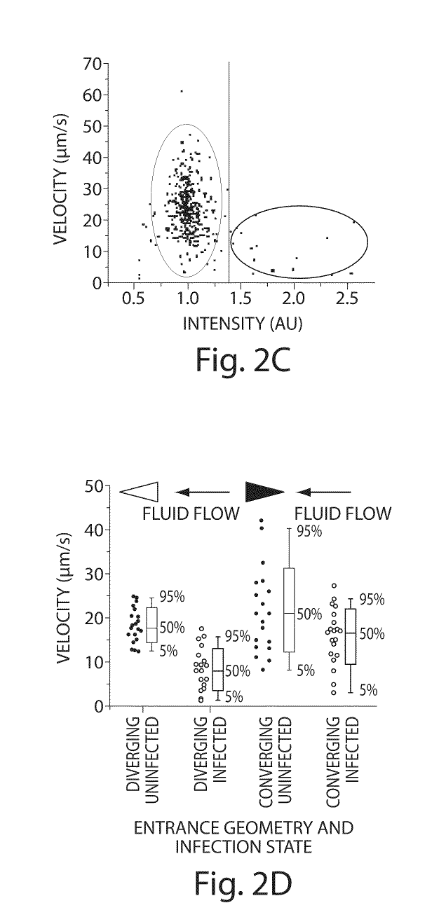

A set of experiments was performed using late ring-stage P. falciparum-infected RBCs that were transfected with a gene encoding green fluorescent protein (GFP) (FIG. 2). Treatment with cell dyes may influence the deformability of the cells (23), though cell dye effects were not evaluated. An image analysis program tracked a shadow with a bright dot inside as an infected RBC, and a shadow without a bright dot inside as an uninfected RBC. Parasitemia was approximately 1-2% with 1000 RBCs tracked for each pressure gradient in the range of 0.24 Pa/μm to 0.37 Pa/μm. Additional RBCs were tested at 0.48 Pa/μm.

In these experiments, negligible pitting or expulsion of the parasite from the RBC was observed. For both converging and diverging geometries, pressure gradients 0.24 Pa/μm and 0.37 Pa/μm, infected RBCs exhibited lower average velocities than uninfected RBCs with a statistically significant p-value less than 0.01. For increasingly higher pressure gradients, mean velocities of infected and healthy RBCs converged. For a pressure gradient of 0.48 Pa/μm, both healthy and infected RBCs moved through the converging geometry at the same velocity (50 μm/s).

FIG. 2D illustrates how a diverging geometry accentuates differences in deformability between ring-stage infected cells and uninfected cells. The median velocity of infected cells in the diverging geometry was 44% of that of the uninfected cells, compared to 80% in the converging geometry.

### Deformability of Reticulocytes Contained in Whole Blood

Reticulocytes are considered immature RBCs and account for 1% of RBCs in a sample of whole blood. In contrast to mature RBCs, reticulocytes contain residual amounts of RNA, are larger, with a 44 μm² greater surface area and 29 fL greater volume (24) and more rigid. Consequently, reticulocytes take longer to enter a single pore (25), and demand a higher driving pressure to compress the reticulocyte membrane and force into a pipette (23). The membrane shear elastic modulus of reticulocytes is almost double that of mature RBCs (26).

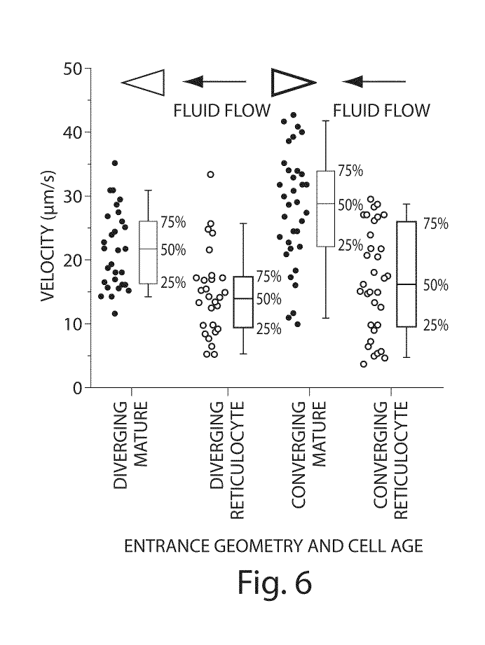

In this set of experiments, whole blood was diluted in phosphate-buffered saline (PBS) containing thiazole orange, a nucleic acid stain for reticulocytes. White blood cells were removed at the inlet of the device and therefore did not interfere with the operation of the device. Reticulocytes exhibited average velocities 67% of mature RBCs in the diverging geometry, and 61% of mature RBCs in the converging geometry as shown in FIG. 6.

### Temperature Dependence on Deformability

Experiments were conducted to ascertain the effects of temperature on deformability for both healthy and malaria infected RBCs. The differences were more prominent with increasing temperature. This difference may be used (e.g., as a biomarker) to clearly delineate between rare, diseased cells and a larger normal cell population.

### Example 2

### Dissipative Particle Dynamics (DPD) Simulation of Cell Deformation Through Different Constriction Geometries

A Dissipative Particle Dynamics (DPD) model was built to translate the experimental measurements from the deformability cytometer into quantitative data describing the mechanical properties of individual RBCs.

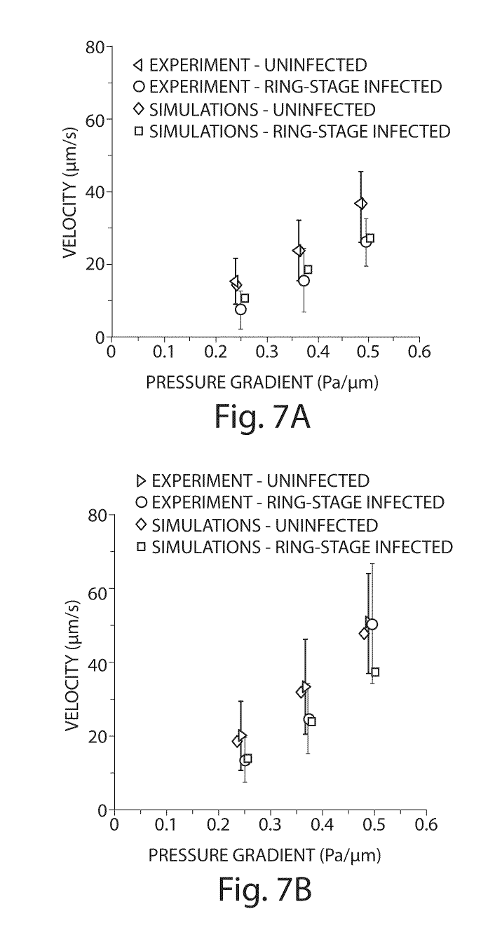

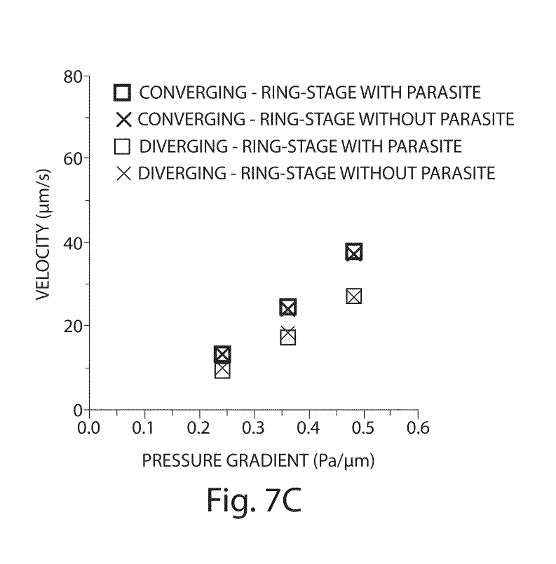

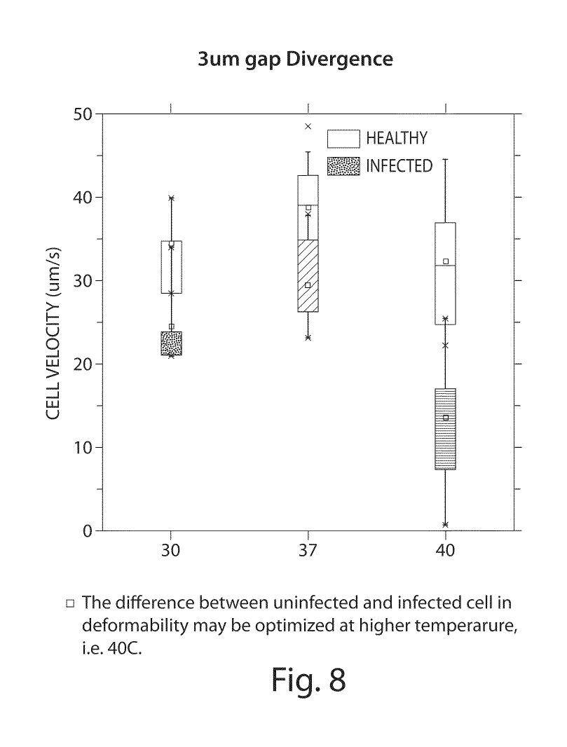

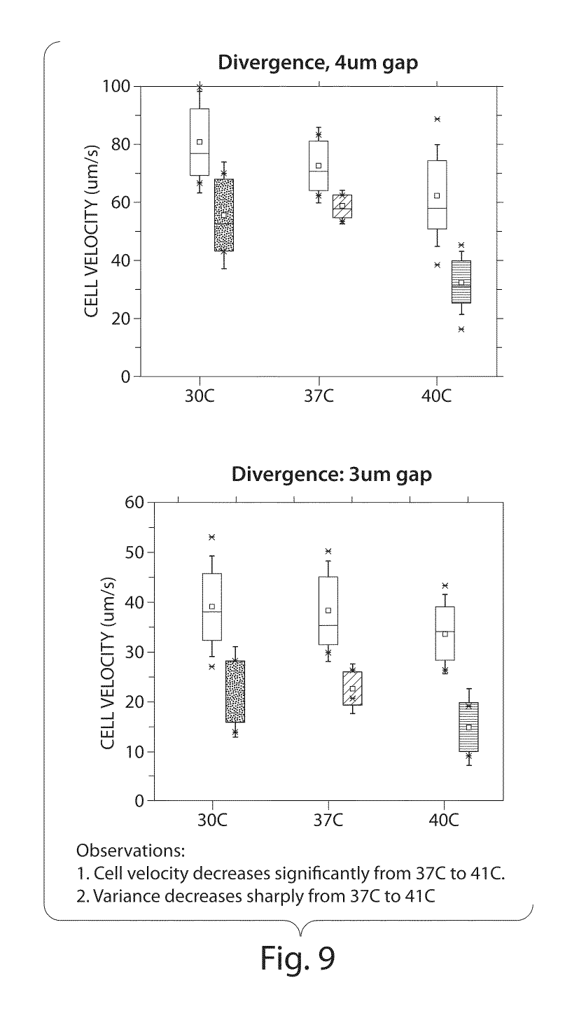

Three-dimensional simulations of healthy and malaria-infected cells were performed using the DPD method. Infected cells were modeled with increased shear modulus and membrane viscosity values obtained from quantitative experimental measurements performed by recourse to optical tweezers stretching of the parasitized RBCs (15). The parasite was modeled as a rigid sphere, 2 microns in diameter (27), placed inside the cell (FIG. 1C). Snapshots from simulations showing passage of an infected RBC through channels with converging and diverging pore geometries are shown in FIG. 1D. Simulations were able to capture the effects of pore geometry and changes of RBC properties arising from parasitization quite well. Quantitative comparison of simulation results with experimental data for healthy and infected cell velocity as a function of applied pressure gradient is shown in FIGS. 7 A and B.

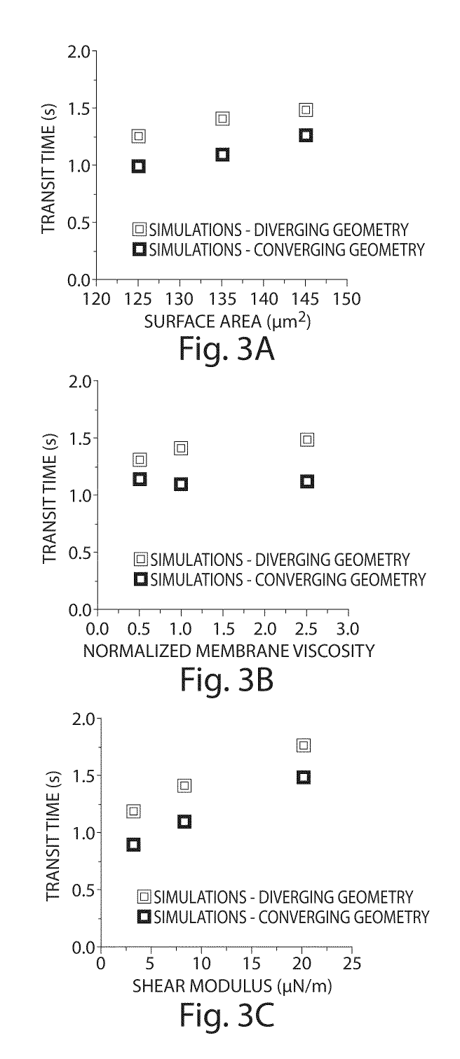

Additional simulations were performed to evaluate contributions of individual mechanical properties of the cell to overall dynamic behavior. Using the DPD model, the flow behavior of infected RBCs in the device was observed to not be affected by the presence of the parasite inside the cell (FIG. 7C). Larger cells were found to travel with lower velocities; however, the velocity variation due to cell size was not found to be significant based on certain model input parameters (FIG. 3A). The decrease in traverse velocity of infected RBCs observed in the cytometry device may be due to the increase of membrane shear modulus and/or membrane viscosity. Additional simulations were performed in which membrane shear modulus and membrane viscosity were varied independently of each other. The results showed that shear modulus was a dominant factor, compared with membrane viscosity, and that variation of membrane viscosity did not contribute significantly to the decrease of velocity of infected cells.

Increased membrane viscosity may increase the time it takes for a RBC to traverse an individual pore. However, it also slows down the recovery of RBC shape when the cell is traveling between pores, making it easier to enter the next pore. As a result, certain device designs may lessen the dependence of cell velocity on membrane viscosity (FIG. 3B). For example, increased membrane shear modulus increases the transit time for an individual pore and also accelerates shape recovery, making it more difficult to enter the next pore, depending on the device configuration. FIG. 3C shows the variation of time it takes a cell to travel from one set of obstacles to a next set of obstacles at a pressure gradient of 0.24 Pa/μm as a function of membrane shear modulus. To a first approximation, the time increases linearly with shear modulus within the range considered in simulations. This dependence can be an advantage if the device is used to estimate the average shear modulus of a cell population based on the average velocity. For higher values of shear modulus, the transit time may become a non-linear function; however, stiffer cells (e.g. shear modulus greater than 30 μN/m, (15)) may be cleared by the spleen and therefore not typically present in free circulation.

Simulation Setup

The Dissipative particle dynamics (DPD) (36) method was employed in simulations. In DPD, the fluid, solid walls, and RBC membrane were represented by collections of particles. The particles interact with each other through soft pairwise forces: conservative, dissipative, and random force. The latter two form the DPD thermostat and are linked through the fluctuation-dissipation theorem. The viscosity of the DPD fluid can be varied by changing the functional form and magnitude of these forces (37). The solid walls were assembled from randomly distributed DPD particles whose positions were fixed during the simulations. In addition, bounce-back reflections were used to achieve no-slip conditions and prevent fluid particles from penetrating the walls (38). A portion of the microfluidic device with dimensions 200 by 120 by 4.2 microns containing 5 rows of pillars (10 pillars in each row) was modeled. The fluid region was bounded by four walls while periodic boundary conditions were used in the flow direction. The RBC was simulated using 5000 DPD particles connected with links (39). The model took into account bending, in-plane shear energy, and membrane viscosity. The effect of membrane viscosity was modeled by adding frictional resistance to each link. The total area and volume were controlled through additional constraints. Parameters of the healthy cell model were derived from RBC spectrin network properties (39-41). In addition, membrane fluctuation measurements and optical tweezer experiments were used to define simulation parameters.

The amplitude of thermal fluctuations of the membrane at rest was required to be within the range of experimental observations (42). The characteristic relaxation time of the RBC model in simulations, was required to be equal to the experimentally measured value of 0.18 seconds. For P. falciparum infected cells, the membrane shear modulus and viscosity were increased 2.5 times (15). The malaria parasite was modeled as a rigid sphere, 2 microns in diameter. The RBC model was immersed into the DPD fluid. The membrane particles interacted with internal and external fluid particles through the DPD forces. The viscosity of the internal fluid was 9 times higher than external fluid viscosity. The flow was sustained by applying a body force to the DPD particles. By changing the direction of the body force, the motion of the cell through channels with converging and diverging pores was simulated using the same channel geometry.

### References for Background and Examples 1 and 2

1. McMillan D, Utterback N, La Puma J (1978) Reduced erythrocyte deformability in diabetes. Diabetes 27: 895-901.
2. Cranston H, et al. (1983) Plasmodium falciparum maturation abolishes physiologic red cell deformability. Science 233: 400-403.
3. Mokken F C, Kedaria M, Henny C P, Hardeman M, Gleb A (1992) The clinical importance of erythrocyte deformability, a hemorrheological parameter. Ann. Hematol. 64: 113-122.
4. WHO (2008) World Malaria Report 2008
5. Maier A, Cooke B, Cowman A, Tilley L (2009) Malaria parasite proteins that remodel the host erythrocyte. Nature Reviews Microbiology 7: 341-354.
6. Suresh S, et al. (2005) Connections between single-cell biomechanics and human disease states: gastrointestinal cancer and malaria. Acta Biomaterialia 1: 16-30.
7. Safeukui I, et al. (2008) Retention of Plasmodium falciparum ring-infected erythrocytes in the slow, open microcirculation of the human spleen. Blood 112: 2520-2528.
8. van der Heyde J, Nolan J, Combes V, Gramaglia I, Grau G (2006) A unified hypothesis for the genesis of cerebral malaria: sequestration, inflammation and hemostasis leading to microcirculatory dysfunction. Trends in Parasitology 22: 503-508.
9. Reid J, Barnes A, Lock P, Dormandy J, Dormandy T (1976) A simple method for measuring erythrocyte deformability. J. Clin. Pharmacol. 29: 855-858.
10. Bessis M, Mohandas N (1977) A diffractometric method for the measurement of cellular deformability. Blood Cells 1: 307-313.
11. Nash G, O'Brien O, Gordon-Smith E, Dormandy J (1989) Abnormalities in the mechanical properties of red blood cells caused by plasmodium falciparum. Blood 74: 855-861.
12. Chien S (1977) Principles and techniques for assessing erythrocyte deformability. Blood Cells 3: 71-99.
13. Lekka M, Formal M, Pyka-Fosciak G, Lebed K, Wizner B (2005) Erythrocyte stiffness probed using atomic force microscopy. Biorheology 42: 307-317.
14. Guck J, et al. (2001) The optical stretcher: a novel laser tool to micromanipulate cells. Biophysical Journal 81: 767-784.
15. Mills J P, et al. (2007) Effect of plasmodial RESA protein on deformability of human red blood cells harboring Plasmodium falciparum. PNAS 104: 9213-9217.
16. Brody J P, Han Y, Austin R H, Bitensky M (1995) Deformation and flow of red blood cells in a synthetic lattice: evidence for an active cytoskeleton. Biophysical Journal 68: 2224-2232.
17. Abkarian M, Faivre M, Stone H (2006) High-speed microfluidic differential manometer for cellular-scale hydrodynamics. PNAS 103: 538-542.
18. Shelby J P, White J, Ganesan K, Rathod P K, Chiu D T (2003) A microfluidic model for single-cell capillary obstruction by plasmodium falciparum-infected erythrocytes. PNAS 100: 14618-14622.
19. Deen W (1998) Analysis of Transport Phenomena (Oxford University Press, New York) p 292.
20. Chien S (1987) Red cell deformability and its relevance to blood flow. Ann. Rev. Physiol. 49: 177-192.
21. Rand R P, Burton A C (1964) Mechanical properties of the red cell membrane. Biophysical Journal 4: 115-135.
22. Tong X, Caldwell K D (1995) Separation and characterization of red blood cells with different membrane deformability using steric field-flow fractionation. Journal of Chromatography B 674: 39-47.
23. Leblond P, LaCelle P, Weed R (1971) Cellular deformability: a possible determinant of the normal release of maturing erythrocytes from the bone marrow. Blood 37: 40-46.
24. Gifford S, Derganc J, Shevkoplyas S, Yoshida T, Bitensky M (2006) A detailed study of time-dependent changes in human red blood cells: from reticulocyte maturation to erythrocyte senescence. British Journal of Haematology 135: 395-404.
25. Waugh R (1991) Reticulocyte rigidity and passage through endothelial-like pores. Blood 78: 3037-3042.
26. Xie L, et al. (2006) Studies on the biomechanical properties of maturing reticulocytes. Journal of Biomechanics 39: 530-535.
27. Enderle T, et al. (1997) Membrane specific mapping and colocalization of malarial and host skeletal proteins in the Plasmodium falciparum infected erythrocyte by dual-color near-field scanning optical microscopy PNAS 94: 520-525.
28. Secomb T, Hsu R (1996) Analysis of red blood cell motion through cylindrical micropores: Effects of cell properties. Biophysical Journal 71: 1095-1101.
29. Bathe M, Shirai A, Doerschuk C, Kamm R (2002) Neutrophil transit times through pulmonary capillaries: the effects of capillary geometry and fMLP-stimulation. Biophysical Journal 83: 1917-1933.
30. Hochmuth R, Worthy P, Evans E (1979) Red cell extensional recovery and the determination of membrane viscosity. Biophysical Journal 26: 101-114.
31. Shirai A, Fujita R, Hayase T (2003) Transit characteristics of a neutrophil passing through two moderate constrictions in a cylindrical capillary vessel (Effect of cell deformation on transit through the second constriction). JSME International Journal 46: 1198-1207.
32. Groisman A, Enzelberger M, Quake S (2003) Microfluidic memory and control devices. Science 300: 955-958.
33. Gifford S C, et al. (2003) Parallel microchannel-based measurements of individual erythrocyte areas and volumes. Biophysical Journal 84: 623-633.
34. Higgins J, Eddington D, Bhatia S, Mahadevan L (2007) Sickle cell vasoocclusion and rescue in a microfluidic device. PNAS 104: 20496-20500.
35. Deitsch K, Driskill C, Wellems T (2001) Transformation of malaria parasites by the spontaneous uptake and expression of DNA from human erythrocytes. Nucleic Acids Res. 29: 850-853.
36. Groot R D, Warren P B (1997) Dissipative particle dynamics: Bridging the gap between atomistic and mesoscopic simulation. J Chem Phys 107: 4423-4435.
37. Fan X J, Phan-Thien N, Chen S, Wu X H, Ng T Y (2006) Simulating flow of DNA suspension using dissipative particle dynamics. Phys Fluids 18: 063102.
38. Pivkin I V, Karniadakis G E (2005) A new method to impose no-slip boundary conditions in dissipative particle dynamics. Journal of Computational Physics 207: 114-128.
39. Pivkin I V, Karniadakis G E (2008) Accurate coarse-grained modeling of red blood cells. Phys Rev Lett 101: 118105.
40. Discher D E, Boal D H, Boey S K (1998) Simulations of the erythrocyte cyto skeleton at large deformation. II. Micropipette aspiration. Biophysical Journal 75: 1584-1597.
41. Li J, Dao M, Lim C T, Suresh S (2005) Spectrin-level modeling of the cytoskeleton and optical tweezers stretching of the erythrocyte. Biophysical Journal 88: 3707-3719.
42. Park Y, et al. (2008) Refractive index maps and membrane dynamics of human red blood cells parasitized by Plasmodium falciparum. PNAS 105: 13730-13735.

### Example 3

### Combined Simulation and Experimental Study of Large Deformations of Red Blood Cells in Microfluidic Systems

The biophysical characteristics of healthy human red blood cells (RBCs) traversing microfluidic channels with cross-sectional areas as small as 2.7μ×3 μm were evaluated. Single RBC flow experiments were combined with corresponding simulations based on dissipative particle dynamics (DPD). Upon validation of the DPD model, predictive simulations and companion experiments were performed in order to quantify pressure-velocity relationships for different, channel sizes and physiologically-relevant temperatures. Conditions associated with the shape transitions of RBCs were examined along with the relative effects of membrane and cytosol viscosity, plasma. environments, and geometry on flow through microfluidic systems at physiological temperatures. A cross-sectional area threshold was determined below which RBC membrane properties begin to influence its flow behavior at room and physiological temperatures.

### Results

High-speed imaging was used to measure and quantify the temperature-dependent flow characteristics (pressure versus velocity relationships) and shape transitions of RBCs as the RBCs traverse microfluidic channels of varying characteristic size. These results were compared to simulated flow behavior using Dissipative Particle Dynamics (DPD). A feature of the modeling approach was that the interaction parameters governing the elastic behavior of the RBC membrane were derived from the properties of the individual components of the RBC cytoskeleton. Therefore, the model was capable of capturing the elastic behavior of the RBC without additional fitting parameters. The viscous parameters were defined using additional independent experimental measurements. As a result, the RBC model accurately matched the behavior measured in three different experiments at both room and physiological temperatures:

1. the force-displacement response as measured with optical tweezers (42);

2. the magnitude of resting membrane thermal fluctuations (40); and

3. the characteristic time scale of membrane relaxation following stretching (19, 35).

The membrane and fluid parameters determined from this diverse combination of experiments were applied for subsequent modeling conditions and were complemented with the results of a single data point from the RBC flow experiments in order to translate non-dimensional simulation results to physical units. More details of the modeling scheme, flow control system, channel geometry, as well as our procedure for determining local pressure gradients across the microfluidic channel are described below.

### Evaluation of RBC Deformation

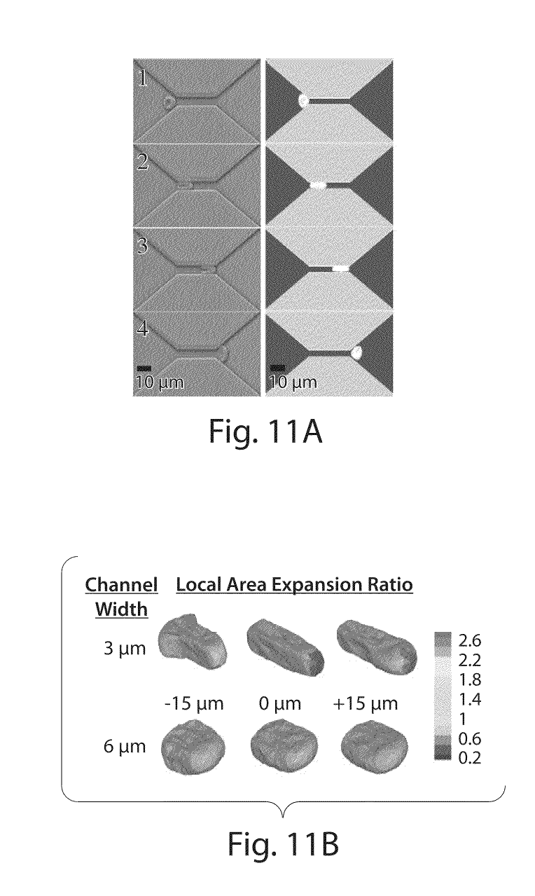

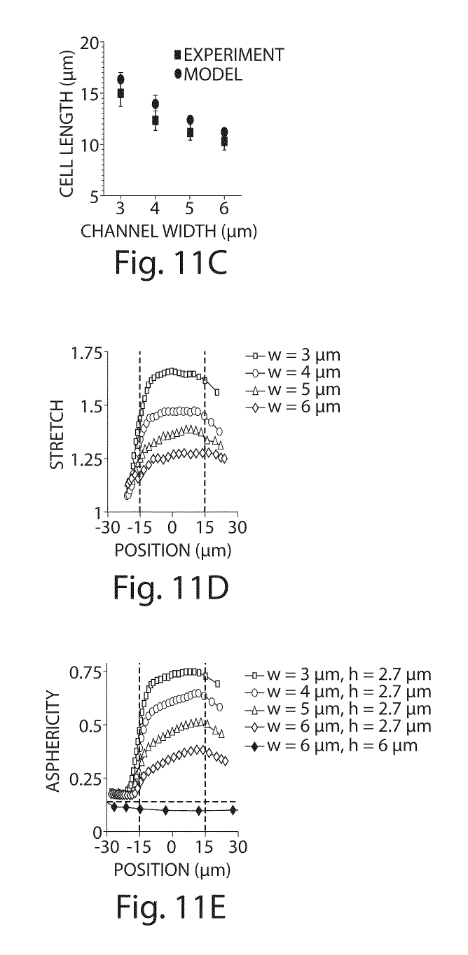

FIG. 11 illustrates shape profiles of the RBC as it traverses channels that are 2.7 μm high, 30 μm long and 3 to 6 μm wide, geometries typical of some of the large deformation conditions in the microvasculature. FIG. 11(a) illustrates a qualitative comparison of experiment with the DPD model for RBC traversal across a 4 μm wide channel. Three time scales were identified:
(Frames 1-2) the time required for the cell to go from its undeformed state to being completely deformed in the channel;
(Frames 2-3) the time it takes the cell to traverse the channel length, and
(Frames 3-4) the time for complete egress from the channel.

Here the cell underwent a severe shape transition from its normal biconcave shape to an ellipsoidal shape with a longitudinal axis up to 200% of the average undeformed diameter. FIG. 11(c) provides an illustration of how the longitudinal axis of the cell, measured at the center of the channel, changed with different channel widths. Experimental and simulated longitudinal axes typically differed no more than 10-15%. During such large deformation, the RBC membrane surface area and volume were assumed to be constant in our DPD model. However, the model allowed for local area changes during passage through the channel. The contours presented in FIG. 11(b) exemplify the evolution of such local gradients in area expansion. These results indicated that, for the smallest length scales, the leading edge of the cell deformed significantly as the cell entered the constriction and deformed further as the cell traversed the channel. Modest area expansion was observed during flow through the 2.7 μm high×6 μm wide channel. The local stretch of the underlying spectrin network scales as the square root of local area expansion. Therefore, this information may be used to estimate the maximum stretch of the spectrin network at a point during this traversal process. This result is exemplified in FIG. 11(d) for the channel widths used in the experiments. At the smallest width channels, the maximum stretch increased to λ≧1.6.

FIG. 11(e) shows a comparison between the results of the shape characteristics and the results of other mesoscale modeling approaches, such as the multiparticle collision dynamics (NIPC) models presented by McWhirter et. al. (32). Deviation of the RBC shape from that of a sphere was quantified by its average asphericity <α>, where <α>=0 for a sphere and <α>=0.15 for an undeformed discocyte. In larger vessels, the asphericity may approach 0.05 as the cell assumes a parachute-like shape (32). The DPD scheme, when used to model flow in larger vessels, indicated a similar trend as shown in FIG. 11(e). However, in the narrowly constricted channels, the average asphericity increased significantly. The computational model was capable of capturing a range of shape deviations in large and small vessels, which correlated well with experimental measurements for the smallest length scales.

### Pressure-Velocity Relationship

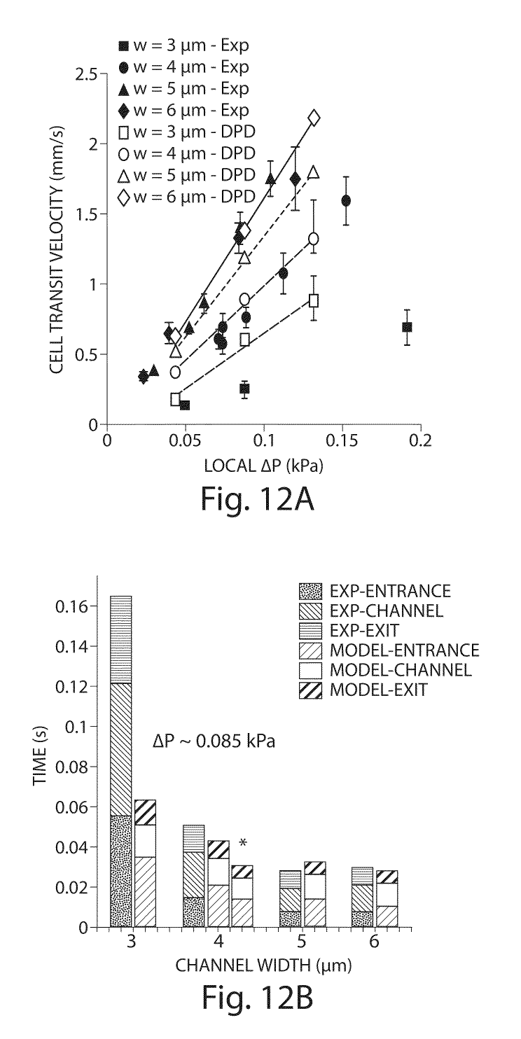

FIG. 12(a) illustrates pressure-velocity relationships for RBC flow across channels of different cross-sectional dimensions. Local average pressure differences were inferred from the velocity of neutrally buoyant beads, which were mixed with RBC suspensions. The experimentally measured average bead velocities were translated to pressure differences using known analytical solutions for flow in rectangular ducts as well as the results of computational fluid dynamics study (37). Further details of these steps are provided elsewhere herein. Average cell velocity measurements were taken between the point just prior to the channel entrance (the first frame in FIG. 11(a)) and the point at which the cell exits the channel (the final frame in FIG. 11(a)). As such, the time scale examined in these studies was a combination of entrance times, traversal and exit times. These individual time scales are plotted in FIG. 12(b). The DPD model adequately captured the scaling of flow velocity with average pressure difference for 4-6 μm wide channels. Overlap in the experimental data for 5-6 μm wide channels was observed. Potential factors giving rise to this overlap were, in part, the subject of the sensitivity study described below.

### Temperature Effects

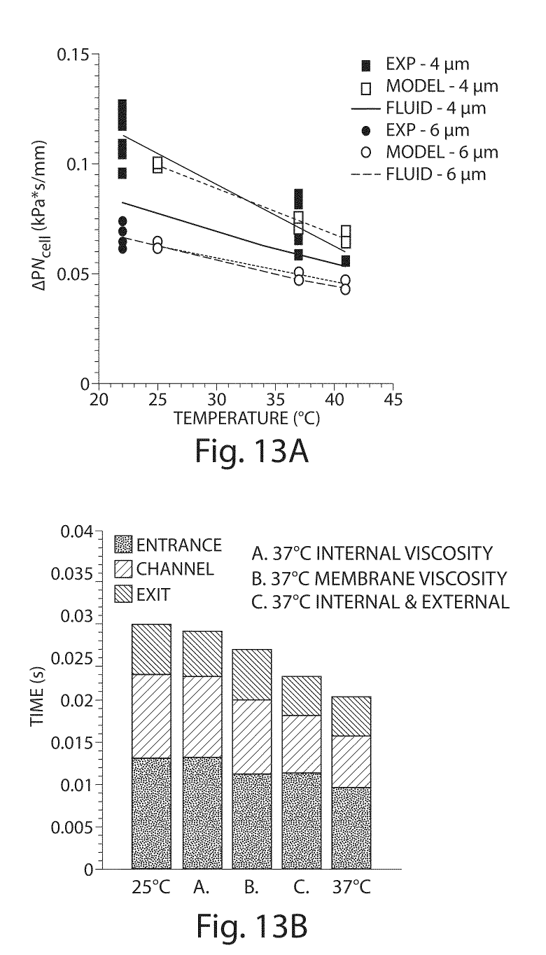

The effect of temperature on the flow dynamics of the RBC is exemplified in FIG. 13(a). The ratio of the local pressure gradient and average cell velocity (ΔP/V) versus temperature was examined for two different, channel geometries. The pressure-velocity ratio for a fluid with the properties of the surrounding media as a function of temperature for each of the respective channel geometries was examined. For a certain channel geometry, ΔP/V was determined to scale with the effective viscosity of the medium (external fluid, cell membrane and internal fluid) and the membrane stiffness. Over this temperature range (22° C.-41° C.), quasi-static experiments revealed a minimal effect of temperature on the stiffness of healthy RBCs (34, 57).

FIG. 13(b) presents results of a series of simulations that were performed to determine the relative contributions of the RBC membrane viscosity and its internal and external fluid viscosities for flow across a 4 μm wide channel. As illustrated, for a 4 μm wide channel, the external fluid and membrane viscosities influenced the transit behavior of the RBC.

### DPD Sensitivity Study

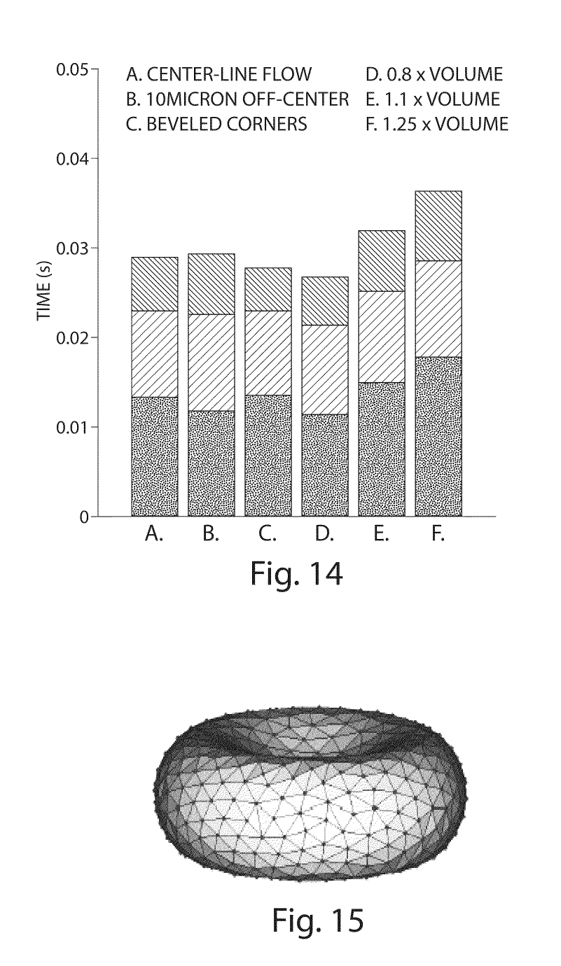

Sensitivity studies were performed, to evaluate the effects of irregular cross-sectional geometries, flow orientations and variations in cell size on flow behavior. Results of these studies are presented in FIG. 14.

### Numerical Methods

The RBC membrane was approximated by a collection of points connected by links. Each point corresponds to the junction complex in the RBC membrane and each link represents spectrin proteins between junction complexes. The coarse-grained RBC model (shown for N=500 points below) was validated against experimental data of the mechanical response of an individual cell (42). The model accounted for bending and in-plane shear energy, viscous effects of the membrane, and constraints of total area and volume. Further details of the modeling approach are provided below.

The surrounding external fluid and RBC internal fluid (hemoglobin) were modeled using Dissipative Particle Dynamics. The DPD particles interact with each other through three soft pairwise forces: conservative, dissipative and random forces. Dissipative and random forces form a DPD thermostat and their magnitudes are related through the fluctuation-dissipation theorem (18). The functional form of these forces can be varied to alter the viscosity of the DPD fluid (20). This approach was used to make the internal RBC fluid more viscous compared to the external fluid.

In the simulations, each point in the RBC membrane was a DPD particle. When the model was immersed into the DPD fluid, each particle experiences membrane elastic and viscous forces in addition to the DPD forces from the internal and external fluid particles. Bounce-back reflection was employed at the membrane surface to ensure no-slip condition and to make the membrane impermeable to internal and external fluids. The channel walls were modeled by freezing DPD particles in combination with bounce-back reflection. Periodic inlet/outlet boundary conditions were employed. The flow was sustained by applying an external body force.

The internal fluid is 9, 8.5 and 7.6 times more viscous than the external fluid in simulations corresponding to temperature of 22° C., 37° C. and 41° C., respectively (14, 26, 43). The effect of temperature in the experiment on the viscosity of the suspending medium was modeled by changing the viscosity of the DPD fluid surrounding the RBC. The viscosity of the external fluid at 37° C. and 41° C. was decreased by 22% and 28% compared to the viscosity at 22° C., while the membrane viscosity was decreased by 50% and 63.5%, respectively, to match the experimentally measured BBC relaxation times at these temperatures.

### Experimental Methods

### Cell Solution and Buffer Preparation

Whole blood from healthy donors was obtained from an outside supplier (Research Blood Components, Brighton, Mass.). Blood was collected in plastic tubing with an ACD preservative added during collection. Upon reception, blood was stored at 4° C. Experiments were performed within 12 hours of acquiring blood samples.

The primary buffer used in all cell solution preparations and experiments was RPMI 1640 with 1% wt of Bovine Serum Albumin (BSA) (pH=7.4). 100 μL of whole blood is suspended in 1 mL of this buffer and centrifuged three times at 1000 rpm. After the final centrifugation, red cells were suspended in BSA/RPMI buffer, resulting in a final hematocrit of approximately 0.4-0.5%. Immediately prior to introduction into the microfluidic channels, 20-30 μl (5% wt) of 1 μm polystyrene beads (Polysciences Inc., Warrington, Pa.) were added to the cell solution. In some cases, fresh cell/bead solutions were periodically introduced over the course of a flow experiment. For all cell solutions, typically no more than 2 hrs. elapsed from the time of its final centrifugation to the time of its flow characterization.

### Microfluidic Channel Fabrication and Experimental Procedures

PDMS-based microfluidic channels were fabricated using soft lithography (56, 58). The master mold was made from SU8 resist using a two mask, two layer process. The first layer defined the region of primary interest in the flow characterization experiments (described below) and the second layer was used to define large reservoirs for input/output ports and easier interfacing with buffer and cell solutions.

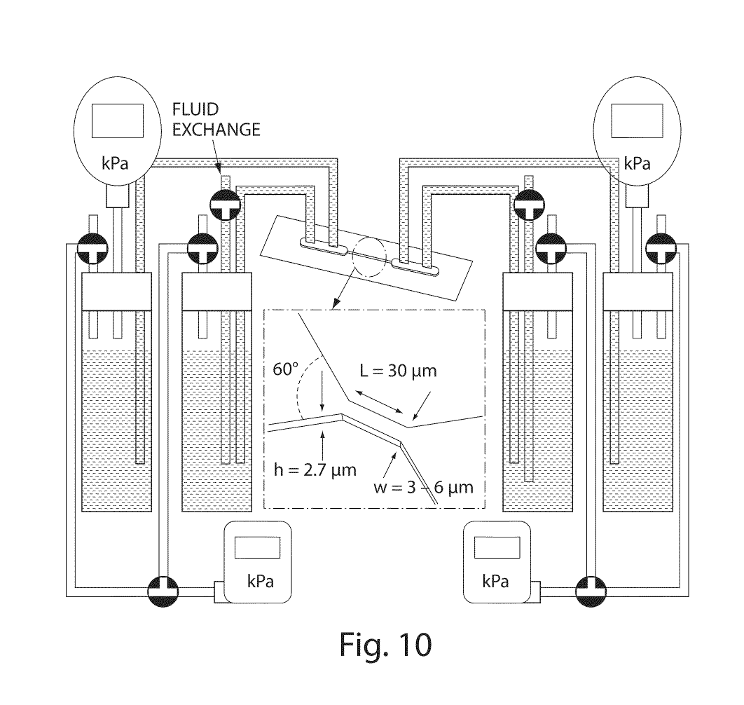

The channel structures and pressure-control system used in this work are illustrated in FIG. 10. At their narrowest point, channels were approximately 30 μm long, 2.7 μm high and had widths ranging from 3-6 μm. A sharply converging/diverging structure was used to ensure that it was possible to observe nearly the entire traversal process (channel entrance deformation, channel flow and channel exit behavior/shape recovery) with the microscope objectives used, typically 20×-50×. In this way, the use of a single channel structure ensured that the hydrodynamics of the experiment was well-controlled and more easily understood. In addition, this approach reduced the physical domain of the experiment so as to allow for a small modeling domain and decrease the computational time required in the evaluation of our modeling approach.

In the pressure-control system, a set of dual input and output ports were utilized in order to allow for periodic exchanges of buffer and priming solutions as well as fresh cell solutions. The applied pressure difference was achieved using a combination of pressurized reservoirs and hydrostatic pressure adjustments. The pressure regulators (Proportion Air Inc., McCordsville, Ind.) utilized a computer-controlled high-resolution solenoid valve and had a range of 0-207 kPa with an applied pressure resolution of approximately 69 Pa (0.01 psi). These regulators exhibited the suitable response and linearity at pressure levels above 20.7 kPa. Therefore, this was typically the minimum pressure level applied at the entrance and exit reservoirs. Applied pressure differences were first set by increasing the regulator pressure above this minimum level. Additional hydrostatic pressure adjustments were made by adjusting the relative heights of the pressure columns using a micrometer stage, giving an applied pressure difference resolution of approximately 1 mmH₂0 (0.001 psi or 9.8 Pa). A secondary set of pressure gauges was used to check the applied pressure difference at the fluid reservoirs in order to ensure there were no significant leaks in the pressure lines leading up to the fluid reservoirs.

Experiments at. 37° C. and 41° C. were carried out using a water bath system in which the channel was bonded into an aluminum dish using a PDMS seal or a paraffin gasket. Pre-heated water was then added to the reservoir to bring the system to the desired temperature. This temperature was maintained by a temperature control system using a flexible heater to radially heat the water bath, a T-type thermocouple temperature probe, and a proportional—integral—derivative (PID) temperature controller (Omega Inc., Stamford, Conn.). Temperature at the coverslip surface was monitored throughout the experiments using a T-type thermocouple. The use of such a water-bath system ensured that the entire device, including the input and output tubing containing the cells under examination, was maintained at the same temperature. In addition, the high thermal mass of the water-bath system ensured temperature stability for the duration of a typical experiment (1-4 h).

During a typical experiment, the channel system was first primed with a 1% wt solution of Pluronic F-108 surfactant (Sigma Inc., St. Louis, Mo.), suspended in PBS (1×). The enhanced wetting properties of the Pluronic solution allowed for easy filling of the channel and purging of air bubbles. After the channel was filled, the Pluronic was allowed to incubate for a minimum of 20 minutes in order to block the PDMS and glass surfaces from further hydrophobic and other non-specific adhesive interactions with the red cell membrane. After this incubation time, the system was flushed with a 1% wt BSA/RPMI buffer solution. The excess buffer was then removed from the entrance reservoir and the cell solution was added and introduced to the channel reservoir area. After an initial flow of cells across the channel was observed (typically by applying a pressure difference of approximately 0.7 kPa (0.1 psi)), the applied pressure difference was set to zero by first equilibrating the applied pressure from the pressure regulators and then stagnating the flow in the channel by trapping a bead in the center of the channel via relative height (i.e. hydrostatic pressure) adjustments. After this process, pressure differences were typically set using the electronically-controlled pressure regulators. However, due to hydrodynamic losses, this applied up-stream and down-stream pressure difference did not correspond to the local pressure difference across the channel. In order to determine this local pressure difference, bead trajectories and velocities were measured using our high speed imaging capabilities and an image processing routine. These measured velocities were used to determine the local pressure difference using a combination of computational fluid dynamics simulations and analytical solutions for flow in rectangular ducts (37). Further details of this procedure are provided below.

Flow experiments were performed on a Zeiss Axiovert 200 inverted microscope (Carl Zeiss Inc. Thornwood, N.Y.) using a halogen source and either a 20× or 40× objective. A dry objective (e.g., not an oil or water-immersion objective) was used in order to ensure that the cover slip was sufficiently thermally isolated for experiments at elevated temperatures. Images were recorded on a PCO.1200 hs high-speed CMOS camera, operated at typical frame-rates of 1000-2000 fps (Cooke Corp., Romulus, Mich.).

### Equations for RBC and DPD Models

The membrane model that was developed consisted of points {rₙ, nε1 . . . N} which were the vertices of surface triangulation (FIG. 15). The area of triangle αε1 . . . II formed by vertices (l, m, n) was given by A_{α}=|(rₘ−rₗ)×(rₙ−rₗ)|/2. The length of the link iε1 . . . S connecting vertices m and n was given by Lᵢ=|rₘ−rₙ|. The in-plane free energy of the membrane

$${F}_{in \text{-} plane} = {\sum }_{i ∈ links} {V}_{WLC} ( {L}_{i} ) + {\sum }_{α ∈ triangles} C / {A}_{α} , \qquad ( 1 )$$

included the worm-like chain (WIC) potential for individual links

$${V}_{WLC} ( L ) = \frac{{k}_{B} {TL}_{max}}{4 p} \times \frac{3 {x}^{2} - 2 {x}^{3}}{1 - x} , \qquad ( 2 )$$

where x=L/Lₘₐₓε(0,1), Lₘₐₓ was the maximum length of the links and p was the persistence length; the parameter C in the hydrostatic elastic energy term was defined as in (5). The bending energy was given by

$${F}_{bending} = {\sum }_{adjacent α , β pair} {k}_{bend} [ 1 - cos ( {θ}_{αβ} - {θ}_{0} ) ] \qquad ( 3 )$$

where k_{bend} was the average bending modulus (4), while θ₀ and θ_{αβ} were the spontaneous and the instantaneous angles between two adjacent triangles, respectively. The total volume and surface area constraints were given by

$$\begin{matrix}{F}_{volume} = \frac{{{k}_{volume} ( Ω - {Ω}_{0} )}^{2} {k}_{B} T}{2 {L}_{0}^{2} {A}_{0}} , \\ and & ( 4 ) \\ {F}_{surface} = \frac{{{k}_{surface} ( A - {A}_{0} )}^{2} {k}_{B} T}{2 {L}_{0}^{2} {A}_{0}} , & ( 5 )\end{matrix}$$

respectively, where L₀ is the average length of the link, Ω and Ω₀ were the instantaneous and equilibrium volumes of the model, and A and Aₒ were instantaneous and equilibrium surface areas. The parameters kᵥₒₗᵤₘₑ and k_{surface} were adaptively adjusted during the simulations to keep the deviations of instantaneous volume and surface area, from the equilibrium values to less than 1%. The elastic contribution to the forces on point nε1 . . . N was obtained as

fₙ^{E}=−∂(Fᵢₙ₋ₚₗₐₙₑ+F_{bending}+Fᵥₒₗᵤₘₑ+F_{surface})/∂rₙ. (6)

The effect of membrane viscosity was modeled by adding frictional resistance to each link. The viscous contribution to the force on point nε1 . . . N was given by

$${f}_{n}^{V} = - {\sum }_{( n , m ) ∈ links} γ RBC ( {v}_{nm} \cdot {\hat{r}}_{nm} ) {r}_{nm} , \qquad ( 7 )$$

where vₙₘ=vₘ−vₙ, rₙₘ=rₘ−rₙ, rₙₘ=|rₙₘ|, \hat{r}ₙₘ=rₙₘ/rₙₘ, and vₙ, was the velocity of point n.

In simulations, the surrounding fluid and RBC internal fluid (hemoglobin) were modeled using Dissipative Particle Dynamics. All particles were assigned the same mass equal to M=1 in simulations. The particles were set to interact with each other through conservative, dissipative and random force. Specifically, the forces exerted on a particle n by particle m were:

fₙₘ^{C}=f^{C}(rₙₘ)\hat{r}ₙₘ, (8)

fₙₘ^{D}=−γω^{D}(rₙₘ)(\hat{r}ₙₘ·vₙₘ)\hat{r}ₙₘ, (9)

fₙₘ^{R}=σω^{R}(rₙₘ)ξₙₘ\hat{r}ₙₘ, (10)

The parameters γ and σ determine the strength of the dissipative and random forces, respectively. Also, ξₙₘ were symmetric Gaussian random variables with zero mean and unit variance, and were independent for different pairs of particles and at different times; ξₙₘ=ξₘₙ was enforced in order to satisfy momentum conservation. Finally, ω^{D} and ω^{R} were weight functions.

All forces act within a sphere of interaction radius r_{c}, which was the length scale of the system. The conservative force was given by

$${f}_{nm}^{C} = \{ \begin{matrix}α ( 1 - {r}_{nm} / {r}_{c} ) {\hat{r}}_{nm} , & {r}_{nm} < {r}_{c} \\ 0 , & {r}_{nm} \geq {r}_{c} ,\end{matrix} \qquad ( 11 )$$

where α was a conservative force coefficient. The requirement of the canonical distribution sets two conditions on the weight functions and the amplitudes of the dissipative and random forces (18, 24)

ω^{D}(rₙₘ)=[ω^{R}(rₙₘ)]², (12)

and

σ²=2γk_{B}T_{DPD}, (13)

where T_{DPD} was the DPD system temperature and k_{B} was the Boltzmann constant. The weight function takes the form (20)

$${ω}^{D} ( {r}_{nm} ) = {[ {ω}^{R} ( {r}_{nm} ) ]}^{2} = \{ \begin{matrix}{( 1 - {r}_{nm} / {r}_{c} )}^{8} , & {r}_{nm} \leq {r}_{c} , \\ 0 , & {r}_{nm} > {r}_{c} ,\end{matrix} \qquad ( 14 )$$

with exponent s≦2 (s=2 for standard DPD). The value of exponent s affected the viscosity of the DPD fluid for fixed parameters σ and γ in dissipative and random forces. Lower values of s typically resulted in a higher viscosity of the fluid. Larger values of dissipative force coefficient γ increased the viscosity of the DPD fluid and lowered the temperature of the DPD fluid.

It was verified that there were no solidification artifacts associated with lower temperatures. This was done by calculating the radial distribution function as well as diffusion coefficient of the DPD fluid. In addition, the Newtonian behavior of the DPD fluid was verified using Poiseuille flow with known exact solution.

When the RBC model was immersed into the DPD fluid, each particle experienced membrane elastic and viscous forces in addition to the DPD forces from the surrounding fluid particles. Therefore, the total force exerted on a membrane particle was:

fₙ=fₙ^{E}+fₙ^{V}+fₙ^{C}+fₙ^{D}+dt^{-1/2}fₙ^{R}, (15)

while for a fluid particle the total force was:

fₙ=fₙ^{C}+fₙ^{D}+dt^{-1/2}fₙ^{R} (16)

Here fₙ^{C}=Σ_{n≠m} fₙₘ^{C} was the total conservative force acting on particle n: fₙ^{D} and fₙ^{R} were defined similarly. The dt^{-1/2} term multiplying random force fₙ^{R} in equations (15) and (16) was there to ensure that the diffusion coefficient of the particles is independent of the value of the timestep dt used in simulations (24). The time evolution of the particles was described by Newton's law

$$\begin{matrix}{dr}_{n} = {v}_{n} dt , & ( 17 ) \\ {dv}_{n} = \frac{1}{M} {f}_{n} dt . & ( 18 )\end{matrix}$$

The simulations were done in non-dimensional units and therefore a link was established between DPD and physical scales. The DPD units of length, time and energy were defined. The unit of length (the DPD cutoff radius r_{c}) in simulations was equal to 1 micron. The equilibrium, persistence and maximum length of the links, as well as other parameters of RBC model were set according to (42). In addition, two independent experimental measurements were used to specify the units of energy and time in DPD. The amplitude of thermal fluctuations of the membrane at rest were set within the range of experimental observations (40). The amplitude of the membrane thermal fluctuations was influenced by the choice of DPD unit of energy in simulations. The characteristic relaxation time of the RBC model in simulations was set to an experimentally measured value of 0.16 s, at room temperature. The relaxation time was influenced by the ratio of membrane elastic and viscous forces. In simulations corresponding to 37° C. and 41° C., the membrane viscosity is decreased by 50 and 63.5 percent, respectively, to match experimentally measured relaxation time at these temperatures. The rest of the simulation parameters were based on these units of length, time and energy.

The fluid domain in simulations corresponds to the middle part of the microfluidic device. The width of the flow domain was 60 μm, the length was 200 μm, the height was 2.7 μm. The central part of the simulation domain was the same as in the experiment. Specifically, the flow was constricted to rectangular cross-section of 4, 5 or 6 μm in width and 2.7 μm in height. The walls were modeled by freezing DPD particles in combination with bounce-back reflection, similar to (41). The flow was sustained by applying an external body force. The passage of the RBC through the microchannel with the dimension smaller than the size of the resting RBC involves large deformations of the cell followed by the recovery of the biconcave shape. Therefore, the ratio of the characteristic relaxation time and the RBC transition time was the same in the simulations as in the microfluidic experiments. A single experimental data point (4 μm wide×2.7 μm high channel, 44 Pa pressure difference, room temperature) was used to estimate this ratio. The unit of the DPD external body force was then calculated to match this ratio and later used to model the remaining experimental conditions.

The material reference state for the in-plane elastic energy of the model was chosen to be a biconcave shape (42) and spectrin network reorganization was not considered in the simulations.

### Measurement of Local Pressure Difference Across Microfluidic Channels

A particle tracking scheme was used to experimentally determine the local pressure gradients in the microfluidic channel. Viscous flow of a Newtonian fluid with viscosity (17) through a channel of rectangular cross-section with width (w), height (h) and length (L) was described by the pressure-velocity relationship:

$$V ( x , y ) = \frac{Δ P}{η L} \frac{4 {h}^{2}}{{π}^{3}} {\sum }_{n = 1 , 3 , 5 , …}^{∞} \frac{1}{{n}^{3}} ( 1 - \frac{cosh ( n π x / h )}{cosh ( n π ω / 2 h )} ) sin ( n π y / h ) \qquad ( 19 )$$

where −w/2≦x≦w/2 and 0≦y≦≦h.

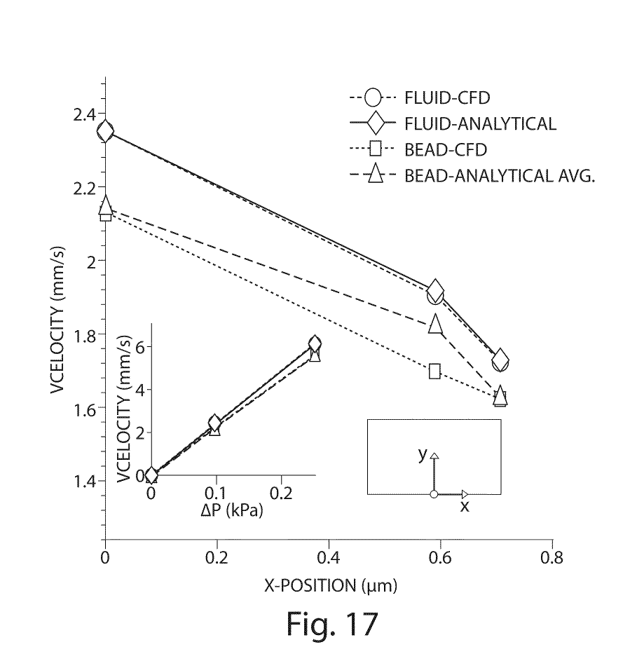

To establish a relationship between the measured bead trajectories and the local pressure gradient, a combination of numerical averaging and computational fluid dynamics studies (CFD) was used. Bead trajectories were limited to the region: −w/2+Dₚ/2≦x≦w/2−Dₚ/2 and D_{P}/2≦y≦h−Dₚ/2. Over this region, a grid of points with coordinates (x_{b}y_{b}) and separation (δx, δy) were selected for which the velocity of the beads at those points were approximated by the average fluid velocity of the circular region of radius Rₚ=Dₚ/2 around that point. These bead velocities were averaged over the bead flow region to establish a relationship between the average bead velocity and the local pressure difference. An example of this relationship, for the channels and temperatures used in the experiments, is depicted in FIG. 7. In calculating these relationships, the fluid was set to have the same temperature-dependent viscous properties as water (11, 38, 60). This relationship was compared to the results of a series of CFD simulations of a flow of 1 μm particles in a 2.7 μm high×4 μm wide channel. These CFD results indicated that for flow off the centerline of the channel, rotational effects were present and beads may not travel along the fluid streamlines. However, as exemplified in FIG. 17, these effects may influence the bead's average velocity in the microfluidic channel.

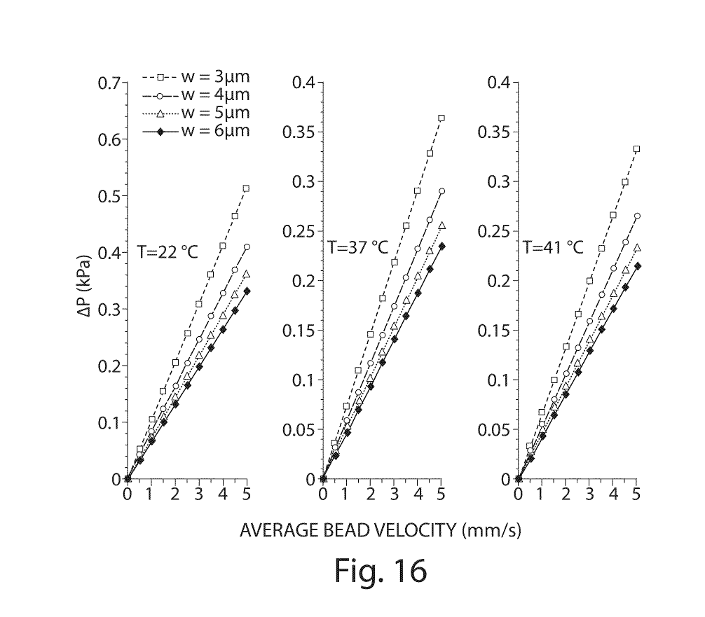

In certain experiments, the minimum depth of field of the imaging system was estimated to be 2.8 μm using the analysis presented in (33). Thus, bead images were taken along essentially the entire channel height. These bead trajectories were tracked and subsequently analyzed using an image segmentation and tracking routine written in MATLAB software. Average velocity measurements were checked by manually tracking a subset of beads from every data-set. The average bead velocity was then translated to a local pressure difference using the relationships presented in FIG. 16.

### References For Example 3

[1] Abkarian, M., M. Faivre, and H. A. Stone. High-speed microfluidic differential manometer for cellular-scale hydrodynamics. Proc Nat. Acad. Sci. USA, 103:538-542, 2006.
[2] Antia, M., T. Herricks, and P. K. Rathod. Microfluidic modeling of cell-cell interactions in malaria pathogenesis. PLoS Path, 3:939-948, 2007.
[3] Bagchi, P., P. C. Johnson, and A. S. Pope'. Computational fluid dynamic simulation of aggregation of deformable cells in a shear flow. J Biomech Eng Trans ASME 127:1070-1080, 2005.
[4] Boal, D. H. and M. Rao. Topology changes in fluid membranes. Phys Rev A, 46:3037-3045, 1992.
[5] Boey, S. K., D. H. Boa!, and D. E. Discher. Simulations of the erythrocyte cytoskeleton at large deformation. i. microscopic models. Biophys 0.1, 75:1573-1583, 1998.
[6] Braasch, D. Red cell deformability and capillary blood flow. Physiol Rev, 51:679-701, 1971.
[7] Brody, J. P., Y. Q. Han, R. H. Austin, and M. Bitensky. Deformation and flow of red-blood-cells in a synthetic lattice—evidence for an active cytoskeleton. Biophys J, 68:2224-2232, 1995.
[8] Buffet, P. A. G. Maori, V. Brousse, 0.1. M. Correas, B. Dousset, A. Couvelard, R. Kianmanesh, 0. Farges, A. Sauvanet, F. Faye, M. N. lingeheuer, C. Ottone, H. Khun, L. Fiette, G. Guigon, M. Huerre, 0. Mercereau-Puijalon, and P. H. David. Ex vivo perfusion of human spleens maintains clearing and processing functions. Blood, 107:3745-3752, 2006.
[9] Canham, P. B. The minimum energy of bending as a possible explanation of the biconcave shape of the human red blood cell. 0.1 Theor Biol, 26:61-81, 1970.
[10] Canham, P. B. and A. C. Burton. Distribution of size and shape in populations of normal human red cells. Circul Res, 22:405-'422, 1968.
[11] Chatterjee, A. Intrinsic viscosity measurements of bovine serum albumin at different temperatures. Nature, 205:386, 1965.
[12] Chien, S. Red cell deformability and its relevance to blood flow. Annu Rev Physiol, 49:177-192, 1987.
[13] Chung, B., S. Kim, P. C. Johnson, and A. S. Popel. Computational fluid dynamics of aggregating red blood cells in postcapillary venules. Comput Methods Biomech Biomed Eng, 12:385-397, 2009.
[14] Cokelet, G. R. and H. J. Meiselman. Rheological comparison of hemoglobin solutions and erythrocyte suspensions. Science, 162:275-277, 1968.
[15] Da iano, E. R. The effect of the endothelial-cell glycocalyx on the motion of red blood cells through capillaries. Microvasc Res, 55:77-91, 1998.
[16] Denling, H. J. and W. Helfrich. Red blood cell shapes as explained on the basis of curvature elasticity. Biophys 0.1, 16:861-868, 1976.
[17] Eggleton, C. D. and A. S. Pope'. Large deformation of red blood cell ghosts in a simple shear flow. Phys Fluids, 10:1834, 1998.
[18] Espanol, P. and P. Warren. Statistical mechanics of dissipative particle dynamics. Europh:ys Lett, 30:191-196, 1995.
[19] Evans, E. A. and R. M. Hochmuth. Membrane viscoelasticity. Biophys J, 16:1-11, 1976.
[20] Fan, X., N. Phan-Thien, S. Chen, X. Wu, and T. Y. Ng. Simulating flow of dna suspension using dissipative particle dynamics. Phys Fluids, 18:63102-63110, 2006.
[21] Fedosov, D., B. Caswell, and G. E Karniadakis. A multiscale red blood cell model with accurate mechanics, ‘theology and dynamics. Biophys 3, 08:2215-2225, 2010.
[22] Frank, R. S. and R. M. Hochmuth. The influence of red cell mechanical properties on flow through single capillary-sized pores. J Biomech Eng Trans ASME, 110:155-160, 1988.
[23] Fujita, T. and M. Kashimura. Scanning electron microscope studies of human spleen. Immunol Res, 2:375-384, 1983.
[24] Groot, R. D. and P. B. Warren. Dissipative particle dynamics: Bridging the gap between atomistic and mesoscopic simulation. J Chem Phys, 107:4423-4435, 1997.
[25] Helfrich, W. Elastic properties of lipid bilayers: theory and possible experiments. Z Naturforsch, 28:693-703, 1973.
[26] Herrmann, A. and P. Muller. Correlation of the internal microviscosity of human erythrocytes to the cell volume and the viscosity of hemoglobin solutions. Biochim Biophys Acta, 885:80-7, 1986.
[27] Higgins. J. M., D. T. Eddington, S, N. Bhatia, and L. Mahadevan. Sickle cell vasoocclusion and rescue in a microfluidic device. Proc Natl Acad Sci USA, 104:20496-20500, 2007.
[28] Hochmuth, R. M., R. N. Marple, and S. P. Sutera. Capillary blood flow. i. erythrocyte deformation in glass capillaries. Microvasc Res, 2:409-419 1970.
[29] Hochmuth, R. M., P. R. Worthy, and E. A. Evans. Red-cell extensional recovery and the determination of membrane viscosity. Biophys 0.1, 26:101-114, 1979.
[30] Hoogerbrugge, P. J. and J. M. V. A. Koelman. Simulating microscopic hydrodynamic phenomena with dissipative particle dynamics. Europhys Lett. 19:155-16(1, 1992.
[31] Li, J., M. Dad, C. T. Lim, and S. Suresh. Spectrin-level modeling of the cytoskeleton and optical tweezers stretching of the erythrocyte. Biophys 0.1, 88:3707-3719, 2005.
[32] McWhirter, 0.1. L. H. Noguchi, and G. Gompper. Flow-induced clustering and alignment of vesicles and red blood cells in microcapillaries. Proc Nat! Acad Sci USA, 106:6039-6043, 2009.
[33] Meinhar C. D., S. T. Wereley, and M. H. B. Gray. Volume illumination for two-dimensional particle image velocimetry. Meas Sci Technol, 11:809-814. 2000.
[34] Mills, J. P., M. Diez-Silva, D. J. Quinn, M. Dao, M. Lang, K. S. Tan, C. T. Lim, G. Milon, P. H. David, O. Mercereau-Puijalon, S. Bonnefoy, and S. Suresh. Effect of plasmodial resa protein on deformability of human red blood cells harboring plasmodium falciparum. Proc Natl Acad Sci USA, 104:9213-9217, 2007.
[35] Mills, J. P., L. Qie, M. Dao, C. T. Lim, and S. Suresh. Nonlinear elastic and viscoelastic deformation of the human red blood cell with optical tweezers. MCB, 1:169-180, 2004.
[36] Mohandas, N. and E. Evans. Mechanical properties of the red cell membrane in relation to molecular structure and genetic defects. Annu Rev Biophys Biomol Struct, 23:787-818, 1994.
[37] Mortensen, N., F. Okkels, and H. Bruns. Reexamination of Hagen-Poiseuille flow: Shape dependence of the hydraulic resistance in microchannels. Phys Rev E, 71:57301-57304, 2005.
[38] Neurath, H., G. R. Cooper, and J. 0. Erickson. The shape of protein molecules ii. viscosity and diffusion studies of native proteins. J Biol Chem, 138:411-436, 1941.
[39] Noguchi, H. and G. Gompper Shape transitions of fluid vesicles and red blood cells in capillary flows. Proc Nat! Acad Sci USA, 102:14159-14164, 2005.
[40] Park, Y., M. Diez-Silva, G. Popescu, G. Lvkotrafitis, W. Choi, M. S. Feld, and S. Suresh. Refractive index maps and membrane dynamics of human red blood cells parasitized by plasmodium falciparurn. Proc Nat! Acad Sci USA, 105:1373013735. 2008.
[41] Pivkin, I. V., and G. E. Karniadakis. A new method to impose no-slip boundary conditions in dissipative particle dynamics. J Comput Phys, 207:114-128, 2005.
[42] Pivkin, I. V. and G. E. Karniadakis. Accurate coarse-grained modeling of red blood cells. Phys Rev Lett, 101:118105, 2008.
[43] Popel, A. and P. Johnson. Microcirculation and hemorheology. Annu Rev Fluid Mech, 37:43-69, 2005.
[44] Pozrikidis, C. Numerical simulation of blood flow through microvascular capillary networks. Bull Math Biol, 71:1520-1541, 2009.
[45] Pries, A. and ‘L W. Secomb Microvascular blood viscosity in vivo and the endothelial surface layer. Am J Physiol Heart Circ Physiol, 289:H2657-H2664, 2005.
[46] Secornb, ‘T. W. and R. Hsu. Analysis of red blood cell motion through cylindrical micropores: effects of cell properties. Biophys J. 71:1095-1101, 1996.
[47] Secomb, T. W., R. Hsu, and A. R. Pries. Motion of red blood cells in a capillary with an endothelial surface layer: effect of flow velocity. Am Physiol Heart Circ Physiol, 281:H629-H636, 2001.
[48] Secomb. T. W., R. Skalak, N. Ozkaya, and J. F. Cross. Flow of axisymmetric red blood cells in narrow capillaries. J Fluid Mech. 163:405-423, 1986.
[49] Shapira, Y., M. Vaturi, and A. Sagie. Hemolysis associated with prosthetic heart valves: a review. Cardiol Rev, 17:121-124, 2009.
[50] Shelby, J. P., J. White, K. Ganesan, P. K. Rathod, and D. T. Chiu. A microfiuidic model for single-cell capillary obstruction by plasmodium falciparum-infected erythrocytes. Proc Natl Acad Sci USA, 100:14618-14622, 2003.
[51] Shevkoplyas, S., S. Gifford, T. Yoshida, and M. Bitensky. Prototype of an in vitro model of the microcirculation. Microvasc Res, 65:132-136, 2003.
[52] Shevkoplyas, S., T. Yoshida, S. Gifford, and M. Bitensky. Direct measurement of the impact of impaired erythrocyte deformability on microvascular network perfusion in a microfluidic device. Lab Chip, 6:914-920, 2006.
[53] Suresh, S., J. Spatz, J. Mills, A. Micoulet, M. Dao, C. Lim, M. Boil, and T. Seufferlein. Connections between single-cell biomechanics and human disease states: gastrointestinal cancer and malaria. Acta, Biomater, 1:15-30, 2005.
[54] Sutton, N., M. C. Tracey, I. D. Johnston, R. S. Greenaway, and M. W. Rampling. A novel instrument for studying the flow behaviour of erythrocytes through mic:rochannels simulating human blood capillaries. Microvasc Res, 53:272-281, 1997.
[55] Tsukada, K., E. Sekizuka, C. Oshio, and H. Minamitani Direct measurement of erythrocyte deformability in diabetes mellitus with a transparent microchannel capillary model and high-speed video camera system. Microvasc Res, 61:231239, 2001.
[56] Voldnian, J., M. L. Gray, and M. A. Schmidt. Microfabrication in biology and medicine. Annu Rev Biomed Eng, 1:401-425, 1999.
[57] Waugh, R. and E. A. Evans. Thermoelasticity of red blood cell membrane. Biophys J. 26:115-131, 1979.
[58] Weibel, D., W. Diluzio. and G. Whitesides. Microfabrication meets microbiology. Nat Rev Microbiol, 5:209-218, 2007.
[59] ANeinbaum, S., J. M. Tarbell, and E. R. Damian°. The structure and function of the endothelial glycocalyx layer. Annu Rev Biomed Eng, 9:121-167, 2007.
[60] Wetzel, R., M. Becker, J. Behlke, H. Billwitz, S. Bohm, B. Ebert, H. Hamann, J. Krumbiegel, and G. Lassman. Temperature behaviour of human serum albumin. Eur J Biochem, 104:469-478, 1980.
[61] Yap, B. and R. Kamm. Mechanical deformation of neutrophils into narrow channels induces pseudopod projection and changes in biomechanical properties. J. Appl. Physiol, 98:1930-4939, 2005.

### Example 3.1

### Validation of the RBC Model

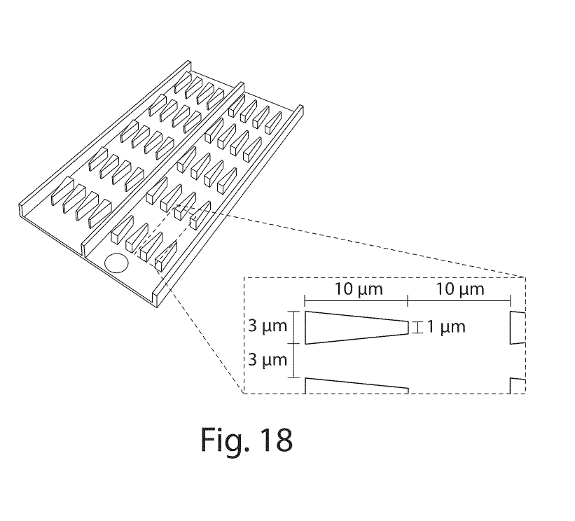

The computational model was validated using quantitative data obtained in microfluidic experiments. The microfluidic device consisted of two microchannels with a series of pores inside them. A representative sketch of the device is shown in FIG. 18. Dilute suspension of healthy and P. Falciparum malaria infected (ring stage) RBCs was pushed through the device. RBC velocities were measured at different pressure differentials driving the flow. Depending on the direction of the fluid flow the RBCs were passed through the pores with converging or diverging geometry.

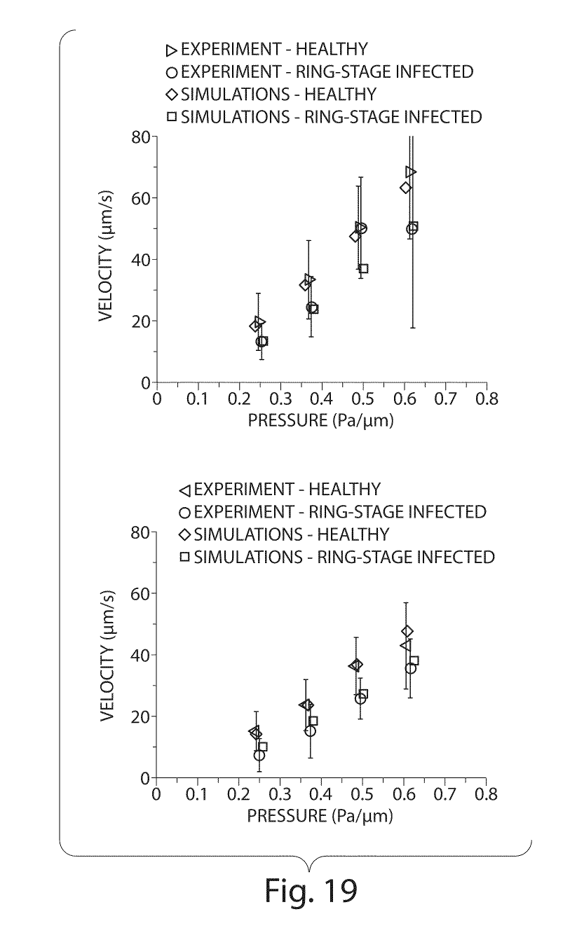

In simulations, parameters of healthy and malaria infected RBCs were specified using optical tweezer experimental data. The time scale was based on the RBC relaxation time. Comparison of simulation results with experimental data are shown in FIG. 19.

### Forward Problem, Construction of the Forward Function.

RBC membrane shear modulus μ, membrane bending modulus μ_{b}, membrane viscosity v, RBC size S, internal vᵢ and external vₑ fluid viscosities and applied pressure differential p can affect the RBC velocity in the device. Thus, RBC traverse velocity was a function of parameters listed above, i.e.

V=V(μ,μ_{b},v,S,vᵢ,vₑ,p). (1)

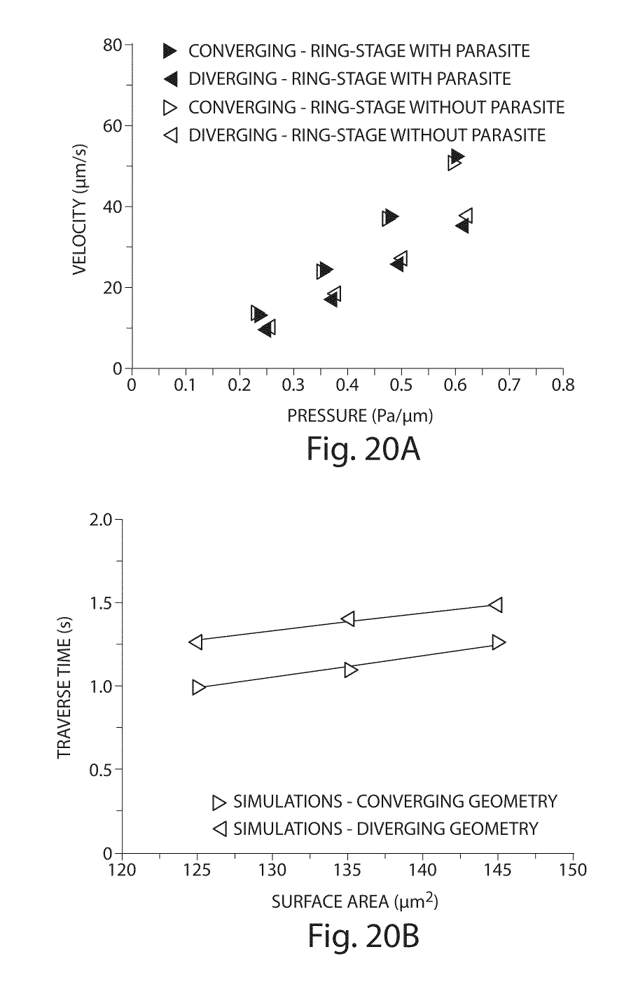

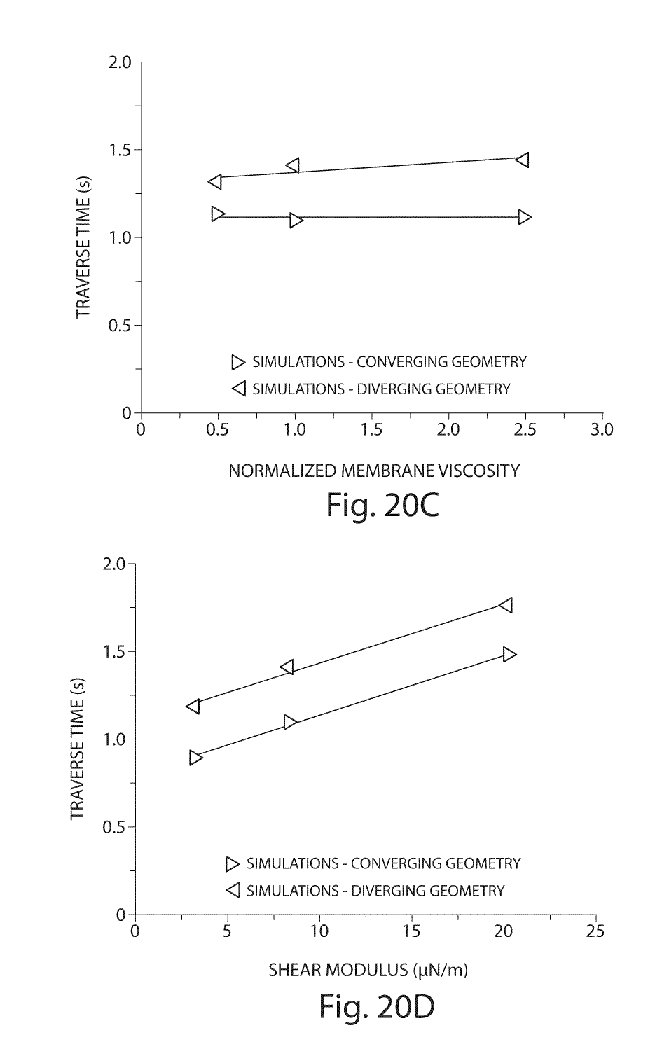

Effects of internal fluid viscosity and bending rigidity were expected to be negligible compared to the effects of membrane viscosity and shear modulus. The main results of simulations where the RBC parameter were varied independently are summarized in FIG. 20. In all cases, simulation parameters were varied within a limited range of values shown on FIG. 20.

From simulations, it was found that for malaria infected RBCs presence of the parasite inside the cell did not affect the ability of the RBC to traverse through the device. Therefore, RBC velocity variation may be due to a change in RBC membrane properties, i.e. membrane viscosity and shear modulus. Due to specific design of the device, membrane viscosity was not significant. Also, RBC size had comparatively small effect on traverse time compared to membrane shear modulus. Therefore, for this device, the function V was simplified to

V=V(μ,p). (2)

The specific form of the function for diverging geometry channel was chosen to be

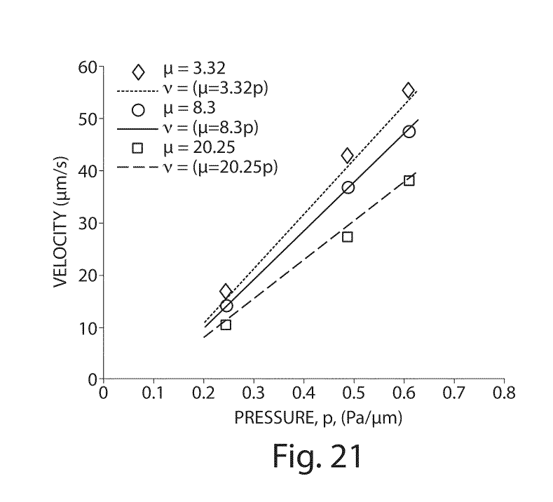

V−V₀*II(μ,p)=v₀*(p/p₀+a₁)/(a₂*μ/μ₀+a₃), (3)

where v₀ is the characteristic RBC velocity expressed in μm/s units, and II(μ,p) is a non-dimensional function. Characteristic values of membrane shear modulus, μ₀, and pressure, p₀, were set to 1 μN/m and 1 Pa/μm, respectively. Parameters a₁=−0.0938291, a₂=0.0002262, a₃=0.0088751 were determined by numerical fitting using simulation results shown in FIG. 21.

### Reverse Problem, Construction of Reverse Function.

The reverse problem consisted of estimating RBC shear modulus μ given RBC traverse velocity v through the device with diverging geometry at specific pressure differential p. The specific function for the reverse problem is obtained using function V for forward problem. It is equal to

μ=p₀[(p/p₀+a₁)/(v/v₀)−a₃]/a₂, (4)

where a₁=−0.0938291, a₂=0.0002262, a₃=0.0088751.

### Tests of Forward and Reverse Problems

Forward and reverse functions were applied to estimate velocities and shear moduli of healthy and infected RBCs in experiments.

The velocities of healthy and infected RBCs at different pressure differentials were calculated using the forward function. Shear modulus was set to be equal to 8.3 μN/m and 20.25 μN/m for healthy and infected RBCs respectively. Comparison of computational results with experimental measurements is shown in Table 3.1.1.

Shear moduli of healthy and infected RBCs were estimated using reverse functions from experimental measurements of traverse velocity of cells at different pressure differentials. The results are summarized in Table 3.1.2.

TABLE 3.1.1 provides velocities of healthy and infected RBCs for different pressures. Comparison of predictions obtained using forward function and experimental result.

| | | Healthy | Deviation | | Ring | Deviation |
|---|---|---|---|---|---|---|
| Pressure | Healthy prediction | experiment | (%) | Ring prediction | experiment | (%) |
| 0.245 | 14.05837108810414 | 15.38029790 | 8.6 | 11.23369422334911 | 7.40234 | 51.8 |
| 0.3675 | 25.45044962491707 | 23.68678822 | 7.5 | 20.33682047097138 | 15.28989643 | 33.0 |
| 0.49 | 36.842528161730011 | 36.22681851 | 1.6 | 29.43994671859366 | 25.77967625 | 14.2 |
| 0.6125 | 48.23460669854294 | 42.93948484 | 12.3 | 38.54307296621593 | 35.68261333 | 8.0 |

TABLE 3.1.2 provides values of shear modulus for healthy and infected RBCs calculated using reverse function.

| Pressure | Healthy prediction | Deviation (%) | Ring experiment | Deviation (%) |
|---|---|---|---|---|
| 0.245 | 4.21526022217163 | 49.2 | 51.03345859767361 | 152.0 |
| 0.3675 | 11.83859422807809 | 42.6 | 39.88162999040211 | 96.9 |
| 0.49 | 9.10773238729773 | 9.7 | 28.69442055368277 | 41.7 |
| 0.6125 | 14.16058352845047 | 70.6 | 25.01774813874417 | 93.5 |

### Example 4

### Assessment of Mechanical Properties of T Lymphocytes of Different Activation States

### Overview

Changes in the mechanical properties (e.g., apparent Young's (elastic) modulus) of T cells as a result of the T cell activation process were evaluated. The apparent Young's modulus of wild-type and WASp T lymphocytes in both the naïve and activated state were investigated using micropipette aspiration. Results from the micropipette aspiration studies showed that naïve T cells had a modulus of about 350 Pa. It was further found that this modulus decreased about three times to about 120 Pa for activated T cells. Compared to naïve wild-type T cells, naïve WASp T cells that exhibit impaired migration as a phenotype of Wiskott-Aldrich Syndrome (WAS) had a modulus of only 196 Pa. Activated WASp T cells decreased their apparent Young's modulus to a level that was comparable to that of activated wild-type T cells.

In order to investigate the viscous response of T cells before and after activation, AFM cell indentation experiments were conducted on naïve and activated wild-type T cells. By varying the indentation speed of the AFM probe, the viscous response of T cells was investigated and the relationship between apparent Young's modulus and indentation rate was characterized. Results from the studies showed that the viscous response of T cells increased with indentation rate regardless of their activation states. The decrease in the stiffness of T cells was recognized to be consistent with observations of a gain of mobility of these cells upon activation. The changes in mechanical properties of T lymphocytes upon activation observed in these studies may facilitate migration to tissue spaces that naïve T cells are unable to access.

### Materials and Methods

Naïve CD8+ T Lymphocytes Preparation

WASp mice on Balb/c background and control Balb/c mice were used in this study. Mice were euthanized and their peripheral lymph nodes and spleen were subsequently harvested and grinded between frosted microscope slides to release the cells into a RPMI medium (RPMI supplemented with 10% FBS and 1% HEPES). After the cell suspension was spun down at 1,200 rpm and the supernatant decanted, the resultant cell pellet was resuspended in 3 mL of red blood cell lysis buffer and incubated for 5 minutes at room temperature to rupture red blood cells. This cell suspension was then washed twice with the RPMI medium, and the cell pellet was eventually resuspended in a PBS buffer (PBS supplemented with 10% FBS, 1% pen/strep, and 5% rat serum) and ready for CD8+ T lymphocyte enrichment.

Naïve CD8+ T cells were enriched using the EasySep Mouse CD8+ T Cell Enrichment Kit from STEMCELL Technologies (British Columbia, Canada). Non-CD8 cells in a 5-mL polystyrene tube were first labeled for 15 minutes at 4° C. with biotinylated monoclonal antibodies against specific markers on their surfaces. A tetrameric antibody complex, which consisted of rat monoclonal antibodies bound to a mouse antibody against biotin on one end and a mouse antibody against dextran on the other end, was added, and the cell/antibody suspension was incubated at 4° C. for 15 minutes. Finally, magnetic dextran iron particles were added to the mixture and the tube was transferred to a EasySep Magnet after a third 15-minute incubation at 4° C.

The tube containing the cell suspension was inserted into the magnet and incubated for 5 minutes at room temperature, then the content was decanted into a clean 5-mL polystyrene tube. This step was repeated once more to improve the purity of the final CD8+ T cell population. Cells were then counted, spun down and resuspended in RPMI medium at 1×10⁶ cells/mL, and stored at 4° C. until ready to be used in an experiment. Successful enrichment of CD8+ T cells was confirmed using fluorescence-activated cell sorting (FACS). Approximately, 5×10⁵ cells were removed from the enriched population, exchanged into FACS buffer, and stained with FITC-anti-Thy1.2 antibody (Biolegend) and PE-anti-CD8 antibody (1:100 dilution) on ice for 30 minutes. The control sample was approximately 5×10⁵ pre-enrichment cells stained in parallel.

### Activated CD8+ T Lymphocytes Preparation

Activation of T lymphocytes was performed in 6-well plates. A plate was first incubated with 3 mL of anti-CD3 antibody (30 μL of 50 mg/mL anti-CD3 antibody in 3 mL of sterile PBS) per well for 2 hours in a 37° C. incubator. Afterward, the wells were washed three times with sterile PBS before each well was seeded with approximately 1.5×10⁶ CD8+ T cells in 3 mL of a T cell activation medium. The activation medium consists of 1) a base medium made up of RPMI supplemented with FBS, sodium pyruvate, 2) 100 units/mL interleukin-2, and 3) 30 μl of 50 mg/mL anti-CD28 antibody (BD Biosciences, USA). Cells were activated for 4 days in a 37° C. incubator. On day 4, the activated cells were counted and approximately 5×10⁵ cells were removed and exchanged into FACS buffer. The cells were stained with APC-anti-CD25 antibody for 30 minutes on ice, washed once with FACS buffer, then analyzed by FACS to confirm the success of activation. Approximately 5×10⁵ naive CD8+ T cells were stained in parallel to provide a control sample.

### Microwell Array Synthesis

Microwell arrays with well diameters of 8 microns and 16 microns were used to confine naïve and activated T cells, respectively, during AFM testing. The arrays were created on glass substrates via soft-stamp printing. Templates of the arrays were made of PDMS by combining PDMS and a cross-linking agent in a 90:10 wt % ratio. After vigorous stirring to ensure even mixing, the PDMS solution was degassed for one hour then poured onto silicon wafers containing the desired microwell array patterns. This assembly was transferred to an 80° C. oven and baked for at least two hours to cross-link the PDMS. After cooling off at room temperature, the PDMS molds of the arrays were cut out and cleaned with scotch tape to remove dust particles.

The glass substrates for the microwell arrays were prepared from 30 mm glass discs. They were plasma-cleaned for 5 minutes then immersed in a bath of 3-(trimethoxysilyl) propyl methacrylate for 5 minutes at room temperature. The 3-(trimethoxysilyl)propyl methacrylate compound served as an adhesion promoter that helped to bind the microwell array to the glass substrate. This solution was prepared by dissolving 200 μL of 3-(trimethoxysilyl)propyl methacrylate in 20 mL of ethanol, then adding 600 μL of 1% glacial acetic acid to the mixture immediately before the glass discs were immersed. Subsequently, the glass discs were washed three times with ethanol, dried in a nitrogen gas stream, and baked in an 80° C. oven for one hour.

The body of the microwell array was made of polyethylene glycol diacrylate (PEG DA) of MW 1000. PEG DA was first dissolved in PBS to result in a 20% polymer solution, then a photoinitiator, 2-hydroxy-2 methyl propiophenone, was added to the solution at an amount that corresponded to 10% (wt) of the PEG DA used. 50 μL of PEG DA solution was used to coat each PDMS mold (area approximately 10 mm×10 mm), then the mold was flipped over and finger-pressed against a pre-treated glass disc for 60 seconds. This assembly was transferred into a 15 mm petri dish, which was then placed under a hand-held UV light source for 30 minutes before the PDMS mold was carefully removed with tweezers to expose the microwell array.

### Micropipette and Glass Chamber Synthesis

Micropipettes were made using a micropipette puller. A microforge was used to trim the resultant micropipettes to different inner diameters depending on the activation state, and thus the size, of the cells tested. For naïve T cells, the inner diameter ranged from 2.5 to 3 micrometers, while for activated T cells the range was 4.5 to 5 micrometers.

Micropipette aspiration experiments were carried out in home-made glass chambers. The bottom of the chamber was a 15 mm×27 mm microscope coverslip. A U-shaped parafilm spacer was used to create the region where cell suspension was injected. A 13 mm×13 mm microscope coverslip was then laid on top of the spacer to seal off the chamber. Finally, the entire assembly was baked for one hour in an 80° C. oven to ensure sufficient adhesion of the parafilm spacer to the coverslips.

### Micropipette Aspiration of CD8+ T Lymphocytes

Approximately 3×10⁵ cells were transferred to an eppendorf tube and spun down at 2,000 rpm for 5 minutes at room temperature. All of the supernatant except 100 μL was removed, and after thoroughly resuspending the cell pellet in the remaining supernatant 10 μL of trypan blue solution was added to the cell suspension. Trypan blue was used to facilitate distinguishing dead cells from live cells. This mixture was subsequently diluted by adding to it 600 μL of either RPMI medium or T cell activation medium supplemented with IL-2 (100 units/mL total volume) for naive and activated T cells, respectively. 500 μL of the final mixture was pipetted into a glass chamber, which was then loaded into the micropipette aspiration system.

In the micropipette aspiration system, an Eppendorf micromanipulator was used to control the movement of the miropipette. The micropipette was connected to a water column that provided a pressure differential between inside the micropipette and the glass chamber, such that a cell could be aspirated into the micropipette. Experiments with the micropipette aspiration system were typically carried out at room temperature. A syringe pump (Harvard Apparatus PHD2000 Series) was used to control the rate at which the cell under study was aspirated into the micropipette, as well as to control the total volume of water withdrawn from the water column during an experiment. For this work, the aspiration rate was 36 mL/hr and the aspirated volume was 2 mL. This corresponded to a total applied pressure of about 400 Pa. For the duration of each experiment the movement of the cell into the micropipette was recorded with a CCD camera with an acquisition rate that corresponds to about 1.2 seconds between frames.

Each experiment typically lasted no more than three hours. This duration was selected to minimize the likelihood of detecting naïve T cells that were starting to die, e.g., as a result of prolonged exposure to a particular temperature (e.g., room temperature). Activated T cells were also tested following this constraint to make the experimental condition consistent for both populations. Since the health of primary T cells deteriorates quickly after they are harvested from tissues, naïve T cells were tested within the 24 hours following their harvest. Activated T cells were tested within the 24 hours of their 4^{th} day of activation.

### AFM Cell Indentation of CD8+ T Lymphocytes

AFM experiments were conducted using a MFP-3D from Asylum Research (CA, USA) together with a bioheater. The bioheater enabled certain AFM experiments to be conducted at relatively high temperatures. Cells were typically tested at room temperature. A glass disc with microwells of the appropriate diameter was placed in the bioheater before 2.5 mL of cell suspension containing approximately 1×10⁶ of either enriched naive CD8+ T cells or activated CD8+ T cells was pipetted into the bioheater. The cell sample was prepared by centrifuging approximately 1×10⁶ cells in an eppendorf at 2,000 rpm for 5 minutes, removing all except 100 μL of the supernatant, and subsequently resuspending the pellet in the remaining supernatant together with 10 μL of trypan blue. This mixture was then added to 2 mL of either RPMI medium or T cell activation medium supplemented with IL-2 for naive and activated T cells.

The AFM stage sits on top of an inverted microscope and is piezo-controlled to move in both the x and the y directions. Cells were viewed with a 40× objective lens. Before each experiment, the spring constant of the AFM probe used was determined in air via the thermal spectrum method. The spring constant ranged from approximately 0.018 nN/nm to 0.027 nN/nm. The sensitivity of the probe was determined in the testing medium on the part of the glass disc without the microwells. After this step, the AFM head was raised up a few turns and the microscope stage translated so that the microwell array containing T cells fell directly below the AFM probe. The probe was then engaged on the surface of the array, retracted, and subsequently engaged on a T cell with a trigger point of 0.2 V.

Indentation speeds spanning about three orders of magnitude were tested. For each speed, the force that the AFM probe exerted on the cell was typically tailored so that the cell displacement was approximately 1 micrometer. Usually this corresponded to ranges of 100 pN to 300 pN and 450 pN to 700 pN for low and high indentation rates, respectively. Five to ten indentations curves were collected for each cell at locations as close to the center of the cell as possible to avoid substrate effect. Some cells were subjected to multiple indentation speeds while others were tested under only a single speed.

### Data Analysis

The Young's modulus of a T cell was estimated from micropipette aspiration studies. Cell images which were recorded during an experiment were analyzed using ImageJ. Specifically, the movement front of a cell was tracked with respect to a fixed reference point on the same image. This information provided inputs for the following mathematical model:

$$E = ϕ ( η ) \frac{3 {r}_{i}}{2 π} ( \frac{Δ p}{L} )$$

$$η = \frac{{r}_{0} - {r}_{1}}{{r}_{1}}$$

which is known as the half-space model. In this expression, E is the Young's modulus of the cell, L is the length measured from the opening of the micropipette to the cell edge that extends into the micropipette, Δp is the pressure differential at a particular L, rᵢ and r₀ are the inner and outer diameter of the micropipette, and φ is a parameter called the wall function that is approximately 0.2. By plotting Δp with respect to the ratio ri/L and finding the linear line that best fits the data points (minimal total error), the slope of the linear line allowed the Young's modulus of the cell to be determined.

Cell indentation curves which were collected during AFM experiments were analyzed by fitting the contact region of the approach curve to the Hertz model, which describes the relationship between the apparent Young's modulus of a cell and the depth of indentation into the cell as:

$${δ}^{2} = \frac{4 F ( 1 - {v}^{2} )}{3 E tan α}$$

In the Hertz model, E is the apparent Young's modulus, δ is the indentation depth that the AFM probe creates on the cell, F is the indentation force, ν is Poisson's ratio of the cell, which is approximated as 0.5 for an incompressible material, and α is the half-angle of the pyramidal AFM indenter. This half-angle is approximately 35°.

An algorithm was developed using a commercially available software platform (MATLAB) to perform a least-squares fit of the contact portion of the approach curve. The fitted parameters were the point of contact and the apparent Young's modulus. In order to investigate how the modulus varied with the indentation depth, the fitted point of contact was substituted back into the Hertz equation to generate an E versus indentation depth curve. The apparent Young's moduli were estimated by computing averaged values from at least five indentation tests per cell.

### Statistics

Student's T test at 95% confidence level was conducted using MATLAB to determine if differences between two data sets was significant.

### Results

### CD8+ T Lymphocytes Enrichment

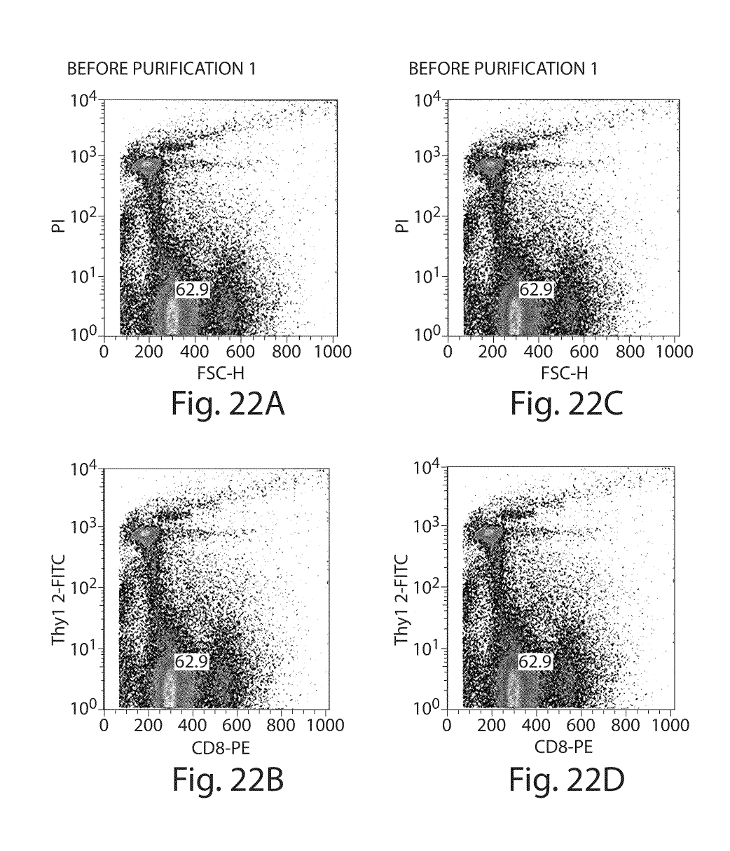

Cells harvested from the peripheral lymph nodes and the spleen were pooled together, and red blood cells were lysed before the single cell suspension was enriched for CD8+ T cells. FACS analyses of the cells before and after enrichment were similar for the control Balb/c mice and the WASp mice on Balb/c background, and a representative set of plots is shown in FIG. 22. Cells were first gated on their PI staining to identify the live cell population (FIG. 22a) and then among this population the staining pattern of Thy1.2 and CD8 was investigated. Thy1.2 identified a cell as being a T cell, and CD8 identified a cell as being a CD8+. The enrichment procedure greatly increased the purity of the CD8+ T cell population from 10% (FIG. 22b)) for the non-enriched sample to 90% for the enriched sample (FIG. 22d)). A purity of approximately 90% was consistently observed for both mouse strains when the enrichment procedure was repeated.

### AFM Indentation of CD8+ T Cells

The health of naïve T cells was observed to gradually deteriorate with prolonged exposure to certain temperatures, such as room temperature. Therefore, some precautions were taken to maximize the chance of selecting healthy cells for indentation. In order to ensure that the cells tested were relatively healthy while taking into account the time necessary for the AFM system to equilibrate before an experiment, each AFM test typically lasted not longer than 3 hours. In addition, all testing with naïve T cells was typically completed within 24 hours following the cell harvest.

It was appreciated in conducting the AFM experiments that cells could become damaged and/or die during the cell-harvest process. Since dead cells are often indistinguishable from live cells under a light microscope, trypan blue, which stains dead cells, was added to cell samples to facilitate identification and avoidance of indenting dead cells. To determine the appropriate amount of trypan blue that would efficiently stain the cell samples, a range of trypan blue concentrations were tested and the stained cells were counted using a hemocytometer. These results were compared to FACS analyses of the same cells stained with PI. It was found that a ratio of 1:10 of trypan blue dye to cell suspension volume was sufficient to effectively separate live cells from dead cells using light microscopy. In spite of precautions taken to eliminate dead cells, naïve T cells undergoing apoptosis were likely included in the population of cells tested at some frequency. In some cases, dying T cells stained a faint blue color that was hard to distinguish from healthy live cells. Even though round shiny cells (characteristic of healthy cells) were typically selected for testing, this dying T cell population could have been picked up and thus may explain some of the scatter in the data.

Experimental conditions were consistent for both populations, activated T cells were tested following the same experimental constraints as naïve T cells. Indentation tests involving activated T cells were typically completed within the 24 hours of their 4th day of activation. T cell activation was a gradual process that lasts several days. Day 4 of activation was chosen to ensure that most of the naïve T cells seeded had been activated.

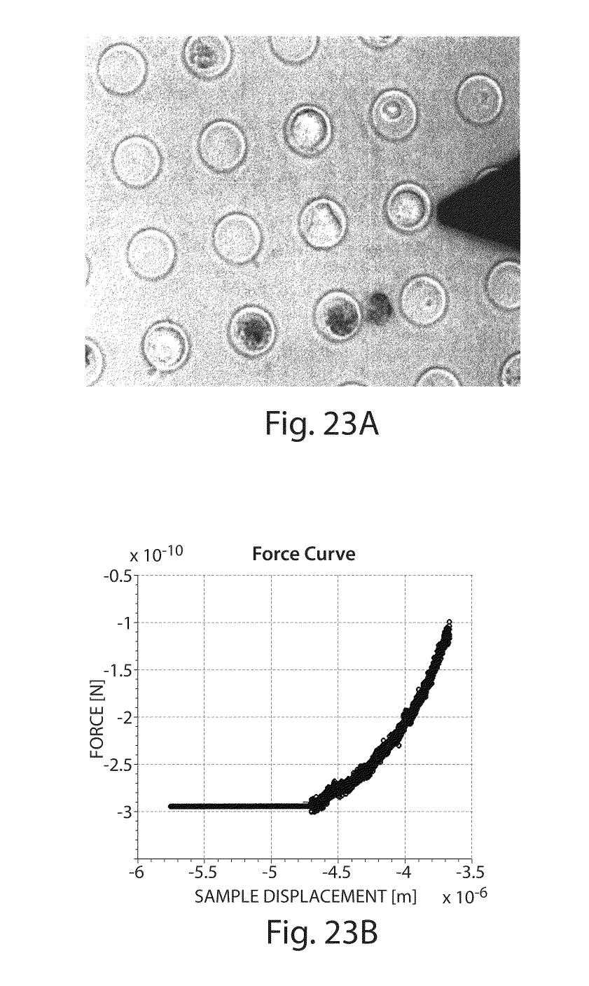

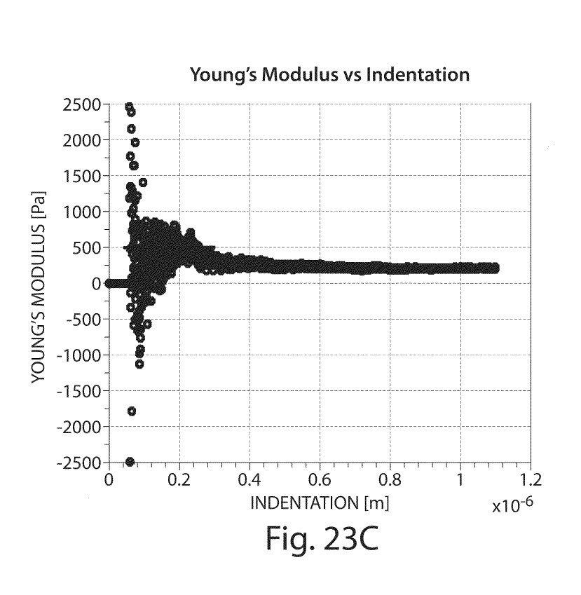

FIG. 23a) shows activated Balb/c T cells confined in microwells 16 μm in diameter. 5-10 indentation curves were collected for every cell at locations as close to the center of the cell as possible. Cell indentations were typically performed close to the cell center because it is at the center where the cells typically exhibit maximal thickness. However, data analyses revealed that even when an indentation was not performed close to the center of the cell, the apparent Young's modulus obtained was still similar to those obtained from center indentations. In fact, substrate effects was typically only observed when the AFM probe landed fairly close to the periphery of the cell. Often this was accompanied by the cell being lifted out of the confining well as the tip retracted.

AFM data were fitted using the Hertz model. In using the Hertz model, the cell structure was approximated as a homogeneous, linearly elastic half-space. The model described the experimental results well (FIG. 23b)). When the calculated apparent Young's modulus was compared with indentation depth, fluctuations in the modulus value were typically observed (FIG. 23c)). This noise may be attributed, in part, to variations in contact area as a result of the geometry of the pyramidal indenter and cell movement within a well in response to initial applied tip pressure. However, stable contact of the cell with the tip was typically established at relatively larger indentation depths. The constant modulus observed at large indentation depths indicated that despite indenting a large fraction of the cell (in the case of naïve cells, 1 μm out of the total cell length of about 7 μm was indented) the substrate effect was not a significant concern.

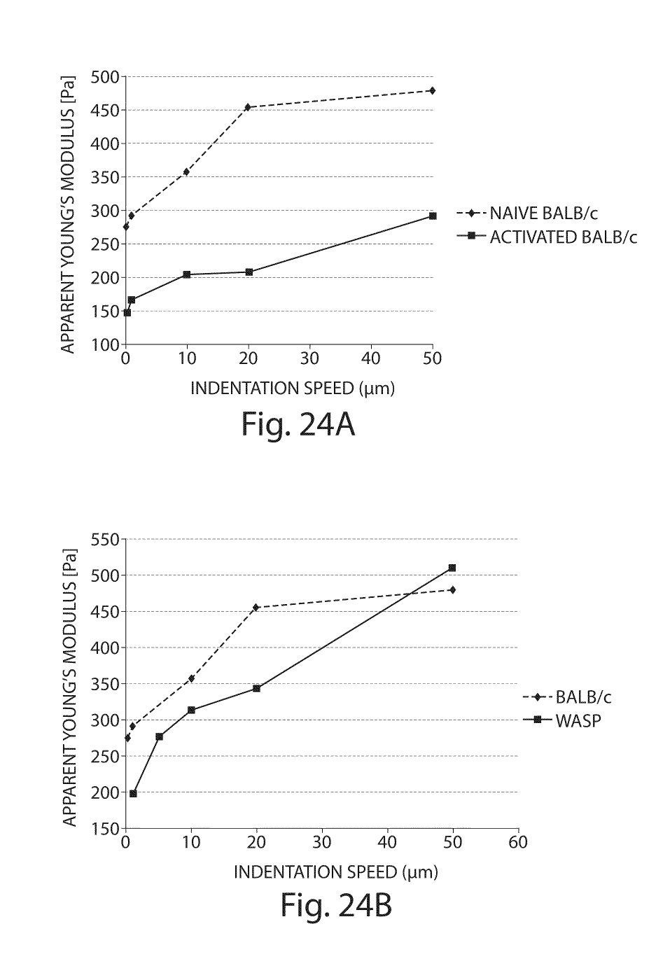

The apparent Young's modulus of both naïve and activated Balb/c T cells was found to increase with increasing indentation speeds (FIG. 24a)). This pattern may be due to an increased contribution of the viscous properties of the cells. As the deformation rate increased, the cells appeared stiffer. The shape of the curve depicted in FIG. 24a is similar for naïve and activated Balb/c T cells. There appeared to be a transition from one regime, in which the apparent Young's modulus increased linearly with indentation speed, to a second regime, in which the same linear increase in the modulus was observed but with a different rate relative to the indentation speed.

Naïve WASp T cells also exhibited an increase in apparent Young's modulus with indentation speed being comparable to wild-type counterparts. However, the transition point at which the slope of the curve changes was shifted toward lower indentation speeds.

### Micropipette Aspiration of CD8+ T Cells

Movement of cells into the micropipette was recorded as the aspiration pressure increased. The moving cell front was tracked and its distance from the pipette opening was measured using ImageJ. By fitting this information and the pressure differential at the moment the image was taken into the aforementioned half-space model, the apparent Young's modulus of the cell under study was calculated. The results are provided below in Table 1.1.

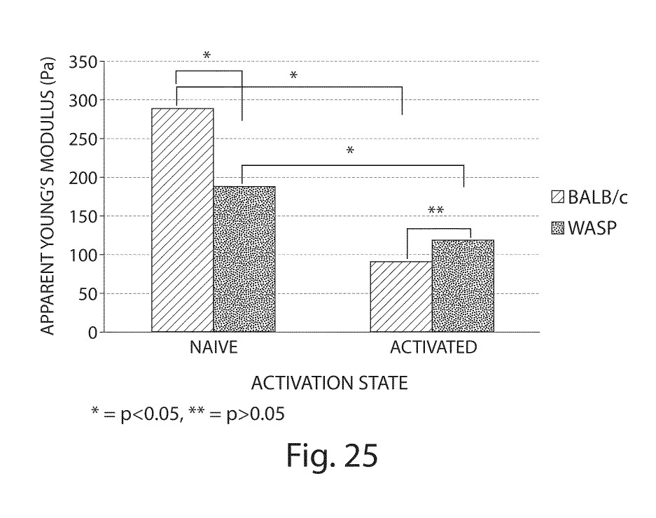

Naïve Balb/c T cells were found to have an averaged apparent Young's modulus of 290+/−102 Pa. Upon activation, this value decreased more than three times to only 94+/−49 Pa. This result indicates that Balb/c T cells became softer as a result of activation. Naïve WASp T cells were found to be softer than their wild-type counterparts (naïve Balb/c). Their modulus was calculated to be 190+/−69 Pa, which represents a 1.5 fold decrease from that of naïve Balb/c T cells. The activation process also reduced the modulus of WASp T cells, although the amount of this reduction was only 1.6 fold, compared to the 3 fold reduction in the case of Balb/c cells. The apparent Young's modulus of activated WASp T cells was calculated to be 121+/−41 Pa. However, Student's T test determined that the difference between the modulus of activated Balb/c and the modulus of WASp T cells did not pass the 95% significance level (FIG. 25).

TABLE 1.1
Apparent Young's moduli of Balb/c and WASp T cells in both the
naïve and the activated state were determined from micropipette
aspiration studies.

| | | Activated | | Activated |
|---|---|---|---|---|
| | Naïve 0T1 | 0T1 | Naïve WASp | WASp |
| Apparent | 290 +/− 102 | 94 +/− 49 | 190 + 1 − 69 | 121 +/− 41 |
| Young's | | | | |
| Modulus (Pa) | | | | |
| # Cells tested | 22 | 26 | 30 | 24 |

### References for Example 4

1. Thrasher, A. J. (2002). “WASP in immune-system organization and function.” Nature Rev. Immuno. 2:635-646.
2. Snapper, S. B., P. Meelu, D. Nguyen, B. M. Stockton, P. Bozza, F. W. Alt, F. S. Rosen, U. H. von Andrian, and C. Klein. (2005). “WASP deficiency leads to global defect of directed leukocyte migration in vitro and in vivo.” J. Leuk. Bio. 77(6):993-998.
3. Schmid-Schonbein, G. W., K. P. Sung, H. Tozeren, R. Skalak, and S. Chien. (1981). “Passive mechanical properties of human leukocytes.” Biophys. J. 36:243-256.
4. Zahalak, G. I., W. B. McConnaughey, and E. L. Elson. (1990). “Determination of cellular mechanical properties by cell poking, with an application to leukocytes.” J. Biomech. Eng. 112:283-294.
5. Hochmuth, R. M. (2000). “Micropipette aspiration of living cells.” J. Biomech. 33:15-22.

### Example 5

### Assessment of Cell Adhesion Properties

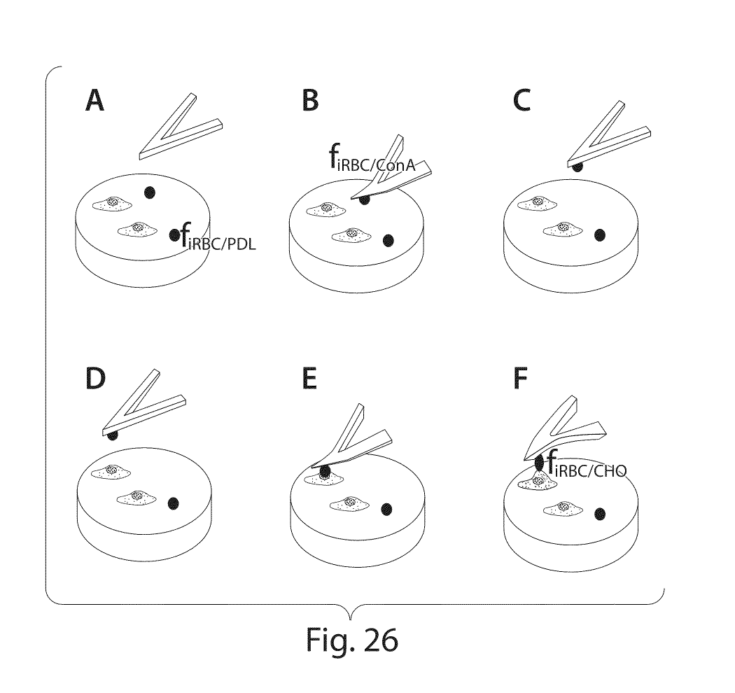

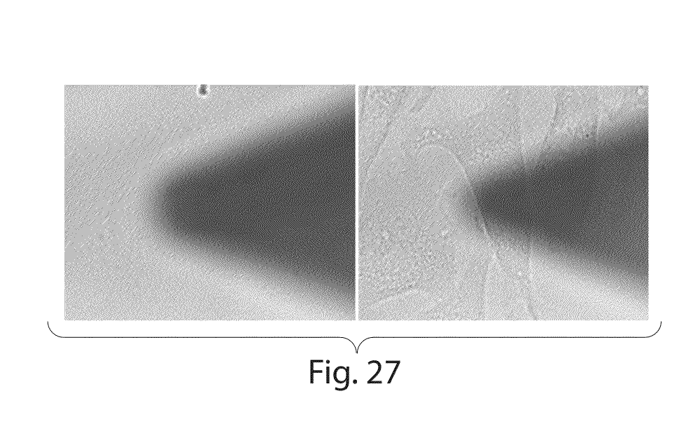

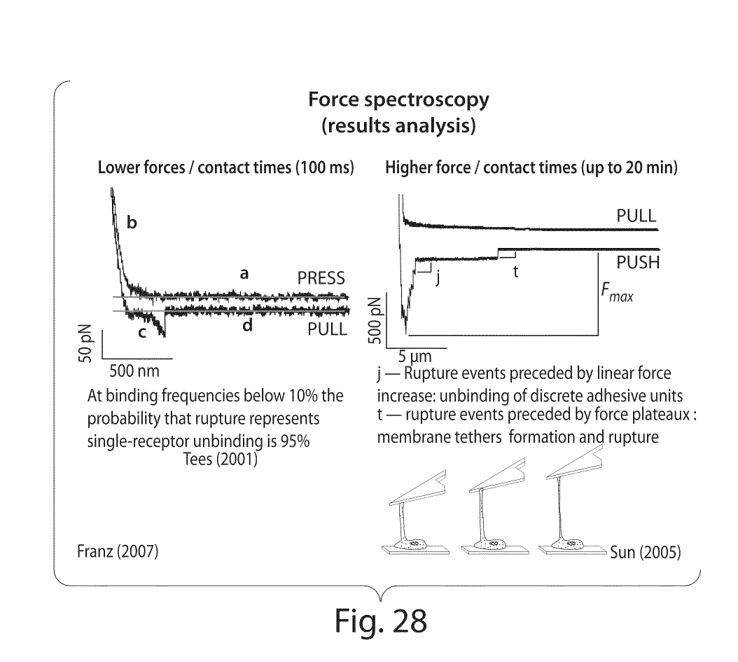

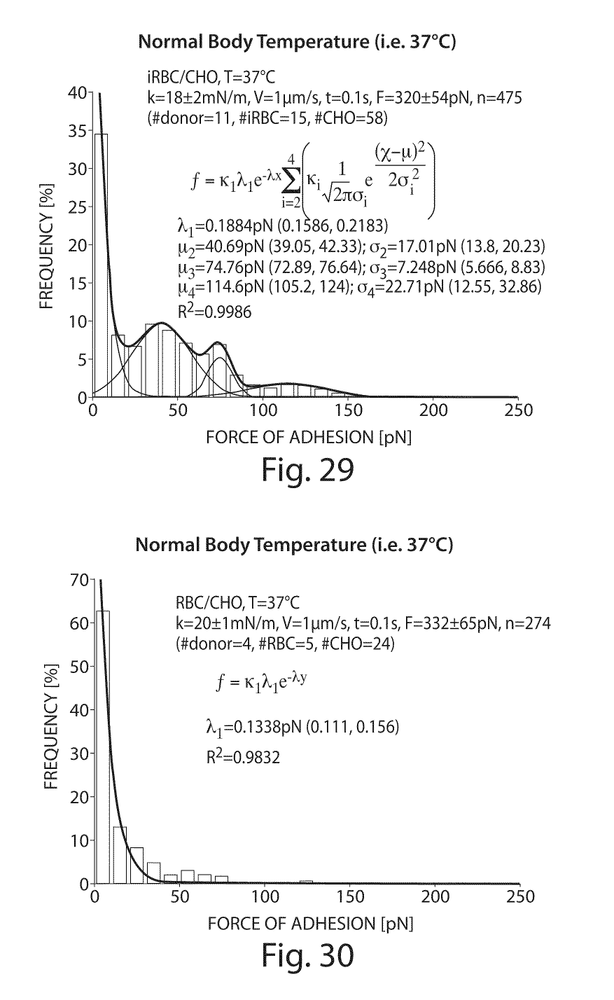

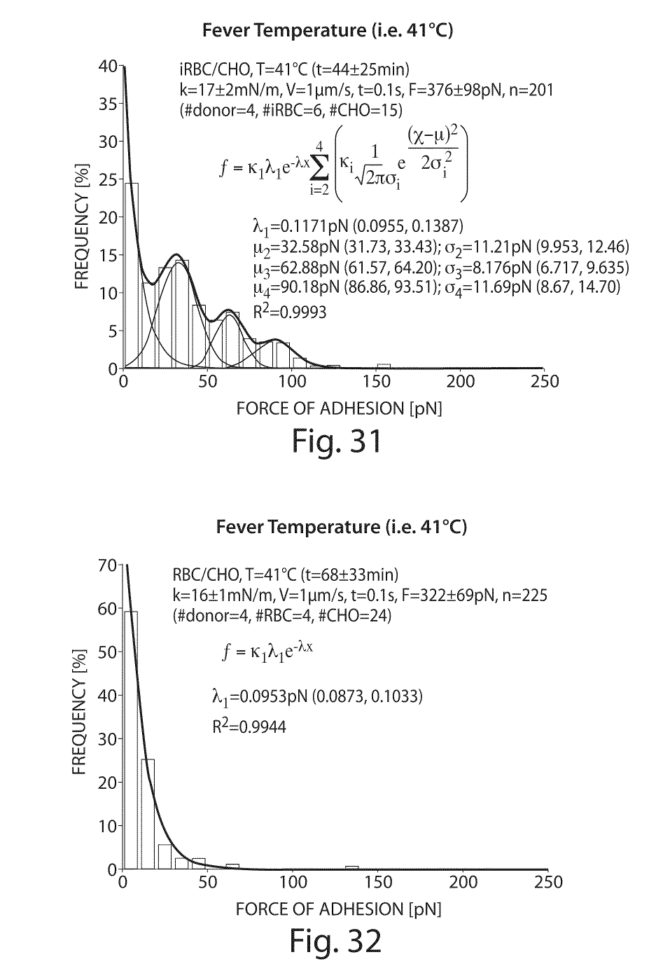

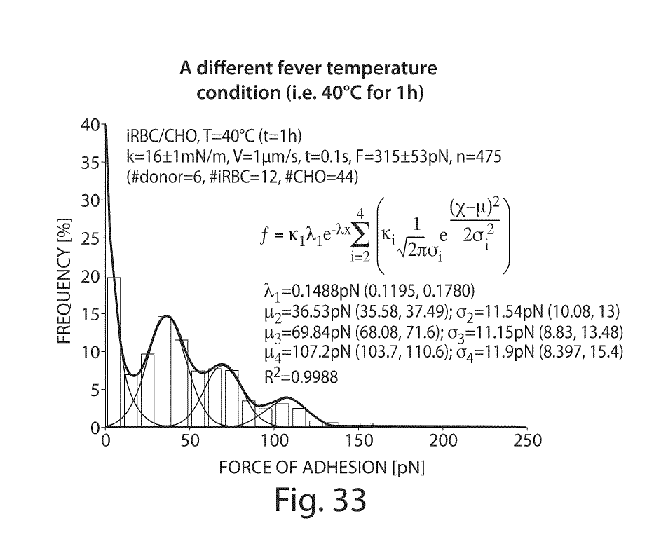

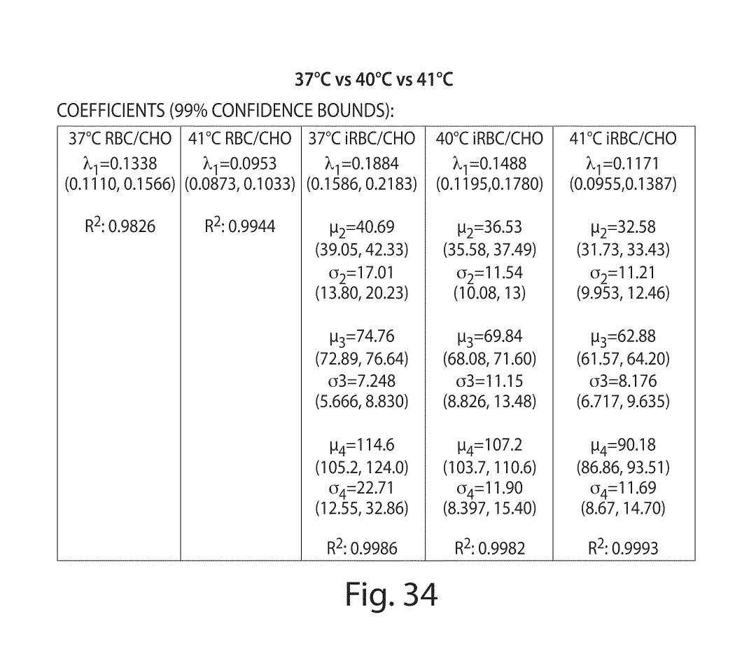

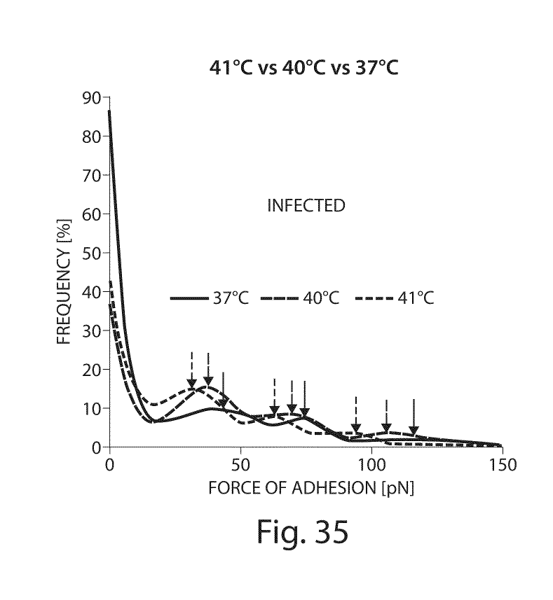

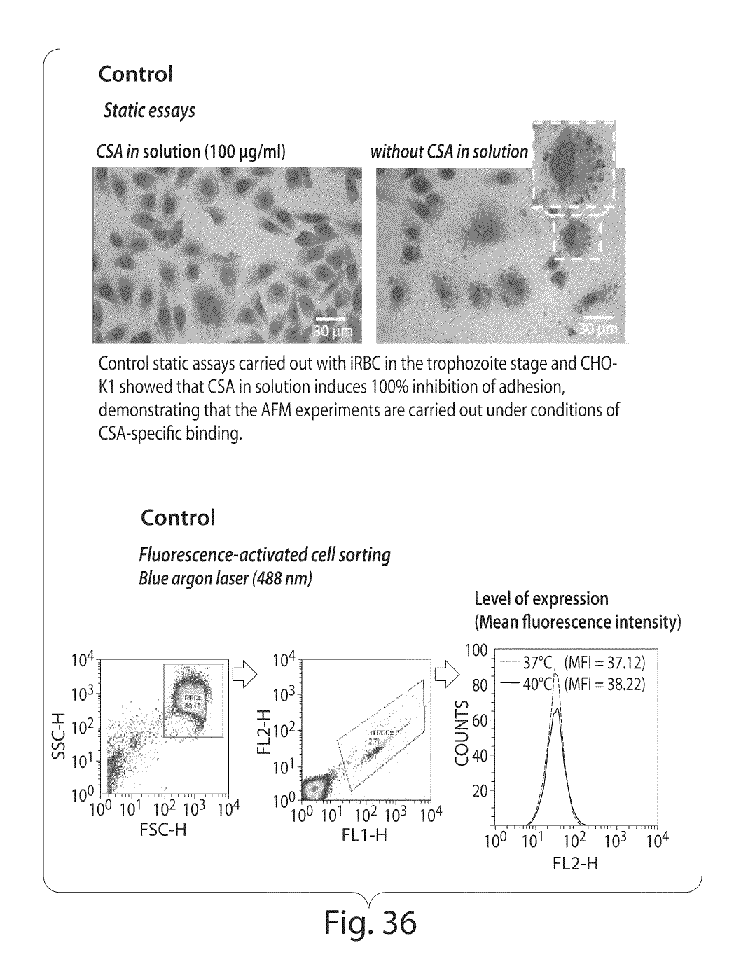

Cell Adhesion properties were investigated by atomic force microscopy. FIG. 26 illustrates schematically the experimental setup used in adhesion force measurements. The experiments involved a previous culture of CHO on glass slides coated with poly-D-lysine (PDL), a mildly adhesive protein. P. Falciparum infected RBC was poured over the culture slide and the blood cells were allowed to weakly bind to the substrate through PDL mediation (step A). A tipless cantilever previously incubated with Concanavalin A (ConA), a strongly adhesive protein, was pressed against a chosen iRBC at late throphozoite stage (step B), which then became solidly attached to the cantilever (step C). The attached iRBC was subsequently positioned above a chosen CHO (step D) and engaged on this cell until the cantilever deflection reached the value corresponding to a preset trigger force (step E), after a defined contact time the cantilever was retracted at a set speed until the two cells are completely separated (step F). The cantilever deflection measured during retraction was used to determine the adhesion force (f) between iRBC and CHO. The adhesive mediators (PDL and ConA) required fine-tuning so that f_{iRBC/substrate}<f_{RBC/cantilever}>f_{RBC/CHO}. Control experiments were also carried out with non-infected RBCs.

### Parasite Culture

A clone derived from P. falciparum FCR3-CSA parasites (strain with CSA binding phenotype) was maintained in leukocyte-free human O+erythrocytes (Research Blood. Components, used no more than two days after collection) and stored at 4° C. for no longer than 2 weeks under an atmosphere of 3% O₂, 5% CO₂, and 92% N₂ in RPMI medium 1640 (Gibco Life Technologies, Rockville, Md.) supplemented with 25 mM Hepes (Sigma, St. Louis, Mo.), 200 mM hypoxanthine (Sigma), 0.209% NaHCO₃ (Sigma), and 0.25% albumax I (Gibco Life Technologies). Cultures were synchronized successively by concentration of mature schizonts using plasmagel flotation and sorbitol lysis 2 h after the merozoite invasion to remove residual schizonts kit The mechanical tests were performed within 24-36 h (trophozoite stage) after merozoite invasion.

CHO culture

Chinese Hamster Ovary cells (CHO-K1, CCL-61 American Type Culture Collection) were grown in an incubator at 37° C. with 5% CO₂ in a F-12K (ATCC) modified medium containing 10% Fetal Bovine Serum (Gibco, 26140-079) neutralized at 56° C. for 30 min, and 1% Penicillin/Streptomycin (Biofluids, 303),

### Slide Preparation

The glass slides were dipped in 0.1 mg/ml PDL (Sigma) for 10 min, drained and dried overnight at room temperature. Adherent CHO growing at 70% confluence were harvested from a cell culture flask after incubation for 5 min with 3 ml of Accutase (Invitrogen), then washed in RPMI medium 1640 (Gibco Life Technologies) and re-suspended to 1×10⁶ cells/ml in the CHO culture buffer. A cell suspension drop of 100 μl was laid on the PDL precoated slide and incubated for 24-48 h at 37° C. with 5% CO₂. A slide with well-spread adherent CHO was gently washed with 1× phosphate buffered saline (PBS)-Ca—Mg (Invitrogen), Malaria culture in trophozoite stage with 2-10% parasitemia was diluted in 1×PBS-Ca—Mg with 0.05% Bovine Serum Albumin (BSA) (Sigma) to 1% hematocryte and was poured over the slide with adherent CHO and allowed to stand for 10 minutes. Non-attached blood cells were washed with 1×PBS-Ca—Mg with 0.05% BSA and the slide with adherent CHO and lightly attached iRBC/RBC was then immersed in the same buffer and transferred to the microscope liquid cell.

### Force Spectroscopy

The force spectroscopy experiments were conducted with an extended-head Asylum Research MFP-3D atomic force microscope (AFM) mounted on an Axiovert Zeiss trans-illuminated microscope. The spring constant (k) of each silicon nitride tipless cantilever (MLCT-O10 Veeco, with nominal k of 30 mN/m) was calibrated in air using the thermal noise method [iii]. A calibrated tipless cantilever was incubated in 1 mg/ml ConA for 30 minutes prior to the force spectroscopy measurements. The liquid cell was loaded with the slide and filled with PBS-Ca—Mg with 0.05% BSA that was kept at 37° C. or 41° C. during the experiments. The ConA-incubated cantilever was immersed in the heated buffer and the measurements were carried out after allowing for minimal thermalization (10-20 min to avoid consuming the minimal time of parasite/RBC/CHO viability). The inverse optical sensitivity was determined by performing an extension/retraction cycle in liquid against the rigid glass slide. The cantilever was subsequently engaged with a contact force of 1 nN for 30 s on a chosen iRBC in late trophozoite stage (or RBC), which became attached to the cantilever through ConA mediation and was withdrawn from the substract upon retraction. This iRBC(RBC) was then used to probe several CHOs around the slide. Each experiment typically involved testing an individual iRBC(RBC) probe for no more than 150 extension/retraction cycles with a displacement rate (V) of 1 μms⁻¹, a trigger force (F) of 300 pN and a dwell time (t) of 0.1 s. All CHO cells tested were typically well spread on the substrate, the shape of the attached iRBC(RBC) was thoroughly checked and the rotation of the hemazoin crystals inside the parasitophorous vacuole was closely monitored during each experiment. Force/displacement curves were obtained by converting the measured deflection into force using the calibrated sensitivity, and the measured piezo-displacement into probe/sample separation through subtraction of the cantilever deflection as described in ref. [iv]. Tilt and curvature induced by hydrodynamic effects on the baselines [iv] were corrected with a polynomial function, which was typically not higher than a 3^{rd} order polynomial. Since the trigger force is imposed as a deflection difference relative to the initial value, the hydrodynamic effects induced some scattering on the used trigger force, which was treated statistically. The offset observed at rupture in the retraction curve was used to quantify the adhesion force (f) associated with each extension/retraction cycle and the values obtained were used to produce force histograms. The effective spring constant (k_{eff}) of the cantilever-iRBC-CHO-bond system was determined for retraction curves exhibiting discrete rupture events from the slope of a line fitted to the region preceding rupture. In order to minimize the influence of tethering, the measurements were carried out for separation distances below 8 μm. Zero separation was typically considered to occur at the point of steep slope change in the extension baseline (jump-in effects were essentially nonexistent and long-range repulsion forces were assumed to be absent in the liquid [v]). The effects of febrile temperature on CHO adhesion behavior were controlled by force spectroscopy experiments carried out at 37° C. after heat treating the iRBC at febrile temperature for 1 h. The fraction of parasites in trophozoite stage, with unambiguously rotating hemazoin crystals after heat treating for 1 hour at 41° C., was profoundly low. Localization of a viable iRBC was essential, yet very time consuming in order to achieve successful experiments. As a result, the control experiments were carried out with an iRBC incubated at 40° C. for 1 h prior to adhesion force measurements at 37° C.

### References for Example 5

[i] Pasvol G, Wilson R J, Smalley M E, Brown J., Ann Trop Med. Parasitol. 1978 February; 72(1):87-8.
[ii] Lambros C, Vanderberg J P., J. Parasitol. (1979) June; 65(3):418-20.
[iii] P. R. Saulsen, Phys. Rev. D 42 (1990) 2437
[iv] C. M. Franz A, Taubenberger, Pucell, D. J. Muller, Sci. SIRE, (2007) 406, p. p 15
[v] Hans-Jurgen Butt, Brunero Cappella, Michael Kappl, Force measurements with the atomic force microscope: Technique, interpretation and applications, Surface Science Reports 59 (2005) 1-152

### Example 6

### Quantifying the Biophysical Characteristics of Plasmodium-falciparum-Parasitized Red Blood Cells in Microcirculation

Red blood cells parasitized by Plasmodium falciparum (Pf-RBCs) undergo irreversible changes in structure and biophysical characteristics. These changes can lead to drastically altered blood circulation. The membrane stiffness of infected RBCs may increase by up to ten-fold causing capillary occlusions [1, 2], thereby resulting in substantial increase in resistance to blood flow. Such effects may be intensified due to the enhanced cytoadherence of Pf-RBCs to the vascular endothelium [3, 4, 5, 6]. This adherence of Pf-RBCs is believed to be the main cause of bleeding complications in cerebral malaria due to blockages of small vessels in the brain [7]. Unlike the extensive research on leukocytes, only very few in vitro experiments [8, 9, 10, 11] have examined the adhesive dynamics of Pf-RBCs. More broadly, there have not been any quantitative studies of the dynamics of RBCs in malaria to investigate the rheology and flow resistance in addition to the reported new adhesive dynamics.

In summary, in the current work using DPD we modeled the RBC membrane as a viscoelastic material, the solid Pf-parasite, the fluid inside the cells and the exterior plasma, as well as the functionalized microchannel walls. The model parameters included the membrane shear modulus μ0, the membrane bending rigidity kc, the membrane viscosity ‘m, and the interior/exterior ‘i/‘o fluid viscosities.

### Methods

### Simulation Method

The DPD method described in [38] is a particle based mesoscopic simulation technique, where a simulated system consists of N point particles. Each particle corresponds to a collection of atoms or molecules rather than an individual atom. DPD particles interact through pairwise softpotentials and move according to the Newton's second law of motion.

### Membrane Model

The RBC membrane was modeled by discrete points between 500 and 30 000, which were the vertices of a triangular network of springs on the membrane surface. The network of fixed connectivity provided the elastic and the viscous response of a RBC since a “dashpot” is attached to each spring. The RBC model also included bending energy between neighboring triangular plaquettes and area and volume constraints.

A “stress-free” model as described in [14] was applied here. This model eliminates existing artifacts of irregular triangulation. It is obtained by simulation annealing such that each spring assumes its own equilibrium spring length adjusted to be the edge length after triangulation. RBC-fluid boundary conditions were enforced through bounce-back reflections of fluid particles on the membrane triangles and by a proper setting of interactions between fluid particles and RBC vertices.

### Adhesive Dynamics

Adhesive dynamics were simulated with the stochastic bond formation/dissociation model similar to that disclosed in [17]. The bonds were modeled as linear springs and their formation kₒₙ and dissociation k_{off} rates depend on the separation distance between the RBC receptors and ligands distributed on the wall as a square lattice with the lattice constant of 0.5 μm. Adhesive dynamics in simulations proceeded by (1) checking for potential dissociation of existing bonds with probability 1−exp(−koff¢t), where ¢t is the time step, (2) testing unbound ligands for potential bond formation with probability 1−exp(−kon¢t), and (3) applying forces of all existing bonds.

### Results

We first validated our RBC model in health and disease with physiologically correct values of all parameters using data from optical tweezer experiments. Subsequently, using the same set of parameters we investigated the dynamics of Pf-RBCs at different parasetimia levels and quantified the different modes of adhesive dynamics in the presence of ICAM-1 coated wall surfaces.

### Increased Stiffness of Pf-Parasitized RBCs

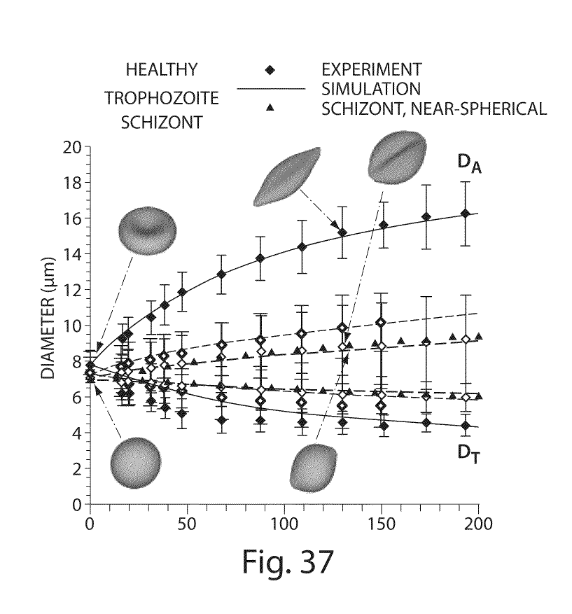

In malaria disease, progression through the parasite development stages (ring→trophozoite→schizont) leads to a considerable stiffening of Pf-RBCs compared to healthy ones [21, 24]. Furthermore, in the schizont stage the RBC shape becomes near spherical whereas in the preceding stages RBCs maintain their biconcavity. FIG. 37 shows simulation results for healthy RBCs and Pf-RBCs at different stages of parasite development compared with optical tweezer experiments [24]. The simulation results were obtained with a stress-free multiscale RBC model (see Methods) with 500 points, shear modulus μ0=6.3 μN/m for the healthy RBC, 14.5 for the ring stage, 29 for the trophozoite, and 60 μN/m for the schizont. The bending rigidity was set to 2.4×10-19 J for all cases. The curve for the schizont stage marked as “near-spherical” corresponds to stretching an ellipsoidal shape with axes aₓ=a_{y}=1.2a_{z}. Here, the membrane shear modulus of 40 μN/m matched the stress-strain response with the experiment, i.e., it is smaller than that for the biconcave-shape simulation. For the near-spherical cell the membrane was subject to stronger local stretching for the same uniaxial deformation compared to the biconcave shape. For the deflated biconcave shape, the inner fluid volume can be deformed in response to stretching, while in the near-spherical shape the fluid volume applies additional resistance onto the stretched membrane. Hence, the cell geometry plays an important role, and hence it has to be closely modeled for accurate extraction of parameters from the optical tweezer experiments.

### Flow Resistance

First we modeled the blood as a suspension of healthy RBCs using the DPD model and simulate blood flow in tubes of diameters ranging from 10 μm to 40 μm. It is important to model carefully the excluded volume (EV) interactions among cells. If we set the repulsive force coefficient between membrane vertices too high we would introduce a non-zero screening length between two membrane surfaces governed by the cutoff radius of the repulsive interactions. Hence, the choice of a smaller cutoff radius can result in overlapping of cells, while a larger one can increase the screening distance between cells, which may strongly affect the results at high volume fractions of RBCs. One approach was to enforce EV interactions among cells by employing reflections of RBC vertices on the membrane surfaces of other cells with small repulsive force coefficient yielding essentially a zero screening length between two RBC surfaces.

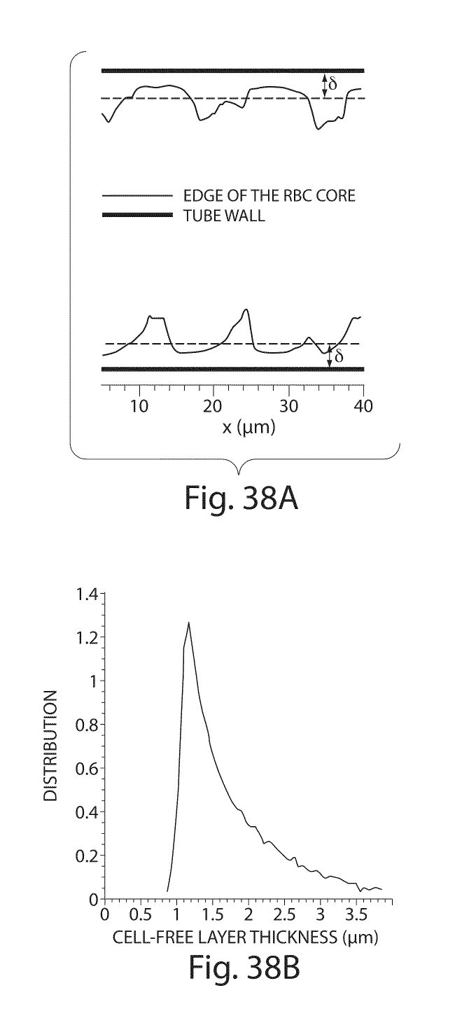

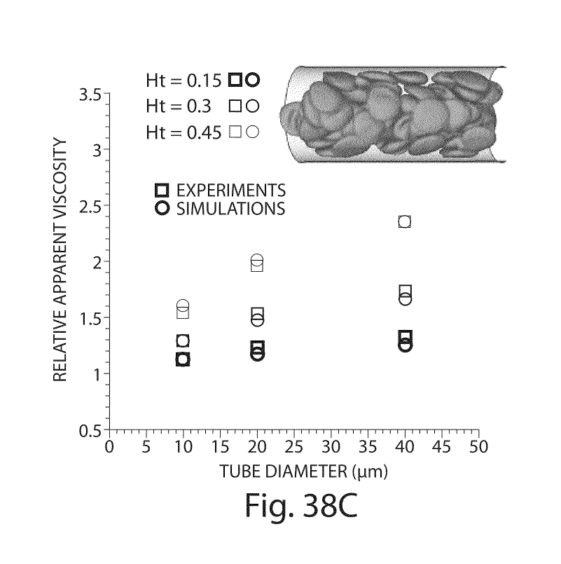

In addition, we employed a net repulsion of RBCs from the wall by properly setting the repulsive force coefficient between the wall particles and the cell vertices. RBCs in Poiseuille flow migrated to the tube center forming a core in the flow. FIG. 38 shows a sample snapshot of RBCs flowing in a tube of diameter D=20 μm. The pressure gradients employed here are 2.633×105 Pa/m and 6.582×104 Pa/m for tubes of diameters 10 μm and 40 μm, respectively. In the case of low hematocrit Ht (e.g., 0.15) the velocity profiles closely follow parabolic curves in the nearwall region. In the central region of the tube a substantial reduction in velocity is found for all volume fractions in comparison with the parabolic profiles indicating a decrease in the flow rate. An RBC core formation was clearly observed with a thin plasma layer next to the tube walls called the cell-free layer (CFL). The thickness of the CFL is directly related to the Fahraeus and the Fahraeus-Lindquist effects, both of which were accurately simulated by our DPD model as described in [14]. To determine the CFL thickness we computed the outer edge of the RBC core. FIG. 38 also shows a sample CFL edge from simulations and CFL thickness distribution for Ht=0.45 and D=20 μm. The fluid viscosity of the CFL region is much smaller than that of the tube core populated with RBCs providing an effective lubrication for the core to flow. The apparent viscosity is defined as follows ηₐₚₚ=¼¢PD⁴/128QL, where ¢P is the pressure difference, Q is the flowrate, and L is the length of the tube. It increases for higher Ht values since higher cell crowding yields larger flow resistance. It is more convenient to consider the relative apparent viscosity defined as ηᵣₑₗ=ηₐₚₚ/ηₛ, where ηₛ is the solvent viscosity. FIG. 38 shows the simulated ‘rel values in comparison with the empirical fit to the experiments described in [31] for the tube diameter range 10-40 μm and Ht values in the range 0.15-0.45. Excellent agreement between simulations and experiments was obtained for the proper EV interactions for all cases tested.

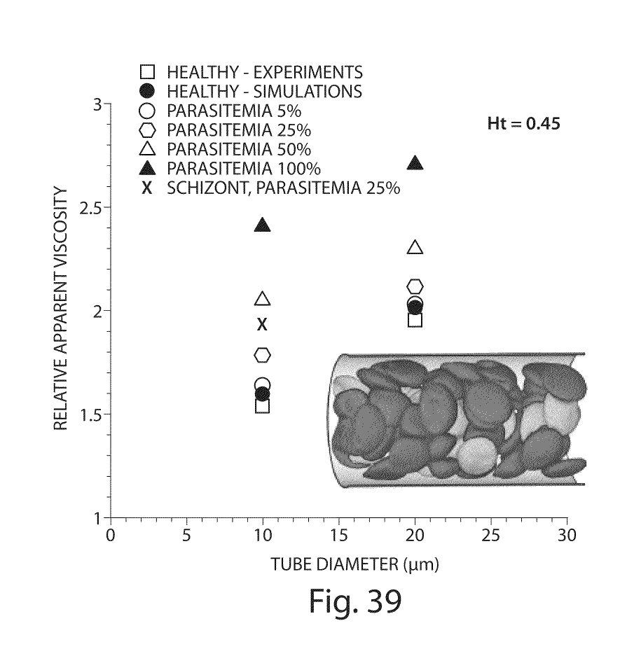

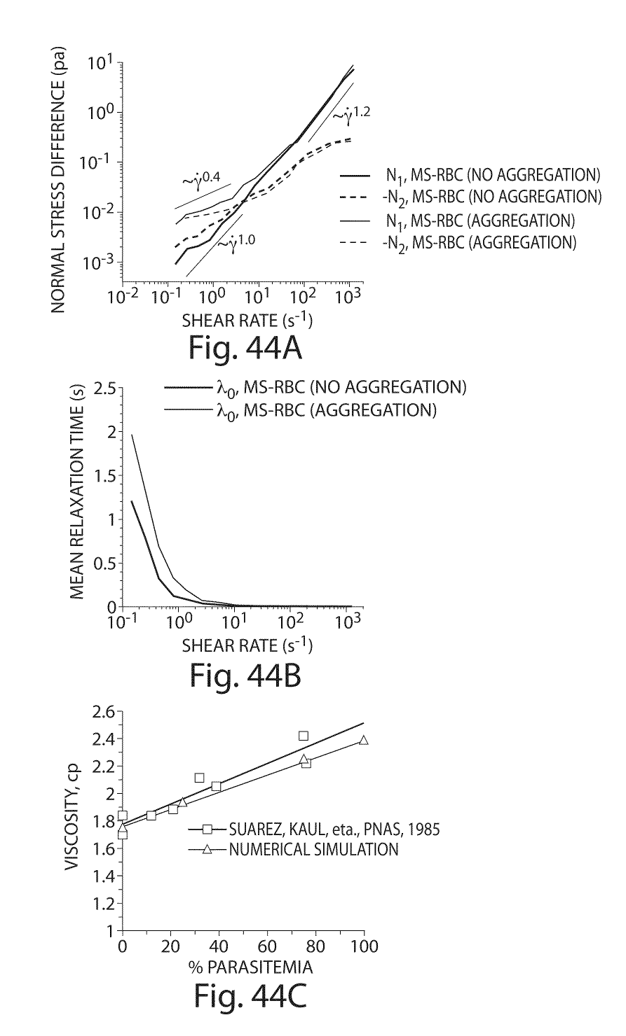

Next we simulated blood flow in malaria as a suspension of healthy and Pf-RBCs at the trophozoite stage and hematocrit Ht=0.45. Several parasitemia levels (percentage of Pf-RBCs with respect to the total number of cells in a unit volume) from 5% to 100% are considered in vessels with diameters 10 and 20 μm. Our results indicate that the parasitemia levels are in a linear correlation with the viscosities of numerically stimulated Pf-RBC suspensions (FIG. 44C). See also Raventos-Suarez et al., PNAS, 82(11):3829-3833, 1985. The inset of FIG. 39 shows a snapshot of RBCs flowing in a tube of diameter 20 μm at a parasitemia level of 25%. The main result in FIG. 3 is given by the plot of the relative apparent viscosity in malaria—a measure of flow resistance—obtained at different parasitemia levels. The effect of parasitemia level appears to be more prominent for small diameters and high Ht values. Thus, at Ht=0.45 blood flow resistance in malaria may increase up to 50% in vessels of diameters around 10 μm and up to 43% for vessel diameters around 20 μm. These increases did not include any contributions from the interaction of Pf-RBCs with the glycocalyx; see [32, 33]. Such important interactions are complex as they may include cytoadhesion which we modeled next.

### Adhesive Dynamics

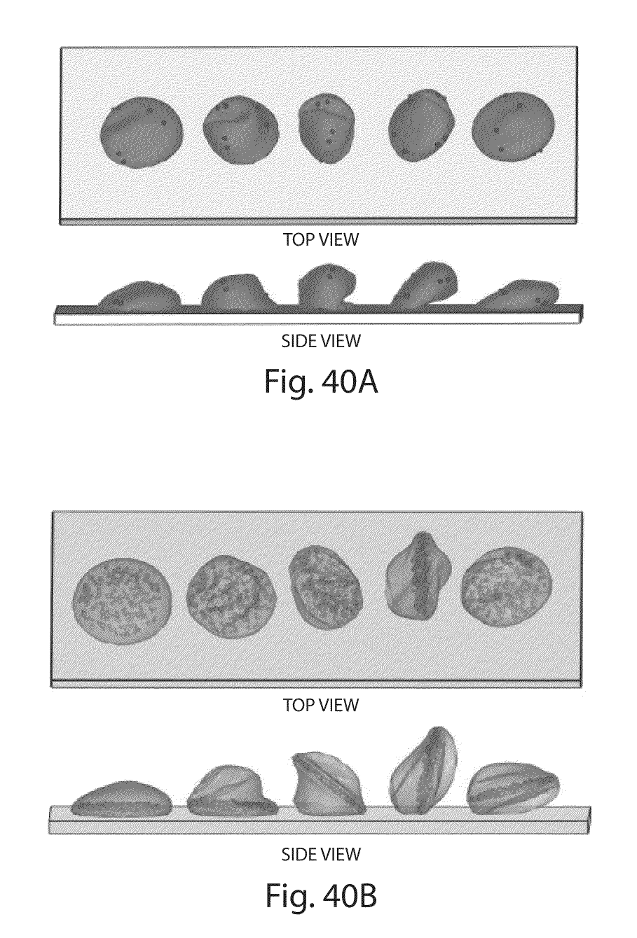

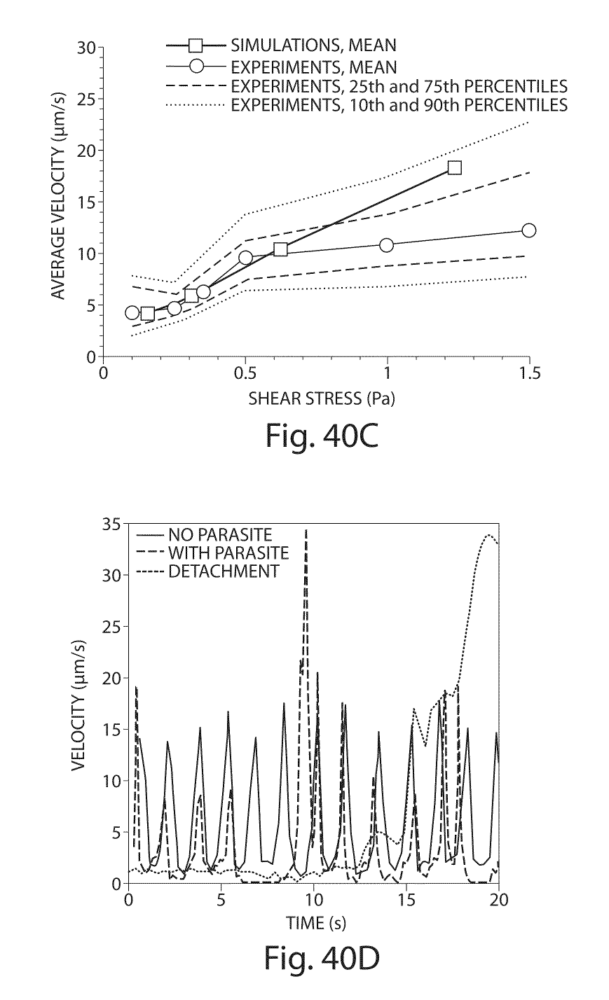

The adhesive dynamics of Pf-RBCs in shear flow was studied for different values of wall shear stress (WSS) and compared with the results from the experiments disclosed in [8] for the wall coated with purified ICAM-1. FIG. 40 (Panel A) shows several successive snapshots of a cell rolling along the wall. Small blue particles are added as tracers for visual clarity, and distinct RBC snapshots are separated by shifting their x coordinate. The dynamics of the Pf-RBCs was characterized by a flipping behavior initiated at first by the cell peeling off the wall due to the hydrodynamic force after flat RBC adhesion (the first snapshot in the plot). After most of the initial cell-wall contact-area was peeled off, the RBC flips over onto its other side facilitated by the remaining small wall contact-area. During these steps, Pf-RBCs undergo strong membrane deformations as illustrated in the plot. Similar flipping behavior and large membrane deformations (including membrane buckling) were also found as described in [8]. WSS appears to be the key parameter governing the Pf-RBC adhesive dynamics, since adhered RBCs are driven by fluid stresses and roll along the wall with a much smaller velocity than the flow velocity. Several initial simulations with varying WSS and other parameters fixed revealed that Pf-RBCs can exhibit firm adhesion at a WSS lower than 0.317 Pa while they can completely detach from the wall at higher values. Systematic visualizations showed that Pf-RBC detachment at high WSS occurs during the relatively fast motion of RBC flipping, since the contact-area is then minimal.

To stabilize RBC binding at high shear stresses we improved the model by allowing the bond spring constant (ks) to vary with WSS. For simplicity, we assume linear dependence. FIG. 40 (Panel C) presents the average rolling velocity of Pf-RBCs compared with experiments of cell rolling on a surface coated with purified ICAM-1 (see [8]). The simulated average velocities show a near-linear dependence on the shear stress, and are in good agreement with the experiments. The discrepancy at the highest simulated shear stress suggests a further strengthening of cell-wall bond interactions. The simulated values remain between the 10th and the 90^{th} percentiles found in experiments.

In general, the adhesive behavior of Pf-RBCs, explored by means of numerical simulation for various parameters, revealed several types of cell dynamics such as firm adhesion, RBC peeling off the surface followed by flipping from one side to the other or by detachment from the wall, and very slow slipping along the wall. However, results from the experiments described in [8] show firm adhesion of Pf-RBCs for some time followed by sudden detachment. In contrast, firm adhesion in simulations appears always to be stable with no detachment within the simulation time of approximately 30 s. In experiments the Pf-RBC motion before the detachment displays very slow slipping along the surface due to the flow and random collisions with other flowing RBCs. Hence, the sudden complete detachment from the wall could be caused by the RBC slipping into a wall region with a limited number of ligands available for binding due to imperfect coating.

To verify this hypothesis, we ran a simulation in which the ligand sites were removed from the wall area between 30 μm and 40 μm in the flow direction. FIG. 40 (Panel D) presents the Pf-RBC instantaneous velocity (green curve) corresponding to slow slipping along the surface continued up to an x coordinate between 30 μm and 40 μm, where a complete cell detachment occurs due to absence of ligands for binding, in agreement with the Pf-RBC dynamics on the mammalian CHO cells found in experiments described in [8]. No other change in physical parameters of cell adhesion have been found to reproduce this dynamics.

Next, we modeled explicitly the effect of the solid parasite inside the Pf-RBCs. To prevent the parasite body from crossing the RBC membrane, we introduced Lennard-Jones interactions between the parasite body particles and membrane vertices. The number of DPD particles to represent the RBC cytosol is reduced according to the volume occupied by the parasite body. FIG. 40 (Panel B) presents successive snapshots of a rolling RBC with a rigid parasite inside the cell. The RBC membrane displays local buckling due to its low bending rigidity, which is consistent with the RBC visualizations in FIG. 40 (Panel A). In addition, a tank-threading motion of the membrane appears caused by the solid parasite. FIG. 40 (Panel C) shows the corresponding instantaneous velocity (red curve), exhibiting a more erratic pattern than the blue curve. For example, the red curve in FIG. 40 (Panel D) indicates several time intervals during which the Pf-RBC shows firm adhesion for several seconds. Furthermore, firm adhesion can be followed by several fast flips of the RBC along the surface characterized by two closely located peaks of velocity around the time of 20 s. Systematic visualizations revealed that the smaller peaks of cell velocity in FIG. 40 (Panel D) correspond to tank-treading like motion facilitated by the parasite body due to the parasite being freely suspended in the RBC cytosol. A proper positioning of the parasite body inside the RBC can result in a stress distribution on the front part of the membrane which forces the RBC into a crawling motion.

We have employed a validated multiscale model to quantify the dynamic properties of Pf-RBCs in typical conditions encountered in the microcirculation. To the best of our knowledge, this is the first such study and represents a paradigm shift in biomedical modeling. Specifically, the simulated mechanical responses of healthy RBCs and Pf-RBCs were found to be in excellent agreement with optical tweezer experiments as did the dynamic responses measured in terms of the cell free layer and the increase in the apparent blood viscosity. Flow resistance was computed at parasitemia levels higher than those often found in clinical blood tests of individuals suffering from malaria, see [36]. At a parasitemia level above 0.2% an immune response is initiated, and levels around 20% are found in very severe cases of malaria with high mortality [37, 9]. Clinical tests are able to detect Pf-parasitized RBCs at a parasitemia level as small as 0.0001-0.0004%. Active malaria in most cases is characterized by levels of 0.5%-20%. The parasitemia levels simulated here are beyond the ranges mentioned above. We indeed attempted to span the full range 0%-100% to evaluate the dependence of blood flow properties on parasitemia levels.

Moreover, our experimental data show a broader scatter of the average RBC velocity for different cells than found in simulations. This is likely to be related to nonuniform distributions of receptors on the RBC membrane and ligands on the wall. In the simulations, distributions of both receptors and ligands are fixed, and are nearly homogeneous with approximately the same area occupied by each receptor or each ligand. A scatter in behavior among distinct RBCs in the simulations is solely related to the stochastic nature of the adhesive model.

### References for Example 6

1. Shelby, J. P., J. White, K. Ganesan, P. K. Rathod, and D. T. Chiu, 2003. A microfluidic model for single-cell capillary obstruction by Plasmodium falciparum-infected erythrocytes. Proceedings of the National Academy of Sciences USA 100:14618-14622.
2. Cranston, H. A., C. W. Boylan, G. L. Carroll, S. P. Sutera, J. R. Williamson, I. Y. Gluzman, and D. J. Krogstad, 1984. Plasmodium falciparum maturation abolishes physiologic red cell deformability. Science 223:400-403.
3. Ho, M., N. J. White, 1999. Molecular mechanisms of cytoadherence in malaria. Am. J. Physiol., 276:C1231-C1242.
4. Brown, H., T. T. Hien, N. Day, N. T. H. Mai, L. V. Chuong, T. T. H. Chau, P. P. Loc, N. H. Phu, D. Bethe, J. Farrar, K. Gatter, N. White, and G. Turner, 1999. Evidence of blood-brain barrier dysfunction in human cerebral malaria. Neuropathology and Applied Neurobiology 25:331-340.
5. Ho, M., M. J. Hickey, A. G. Andonegui, P. Kubes, 2000. Visualization of Plasmodium falciparum-endothelium interactions in human microvasculature: mimicry of leukocyte recruitment. J. Exp. Med., 192:1205-1211.
6. Dondorp, A. M., E. Pongponratn, and N. White, 2004. Reduced microcirculatory flow in severe falciparum malaria: Pathophysiology and electron-microscopic pathology. Acta Tropica 89:309-317.
7. Adams, S., H. Brown, and G. Turner, 2002. Breaking down the blood.brain barrier: signaling a path to cerebral malaria? Trends in Parasitology 18:360-366.
8. Antia, M., T. Herricks, and P. K. Rathod, 2007. Microfluidic modeling of cell-cell interactions in malaria pathogenesis. PLoS Pathogens 3:939-945.
9. Roy, S., J. A. Dharmadhikari, A. K. Dharmadhikari, D. Mathur and S. Sharma, 2005. Plasmodium-infected red blood cells exhibit enhanced rolling independent of host cells and alter flow of uninfected red cells. Current Science 89:1563-1570.
10. Cooke, B. M., A. R. Berendt, A. G. Craig, J. MacGregor, C. I. Newbold, and G. B. Nash, 1994. Rolling and stationary cytoadhesion of red blood cells parasitized by Plasmodium falciparum: separate roles for ICAM-1, CD36 and thrombospondin. British Journal of Haematology 87:162-170.
11. Yipp, B. G., S. Anand, T. Schollaardt, K. D. Patel, S. Looareesuwan, and M. Ho, 2000. Synergism of multiple adhesion molecules in mediating cytoadherence of Plasmodium falciparum-infected erythrocytes to microvascular endothelial cells under flow. Blood 96:2292-2298.
12. Pivkin, I. V., and G. E. Karniadakis, 2008. Accurate coarse-grained modeling of red blood cells. Physical Review Letters 101:118105.
13. McWhirter, J. L., H. Noguchi, and G. Gompper, 2009. Flow-induced clustering and alignment of vesicles and red blood cells in microcapillaries. Proceedings of the National Academy of Sciences USA 106:6039-6043.
14. Fedosov, D. A., 2010. Multiscale modeling of blood flow and soft matter. PhD thesis, Division of Applied Mathematics, Brown University
15. Quinn, D., 2010. Dynamic behavior of healthy and malaria infected human red blood cells. PhD thesis, Department of Mechanical Engineering, Massachusetts Institute of Technology.
16. Li, J., M. Dao, C. T. Lim, and S. Suresh, 2005. Spectrin-level modeling of the cytoskeleton and optical tweezers stretching of the erythrocyte. Biophysical Journal 88:3707-3719.
17. Hammer, D. A., and S. M. Apte, 1992. Simulation of cell rolling and adhesion on surfaces in shear flow: general results and analysis of selectin-mediated neutrophil adhesion. Biophysical Journal 63:35-57.
18. King, M. R., and D. A. Hammer, 2001. Multiparticle adhesive dynamics: Hydrodynamic recruitment of rolling leukocytes. Proceedings of the National Academy of Sciences USA 98:14919-14924.
19. Alon, R., D. A. Hammer, and T. A. Springer, 1995. Lifetime of the P-selectincarbohydrate bond and its response to tensile force in hydrodynamic flow. Nature (London) 374:539-542.
20. Chen, S., and T. A. Springer, 1999. An automatic braking system that stabilizes leukocyte rolling by an increase in selectin bond number with shear. Journal of Cell Biology 144:185-200.
21. Park, Y.-K., M. Diez-Silva, G. Popescu, G. Lykotrafitis, W. Choi, M. S. Feld, and S. Suresh, 2008. Refractive index maps and membrane dynamics of human red blood cells parasitized by Plasmodium falciparum. Proceedings of the National Academy of Sciences USA 105:13730-13735.
22. Maeda, N., Y. Suzuki, J. Tanaka, and N. Tateishi, 1996. Erythrocyte flow and elasticity of microvessels evaluated by marginal cell-free layer and flow resistance. American Journal of Physiology, 271:H2454-H2461.
23. Kim, S., L. R. Long, A. S. Popel, M. Intaglietta, and P. C. Johnson, 2007. Temporal and spatial variations of cell-free layer width in arterioles. American Journal of Physiology, 293:H1526-H1535.
24. Suresh, S., J. Spatz, J. P. Mills, A. Micoulet, M. Dao, C. T. Lim, M. Beil, and T. Seufferlein, 2005. Connections between single-cell biomechanics and human disease states: gastrointestinal cancer and malaria. Acta Biomaterialia 1:15-30.
25. Marinkovic, M., M. Diez-Silva, I. Pantic, J. J. Fredberg, S. Suresh, and J. P. Butler, 2009. Febrile temperature leads to significant stiffening of Plasmodium falciparum parasitized erythrocytes. American Journal of Physiology: Cell Physiology 296:C59-C64.
26. Gov, N. S., and S. A. Safran, 2005. Red blood cell membrane fluctuations and shape controlled by ATP-induced cytoskeletal defects. Biophysical Journal 88:1859-1874.
27. Dimova, R., B. Pouligny, and C. Dietrich, 2000. Pretransitional effects in dimyristoylphosphatidylcholine vesicle membranes: Optical dynamometry study. Biophysical Journal 79:340-356.
28. Lee, C.-H., W.-C. Lin, and J. Wang, 2001. All-optical measurements of the bending rigidity of lipid-vesicle membranes across structural phase transitions. Physical Review E 64:020901.
29. Discher, D. E., and P. Carl, 2001. New insights into red cell network structure, elasticity, and spectrin unfolding. Cellular and Molecular Biology Letters 6:593-606.
30. Evans, J., W. Gratzer, N. Mohandas, K. Parker, and J. Sleep, 2008. Fluctuations of the red blood cell membrane: relation to mechanical properties and lack of ATP dependence. Biophysical Journal 94:4134-4144.
31. Pries, A. R., D. Neuhaus, and P. Gaehtgens, 1992. Blood viscosity in tube flow: dependence on diameter and hematocrit. American Journal of Physiology 263:H1770-H1778.
32. Weinbaum, S., J. M. Tarbell, and E. R. Damiano, 2007. The structure and function of the endothelial glycocalyx layer. Annual Review of Biomedical Engineering 9:121-167.
33. Popel, A. S. and P. C. Johnson, 2005. Annual Review of Fluid Mechanics 37:43-69.
34. Springer, T. A., 1995. Traffic signals on endothelium for lymphocyte recirculation and leukocyte emigration. Annual Review of Physiology 57:827-872.
35. Finger, E. B., K. D. Puri, R. Alon, M. B. Lawrence, U. H. von Andrian, and T. A. Springer, 1996. Adhesion through L-selectin requires a threshold hydrodynamic shear. Nature (London) 379:266-269.
36. Haenscheid, T., 1999. Diagnosis of malaria: a review of alternatives to conventional microscopy. Clinical and Laboratory Haematology 21:235-245.
37. Wilkinson, R. J., J. L. Brown, G. Pasvol, P. L. Chiodini, and R. N. Davidson, 1994. Severe falciparum malaria: predicting the effect of exchange transfusion. Quarterly Journal of Medicine 87:553-557.
38. Hoogerbrugge, P. J., and J. M. V. A. Koelman, 1992. Simulating microscopic hydrodynamic phenomena with dissipative particle dynamics. Europhysics Letters 19:155-160.

### Example 7

### Direct Computation of Human Blood Viscosity

Virtually all blood viscosity measurements are made “in vitro”, such that newly drawn blood is first stabilized with an anti-coagulant, then introduced into a viscometer. The sample is referred to as “whole blood” which consists of, in decreasing order by volume, red blood cells (RBCs), leukocytes, and platelets suspended in plasma. Under flow conditions at small deformation rates, the RBCs in whole blood have been observed to aggregate into structures called “rouleaux”, which resemble stacks of coins (1-4). To clarify the role of plasma proteins in the aggregation process (2,4), RBCs were separated from other particles and plasma, washed with solutions designed to remove all proteins adsorbed on their surfaces and re-suspended in pure saline. Fibrinogen was added progressively (2) while simultaneously measuring suspension viscosity. This revealed an increase in viscosity with increasing fibrinogen concentration at low deformation rates. Prolonged exposure of clean RBCs to acetaldehyde (5) sufficiently caused hardening such that re-suspension in Ringer solution resulted in constant suspension viscosity over the full range of hematocrit and shear rates.

Henceforth, fluids consisting of re-suspended RBCs will be referred to as erythrocyte suspensions (ES). Studies of ESs have demonstrated that: (i) the formation of rouleaux in healthy blood is mediated mainly by fibrinogen, (ii) the presence of fibrinogen is necessary for an ES to exhibit a measurable yield stress, and (iii) the hardening of RBCs increases ES viscosity, but reduces its shear-rate dependence.

These properties motivate an ES model of blood rheology since it is inherently simpler than a whole blood model, yet maintains a reasonable approximation for whole blood. The first suspension theory to be invoked for interpretation of the measured viscosity of blood is Casson's (6) model of mutually attractive pigment particles suspended in Newtonian oils. These particles aggregate at low shear rates to form rigid, rod-like structures whose length varies inversely with the shear rate. Casson's relation between the shear stress Γ_{xy} and the strain rate γ is given by

$${𝒯}_{xy}^{1 / 2} = {𝒯}_{y}^{1 / 2} + {η}^{1 / 2} {\dot{γ}}^{1 / 2} , \qquad ( 1 )$$

where T_{y} is a yield stress and η is the viscosity at large \dot{γ}. Casson's equation is one of several rheological relations which introduces a yield stress, a controversial concept since its determination rests on extrapolation of measurements at the lowest detectable shear stresses and shear rates. Merrill et al. (1) and others have employed Casson plots, i.e. T_{xy}^{1/2} vs. \dot{γ}^{1/2}, to extrapolate the yield stress with fair consistency.

### Models Employed

In our simulations, we employed two different models based on the dissipative particle dynamics (DPD), a coarse-grained molecular dynamics method for modeling seamlessly, liquids and soft matter (7-9). The multi-scale RBC model (MS-RBC) described in (12) represents the RBC membrane with hundreds or even thousands of DPD-particles (down to spectrin level) connected by springs into a triangular network in combination with out-of-plane elastic bending resistance. Extra dissipation within the network accounts for membrane viscosity, while the characteristic bi-concave RBC shape is achieved by imposition of constraints for constant membrane area and constant cell volume. Data from optical tweezers and dynamic experiments on single real RBCs are used to fit the model parameters, and no further adjustment is made for the RBCs in suspension. Because simulations with the MS-RBC model are time-consuming, we also employed a recently developed, low-dimensional model (LD-RBC) of a RBC, see (13). Tests of this model against the MS-RBC have already shown to accurately capture the mechanical response of single real RBCs. In contrast to the MS-RBC model, the LD-RBC model is constructed as a closed torus-like ring of only ten large, hard DPD-particles previously employed (14) to represent colloids in suspension. They are connected into a ring by springs in combination with bending resistance between two neighboring connections. We found that, as with the MS-RBC model, the LD-RBC model can be fitted to represent the entire range of elastic deformations as measured by optical-tweezers (15) for healthy and for malaria infected RBCs.

In addition to the LD-RBC and MS-RBC models, we developed an aggregation model, described in Methods, which we incorporated into the RBC suspension models to simulate the reversible rouleaux formation and destruction, which is essential in capturing the blood flow behavior, especially at low shear rates.

### Results

### In-Silico Versus In-Vitro Blood Viscosity

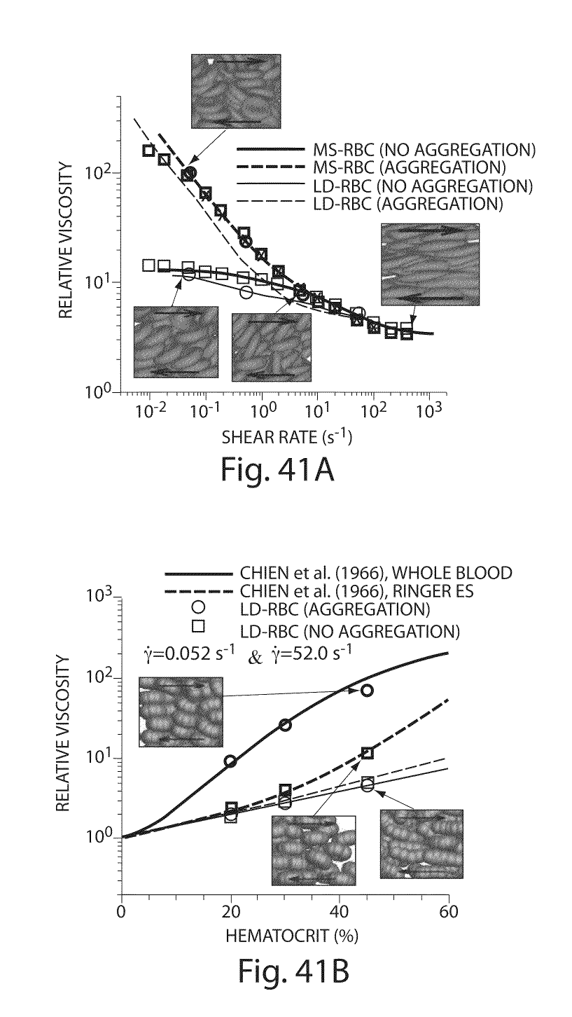

In this work, viscosity was derived from simulations of plane Couette flow using the Lees-Edwards periodic boundary conditions, in which the shear rate and the density of cells were verified to be spatially uniform. The experimental viscosities of well-prepared ESs without rouleaux and of whole blood were measured at hematocrit H=45% and at temperature 37° C. following the methods described in (1, 16, 17) using rotational Couette viscometers. At the same conditions for both the MS-RBC and the LD-RBC suspensions, the viscosities were computed, with and without rouleaux, as functions of the shear rate over the range 0.005 s⁻¹ to 1000.0 s⁻¹. RBC suspension viscosities were normalized by the viscosity values of their suspending media. These data are compared in FIG. 41(a) as relative viscosity against shear at constant hematocrit. The MS-RBC model viscosity curves lie very close to the viscosities measured in three different laboratories. The model, consisting only of RBCs in suspension, clearly captures the effect of aggregation on the viscosity at low shear rates, and suggests that particles other than RBCs have little effect on the viscosity. The measured values for whole blood are more consistent than those for ESs, which may reflect differences in ES preparation. The LD-RBC model underestimates somewhat the experimental data, but is generally in good agreement over the whole range of shear rates, and again demonstrates the effect of aggregation. This is remarkable in view of the simplicity and economy of that model.

The dependence of whole blood and ES viscosity on hematocrit (H) is shown in FIG. 41(b). The curves are measured viscosities correlated with H at constant shear rate by Chien et al. (16), and the points are calculated with the LD-RBC model. This clearly shows how the model captures the H dependence on viscosity, and again demonstrates aggregation to be crucial for a quantitative account of the difference between the viscosity of whole blood and that of washed erythrocyte suspensions.

Recent attempts in modeling (18, 19) of two-cell, and of multiple cell aggregates (20) simulated only their flow behavior while Liu et al. (21) attempted to link viscosity with RBC aggregation. Their three-dimensional continuum model couples the Navier-Stokes equations with cell interactions; however, its viscosity prediction fails to capture the steep rise of that function at low shear rates. It appears that their system of only ten RBCs is inadequate to represent the bulk flow of a suspension.

### Reversible Rouleaux Formation

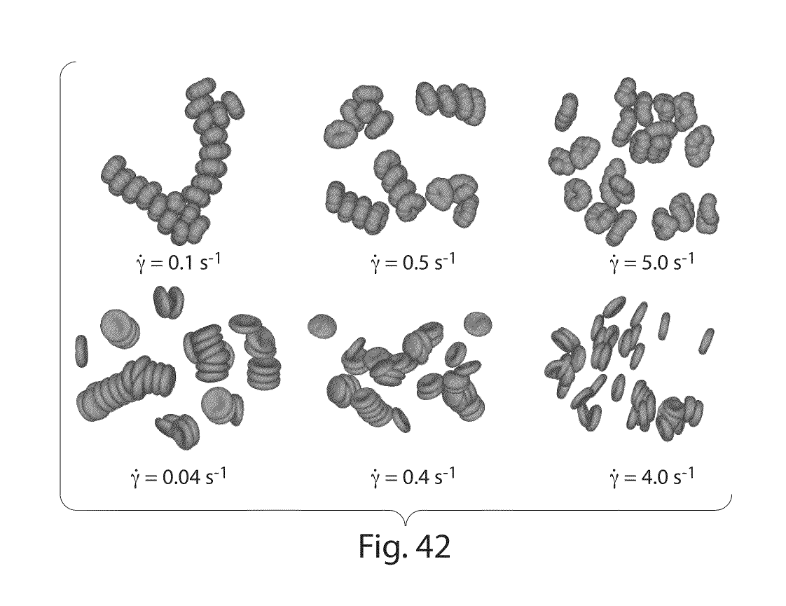

The formation of rouleaux, requires shear rates sufficient for frequent RBC collisions, yet gentle enough to avoid immediate dispersion. Experimentally, aggregation is observed to be a two-step process: the formation of a few RBCs into short linear stacks, followed by coalescence into long linear and branched rouleaux, see (1, 22). As the shear rate increases, the large rouleaux break up into smaller ones, and at higher values the suspension ultimately becomes one of mono-dispersed RBCs (23). This process then reverses as the shear rate is decreased. This typical formation-destruction behavior of rouleaux is consistent with the results of simulations using both the LD-RBC and the MS-RBC model as shown in FIG. 42. At low shear rates (left frames), the initially dispersed RBCs aggregate into large rouleaux of up to about 20 RBCs; as the shear rate is increased to moderate values (middle frames), these structures are reduced in size until, at high rates (right frames) they are dispersed almost completely into individual RBCs. Reversibility is demonstrated by reduction of the shear rate to the formation value at which point individual RBCs begin to re-aggregate. The attractive forces required for rouleaux formation were adjusted at one point only to realize the behavior shown in FIG. 42, and no further adjustments were made in the subsequent calculation of suspension viscosity.

### Yield Stress and Aggregation

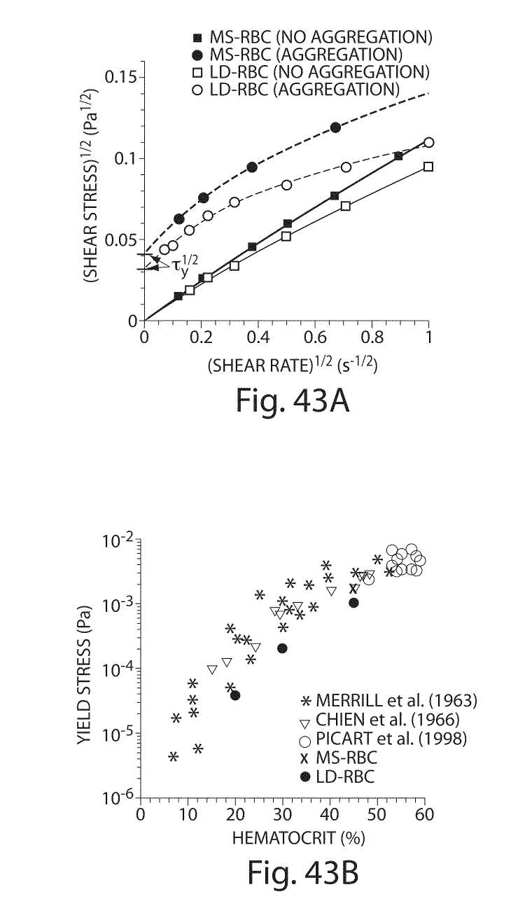

For whole blood, the most reproducible yield stresses are those extrapolated to zero shear rate from viscometric data on the basis of the Casson's equation (1). The assumptions of Casson's relation hold most likely, if anywhere, at the very low shear-rate region. At high shear rates, the Casson constant η is the suspension viscosity, which depends on the particle type and the viscosity of the suspending medium. When the yield stress T_{y} vanishes, equation (1) reduces to Newtonian liquid. Similar to the plots for pigment-oil suspensions (see (6)), the ES data provided in (2) follow equation (1) approximately at moderate and at high shear rates, but deviate at low shear rates. FIG. 43(a) is a Casson plot of simulated data for H=45% suspension, which shows clearly that T_{y} obtained by extrapolating the data to zero shear rate is absent without aggregation. The simulation data are those of FIG. 41 which show how the Casson coordinates tend to mask the non-Newtonian character of the suspensions. The curves of FIG. 43(a) were fitted from the simulation data, and their extrapolation to zero shear rate determines T_{y} without further assumptions. Support for the Casson extrapolation procedure is provided by the viscometric data for suspensions of Chinese ovary hamster cells (CHO), which differ geometrically from RBCs. These data have been reduced to a single master curve of dimensionless shear-stress versus dimensionless shear-rate over a wide range of volume fractions and shear rates, see (24). At low shear-rates the master curve is very close to the low shear-rate asymptotic limit of equation (1).

The yield stress for blood has been attributed to the presence of rouleaux by several authors (1, 25, 26). The measurement of yield stress is complicated by the nature of blood and type of instruments used (27). At the lowest shear-rates, sedimentation and viscometer wall-effects are complicating factors, and yield stresses derived from viscometric data are not consistent with those derived from non-rheological measurements (28). Merrill et al (1) found T_{y} of normal human blood to lie between 0.0015 and 0.005 Pa at H=45%, and to vary as H_{1/3}, similar to the dependence Thurston described in (29) for the elastic modulus of normal blood. Evidence for a yield stress was found in normal blood down to shear rates of 0.001 s⁻¹ (26). FIG. 43(b) shows that the T_{y} of FIG. 43(a) are in good agreement with viscometric data also obtained by Casson extrapolation, which is to be expected in view of the agreement between the calculated and the measured viscosities.

We also computed the normal-stress differences, which are displayed in FIG. 44. They are the only known estimates of these functions, and their validity rests on the accurate prediction by the MS-RBC model of the measured viscosity functions of FIG. 1(a). Interestingly, N₁, N₂ are typically of the same order of magnitude as the shear stress over the entire shear rate range. There appear to be no experimental data available for comparison with the calculated N₁ and N₂ functions presented here. For whole blood Copley and King (30) found N₁ to lie below the detection threshold of the Weissenberg rheogoniometer, which employed the plate pressure distribution to deduce N₁. Modern total-force cone-plate instruments can detect normal stresses as low as 2-3 Pa, i.e. the upper end of the N₁ curves of FIG. 44(a), where for blood inertia overwhelms N₁ in rotational instruments.

### Discussion

Accurate prediction of the relatively non-Newtonian viscosity from simulations of suspensions of model RBCs (FIG. 41) suggests a new paradigm for blood rheology. It has been shown that departures of blood viscosity from normal values correlate with various diseases and abnormal blood conditions. See, L. Dintenflass, Molecular rheology of human blood: its role in health and disease (to day and to morrow?), Proceedings of the 8^{th} International Congress on Rheology Italy 3 467-480 (1980). However, hereto such correlations have had few theoretical guidelines for their interpretation. The predictions of FIG. 41 show that a suspension of model RBCs, characterized with single-cell experiments, captures the viscosity of healthy whole blood provided plausible forces of aggregation are included in the model.

The physical basis of spontaneous rouleaux formation or RBC aggregation is not yet determined, though it has been a subject of investigation in several theoretical studies. Two theoretical models attempt to explain the RBC aggregation mechanism: the bridging model (2,31), and the depletion model (32,33). The former assumes that macromolecules, such as fibrinogen, can adsorb on RBC surfaces and bridge them together. The latter proposes that polymer depletion adjacent to RBC surfaces, results in a reduction of space for polymer conformations and, therefore in an osmotic pressure which drives two neighboring RBCs to aggregate.

The main focus of this study is to quantify the influence of aggregation on the rheological properties of human blood. Aggregation of RBCs into rouleaux structures can be mediated by the Morse potential; see Example 9. The plausibility of the Morse parameter values was checked by calculation of the maximum force needed to break up two aggregated RBCs. The break-up pulling force in the normal direction is approximately 3.0 pN-7.0 pN, such that the lower value corresponds to a peeling breakup. Tangential or sliding breakup requires a force in the range of 1.5 pN-3 pN. These forces are much smaller than those imposed on single RBCs in stretching tests with optical tweezers described in (15), and are consistent with observations of rouleaux, which do not show any large cell deformations. In addition, measurements of a disaggregation force in shear flow (34) indicate that the shear stress required to break up rouleaux structure lies approximately between 0.01 Pa and 0.1 Pa, while the analogous simulations with the MS-RBC model yield the value of about 0.02 Pa.

The steady state normal-stress differences of shear flow are usually understood as a measure of the elasticity of a viscoelastic fluid. In unsteady shear flows, the dynamic shear stresses allow elastic effects to be more easily detected, such as Thurston's dynamic measurements (see (29)), which show blood to have measurable elasticity. Theories addressing heterogeneous continual gradients of normal-stress differences suggest that they may induce secondary motions and migration (35,36). The computed normal-stress differences, when known as functions of both shear rate and H, provide a means to verify the applicability of these theories to pressure-driven blood flows, where stress gradients are known to induce migration.

### References for Example 7

1. E. W. Merrill et al., Rheology of human blood near and at zero flow, Biophysical Journal 3:199-213 (1963).
2. E. W. Merrill, E. R. Gilliland, T. S. Lee, E. W. Salzman, Blood rheology: effect of fibrinogen deduced by addition, Circulation Research 18 437-446 (1966).
3. S. Chien et al., Blood viscosity: influence of erythrocyte aggregation, Science 157 829-831 (1967).
4. S. Chien, S. Usami, R. J. Kellenback, M. I. Gregersen, Shear-dependent interaction of plasma proteins with erythrocytes in blood rheology, American Journal of Physiology 219 143-153 (1970).
5. S. Chien, S. Usami, R. J. Dellenback, M. I. Gregersen, Blood Viscosity: influence of erythrocyte deformation, Science 157 827-829 (1967).
6. N. Casson, A flow equation for pigment-oil suspensions of the printing ink type, Rheology of Disperse Systems, Mill C C ed., Pergamon Press, New York-London-Paris-Los Angeles 84-104 (1992).
7. P. J. Hoogerbrugge, J. M. V. A. Koelman, Simulating microscopic hydrodynamic phenomena with dissipative particle dynamics, Europhysics Letters 19 155-160 (1992).
8. P. Espanol, P. Warren, Statistical mechanics of dissipative particle dynamics, Europhysics Letters 30 191-196 (1995).
9. I. V. Pivkin, G. E. Karniadakis, Accurate coarse-grained modeling of red blood cells, Physical Review Letters 101 118105 (2008).
10. H. Noguchi, G. Gompper, Shape transitions of fluid vesicles and red blood cells in capillary flows, Proceedings of the National Academy of Sciences USA 102 14159-14164 (2005).
11. J. L. McWhirter, H. Noguchi, G. Gompper, Flow-induced clustering and alignment of vesicles and red blood cells in microcapillaries, Proceedings of the National Academy of Sciences USA 106 6039-6043 (2009).
12. D. A. Fedosov, B. Caswell, G. E. Karniadakis, A multiscale red blood cell model with accurate mechanics, rheology, and dynamics, Biophysical Journal 98 2215-2225 (2010).
13. W. Pan, B. Caswell and G. E. Karniadakis, A low-dimensional model for the red blood cell, Soft Matter DOI: 10.1039/COSM00183J (2010).
14. W. Pan, B. Caswell, G. E. Karniadakis, Rheology, microstructure and migration in Brownian colloidal suspensions, Langmuir 26 133-142 (2009).
15. S. Suresh et al., Connections between single-cell biomechanics and human disease states: gastrointestinal cancer and malaria, Acta Biomaterialia 1 15-30 (2005).
16. S. Chien, S. Usami, H. M. Taylor, J. L. Lundberg, M. I. Gregersen, Effects of hematocrit and plasma proteins on human blood rheology at low shear rates, Journal of Applied Physiology 21 817 (1966).
17. R. Skalak, S. R. Keller, T. W. Secomb, Mechanics of blood flow, Journal of Biomechanical Engineering 103 102-115 (1981).
18. P. Bagchi, A. S. Popel, P. C. Johnson, Computational fluid dynamic simulation of aggregation of deformable cells in a shear flow, Journal of Biomechanical Engineering 127 1070-1080 (2005).
19. T. Wang, T. W. Pan, Z. W. Xing, R. Glowinski, Numerical simulation of rheology of red blood cell rouleaux in microchannels, Physical Review E 79 041916 (2009).
20. Y. Liu, L. Zhang, X. Wang, W. K. Liu, Coupling of Navier-Stokes equations with protein molecular dynamics and its application to hemodynamics, International Journal for Numerical Methods in Fluids 46 1237-1252 (2004).
21. Y. Liu, W. K. Liu, Rheology of red blood cell aggregation by computer simulation, Journal of Computational Physics 220 139-154 (2006).
22. R. W. Samsel, A. S. Perelson, Kinetics of rouleau formation: I. A mass action approach with geometric features, Biophysical Journal 37 493-514 (1982).
23. Q. Zhao, L. G. Durand, L. Allard, G. Cloutier, Effects of a sudden flow reduction on red blood cell rouleau formation and orientation using RF backscattered power, Ultrasound in Medicine & Biology 24 503-511 (1998).
24. A. Iordan, A. Duperray, C. Verdier, Fractal approach to the rheology of concentrated suspensions, Physical Review E 77 011911 (2008).
25. G. Cokelet, et al., The rheology of human blood-measurement near and at zero shear rate, Transaction of the Society of Rheology 7 303-317 (1963).
26. A. L. Copley, C. R. Huang, R. G. King, Rheogoniometric studies of whole human blood at shear rates from 1,000-0.0009 sec-1. Part I, experimental findings, Biorheology 10 17-22 (1973).
27. H. J. Meiselman, Measures of blood rheology and erythrocyte mechanics, Erythrocyte Mechanics and Blood Flow, (Alan R. Liss Inc., New York, 1980).
28. C. Picart, J. M. Piau, H. Galliard, Human blood shear yield stress and its hematocrit dependence, Journal of Rheology 42 1-12 (1998).
29. G. B. Thurston, Viscoelasticity of human blood, Biophysical Journal 12 1205-1217 (1972).
30. A. L. Copley, R. G. King, On the viscoelasticity of anticoagulated whole human blood in steady shear as tested by rheogoniometric measurements of normal force, Biorheology 12 5-10 (1976).
31. S. Chien, K.-M. Jan, Ultrastructural basis of the mechanism of rouleaux formation, Microvascular Research 5 155-166 (1973).
32. H. Baumler, B. Neu, E. Donath, H. Kiesewetter, Basic phenomena of red blood cell rouleuax formation biorheology, Biorheology 36 439-442 (1999).
33. B. Neu, H. J. Meiselman, Depletion-mediated red blood cell aggregation in polymer solutions, Biophysical Journal 83 2482-2490 (2002).
34. S. Chien, L. A. Sung, S. Kim, A. M. Burke, S. Usami, Determination of aggregation force in rouleaux by fluid mechanical technique, Microvascular Research 13 327-333 (1977).
35. B. Debbaut, T. Avalosse, J. Dooley, K. Highes, On the development of secondary motions in straight channels induced by the second normal stress difference: experiments and simulations, Journal of Non-Newtonian Fluid Mechanics 69 255-271 (1997).
36. M. Frank, D. Anderson, E. R. weeks, J. F. Morris, Particle migration in pressure-driven flow of a Brownian supsension, Journal of Fluid Mechanics 493 363-378 (2003).
37. This work was supported by NIH Grant number R01HL094270 and simulations were performed on the Cray XT5 at NSF/NICS and at the Julich Supercomputing Center.

### Example 8

### Analysis of White Blood Cell Dynamics

### Adhesive Dynamics of Leukocytes and Pf-Parasitized RBCs

Simulations of adhesive dynamics of leukocytes and Pf-parasitized RBCs with the endothelium lining blood vessel walls were performed. The adhesive dynamics model was based on a stochastic formation/dissociation of bonds which correspond to receptor/ligand interactions. The model was able to successfully reproduce different types of the adhesive dynamics of cells such as firm adhesion, continuous rolling over a surface, and rolling in a “stop-and-go” manner. Cytoadhesive dynamics depended on a number of factors such as density of the available receptors and ligands, their interactions (e.g., bond formation/dissociation rates, bond strength), cell properties (e.g., cell shape, elasticity, bending rigidity), and flow conditions (e.g., shear rate, shear stress). The effect of some of those conditions was examined for leukocytes and infected RBCs in malaria, in particular, Pf-parasitized RBCs showed a “flipping” rather than “rolling” behavior attributed to an increased cell stiffness in comparison with that of healthy RBCs.

### Adhesion Model

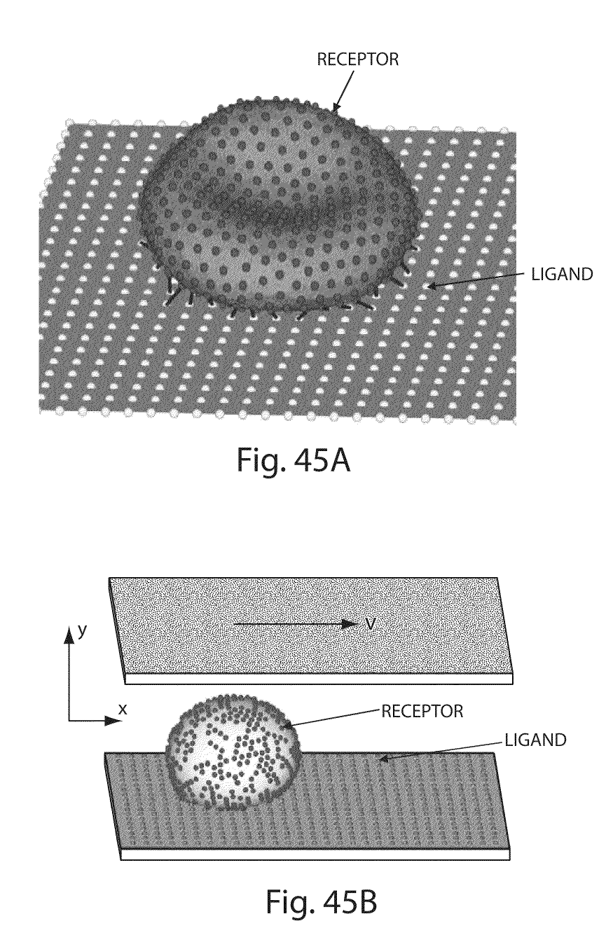

The adhesion model provided rules of formation and dissociation of bonds between receptors and ligands. Receptors were the bonding sites on the surfaces of cells, while ligands represented adhesion sites distributed on a wall. FIG. 45(a) shows a sketch of RBC adhesion.

A potential bond was formed only if it was close enough to a free ligand, which was characterized by the reactive distance dₒₙ. A ligand was called free if it was not bound to any receptors. During the time a receptor was within the distance dₒₙ. to a free ligand a bond could be formed with the on-rate kₒₙ. Reversely, existing bonds were ruptured with the off-rate k_{off} or if their length exceeded the rupture distance d_{off}. The rates kₒₙ and k_{off} were defined as follows

$${k}_{on} = {k}_{on}^{0} exp ( - \frac{{{σ}_{on} ( l - {l}_{0} )}^{2}}{2 {k}_{B} T} ) . \\ {k}_{off} - {k}_{off}^{0} exp ( \frac{{{σ}_{off} ( 1 - {l}_{0} )}^{2}}{2 {k}_{B} T} ) . \qquad ( 4.12 )$$

where kₒₙ⁰ and k_{off}⁰ were the reaction rates at the distance l=l₀ between a receptor and a ligand with the equilibrium spring length lₒ. The effective on and off strengths σₒₙ and σ_{off} defined a decrease or an increase of the corresponding rates within the interaction lengths dₒₙ and d_{off}, and k_{B}T was the unit of energy. The force exerted on the receptors and ligands by an existing bond was given by

F(l)=kₛ(l−l₀), (4.13)

where kₛ was the spring constant. The probabilities of bond formation and dissociation were defined as follows

$${P}_{on} = \{ \begin{matrix}1 - {e}^{- {k}_{on} Δ t} & for l < {d}_{on} \\ 0 & for l \geq {d}_{on} ,\end{matrix} \\ {p}_{off} = \{ \begin{matrix}1 - {e}^{- {k}_{off} Δ t} & for l < {d}_{off} \\ 0 & for l \geq {d}_{off}\end{matrix} \qquad ( 4.14 )$$

where Δt was the time step in simulations.

During the course of a simulation the receptor/ligand interactions were considered during essentially every time step. First, all existing bonds between receptors and ligands were checked for a potential dissociation according to the probability P_{off}. A bond was ruptured if ξ<P_{off} and left unchanged otherwise, where ξ was a random variable uniformly distributed on [0,1]. If a bond was ruptured the corresponding ligand was available for new binding. Second, all free ligands were examined for possible bond formations. For each free ligand the receptors were looped over within the distance dₒₙ, and the bond formation was attempted for each found receptor according to the probability Pₒₙ. This loop was terminated when a bond was formed. This algorithm permitted only a single bond per ligand, while receptors could establish several bonds if several ligands were free within their reaction radius. The forces of essentially all remaining bonds were calculated and applied.

### Scaling of Model and Physical Units

To relate DPD non-dimensional parameters of the adhesive model to those in physical units length and time scales were defined. The length scaling was based on the cell diameter and was defined. The time scale was given as follows

$$r = \frac{{\dot{γ}}^{M}}{{\dot{γ}}^{P}} s , \qquad ( 4.15 )$$

where \dot{γ} was the characteristic shear rate of a flow, and the superscripts “P” and “M” corresponded to physical and model units, respectively. Simulation parameters are chosen in such a way that the following equality was satisfied

$$\frac{\dot{γ} M}{\dot{γ} P} = \frac{{D}_{0}^{P}}{{D}_{0}^{M}} \frac{{η}_{o}^{P}}{{η}_{o}^{M}} \frac{{Y}_{0}^{M}}{{Y}_{0}^{P}} , \qquad ( 4.16 )$$

where D₀ was the cell diameter, η₀ was the external fluid viscosity, and Y was the cell Young's modulus. The scales of force and energy were then defined as follows

$${N}^{M} = \frac{{η}_{o}^{P}}{{η}_{o}^{M}} {( \frac{{D}_{o}^{P}}{{D}_{o}^{M}} )}^{2} \frac{\dot{γ} M}{\dot{γ} P} {N}^{P} , \\ {( {k}_{B} T )}^{M} = \frac{{η}_{o}^{P}}{{η}_{o}^{M}} {( \frac{{D}_{0}^{P}}{{D}_{0}^{M}} )}^{3} \frac{\dot{γ} M}{\dot{γ} P} {( {k}_{B} T )}^{P} , \qquad ( 4.17 )$$

where N denotes “Newton”.

### Analysis of Adhesive Dynamics of Leukocytes in Shear Flow

Leukocyte or white blood cell (WBC) adhesion to the vascular endothelium is involved in the immune response. To further understand the process of Leukocyte adhesion, the dynamics of adhesion were modeled.

A sketch of the simulation setup is shown in FIG. 45(b). WBC membrane was represented by a network on a sphere with the radius R=5 μm. The total number of receptors was Nᵣ=1000. Ligands were placed on the lower wall on a square lattice with the lattice constant d=0.25 μm. Linear shear flow was generated by the upper wall moving with velocity V, while the lower wall was kept stationary. The domain dimensions were set to 40×30×20 μm with periodicity in x (flow) and z directions. Simulation (in DPD units) and physical (in SI units) parameters were tabulated (See Table 8.1 below). The receptor/ligand interactions in simulations correspond to effective bonds that may represent. several physical bonds.

TABLE 8.1
Simulation (in DPD units) and physical (in SI units) parameters for leukocyte adhesive
dynamics.

| Parameters | Simulations | Physical | | Typical values | | Ref. |
|---|---|---|---|---|---|---|
| WBC radius (R) | 5 | 5 × 10⁻⁶ | m | 4.5-5 × 10⁻⁶ | m | [7] |
| Young's modulus (Y) | 7720 | 0.4 × 10⁻³ | N/m | 0.3-1.2 × 10⁻³ | N/m | [38, 101] |
| bending rigidity (k_{c}) | 60 | 3 × 10⁻¹⁸ | J | 1-3 × 10⁻¹⁸ | J | [203] |
| shear rate (\dot{γ}) | 0.1 | 100 | s⁻¹ | 50-300 | s⁻¹ | [26] |
| temperature (T) | 0.0828 | 310 | K | 293-310 | K | |
| external fluid | 20 | 10⁻³ | Pa · s | 1-3 × 10⁻³ | Pa · s | [26] |
| viscosity (nₒ) | | | | | | |
| internal fluid | 54 | 2.7 × 10⁻³ | Pa · s | | | |
| viscosity (77 ) | | | | | | |
| spring constant (kₛ) | 20000 | 10⁻³ | N/m | 10⁻⁵-10⁻² | N/m | [92, 78] |
| equilibrium spring | 0.025 | 25 × 10⁻⁹ | m | 10-40 × 10⁻⁹ | m | [43] |
| length (l₀) | | | | | | |
| reactive distance (dₒₙ) | 0.1 | 10⁻⁷ | m | | | |
| rupture distance (d_{off}) | 0.1 | 10⁻⁷ | m | <1.5 × 10⁻⁷ | m | [128] |
| on strength (σₒₙ) | 10.0 | 5 × 10⁻⁷ | N/m | −5-5 × 10⁻³ | N/m | [43] |
| off strength (σ_{off}) | 1.0 | 5 × 10⁻⁸ | N/m | −5-5 × 10⁻³ | N/m | [43] |
| unstressed on rate (kₒₙ⁰) | 10⁻³-10 | 1-10⁴ | s⁻¹ | 10³-10⁴ | s⁻¹ | [164] |
| unstressed off rate (k_{off}⁰) | 10⁻⁵-10 | 10⁻²-10⁴ | s⁻¹ | 0.5-300 | s⁻¹ | [7] |
| receptor density (nᵣ) | 3.18 | 3.18 | mol/μn² | 200-500 | mol/μn² | [114] |
| ligand density (nₗ) | 16 | 16 | mol/μn² | 200-500 | mol/μn² | [114] |

A WBC was placed at a distance of 50 ion from the lower wall. Before the flow startup, each simulation was run for 0.5 s in equilibrium (V=0) to allow for initial binding of the WBC. After that the shear flow was started and WBC dynamics were monitored for 10 s. Besides receptor/ligand interactions a WBC was subjected to the buoyant force ΔpV_{w BC}g, where V_{W BC} was the WBC volume, g was the gravitational acceleration, and Δp was the density difference between the internal and external fluids which was equal to 50 kg/m³. Table 8.2 presents additional DPD parameters for interactions among particles representing external solvent. (Sₒ). internal fluid (Sᵢ), WBC vertices (V), and walls (W). DPD interactions not included in table 8.1 were turned off. The WLC-POW model was employed for WBCs with the parameters:

μ₀^{M}=2000, x₀=2.2, kₐ=50000, k_{d}=1000, kᵥ=50000, and m=2 (see section 3.2 for details).

TABLE 8.2
DPD parameters used in simulations of WBC dynamics.

| Interaction | α | γ | r_{c} | k(eq.(2.11)) |
|---|---|---|---|---|
| Sₒ-Sₒ, Sₒ-W | 4.0 | 9.15 | 1.5 | 0.25 |
| Sᵢ-Sᵢ, | 4.0 | 20.0 | 1.5 | 0.25 |
| Sₒ-V, Sᵢ-V, W-V | 2.0 | 20.0 | 1.5 | 0.25 |
| V-V | 0.0 | 9.15 | 1.0 | 0.25 |

### Simulation Results of Leukocyte Dynamics

The simulations of WBC adhesive dynamics were performed for ranges of unstressed on and off rates shown in Table 8.1. The WBC dynamics was divided into four states according to the average pause time T̅ₚ and cell velocity v̅_{c}:
1) Firm adhesion: the state of the WBC arrest which was characterized τ̅ₚ>0.5 s. Infrequent small jumps in the cell velocity were possible due to rare bond τ̅ₚ≦0.1 s and v̅<0.8Vₘ, where Vₘ=V/2 dissociation.
2) Stop-and-go rolling: the cell motion was described by frequent interchanges between WBC arrest and mobility. This state was defined by s< τ̅≦0.5 s.
3) Stable rolling: the state corresponds to WBC motion with a relatively stable rolling velocity. It was established if τ̅ₚ≦0.1 s and v̅_{c}<0.8Vₘ, where Vₘ=V/2 was the flow velocity at the channel center.
3) Free motion: the WBC was moving freely with the channel flow, when adhesion interactions were not able to resist a lift on the cell due to the hydrodynamic flow. This state was characterized by τ̅ₚ≦0.1 s and v̅_{c}≧0.817Vₘ.
   The average pause time τ̅ₚ was calculated from the time sequence {Λᵢ}_{i=1 . . . T} of WBC: motion defined as

Λᵢ=1 if v_{c}ⁱ>0.01Vₘ, in motion, 0 if V_{c}ⁱ≦0.01Vₘ. arrest (4.18)

where i denotes a step in time, T was the total number of steps, and v_{c}ⁱ=(x_{c}ⁱ−x_{c}ⁱ⁻¹)/Δt was the WBC center-of-mass velocity while X_{c}ⁱ was the cell center-of-mass and Δt was the time interval. This sequence was then analyzed to calculate the average length of an arrest (average Pause time) which was equivalent to the average length of continuous subsequences of zeros multiplied by Δt. The time interval was chosen to be Δt=0.01 s. The simulations were run for 10 s, while data analysis was performed for times after 1 s to exclude flow startup effects.

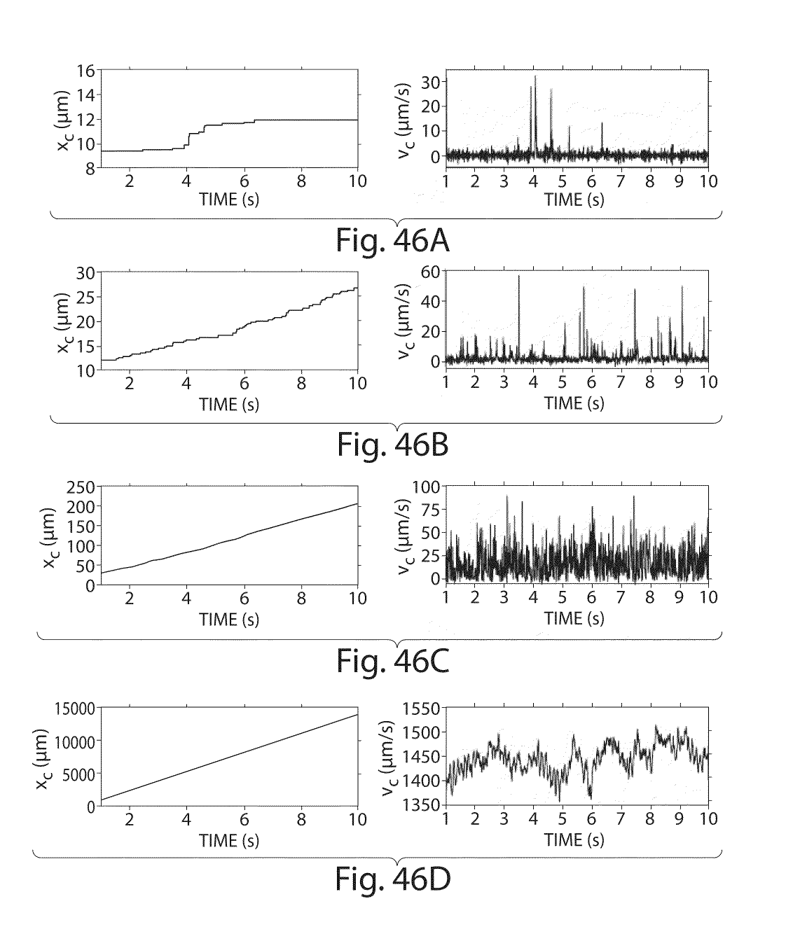

FIG. 46 presents the center-of-mass displacements (x_{c}) and velocities (v_{c}) for different WBC adhesion states. The “A” plots show that firm adhesion was characterized by relatively long times of cell arrests. However, rare events of sudden motion may have been present due to erratic bond dissociation. They were represented by several submicron steps in the WBC displacement and the corresponding peaks in the cell velocity shown in FIG. 46 “A”. WBC velocity fluctuated around the zero value and frequently displayed small negative values; however, no net motion in the negative x direction was observed. This may have been due to the presence of thermal fluctuations or a retraction of a WBC and its bonds to the surface after deformation by hydrodynamic flow. since the center-of-mass velocity was measured based on current and previous positions with the time interval Δt=0.01 s. The stop-and-go rolling shown in FIG. 46 “B” was described by a staircase-like displacement directly related to frequent peaks in the cell velocity and intermittent WBC stops. In contrast, stable rolling was characterized by a near linear WBC displacement shown in FIG. 46 “C”. Under free motion (FIG. 46 “D”) WBCs move in shear flow near the channel center with the average velocity slightly lower than Vₘ=1500 μm/s. The adhesive interactions were not strong enough to counterbalance cell-wall hydrodynamic interactions, which force WBCs to migrate to the channel center. After WBC detachment from the wall, no further adhesive interactions were encountered.

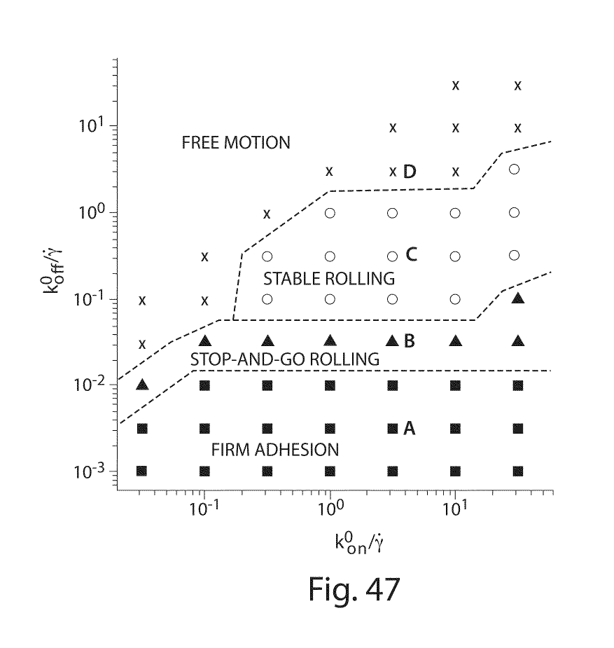

FIG. 47 shows the WBC adhesion dynamics states for wide ranges of unstressed on kₒₙ⁰ and off k_{off}⁰ rates from table 8.1 normalized by the shear rate. This plot was called an on-off state diagram. Firm adhesion occurred if the bond dissociation rate was small. Under this condition bond rupture was a rare event and bonds were formed with a faster rate to keep a WBC in arrest. At low values of kₒₙ⁰ the border between firm adhesion and stop-and-go rolling motion (black dashed line in FIG. 47) was achieved by a proper balance between the association and dissociation rates. However, this border showed no dependence on the rate kₒₙ⁰ at its high values. This behavior was due to a limited number of available receptors and ligands for binding. Thus, if there were no free receptors or analogously no free ligands left for binding, a further increase of kₒₙ⁰ had no effect on the firm adhesion of a WBC.

The bond dissociation rate k_{off}⁰ was increased for a fixed kₒₙ⁰, WBC firm adhesion transitioned into the stop-and-go rolling state. This behavior was observed in a thin stripe region of the on-off state diagram in FIG. 47 right above the “firm adhesion” region. The stop-and-go rolling was considered indicative of an unstable firm adhesion. Hence, if the rate k_{off}⁰ became significant enough in comparison with kₒₙ⁰ to allow relatively frequent random ruptures of bonds, a WBC was subjected to a stop-and-go motion characterized by step-like displacements and velocity jumps shown in FIG. 46 “B”.

Upon a further increase in k_{off}⁰ with respect to kₒₙ⁰ a WBC showed stable rolling or detached from the wall and underwent free motion in hydrodynamic flow. Stable rolling was observed if the association rate was large enough to facilitate fast bond formation. Thus, stable WBC rolling on the wall was described by a dynamic rupture of bonds at the back of the cell contact area and their quick formation at the front of a WBC. As depicted in FIG. 47, for small kₒₙ⁰ values, a WBC transitted into a free motion above the border of the stop-and-go rolling region (blue dashed line). In addition. a WBC detached from the wall if the bond dissociation rate was comparable with the rate of bond formation.

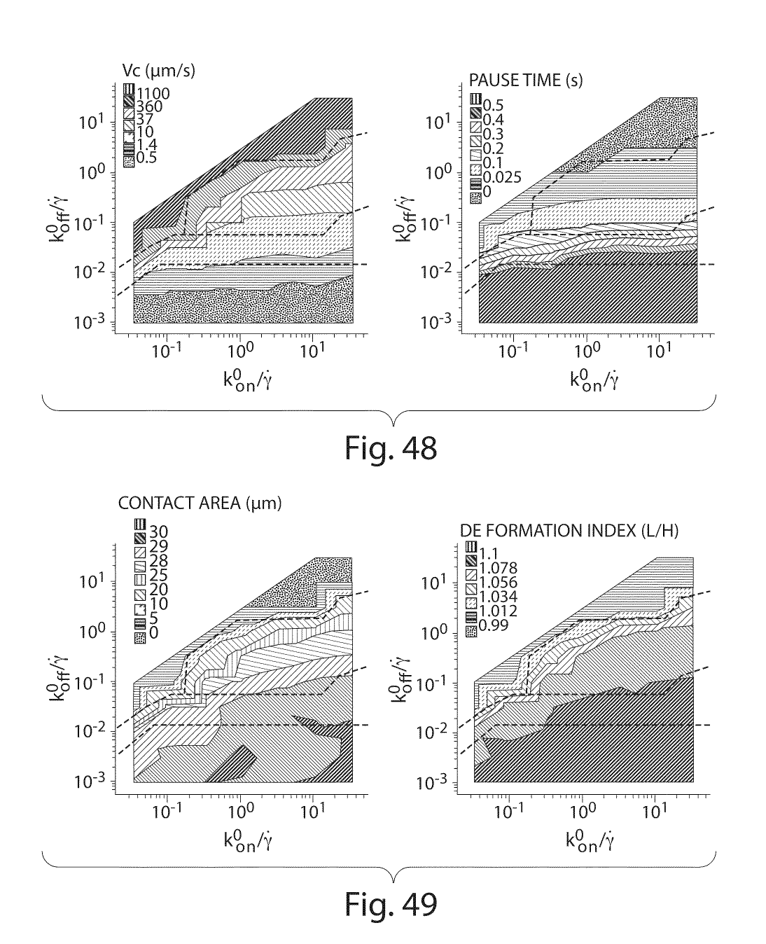

FIG. 48 presents the corresponding on-off diagrams of the average WBC velocity (left) and the average pause time (right) for various states of leukocyte adhesive dynamics. The average cell velocity in the free motion region was above 1000 μm/s confirming no adhesive interactions between the WBC and the wall. In accordance, the average WBC pause time was zero in this region. In the region of stable rolling, the average velocity was in the range of 10 μm/s to 400 μm/s depending on the relative interplay between kₒₙ⁰ and k_{off}⁰, while the pause time was below 0.1 s. The stop-and-go motion yielded the rolling velocity in the range of 1 μm/s to 70 μm/s and the pause time in the range of 0.1 s to 0.5 s. Finally, in the firm adhesion state, the average velocity of WBCs was below 1.5 μm/s with the pause times larger than 0.5 s. The stable rolling region in FIG. 48 (left) with k_{off}⁰ in the range of 10 s⁻¹ to 20 s⁻¹ and kₒₙ⁰ in the range of 100 s⁻¹ to 1000 s⁻¹ is comparable to results obtained from in vivo experiments. The range of the stop-and-go WBC region in FIG. 48 (right) is also comparable with experimental results.

FIG. 49 shows the on-off diagrams of the WBC contact area (left) and the deformation index (right). The contact area A_{c} and deformation index δ were defined as follows:

$${A}_{c} = {N}_{c} \frac{4 π {R}^{2}}{{N}_{r}} , \\ δ = \frac{L}{H} , \qquad ( 4.19 )$$

where N_{c}, was the number of receptors the distance of which from the wall was smaller than dₒₙ=100 nm, L was the WBC length, and H was its height. The maximum contact area of about 30 μm was found for the firm adhesion. Consistently, states of firm adhesion corresponded to the maximum in the deformation index of approximately 1.1. A rolling WBC showed a smaller contact area and deformation index, while a freely moving WBC had zero contact area and a deformation index close to 1 indicated that the WBC remained spherical. A contact area of about 20 μm was found in in vivo experiments at a shear rate of \dot{γ}=100 s⁻¹, which falls into the stable rolling region in FIG. 49 in agreement with the previously mentioned average cell velocity in the range of 30-50 μm/s.

Leukocyte adhesive dynamics typically depend on the medium viscosity (η₀), bond spring constant (kₛ), and densities of receptors (nᵣ) and ligands (n₁). An increase in the solvent viscosity for a fixed shear rate was shown to shift the border of the firm adhesion region to lower off rate values, since cell arrest was sensitive to shear stress. The effect of η₀ on rolling behavior was found to be insignificant because it mainly depended on the shear rate. A change in the bond spring constant may affect WBC adhesive dynamics. For example, a decrease in kₛ may result in a shrinking of the stable rolling behavior region, while an increase of kₛ may alter the firm adhesion region.

An increase in nᵣ or nₗ could shift the borders of regions of different adhesion states to higher k_{off}⁰ values, since more bonds can potentially be formed. However, if nᵣ was several times smaller than nₗ asin the disclosed simulations (see table 8.1), a further increase in nₗ may not have a significant effect on the WBC adhesive dynamics, since there may be no available receptors for binding.

WBC adhesive dynamics appears to depends on cell deformability. Softer cells have a larger contact area yielding an expanded firm adhesion region. In addition, a larger contact area may have a stabilizing effect on rolling adhesion. More compliant cells may be subject to stronger deformations under hydrodynamic flow showing a larger deformation index δ. This may result in a lower hydrodynamic force on the cell due to the flow which stabilizes adhesive interactions.

The WBC adhesive dynamics model was able to capture various states of cell adhesion.

### Example 9

### Predicting Human Blood Viscosity In Silico

In this example, we first describe the two formulations of dissipative partice dynamics (DPD) that we employed in the simulations discussed in this application. We then provide specific details on the multiscale RBC model (MS-RBC) and subsequently on the low-dimensional RBC model (LD-RBC), including the aggregation models. In the last section we present details on the scaling from DPD units to physical units.

### 1 Dissipative Particle Dynamics

### 1.1 Original Method

Dissipative Particle Dynamics (DPD) (11, 13) is a mesoscopic particle method, where each particle represents a molecular cluster rather than an individual atom, and can be thought of as a soft lump of fluid. The DPD system consists of N point particles of mass mᵢ, position rᵢ and velocity vᵢ. DPD particles interact through three forces: conservative (Fᵢⱼ^{C}), dissipative (Fᵢⱼ^{D}), and random (Fᵢⱼ^{R}) forces given by:

$${F}_{ij}^{C} = {F}_{ij}^{C} ( {r}_{ij} ) {\hat{r}}_{ij} , \\ {F}_{ij}^{D} = - {γω}^{D} ( {r}_{ij} ) ( {v}_{ij} \cdot {\hat{r}}_{ij} ) {\hat{r}}_{ij} , \\ {F}_{ij}^{R} = {σω}^{R} ( {r}_{ij} ) \frac{{ξ}_{ij}}{\sqrt{dt}} {\hat{r}}_{ij} , \qquad ( 1 )$$

where \hat{r}ᵢⱼ=rᵢⱼ/rᵢⱼ, and vᵢⱼ=vᵢ−vⱼ. The coefficients γ and σ define the strength of dissipative and random forces, respectively. In addition, ωD and ωR are weight functions, and ξij is a normally distributed random variable with zero mean, unit variance, and ξij=ξji. All forces are truncated beyond the cutoff radius rc, which defines the length scale in the DPD system. The conservative force is given by:

$${F}_{ij}^{C} ( {r}_{ij} ) = \{ \begin{matrix}{a}_{ij} ( 1 - {r}_{ij} / {r}_{c} ) & for & {r}_{ij} \leq {r}_{c} , \\ 0 & for & {r}_{ij} > {r}_{c} ,\end{matrix} \qquad ( 2 )$$

Where aij is the conservative force coefficient between particles i and j. The random and dissipative forces form a thermostat and must satisfy the fluctuation-dissipation theorem in order for the DPD system to maintain equilibrium temperature T (3). This leads to:

ω^{D}(rᵢⱼ)=[ω^{R}(rᵢⱼ)]², σ²=2γk_{B}T, (3)

where kB is the Boltzmann constant. The choice for the weight functions is as follows:

$${ω}^{R} ( {r}_{ij} ) = \{ \begin{matrix}{( 1 - {r}_{ij} / {r}_{c} )}^{k} & for & {r}_{ij} \leq {r}_{c} , \\ 0 & for & {r}_{ij} > {r}_{c} ,\end{matrix} \qquad ( 4 )$$

where k=1 for the original DPD method. However, other choices (e.g., k=0.25) for these envelopes have been used (5, 10) in order to increase the viscosity of the DPD fluid. The time evolution of velocities and positions of particles is determined by the Newton's second law of motion

$$\begin{matrix}{dr}_{i} = {v}_{i} dt , & ( 5 ) \\ {dv}_{i} = \frac{1}{{m}_{i}} {\sum }_{j \neq i}^{ } ( {F}_{ij}^{C} + {F}_{ij}^{D} + {F}_{ij}^{R} ) dt . & ( 6 )\end{matrix}$$

The above equations of motion were integrated using the modified velocity-Verlet algorithm (11).

### 1.2 DPD Method for Colloidal Particles

To simulate colloidal particles by single DPD particles, we use a new formulation of DPD, in which the dissipative forces acting on a particle are explicitly divided into two separate components: central and shear (non-central) components. This allows us to redistribute and hence balance the dissipative forces acting on a single particle to obtain the correct hydrodynamics. The resulting method was shown to yield the quantitatively correct hydrodynamic forces and torques on a single DPD particle (20), and thereby produce the correct hydrodynamics for colloidal particles (18). This formulation is reviewed below.

We consider a collection of particles with positions ri and angular velocities i. We define rᵢⱼ=rᵢ−rⱼ, rᵢⱼ=|rᵢⱼ|, eᵢⱼ=rᵢⱼ/rᵢⱼ, vᵢⱼ=vᵢ−vⱼ. The force and torque on particle i are given by

$${F}_{i} - {\sum }_{j}^{ } {F}_{ij} , \\ {T}_{i} = - {\sum }_{j}^{ } {λ}_{ij} {r}_{ij} \times {F}_{ij} . , \qquad ( 7 )$$

Here the factor λij (introduced in (21)) is included as a weight to account for the different contributions from the particles in different species (solvent or colloid) differentiated in sizes while still conserving the angular momentum. It is defined as

$${λ}_{ij} = \frac{{R}_{i}}{{R}_{i} + {R}_{j}} , \\ and {λ}_{ij} = 1 / 2 when {R}_{i} = {R}_{j} \qquad ( 8 )$$

where Ri and Rj denote the radii of the particles i and j, respectively. The force exerted by particle j on particle i is given by

Fᵢⱼ=Fᵢⱼ^{U}+Fᵢⱼ^{T}+Fᵢⱼ^{R}+\tilde{F}ᵢⱼ. (9)

The radial conservative force can be that of standard DPD, i.e.,

$${F}_{ij}^{U} = {a}_{ij} ( 1 - \frac{{r}_{ij}}{{r}_{c}} ) {e}_{ij} , \qquad ( 10 )$$

with rc being the cut-off distance. The translational force is given by

Fᵢⱼ^{T}=−[γᵢⱼ^{⊥}f²(r)1+(γᵢⱼ^{∥}−γᵢⱼ^{⊥})f²(r)eᵢⱼeᵢⱼ]·vᵢⱼ

=−γᵢⱼ^{∥}f²(rᵢⱼ)(vᵢⱼ·eᵢⱼ)eᵢⱼ−γᵢⱼ^{⊥}f²(rᵢⱼ)[vᵢⱼ−(vᵢⱼ·eᵢⱼ)eᵢⱼ]. (11)

It accounts for the drag due to the relative translational velocity vij of particles i and j. This force is decomposed into two components: one along and the other perpendicular to the lines connecting the centers of the particles. Correspondingly, the drag coefficients are denoted by γᵢⱼ^{∥} and γᵢⱼ^{⊥} for a “central” and a “shear” components, respectively. We note that the central component of the force is identical to the dissipative force of standard DPD.

The rotational force is defined by

$${F}_{ij}^{R} = - {γ}_{ij}^{⊥} {f}^{2} ( {r}_{ij} ) [ {r}_{ij} \times ( {λ}_{ij} {Ω}_{i} + {λ}_{ji} {Ω}_{j} ) ] . \qquad ( 12 )$$

while the random force is given by

$${\tilde{F}}_{ij} dt = f ( {r}_{ij} ) [ \frac{1}{\sqrt{3}} {σ}_{ij}^{∥} tr [ {dW}_{ij} ] 1 + \sqrt{2} {σ}_{ij}^{⊥} {dW}_{ij}^{A} ] \cdot {e}_{ij} , \qquad ( 13 )$$

where

$${σ}_{ij}^{\vert \vert} = √ 2 {k}_{B} T {γ}_{ij}^{\vert \vert} and {σ}_{ij}^{⊥} = \sqrt{2 {k}_{B} T {γ}_{ij}^{⊥}}$$

are chosen to satisfy the fluctuation-dissipation thereom, dWij is a matrix of independent Wiener increments, and dWᵢⱼ^{A} is defined as dWᵢⱼ^{Aμv}=½(dWᵢⱼ^{μv}−dWᵢⱼ^{vμ}). We used the generalized weight function

$$f ( r ) = {( 1 - \frac{r}{{r}_{c}} )}^{k}$$

as in the previous section with k=0.25 (6) in equations (11)-(13). Our numerical results in previous studies (19, 20) showed higher accuracy with k=0.25 compared to the usual choice k=1. The standard DPD is recovered when

$${γ}_{ij}^{⊥} ≡ 0 ,$$

i.e., when the “shear” components of the forces are ignored.

Colloidal particles are simulated as single DPD particles, similarly to the solvent particles but of larger size. The particle size can be adjusted with the coefficient aij of the conservative force (see eq. (10)). However, the standard linear force in DPD defined as in eq. (10) is too soft to model any hard-sphere type of particles. To resolve this problem, we adopt an exponential conservative force for the colloid-colloid and colloid-solvent interactions, but keep the conventional DPD linear force for the solvent-solvent interactions. We have found that these hybrid conservative interactions produced colloidal particles dispersed in solvent without overlap, which was quantified by calculating the radial distribution function of colloidal particles (18). Moreover, the timestep is not significantly decreased, in contrast to the small timesteps required for the Lennard-Jones potential (21). The radial exponential conservative force is defined as

$${F}_{ij}^{U} = \frac{{a}_{ij}}{1 - {e}^{{b}_{ij}}} ( {e}^{{b}_{ij} {r}_{ij} / {r}_{c}^{e}} - {e}^{{b}_{ij}} ) , \qquad ( 14 )$$

where aij and bij are adjustable parameters, and r_{c}^{e} is its cutoff radius. The size of a colloidal particle can thus be controlled by adjusting the value of aij in eq. (14).

### 2 MS-RBC Model

The average equilibrium shape of a RBC is biconcave as measured experimentally (4), and is represented by

$$z = \pm {D}_{0} \sqrt{1 - \frac{4 ( {x}^{2} + {y}^{2} )}{{D}_{0}^{2}}} [ {a}_{0} + {a}_{1} \frac{{x}^{2} + {y}^{2}}{{D}_{0}^{2}} + {a}_{2} \frac{{( {x}^{2} + {y}^{2} )}^{2}}{{D}_{0}^{4}} ] , \qquad ( 15 )$$

where D0=7.82 μm is the average diameter, a0=0.0518, a1=2.0026, and a2=−4.491. The surface area and volume of this RBC are equal to 135 μm2 and 94 μm3, respectively.

In the simulations, the membrane network structure is generated by triangulating the unstressed equilibrium shape described by (15). The cell shape is first imported into a commercial grid generation software to produce an initial triangulation based on the advancing-front method. Subsequently, free-energy relaxation is performed by flipping the diagonals of quadrilateral elements formed by two adjacent triangles, while the vertices are constrained to move on the prescribed surface. The relaxation procedure includes only elastic in-plane and bending energy components described below.

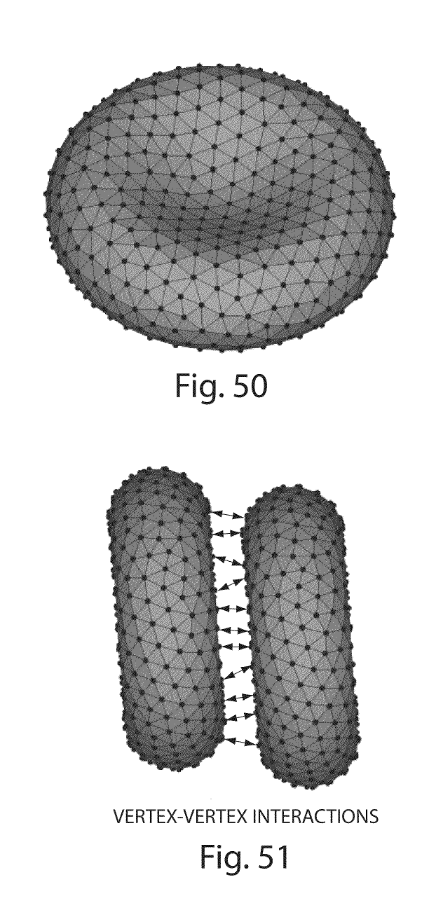

FIG. 50 shows the membrane model represented by a set of points {xᵢ}, iε1 . . . Nᵥ that are the vertices of a two-dimensional triangulated network on the RBC surface described by equation (15). The vertices are connected by Ns edges which form Nt triangles. The potential energy of the system is defined as follows

V({xᵢ})=Vᵢₙ₋ₚₗₐₙₑ+V_{bending}+Vₐᵣₑₐ+Vᵥₒₗᵤₘₑ. (16)

The in-plane elastic energy mimics the elastic spectrin network, and is given by

$${V}_{in - plane} = {\sum }_{j ∈ 1 … {N}_{s}}^{ } [ \frac{{k}_{B} {Tl}_{m} ( 3 {x}_{j}^{2} - 2 {x}_{j}^{3} )}{4 p ( 1 - {x}_{j} )} + \frac{{k}_{p}}{( n - 1 ) {l}_{j}^{n - 1}} ] , \qquad ( 17 )$$

where lj is the length of the spring j, lm is the maximum spring extension, xj=lj/lm, p is the persistence length, kBT is the energy unit, kp is the spring constant, and n is a power. Note that the spring forces in membrane are a combination of conservative elastic forces, that may be expressed in terms of the energy potential above, and dissipative forces to be defined below. The first term in (17) corresponds to the attractive wormlike chain (WLC) potential, and the second term defines a repulsive force for n>0 to be called the power force (POW), so that we abbreviate this spring model as WLC-POW. Note that if n=1 the power force energy should be defined as −kₚ log(lⱼ). A non-zero equilibrium spring length is defined by the balance of these two forces.

The bending energy represents the bending resistance of the lipid bilayer and is defined as

$${V}_{bending} = {\sum }_{j ∈ 1 … {N}_{s}}^{ } {k}_{b} [ 1 - cos ( {θ}_{j} - {θ}_{0} ) ] , \qquad ( 18 )$$

where kb is the bending constant, θj is the instantaneous angle between two adjacent triangles having the common edge j, and θ0 is the spontaneous angle.

The area and volume conservation constraints which account for area-incompressibility of the lipid bilayer and incompressibility of the inner cytosol, respectively, are expressed as

$$\begin{matrix}{V}_{area} = \frac{{{k}_{a} ( A - {A}_{0}^{tot} )}^{2}}{2 {A}_{0}^{tot}} + {\sum }_{j ∈ 1 … {N}_{t}} \frac{{{k}_{d} ( {A}_{j} - {A}_{0} )}^{2}}{2 {A}_{0}} , & ( 19 a ) \\ {V}_{volume} = \frac{{{k}_{v} ( V - {V}_{0}^{tot} )}^{2}}{2 {V}_{0}^{tot}} , & ( 19 b )\end{matrix}$$

where ka, kd and kv are the global area, local area and volume constraint coefficients, respectively. The terms A and V are the total area and volume of RBC, while A_{θ}^{tot} and V₀^{tot} are the specified total area and volume, respectively. Note, that the above expressions define global area and volume constraints, and the second term in equation (19a) incorporates the local dilatation constraint. Detailed description and discussion of the RBC model can be found in (8, 9).

Particle forces are derived from the above energies as follows

fᵢ=−∂V({xᵢ})/∂xᵢ, iε1 . . . Nᵥ, (20)

Exact force expressions can be found in (7).

### 2.1 Mechanical Properties

Linear analysis of the regular hexagonal network having the above energies yields a relationship between macroscopic elastic properties (shear, area-compression, and Young's moduli) of the network and model parameters (8, 9). The membrane shear modulus is thus given by:

$${μ}_{0} = \frac{\sqrt{3} {k}_{B} T}{4 {pl}_{m} {x}_{0}} ( \frac{{x}_{0}}{2 {( 1 - {x}_{0} )}^{3}} - \frac{1}{4 {( 1 - {x}_{0} )}^{2}} + \frac{1}{4} ) + \frac{\sqrt{3} {k}_{p} ( n + 1 )}{4 {l}_{0}^{n + 1}} , \qquad ( 21 )$$

where l0 is the equilibrium spring length and x₀=l₀/lₘ. The corresponding area-compression and Young's moduli are found as follows:

$${K}_{0} = 2 {μ}_{0} + {k}_{a} + {k}_{d} , \\ {Y}_{0} = \frac{4 {K}_{0} {μ}_{0}}{{K}_{0} + {μ}_{0}} . \qquad ( 22 )$$

The bending coefficient kb of equation (18) can be expressed in terms of the macroscopic bending rigidity kc of the Helfrich model (12) as k_{b}=2k_{c}/\sqrt{3}.

### 2.2 Membrane Viscoelasticity

The above model defines a purely elastic membrane, however the RBC membrane is known to be viscoelastic. To incorporate viscosity into the model, the spring definition is modified by adding viscous contribution through dissipative and random forces. Such a term fits naturally in the DPD method (13), where interparticle dissipative interactions are an intrinsic part of the method. Straightforward implementation of the dissipative interactions as Fᵢⱼ^{D}=−γ(vᵢⱼ·eᵢⱼ)eᵢⱼ (γ is the dissipative parameter, vij=vi−vj is the relative velocity of vertices i and j connected by a spring, and eij is the direction along the spring with unit length) appears to be insufficient. Experience shows that small γ results in a negligible viscous contribution since vij·eij˜0, while large values of γ require considerably smaller time steps to overcome the numerical instability. Better performance is achieved with a viscous spring dissipation term −γvij, for which the fluctuation-dissipation balance needs to be imposed to ensure the maintenance of the equilibrium membrane temperature kBT. We follow the general framework of the fluid particle model (2), and define Fᵢⱼ^{D}=−Tᵢⱼ·Vᵢⱼ and T̅ᵢⱼ=γ^{T}1+γ^{C}eᵢⱼeᵢⱼ, where γT and γC are the dissipative coefficients. This definition results in the dissipative interaction term of the kind

Fᵢⱼ^{D}=−[γ^{T}1+γ^{C}eᵢⱼeᵢⱼ]·vᵢⱼ=−γ^{T}vᵢⱼ−γ^{C}(vᵢⱼ·eᵢⱼ)eᵢⱼ, (23)

where the second term is analogous to the dissipative force in DPD. From the fluctuation-dissipation theorem, random interactions are given by

Fᵢⱼ^{R}dt=\sqrt{2k_{B}T}(\sqrt{2γ^{T}} d̅W̅ᵢ̅ⱼ̅^̅{̅S̅}̅+\sqrt{3γ^{C}−γ^{T}}tr[dWᵢⱼ]/31)·eᵢⱼ, (24)

where tr[dWij] is the trace of a random matrix of independent Wiener increments dWij, and d̅W̅ᵢ̅ⱼ̅^̅{̅S̅}̅=dWᵢⱼ^{S}−tr[dWᵢⱼ^{S}]⅓ is the traceless symmetric part, while dWᵢⱼ^{S}=[dWᵢⱼ+dWᵢⱼ^{T}]/2 is the symmetric part. Note, that the last equation imposes the condition 3γC>γT. The defined dissipative and random forces in combination with an elastic spring constitute a viscoelastic spring whose equilibrium temperature kBT is constant. To relate the membrane shear viscosity ηm and the dissipative parameters γT, γC we employ the idea used for the derivation of membrane elastic properties (see (7, 8) for details) and obtain the following relation

$${η}_{m} = \sqrt{3} {γ}^{T} + \frac{\sqrt{3} {γ}^{C}}{4} . \qquad ( 25 )$$

Our experience indicates that γT accounts for a large portion of viscous contribution, and therefore γC is set to γT/3 in all simulations.

### 2.3 RBC-Solvent Boundary Conditions

RBCs are suspended in a solvent, which is represented by a collection of interacting DPD particles. To impose no-slip boundary conditions at the membrane, the DPD dissipative force between fluid particles and membrane vertices needs to be properly set based on the idealized case of linear shear flow over a flat plate. In continuum, the total shear force exerted by the fluid on the area A is equal to Aη\dot{γ}, where η is the fluid's viscosity and \dot{γ} is the local wall shear-rate. In DPD, we distribute a number of particles on the wall to mimic the membrane vertices. The force on a single wall particle exerted by the sheared fluid can be found as follows:

Fᵥ=∫_{V}_{ₕ}ng(r)F^{D}dV, (26)

where FD is the DPD dissipative force (2) between fluid particles and membrane vertices, n is the fluid number density, g(r) is the radial distribution function of fluid particles with respect to the wall particles, and Vh is the half sphere volume of fluid above the wall. Here, the total shear force on the area A is equal to NAFv, where NA is the number of wall particles enclosed by A. The equality of N_{A}Fᵥ=Aη\dot{γ} results in an expression of the dissipative force coefficient in terms of the fluid density and viscosity, and the wall density NA/A, while under the assumption of linear shear flow the shear rate \dot{γ} cancels out. This formulation results in satisfaction of the no-slip BCs for the linear shear flow over a flat plate. It also serves as an excellent approximation for no-slip at the membrane surface in spite of the assumptions made. Note that in simulations we turn off the conservative interactions between fluid and wall particles which results in g(r)=1.

### 2.4 RBC Aggregation Interactions

For a blood suspension the attractive cell-cell interactions are crucial for simulation of aggregation into rouleaux. These forces are approximated phenomenologically with the Morse potential given by

U_{M}(r)=Dₑ[e^{2β(r}^{₀}^{-r)}−2e^{β(r}^{₀}^{-r)}], (27)

where r is the separation distance, r0 is the zero force distance, De is the well depth of the potential, and β characterizes the interaction range. For the MS-RBC model the Morse potential interactions are implemented between every two vertices of separate RBCs if they are within a defined potential cutoff radius rM as shown in FIG. 51. The Morse interactions consist of a short-ranged repulsive force when r<r0 and of a long-ranged attractive force for r>r0. However, such repulsive interactions cannot prevent two RBCs from an overlap. To guarantee no overlap among RBCs we employ a short range Lennard-Jones potential and specular reflections of RBC vertices on membranes of other RBCs. The Lennard-Jones potential is defined as

$${U}_{LJ} ( r ) = 4 ε [ {( \frac{{σ}_{LJ}}{r} )}^{12} - {( \frac{{σ}_{LJ}}{r} )}^{6} ] , \qquad ( 28 )$$

where [symbol] and σLJ are energy and length characteristic parameters, respectively. These interactions are repulsive and vanish beyond r>21/6σLJ. In addition, specular reflections of RBC vertices on surfaces of other RBCs are necessary due to coarseness of the triangular network which represents the RBC membrane.

### 2.5 Simulation Setup and Parameters

RBC suspension (blood) is subjected to linear shear flow with periodic Lees-Edwards boundary conditions (14). The computational domain has the size of 45.0×32.0×27.222 in DPD units, where 168 RBCs and 117599 solvent particles are placed. RBCs are represented by 500 DPD particles forming a triangulated network on the surface defined in equation (15). The RBC diameter and the membrane Young's modulus are D0=8.06 and Y0=415.5 in model units, respectively, which correspond to D0=7.82 μm and Y0=18.9 μN/m in physical units. The membrane shear modulus is μ0=106, while x0=2.2 and n=2 in equation (21). We employ the stress-free model (8, 9) which eliminates local membrane artifacts (stresses) due to the membrane triangulation. Thus, each spring assumes its own equilibrium length l₀ⁱ, i=1 . . . Nₛ, which is set to the edge lengths after the RBC shape triangulation, since we assume it to be the equilibrium state. Accordingly we define lₘₐₓⁱ=l₀ⁱ×x₀ and Ā₀^{j}, j= 1̅ . . . Nₜ for each triangular plaquette. The total RBC area A₀^{tot}=Σ_{j=1 . . . N}_{ₜ}A₀^{j} and the total volume V₀^{tot} are calculated from the RBC triangulation. Then, for each spring we can calculate pi and kₚⁱ (eq. (17)) for the given parameters μ0, l₀ⁱ, and lₘⁱ using equation (21) and the equality F_{spring}(l₀ⁱ)=0. The area and volume constraints coefficients were set to ka=4900, kd=100, and kv=5000 (eqs. (11a,b)). The bending rigidity kc is set to 3×10-19J, which is equal to approximately 70 kBT at physiological temperature T=37° C. The membrane viscosity is set to be approximately 12η0, where η0 is the suspending fluid viscosity.

Interactions between different RBCs include the short range repulsive Lennard-Jones potential defined in equation (28). The corresponding potential parameters were set to [symbol] =1.0 and σLJ=0.3. These interactions result in a thin layer next to a RBC membrane which cannot be accessed by other cells. This layer can be interpreted as a slight increase of the RBC volume. Therefore, the RBC volume was assumed to be slightly larger than that of the triangulated network (V0=92.45) due to the repulsive RBC-RBC interactions. The effective RBC volume was estimated from an analysis of the distance between surfaces of several RBCs in equilibrium and was equal to V′=105. The cell volume fraction or hematocrit was calculated as follows

$$H = \frac{{N}_{c} {V}^{′}}{{V}_{t}} , \qquad ( 29 ) ,$$

where Nc is the number of RBCs in the volume Vt.

RBC aggregation interactions were mediated by the Morse potential (eq. (27)). The Morse potential parameters were set to De=0.3, r0=0.3, β=1.5, and rM=1.1. The choice of r0 was correlated with the Lennard-Jones characteristic length σLJ=0.3. Other parameters were calibrated for a single point of the viscosity-shear rate curve, while all other simulations were performed for the same set of parameters.

RBCs are suspended in a solvent simulated by a collection of free DPD particles which correspond to small fluid volumes of blood plasma. Three fluids with different viscosities were employed in simulations:

1) η0=8.1; 2) η0=26.3; 3) η0=126.0, where η0 is given in DPD units. Different viscosities allow us to be able to simulate different ranges of shear rates in physical units since they affect the time scale defined in section 4. For example, a fixed shear rate in simulations in DPD units corresponds to distinct shear rates in physical units if different fluid viscosities are used. Table 1 presents the DPD interactions between different particle types (solvent (S) and RBC vertices (V)).

TABLE 1
MS-RBC: DPD Simulation parameters.

| η₀ | interaction | α | γ | r_{c} | k(eq.(4)) |
|---|---|---|---|---|---|
| 8.1 | S-S | 6.0 | 20.0 | 1.0 | 0.15 |
| 8.1 | S-V | 0.0 | 15.6 | 1.0 | 0.2 |
| 26.3 | S-S | 4.0 | 8.0 | 1.5 | 0.15 |
| 26.3 | S-V | 0.0 | 10.0 | 1.5 | 0.2 |
| 126.0 | S-S | 4.0 | 40.0 | 1.5 | 0.15 |
| 126.0 | S-V | 0.0 | 47.9 | 1.5 | 0.2 |

The random force coefficients for different interactions were obtained using the energy unit kBT=0.1 calculated according to the energy scale defined in section 4. The number density of all fluids is n=3. Note that the membrane viscosity has to be also changed with respect to η0 and is always equal to 12η0. The dissipative coefficient γ for the S-V interactions defines RBC-solvent boundary conditions and its calculation is described in section 2.3. Note that the calculated γ for the S-V interactions is multiplied by the factor of two to account for an additional viscous dissipation from RBC cytosol, since its viscosity is several times larger that that of blood plasma. In simulations a single solvent for the blood plasma and cytosol is used. This simplification allows us to substantially reduce the computational cost and to be able to calculate blood viscosity over five orders of magnitude of shear rates.

To cover a wide range of shear rates several viscosities were required. Limitations of the DPD method do not allow us to simulate high shear rates, while at very low shear rates simulation results obtained by statistical averaging contain large errors. The maximum shear rate (\dot{γ}) is limited by the local Reynolds number defined as

$$Re = \frac{n {γD}_{0}^{2}}{{η}_{0}} . \qquad ( 30 ) ,$$

where n is the fluid's density. Table 2 shows the simulated flow regimes and the corresponding shear rate ranges in physical units. The Re number in all simulations remains below 0.5. The corresponding shear rates in physical units were calculated using the value of plasma viscosity η0=0.0012 Pas at physiological temperature T=37° C.

TABLE 2
MS-RBC: Simulated flow regimes and the corresponding shear rate ranges in physical units.

| η₀ | \dot{γ} in DPD | Re | \dot{γ} (s⁻¹) |
|---|---|---|---|
| 8.1 | 5 × 10⁻⁵-0.01 | 0.0012-0.24 | 0.014-3.2 |
| 26.3 | 0.003-0.056 | 0.022-0.41 | 3.1-58 |
| 126.0 | 0.017-0.95 | 0.026-0.4 | 83-01200 |

### 2.6 Maximum RBC Aggregation Force

The maximum aggregation force between two RBCs is measured in simulations with the aggregation parameters described above. The first (lower) RBC is adhered to a wall, which is simulated by holding stationary 100 vertices at the RBC bottom. The other (upper) RBC is placed on top of the adhered RBC and is allowed to aggregate in equilibrium simulation. Then, the force is applied to the upper RBC in order to separate them.

Several cases of the separation of two RBCs were considered. In the first case the upper RBC was pulled up in the normal direction, where the force was applied to 200 RBC vertices on the RBC top. This setup corresponds to a uniform separation, which is characterized by a nearly homogeneous and full separation of the two RBC surfaces in contact. The maximum force needed to break up the two aggregated RBCs in this case was approximately 7 pN. In the second case the upper RBC was pulled up in the normal direction through 50 RBC vertices on the RBC top. Such disaggregation of two RBCs resembles peeling off the upper cell of the other with the maximum force required for disaggregation to be approximately 3 pN. Finally, in the third setup the upper RBC was pulled along the tangential direction with the force applied to 50-150 RBC vertices on the RBC side. Such separation of two RBCs can be described as sliding of the upper cell on the lower RBC and requires the force of about 1.5-3 pN.

To measure the disaggregation force in shear flow we used the same simulation setup. A fluid is confined between two parallel plates, while the lower RBC is attached to the lower plate, and the upper plate is moving with constant velocity. Then, we find the minimum shear rate \dot{γ} required for the disaggregation of RBCs, and the corresponding shear stress is calculated as \dot{γ}η0 and is equal to approximately 0.02 Pa.

### 3 LD-RBC Model

The LD-RBC is modeled as a ring of 10 colloidal particles connected by wormlike chain (WLC) springs. The intrinsic size of colloidal particle is determined by the radius of the sphere effectively occupied by a single DPD particle (18), which is depicted by the distribution of its surrounding solvent particles.

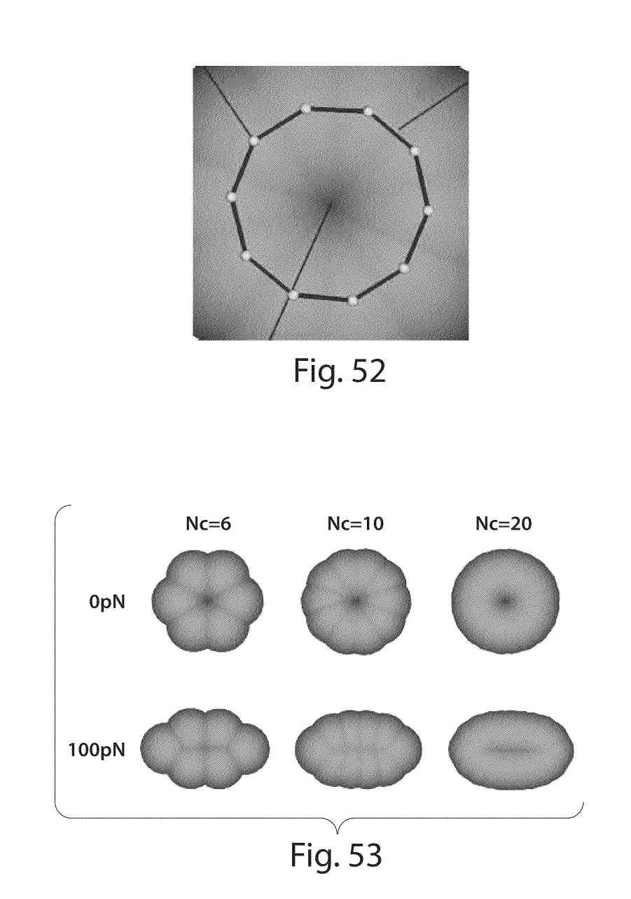

To construct the cell model, however, we allow particles in the same RBC to overlap, i.e., the colloidal particles in the same cell still interact with each other through the soft standard DPD linear force (see eq. (10)). The radius of each colloidal particle is chosen to be equal to the radius of the ring, and hence the configuration of RBC is approximately a closed-torus as shown in FIG. 52.

The WLC spring force interconnecting all cell particles in each RBC is given by

$${F}_{WLC}^{U} = \frac{{k}_{B} T}{{λ}_{p}} [ \frac{1}{4 {( 1 - \frac{{r}_{ij}}{{L}_{max}} )}^{2}} - \frac{1}{4} + \frac{{r}_{ij}}{{L}_{max}} ] , \qquad ( 31 )$$

where rij is the distance between two neighbor beads, λp is the persistence length, and Lmax is the maximum allowed length for each spring. Since the cell has also bending resistance, we incorporate into the ring model bending resistance in the form of “angle” bending forces dependent on the angle between two consecutive springs. The bending forces are derived from the COS bending potential given by

Uᵢⱼₖ^{COS}=k_{b[}1−cos θᵢⱼₖ], (32)

where kb is the bending stiffness, and θijk is the angle between two consecutive springs, which is determined by the inner product of rij and rjk. Then the bending force on particle j is derived as

$${F}_{j}^{COS} = - \frac{\partial {U}_{ijk}^{COS}}{\partial {r}_{j}} .$$

Here, λp determines the Young's modulus, and along with Lmax and a give the right size of RBC. To match both axial and transverse RBC deformations with the experimental data (22), kb is adjusted to reach a good agreement, which also gives some contributions to the Young's modulus. The LD-RBC model does not have the membrane shear modulus.

Since the thickness of LD-RBC model is constant, we estimate the variations of the RBC volume and surface area under stretching by calculating the relative change of the area formed by the ring under stretching. For healthy RBCs we find that it varies within only 8% in the range of all stretching forces (17). Therefore, the surface-area and hence the volume of RBCs remain approximately constant in the LD-RBC model.

### 3.1 Number of Particles in LD-RBC Model

We examine the effect of coarse-graining on stretching response by varying the number of particles (Nc) to model the RBC. FIG. 53 shows the RBC shape evolution from equilibrium (0 pN force) to 100 pN stretching force at different Nc. Note that an increase of the number of particles making up the RBC results in a smoother RBC surface. However, this feature seems to be less pronounced for higher Nc. Also, when we stretch the RBCs with different Nc, we find that an increase of Nc results in better agreement with the experimental data (22), but after Nc=10, the change becomes very small (17). To gain sufficiently good agreement and keep the computation cost low, we choose Nc=10 for all rest simulations shown herein; this is the accurate minimalistic model that we employ in our studies.

### 3.2 Aggregation Model

For LD-RBCmodel, we also employ the Morse potential to model the total intercellular attractive interaction energy.

The Morse potential and force are defined as

$$\begin{matrix}φ ( r ) = {D}_{e} [ {e}^{2 β ( {r}_{0} - r )} - 2 {e}^{β ( {r}_{0} - r )} ] , & ( 33 ) \\ f ( r ) = - \frac{\partial φ ( r )}{\partial r} = 2 {D}_{e} β [ {e}^{2 β ( {r}_{0} - r )} - {e}^{β ( {r}_{0} - r )} ] . & ( 34 )\end{matrix}$$

Here, r is the cell-cell surface distance, r0 is the zero force distance between two cells' surface, De is the well depth of the potential, and β characterizes the interaction range. The interaction between RBCs derived from the Morse potential includes two parts: a strong short-ranged repulsive force and a weak long-ranged attractive force. The repulsive force is in effect when r=0 (cells's surface contact) until their surface is separated by a distance of r0 (r=r0); usually r0 is in nanometer scale (1, 15, 16). In our simulations, r0 is chosen as 200 nm.

Here, r is calculated based on the center of mass of RBCs, i.e., r is equal to the distance between the center of mass of two RBCs minus the thickness of RBC. We also calculate the normal vector of each RBC (˜nc), which is used to determine if the aggregation occurs between two RBCs according to the angles formed by the normal vectors of two RBCs with their center line. The RBC normal vector is defined as:

$${\vec{n}}_{c} = \frac{\sum {\vec{υ}}_{k} \times {\vec{υ}}_{k + 1}}{{N}_{c}} , {\vec{υ}}_{k} = {x}_{k} - {x}_{c} . \qquad ( 35 )$$

Here, xk is the position of the kth particle in each RBC, xc is the position of the center of mass, and Nc is the number of particles in each RBC. The center line ˜vcij of two RBCs (cell i and cell j) is defined as xci−xcj. The angle formed by the normal vector of one cell with the center line is determined by their dot product

$${d}_{i} = \frac{{\vec{n}}_{ci}}{‖ {\vec{n}}_{ci} ‖} \cdot \frac{{\vec{υ}}_{cij}}{‖ {\vec{υ}}_{cij} ‖} . \qquad ( 36 )$$

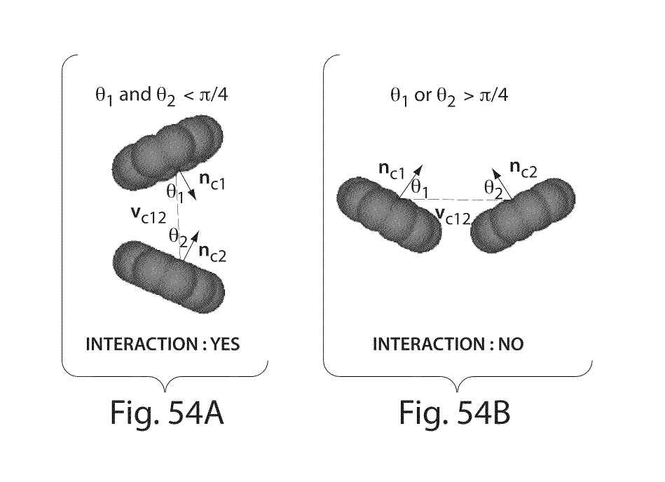

The Morse interaction is turned on if di>dc and dj>dc, otherwise, it is kept off. The critical value, dc, is chosen to be equal to cos(π/4), i.e., the critical angle (θc) to turn on/off the aggregation interaction is π/4. This value is found to be suitable to induce rouleaux formation, but exclude the disordered aggregation. The proposed aggregation algorithm can be further illustrated by a sketch in FIG. 54, where the aggregation between two neighbor RBCs is decided to be on/off according to their relative orientation.

### 3.3 Simulation Setup and Parameters

The DPD interactions among different particle types (solvent (S), and cell (C) particles) are listed in table 3. Random force coefficients for different interactions were obtained according to σᵢⱼ=\sqrt{2k_{B}Tγᵢⱼ} with kBT=0.1. The number densities of solvent particles is set to be nS=3.0. Lmax=1.3, λp=0.0005 and kb=50. The Morse potential parameters are chosen as: De=500, β=3.0 and r0=0.1.

TABLE 3
LD-RBC: Parameters of DPD interactions in simulations.

| interaction | | γ^{∥} = γ^{⊥} | r_{c} |
|---|---|---|---|
| | radial conserative force | | |
| | linear (eq. (10)) | | |
| S-S | a = 2.5 | 4.5 | 1.5 |
| C-C (same cell) | α = 500 | 4.5 | 1.2 |
| | radial conservative force | | |
| | exponential (eq. (14)) | | |
| C-C (different cell) | α = 2500, b = 20, r_{c}^{e} = 2.0 | 4.5 | 2.0 |
| S-C | α = 2500, b = 20, r_{c}^{e} = 1.0 | 900 | 1.5 |

### 4 Scaling of Model and Physical Units

The dimensionless constants and variables in the DPD model must be scaled with physical units. The superscript M denotes that a quantity is in “model” units, while P identifies physical units (SI units). We define the length scale as follows

$${r}^{M} = \frac{{D}_{0}^{P}}{{D}_{0}^{M}} m , \qquad ( 37 ) ,$$

where rM is the model unit of length, D0 is the cell diameter, and m stands for meters. The energy per unit mass (kBT) and the force unit (“N” denotes Newton) scales are given by

$${( {k}_{B} T )}^{M} = \frac{{Y}^{P}}{{Y}^{M}} {( \frac{{D}_{0}^{P}}{{D}_{0}^{M}} )}^{2} {( {k}_{B} T )}^{P} , \\ {N}^{M} = \frac{{Y}^{P}}{{Y}^{M}} \frac{{D}_{0}^{P}}{{D}_{0}^{M}} {N}^{P} , \qquad ( 38 )$$

where Y is the membrane Young's modulus. The time scale is defined as

$$τ = \frac{{D}_{0}^{P}}{{D}_{0}^{M}} \frac{{η}^{P}}{{η}^{M}} \frac{{Y}^{M}}{{Y}^{P}} s . \qquad ( 39 )$$

where η is a characteristic viscosity (e.g., solvent or membrane).

### References for Example 9

1. S. Chien and K.-M. Jan. Ultrastructural basis of the mechanism of rouleaux formation. Microvascular Research, 5:155-166, 1973.
2. P. Espanol. Fluid particle model. Physical Review E, 57(3):2930, 1998.
3. P. Espanol and P. Warren. Statistical mechanics of dissipative particle dynamics. Europhysics Letters, 30(4):191-196, 1995.
4. E. A. Evans and R. Skalak. Mechanics and thermodynamics of biomembranes. CRC Press, Inc., Boca Raton, Fla., 1980.
5. X. Fan, N. Phan-Thien, S. Chen, X. Wu, and T. Y. Ng. Simulating flow of DNA suspension using dissipative particle dynamics. Physics of Fluids, 18(6):063102, 2006.
6. X. J. Fan, N. Phan-Thien, S. Chen, X. H. Wu, and T. Y. Ng. Simulating flow of DNA suspension using dissipative particle dynamics. Physics of Fluids, 18(6):063102, 2006.
7. D. A. Fedosov. Multiscale modeling of blood flow and soft matter. PhD thesis, Brown University, USA, 2010.
8. D. A. Fedosov, B. Caswell, and G. E. Karniadakis. A multiscale red blood cell model with accurate mechanics, rheology, and dynamics. Biophysical Journal, 98(10):2215-2225, 2010.
9. D. A. Fedosov, B. Caswell, and G. E. Karniadakis. Systematic coarse-graining of spectrin-level red blood cell models. Computer Methods in Applied Mechanics and Engineering, 199:1937-1948, 2010.
10. D. A. Fedosov, I. V. Pivkin, and G. E. Karniadakis. Velocity limit in DPD simulations of wall-bounded flows. Journal of Computational Physics, 227(4):2540-2559, 2008.
11. R. D. Groot and P. B. Warren. Dissipative particle dynamics: Bridging the gap between atomistic and mesoscopic simulation. Journal of Chemical Physics, 107(11):4423-4435, 1997.
12. W. Helfrich. Elastic properties of lipid bilayers: theory and possible experiments. Z. Naturforschung C, 28:693-703, 1973.
13. P. J. Hoogerbrugge and J. M. V. A. Koelman. Simulating microscopic hydrodynamic phenomena with dissipative particle dynamics. Europhysics Letters, 19(3):155-160, 1992.
14. A. W. Lees and S. F. Edwards. The computer study of transport processes under extreme conditions. Journal of Physics C, 5:1921-1928, 1972.
15. Y. Liu and W. K. Liu. Rheology of red blood cell aggregation by computer simulation. Journal of Computational Physics, 220:139-154, 2006.
16. B. Neu and H. J. Meiselman. Depletion-mediated red blood cell aggregation in polymer solutions. Biophysical Journal, 83:2482-2490, 2002.
17. W. Pan, B. Caswell, and G. E. Karniadakis. A low-dimensional model for the red blood cell. Soft Matter, 6:4366-4376, 2010.
18. W. Pan, B. Caswell, and G. E. Karniadakis. Rheology, microstructure and migration in brownian colloidal suspensions. Langmuir, 26(1):133-142, 2010.
19. W. Pan, D. A. Fedosov, B. Caswell, and G. E. Karniadakis. Hydrodynamic interactions for single dissipative-particle-dynamics particles and their clusters and filaments. Physical Review E, 78(4):046706, 2008.
20. W. Pan, I. V. Pivkin, and G. E. Karniadakis. Single-particle hydrodynamics in dpd: A new formulation. Europhysics Letters, 84(1):10012, 2008.
21. V. Pryamitsyn and V. Ganesan. A coarse-grained explicit solvent simulation of rheology of colloidal suspensions. Journal of Chemical Physics, 122(10):104906, 2005.
22. S. Suresh, J. Spatz, J. P. Mills, A. Micoulet, M. Dao, C. T. Lim, M. Beil, and T. Seufferlein. Connections between single-cell biomechanics and human disease states: gastrointestinal cancer and malaria. Acta Biomaterialia, 1:15-30, 2005.

### Example 10

### Assessment of Febrile Temperature and Antimalarial Drug-Treatment for Enhanced Splenic Parasite Clearance

### Introduction

Malaria is a deadly parasitic disease which affects approximately three billion people worldwide and accounts for nearly one million deaths annually (1). A virulent malarial parasite Plasmodium falciparum can lead to severe complications and has the highest mortality rate (2). The malaria parasites, during their asexual stage, infect red blood cells (RBCs), which then undergo notable morphological and rheological changes from the ring-form (rings) to trophozoite and finally schizont.

Cyclic febrile attack is a characteristic clinical feature of P. faciparum malaria, which corresponds to the release of merozoites (free parasites) following schizont rupture. During intra-erythrocytic development, the invasion of merozoites to other red blood cells (RBCs) reinitiates a 48 hour asexual reproduction cycle (3). In addition to being a fatal and acute complication of cerebral malaria, malarial anemia is a frequent and severe manifestation of malaria (4). Massive loss of red blood cells, which causes malarial anemia, cannot be entirely attributed to the destruction of infected RBCs (iRBCs), which usually constitute a small fraction of total RBCs in patients. A major cause of malarial anemia is that many uninfected RBCs (uRBCs) are lost in patients' blood, mostly in the spleen and/or the liver (5). Malaria-related dyserythropoiesis is likely a minor factor, because a complete removal of erythropoiesis brings about a minor decrease in RBC population (6). On the other hand, uRBCs are slightly less deformable, and/or “decorated” with parasite molecules (7), both of which could potentially lead to splenic retention and clearance of large number of uRBCs, exacerbating malarial anemia.

It is very likely that RBC filtration in the spleen has a role in shaping diverse pathophysiological outcomes of malaria. Splenomegaly (enlarged spleen, probably caused by excess amount of iRBCs and uRBCs retained) is one of the clinical markers for malaria infection. The narrow splenic inter-endothelial slits (˜1 um) provide a stringent mechanical filter, through which only the RBCs with adequate deformability can pass. While rings are only moderately stiffer than uRBCs, later-stages of infected cells (trophozoites and schizonts) can be 10 to 50 times stiffer (8). As a result, typically, only rings can be seen in the peripheral blood circulation in vivo, whereas stiffer iRBCs such as trophozoites and schizonts are typically sequestered during microcirculation, to be phagocytosed by macrophages. A substantial (˜50%) proportion of rings may also be retained by human spleen, as demonstrated using isolated human spleens (9). The attachment of parasite-derived protein, Ring-infected Erythrocyte Surface Antigen (RESA), may be responsible for the stiffening of infected cells (10).

Clinical studies on several antimalarial drugs revealed that patients treated with artemisinin and its derivatives exhibit a more rapid decline in parasitemia. However, the accelerated parasite clearance is delayed or even obscured in splenectomized patients (11). Therefore, active splenic retention may be the underlying mechanism allowing rapid parasite clearance after anti-malarial treatment (5). As the spleen could mechanically filter stiffer cells from microcirculation; the more efficient splenic clearance after drug treatment may indicate that artemisinin and its derivative may be able to modify the mechanical properties of healthy or parasitized cells.

Because even a subtle change in deformability could lead to significant shift in RBCs' splenic retention efficiency (5), it is informative to characterize cells' dynamic deformability carefully and quantitatively, to shed light on the clinical problem of malarial anemia. Earlier bulk measurements such as ektacytometry (12,13) provides averaged cell deformability information (EI: elongation index), which may not reflect individual cells' mechanical properties. The deformability of individual RBCs can be measured by a number of methods, including micropipette aspiration (14,15), atomic force microscopy (16), and optical tweezers (17). Many of these measurements apply quasi-static loads to attain notable deformation. The deformability of the cells is, in these methods, characterized by the shear and bending moduli of the cell membrane. However, when RBCs circulate in the blood capillaries and splenic cordal meshwork, their ability to deform is also a time-dependent response, which conventional static measurements may not assess directly. A method to evaluate cells' ability to pass constrictions posed by the spleen and microcapillary blood vessel is to simulate in vivo RBC deformations during circulation using microfluidic artificial filter structures (18).

Whereas the intermittent fever paroxysm is a characteristic feature of P. falciparum malaria, the artesunate anti-malarial drug-treatment may actively interact with the infected cells at molecular level. Both environmental stimuli seem to have direct or indirect relevance with the parasite retention in the human spleen (11). A microfabricated deformability cytometer can measure dynamic mechanical responses of thousands RBCs in a population (19). Distinct from conventional tools for single cell analysis, microfluidic devices described herein can process approximately 10 cells per second. The high throughput enables the measurement of a considerable proportion of cells, permitting high sensitivity and low sampling error. It was found that subtle deformability shifts of RBCs, in response to shifts in temperature or drug concentration, were measured quantitatively, providing information about the factors involved in the process of splenic clearance of drug-treated iRBCs and malarial anemia.

### Results

Malarial anemia is associated with a massive loss of red blood cells (RBCs) in patients and is a common malaria-related complication among children. Splenic clearance of both infected and uninfected RBCs is a factor contributing to the blood loss. During the intra-erythrocytic development of malaria, several environmental stresses including cyclic febrile attacks and external anti-malarial drug-treatment may influence splenic cell filtration. In this example, a microfluidic system was used to mimic splenic cords and to measure the dynamic mechanical response of RBCs under different conditions. At febrile temperature, infected RBCs stiffened by approximately 35% whereas uninfected RBCs exhibited a relatively minor deformability decrease. Similar trends were observed in RBCs with drug-treatment. The results in this example indicate that efficiency and specificity of splenic clearance of infected RBCs may be enhanced at febrile temperature or with drug treatment.

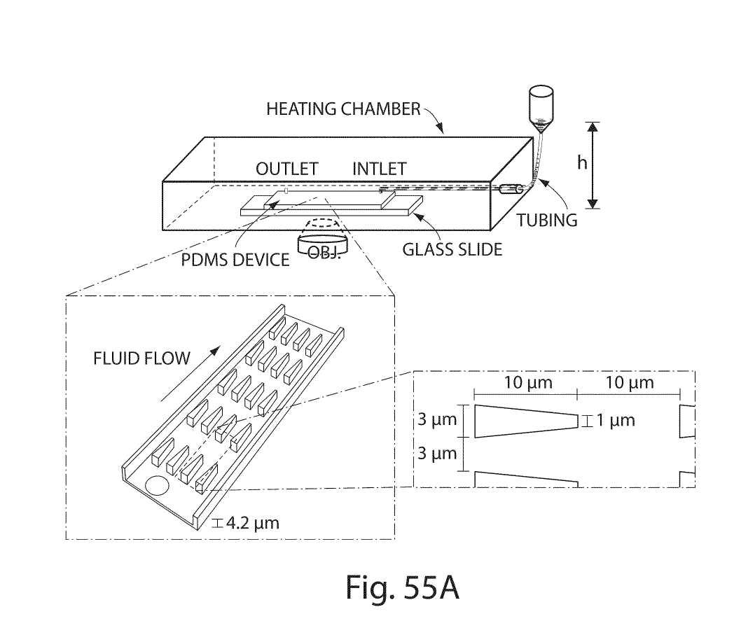

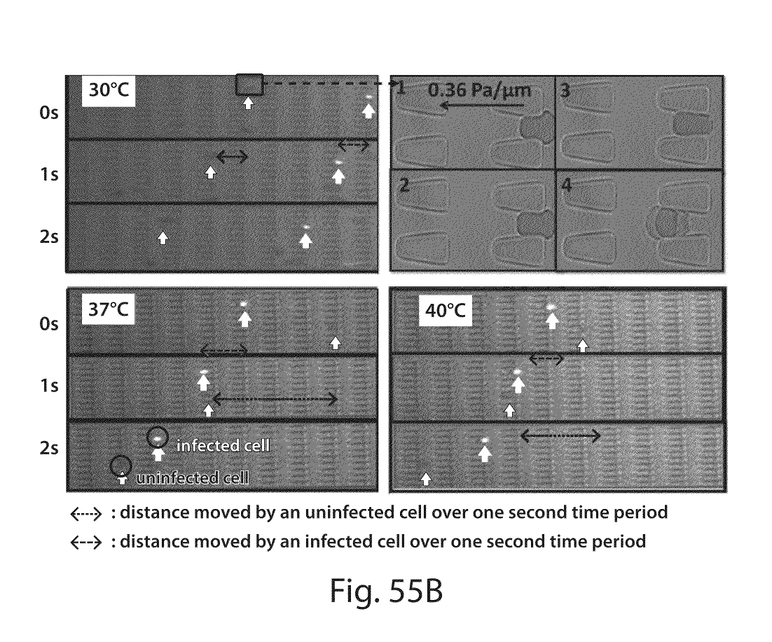

The device used in this example comprises a series of equally spaced triangular pillar arrays with pore sizes ranging from 2.5 to 4 um (FIG. 55A illustrates the case of 3 um pore size). Compared to the diameter of an average RBC (˜7.5 um), the smaller pore sizes are designed to impose similar mechanical constraints on the cells as if they are passing through blood capillaries and splenic meshwork. Driven by constant pressure gradient in the sub-Pascal-per-micrometer range, RBCs are able to deform substantially at each constriction and traverse along the channel. The dynamic deformability of RBCs is then characterized by their mobility: the ability to deform repeatedly in order to pass through multiple constrictions in series.

FIG. 55A depicts an experimental schematic of the microfluidic system. A heating chamber (Olympus) was mounted to the inverted microscope stage. Four heaters for the inner water bath, microscope stage, chamber top, and lens were designed to accurately control the ambient temperature inside the chamber. The PDMS-glass bonded device consists of the inlet and outlet reservoirs and main channels with triangular pillar arrays. It was placed inside the heating chamber with only the inlet reservoir connecting to an external syringe via a 20 cm-tubing. The reservoirs were 500×500 μm² squares with 20 μm-interspacing cylindrical pillar arrays; these pillar arrays could pre-filter white blood cells from whole blood, allowing only RBCs to pass through the main channels. Each of the parallel channels was 10 pillars wide and 200 pillars long. Along the flow direction, the inter-pillar spacing was 10 μm. This spacing allows deformed cells to recover and ready for subsequent deformations. Perpendicular to the flow direction, the pore size varied from 2.5 to 4 μm for different channels. Cells experience different levels of deformation when passing through these pores.

FIG. 55B presents microscopic screenshots illustrating both iRBCs and co-cultured uRBCs moving in the microchannel at different temperature conditions. The uninfected cells appear as dark shadows indicated by blue arrows, and the infected cells with thiazole orange (TO) staining appear as bright dots indicated by white arrows. The mobility of individual cells can then be derived by recording the time period for each cell passing through 10 constrictions in series (i.e. equivalent to 200 μm distance travelled). In this example, cell mobility is used to characterize the dynamic deformability of individual RBCs. The typical pressure gradient (0.1˜0.5 Pa/μm) and shear rate (50˜500 s⁻¹) applied in this device as well as the resulting RBC flow rate (20˜200 μm/s) are comparable to the physiological flow conditions during microcirculation; they are also in the same order of magnitude as RBCs passing through splenic interendothelial slits (IES).

Temperature-Dependent iRBC Deformability

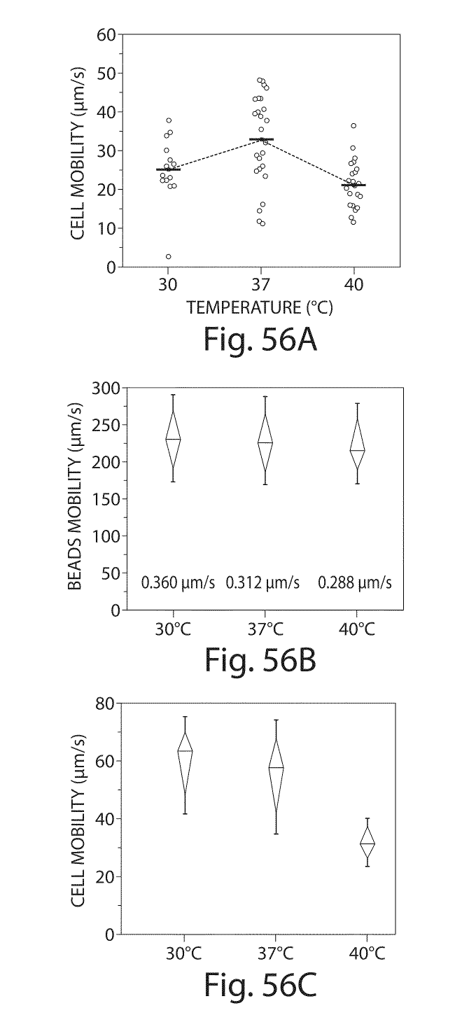

FIG. 56A demonstrates the temperature-dependent modification on iRBC deformability. The mobility of infected cells exhibited a significant increase from 30° C. to 37° C. followed by a notable drop at 40° C. The peak at 37° C. marked an optimum temperature for maximum iRBC deformability in this example.

To investigate the increase from 30° C. to 37° C., several factors were taken into consideration including cell membrane viscosity, intracellular fluid viscosity, buffer solution viscosity as well as possible confounding effects caused the modification of cytoskeletal structure and membrane proteins. PBS buffer viscosity decreases by 33% from 19° C. to 37° C., and the blood viscosity decreases by 2% for every 1° C. temperature increment (i.e. ˜31% decrease from 19° C. to 37° C.) (22). At a given pressure gradient, elevated temperature increases the bulk fluid flow, leading to a increase in cell mobility measurement. For another comparison, normalized cell deformability was measured by performing the experiment at equalized bulk fluid velocity over all temperatures. Assuming the combined viscosity shift in iRBC and PBS buffer solution is linear and can be resembled by bulk fluid velocity, the pressure gradient to be applied at each temperature for constant fluid flow was computed. This calibration was experimentally verified by measuring fluid velocity via 200 nm non-deformable polystyrene beads (FIG. 56B). With constant beads mobility of 226 μm/s, the normalized cell deformability (FIG. 56C) inside 4 μm-channel was found to be fairly constant from 30° C. to 37° C. This is consistent with measurements using micropipette aspiration (14) and optical tweezers (17).

The significant drop in iRBC mobility between 37° C. and 40° C. was preserved at constant local fluid velocity (FIG. 56C). This stiffening effect at febrile temperature agrees with measurement by optical tweezers (10). While RESA would be necessary for the parasitized cells to survive heat-induced damages, the protein-related stiffening also facilitates more efficient splenic clearance. The role of RESA in iRBC stiffening was confirmed with static (17) and dynamic measurements.

Temperature-Dependent uRBC Deformability

The temperature dependent modification on co-cultured uRBC deformability has been overlooked. If uRBCs were retained (to a higher degree than normal) in the spleen of a P. Falciparum patient, their deformability may have been (however minutely) decreased.

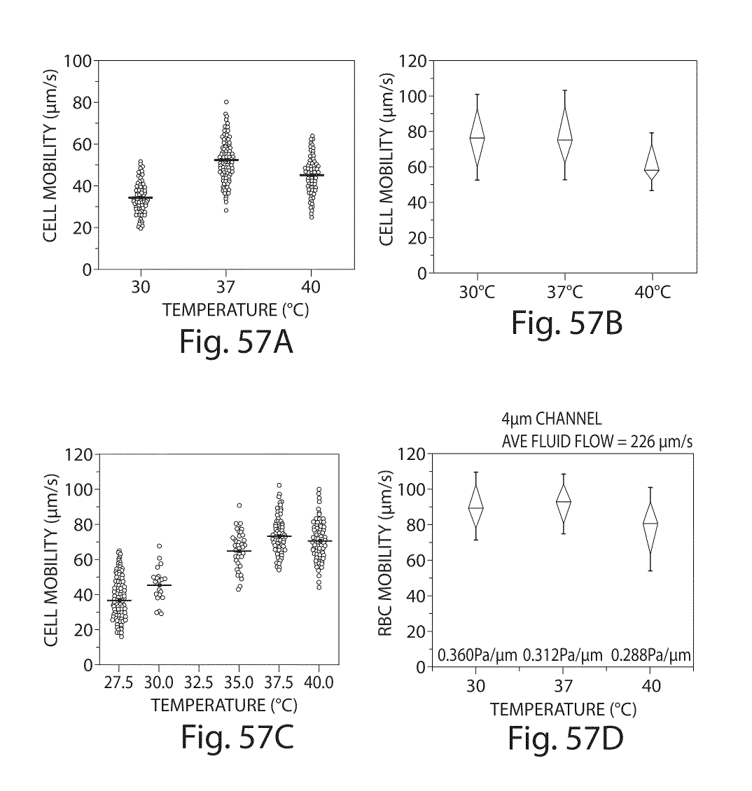

FIG. 57A demonstrates the temperature-dependent modification on (co-cultured) uRBC deformability. Similar to that of iRBCs, the uRBC mobility increased significantly from 30° C. to 37° C. From 37° C. to 40° C., instead of a 40% drop displayed by iRBCs, the decrease in uRBC deformability was statistically significant (p<0.01). Normalized uRBC mobility was also measured at equalized bulk fluid velocity inside 4 μm-channel (FIG. 57B). The result was similar to that of normalized iRBC deformability: no significant change in the normalized uRBC deformability was observed from 30° C. to 37° C., and the mobility drop from 37° C. to 40° C. was preserved.

The result demonstrates the role of viscosity in influencing the RBC deformability from 30° C. to 37° C. This result also demonstrates that the drop in iRBC mobility at febrile condition is associated with an effect of the parasite-derived protein RESA, and is not something inherent in non-parasitized cells. This result is also consistent with similar measurement using other techniques (17,23).

### Temperature-Dependent Healthy RBC Deformability

The dynamic deformability of healthy RBCs (hRBCs) from room temperature (25° C.) to febrile temperature (40° C.) was measured to compare with the result for uRBCs. This test assessed whether a deformability change of uRBC is associated with a biochemical factor present in the co-culture environment. Under a constant pressure gradient scheme, the hRBCs became more deformable from room to body temperature, and the deformability peaked at 37° C. The measured temperature-dependent RBC deformability was independent of the thermal history of the sample, which was assessed by changing the order in which measurements were made at different temperature values. In addition, temperature-induced deformability changes were reversible under the conditions tested (from 25 to 40° C.), meaning that returning a sample to its original temperature restored the deformability value measured at that temperature.

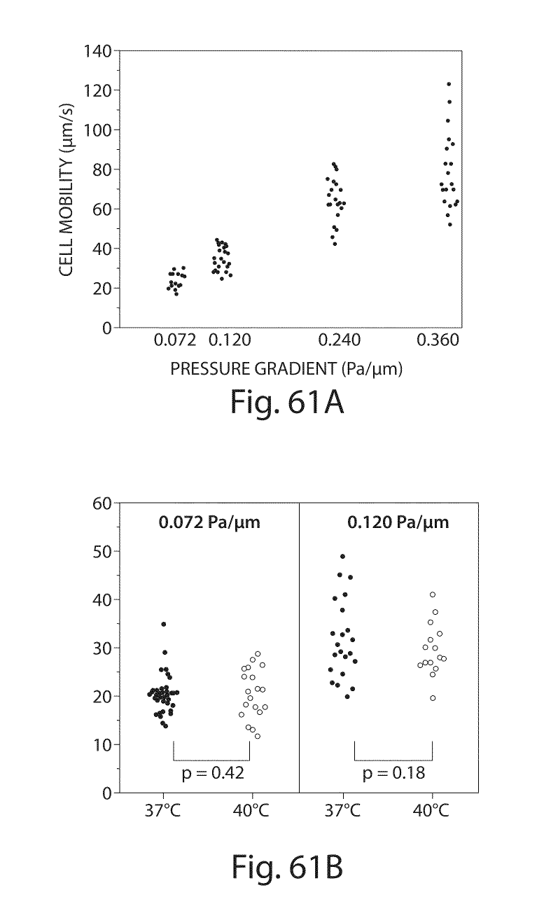

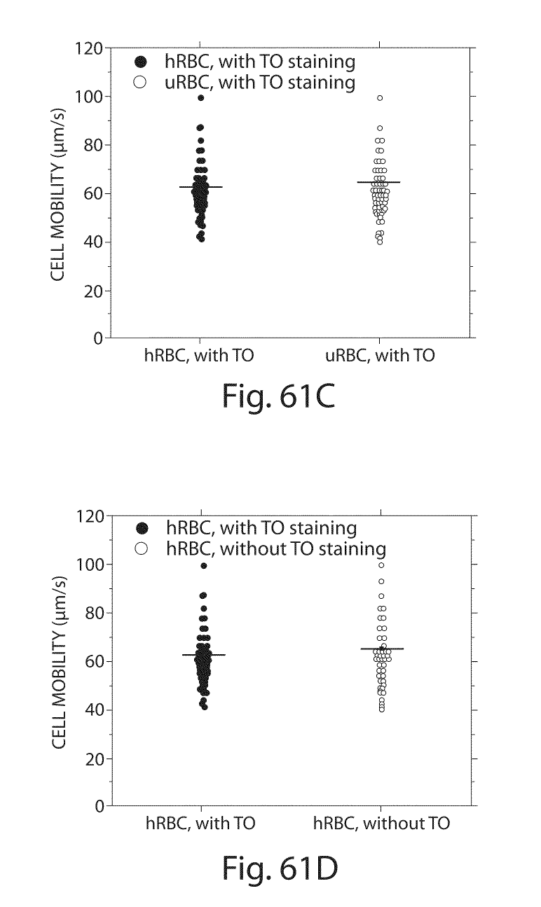

In a control experiment with constant bulk fluid flow velocity, for a constant beads velocity of 226 μm/s, the hRBC mobility appear to be significantly higher than that of uRBCs. Several factors could account for such disagreement such as the source of the cells and incubation conditions. The healthy RBCs were obtained from fresh blood cells within 2-days whereas the uninfected cells analyzed were parasite co-cultured RBCs and are on average much older than fresh cells. Some studies revealed that a proportion of the co-cultured but non-parasitized cells are invaded by parasite molecules (5, 24-26), which may consequently stiffen the cells. To investigate further the deformability differences between hRBCs and uRBCs, a control experiment was performed in which both hRBCs and uRBCs were prepared in essentially the same way except that uRBCs were exposed to malarial parasites (FIG. 61).

Febrile Condition Enhances the Separation Resolution Between iRBCs and uRBCs

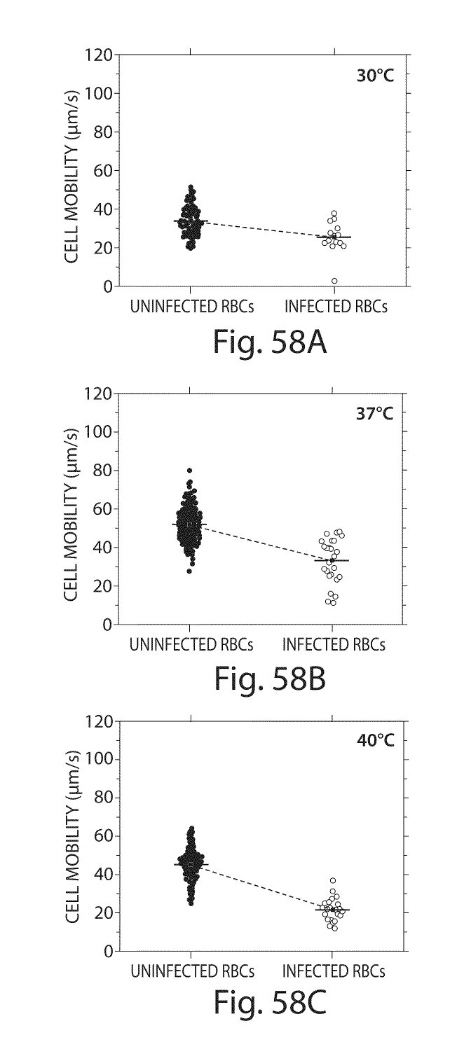

The deformability separation resolution between normal and parasitized cells is a parameter for efficient splenic filtration of infected RBCs. The temperature-dependent cell deformabilities for both iRBCs and co-cultured uRBCs were simultaneously measured at 30° C., 37° C., and 40° C. (FIG. 58). The deformability separation resolution Rₛ between normal and infected cells was analyzed using the formula below, where X₁, X₂ and σ₁, σ₂ denote the mean and standard deviation of normal and infected cell mobilities. A higher Rₛ value implies better separation.

$${R}_{S} = \frac{{X}_{2} - {X}_{1}}{2 ( {σ}_{1} + {σ}_{2} )}$$

While at all tested temperatures the infected RBCs displayed statistically significant stiffening compared to uninfected cells (p<0.001), the deformability separation resolution between uRBCs and iRBCs enhanced with increasing temperature (Table 10.1). At 30° C., the separation resolution was only 0.28; the value increased to 0.46 at body temperature, and to 0.94 at febrile condition. Furthermore, at 40° C., the average iRBC mobility was 3.02σ (σ: standard deviation of uRBC mobility distribution) away from the average uRBC mobility. This result indicates a sensitive and specific deformability differentiation between normal and parasitized RBCs at febrile condition. The result is consistent with measurements using other techniques (17, 23) and indicates that a febrile condition facilitates efficient (stiffening of iRBC) and specific (separation between iRBC and uRBC) splenic retention.

TABLE 10.1

| Temperature | Resolution Rs |
|---|---|
| 30° C. | 0.28 |
| 37° C. | 0.46 |
| 40° C. | 0.94 |

Effect of Anti-Malarial Drug on the Deformability of P. falciparum-Infected RBCs

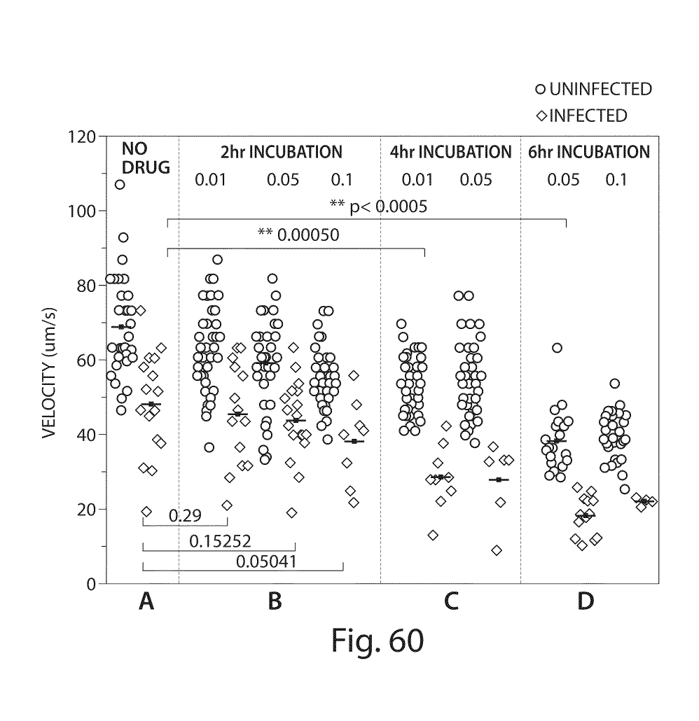

The deformability of both iRBCs and uRBCs were measured at 2, 4 and 6 hours after artesunate drug treatment (FIG. 60). A synchronized culture of rings with ˜15% parasitemia was resuspended at 0.1% hematocrit in malaria culture medium containing 0.01, 0.05 or 0.1 μg/ml of artesunate.

A stiffening effect on both iRBCs and uRBCs resulted from artesunate treatment. At 6 hours after artesunate treatment, a 30%-50% mobility decrease is observed, while smaller and less pronounced mobility decrease is found at both 2 and 4 hours after artesunate treatment. After 4 hour drug treatment, within the effective dosage range of 0.01 to 0.1 μg/ml, no statistically significant dose dependence is found in terms of mobility chances.

The trend exhibited by artesunate-treated RBCs was similar to the cells exposed to febrile conditions. After the drug treatment, though both iRBCs and uRBCs stiffened considerably, the drop in iRBC deformability is more precipitous than in uRBC. The separation resolution was almost doubled. The significant decrease in cell mobility due to drug treatment is expected to effectively promote spleen clearance of infected RBCs.

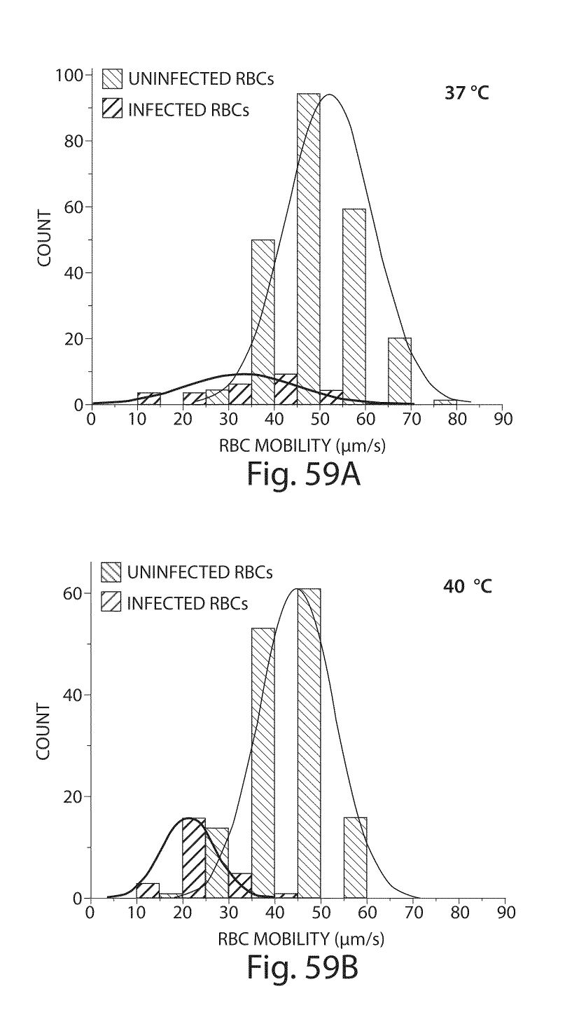

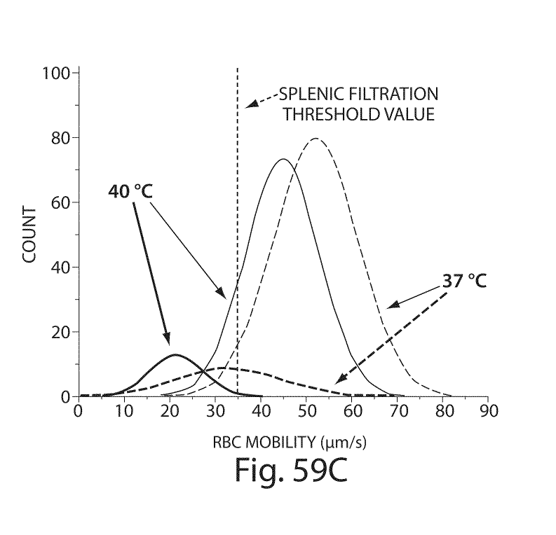

By reducing the deformability of parasitized cells, fever (which is a common, recurring symptom for any malaria patient) increases the separation resolution between uninfected and infected RBCs (FIG. 59), facilitating more efficient and specific splenic retention of parasitized RBCs.

The results also indicate a role of fever in malarial anemia, because average deformability of uRBCs was decreased by 30% on average at febrile temperature, compared with body temperature.

These results support two hypotheses regarding parasite clearance and malarial anemia. The first is the role of fever in enhancing splenic clearance of parasite. It is thought that subtle modifications on uRBC stiffness (5) (much subtler than a drastic shift of iRBC stiffness) may render additional support on the splenic retention model for malarial anemia. The results show the importance of febrile temperature in this subtle balance. By reducing the deformability of parasitized cells, fever (which is a common, recurring symptom for any malaria patient) increases the separation resolution between uninfected and infected RBCs (FIG. 59), facilitating more efficient and specific splenic retention of parasitized RBCs. The uRBC population is inherently diverse, due to age and other factors (34) and the wide mobility distribution uRBCs are likely attributed by this diversity. Still, separation between the uninfected and infected population is sufficiently high that infected cells can be distinguished even though they constitute only a minor fraction (≦1%). The separation resolution between uRBCs and iRBCs can serve as a non-chemical biomarker in malarial diagnosis.

### Discussion

The temperature- and drug-dependent deformability measurement for healthy (hRBC), co-cultured but unparasitized (uRBC), and parasitized RBCs (iRBC, predominantly synchronized rings) in this study yielded several interesting and important observations. In sum, the in vitro results signify the importance of cell deformability shifts caused by either febrile temperature or drug treatment, in the progression of P. falciparum infection.

The physiological body temperature (37° C.) seems to be an optimum temperature for maximum deformability (maximum passage through the spleen) for all cell types. This was caused by two distinct trends, below and above the body temperature. An approximately 50% increase in cell deformability from room to body temperature was predominantly caused by temperature related viscosity change in both RBCs and PBS buffer solution. Other factors such as the cytoskeletal structural modification (27, 28) membrane protein alternation seem to have played a minor role within this temperature range.

From body to febrile temperature, an approximately 40% drop in the average deformability was observed among malaria-infected cell population (17,23). The role of RESA was well established to be responsible to alter the deformability of infected cells (29), and to prevent spectrin from undergoing heat-induced conformational changes, thereby increasing infected RBC survival at febrile temperatures. It has been found that a subtle change in uRBCs/hRBCs at febrile temperature is small but quantifiable. The minute RBC stiffening at febrile condition may involve several important biological mechanisms such as heat induced structural transformation in the membrane lipid bilayers (30-33) and hemoglobin molecules (27).

Experiment results from hRBCs (which have never interacted with parasite cells) show the same temperature-dependent trend on RBC deformability. This suggests that the measured stiffening of RBCs at febrile temperature may be due to inherent structural changes in RBC cytoskeleton (such as spectrin networks), although it does not preclude the possibility of uRBC stiffening caused by the release of exoantigens from parasites that bind to normal RBCs (36).

In the experiment assessing anti-malarial drug effect on RBC deformability, the separation resolution between iRBCs and uRBCs was doubled after Artesunate drug treatment. The result suggests that Artesunate may be responsible for enhanced specificity and efficiency in splenic parasite filtration. Clinical studies performed in patients with and without a spleen confirmed that Artemisinin or one of its derivatives is actively involved in the process of splenic parasite clearance. Several mechanisms of the drug action has been proposed including the involvement of reactive oxygen free radicals, haeme metabolism, as well as specific target proteins (44-46); however, the specific role of Artemisinin is still unclear. The results provided add to the understanding of the drug mechanism. With the tool of microfluidics, iRBC deformability shift was quantitatively measured by Artemisinin and its derivatives. If the drug-treated RBC deformability trend is compared with aforementioned temperature dependent deformability measurement, the results are surprisingly similar. This suggests that Artesunate may be able to result in similar stiffness changes to the RBCs as febrile temperatures do. On the other hand, drug-induced “pitting” (i.e., removing intraerythrocytic parasite without destroying the host RBC) may be an alternative mechanism of the artesunate drug action (Chotivanich et al.). This “pitting” effect can be investigated carefully by optimizing the devices and flow conditions provided herein.

In conclusion, the results demonstrate that the efficiency with which diseased RBCs are cleared by the spleen may be directly dependent on elevated body temperature. Our findings suggest an important role of fever in enhancing splenic clearance of less deformable parasite-infected RBCs from the circulation at febrile temperatures. On the other hand, fever may aggravate the loss of uninfected RBCs which in the worst case may inadvertently lead to malarial anemia. These measurements could provide a well-controlled in vitro experimental platform to test novel anti-malarial compounds, or elucidate the mechanism of drug action in relation to splenic clearance, which is generally difficult to do in vivo due to ethical and other considerations.

### Materials and Methods

### Device Fabrication

Layout program CleWin3.0 was used to design the microfluidic device, which consists of a 500×500 μm² inlet reservoir, a 500×500 μm² outlet reservoir, and parallel capillary channels with triangular pillar arrays (FIG. 55A). Three different pore-size of 2.5, 3.0 and 4.0 μm were designed for the capillary channels to test optimum deformation condition for the experiment. A silicon mold of the device was made using standard silicon processing techniques. The photolithography step was done using a 5× reduction step-and-repeat projection stepper (Nikon NSR2005i9, Nikon Prevision) and reactive-ion etching (RIE) techniques were used to give the mold a final depth of 4.2 μm. Final device was then casted from the silicon mold using polydimethylsiloxane (PDMS) and was sealed by a glass slide using oxygen plasma.

### Parasite Culture

P. falciparum 3D7A parasites (from Malaria Research and Reference Reagent Source, American Type Culture Collection) were cultured in leukocyte-free human RBCs (Research Blood Components, Brighton Mass.) in RPMI 1640 complete medium as described (Trager and Jensen J. parasitol. 2005). Parasites cultures were synchronized by sorbitol lysis (Lambros, C 1979 J. parasitol.) two hours after merozoite invasion.

### Solution Preparation

1× Phosphate buffered saline (PBS) was mixed with 1% w/v Bovine Serum Albumin (BSA) (Sigma-Aldrich, St. Louis, Mo.) as a stock solution and was fresh made on every experimental day. For experiments tracking fluid flow velocity, 200 nm FluoSpheres europium luminescent microspheres (Molecular Probes, Eugene, Oreg.) were used at a final concentration of 5.0×10⁻⁴ percent solids. For experiments involving only healthy RBCs, fresh whole blood (Research Blood Components, Brighton, Mass.) was used at 100 times dilution (i.e. 1 μl whole blood with 99 μl stock solution). For experiments involving parasitized cells, 1 ml of cultured cells were spun down at 1,000 rpm for 5 minute; 1 μl of the pellet was then aliquot to 200 μl stock solution.

1 μl of 50 μg/ml Cell Tracker Orange (Invitrogen, Carlsbad, Calif.), which stains the membrane of the cell, was added to the afore-mentioned 100 μl healthy RBC solution 20 minutes before the experiment for better imaging. To ensure no adverse effect on the cell deformability was induced by the cell tracker dye, a control experiment of same RBC solution without staining was performed. No statistical significant difference was found between the sample with and without staining.

10 μl of 1×10⁻⁶M thiazole orange Orange (Invitrogen, Carlsbad, Calif.), which stains the RNA of the cell, was added to the aforementioned 200 μl iRBC solution 20 minutes before the experiment. The infected cells would appear fluorescent under the GFP filter set whereas the uninfected cells were seen as dark shadows.

### Experimental Protocol

To control the ambient temperature, the microscope surface was replaced by a heating chamber (Olympus), which was preheated to a desired temperature range (i.e. 30-40° C.) for 30 minutes before the beginning of every experiment. Meanwhile, the PBS-BSA stock solution was injected into the device to coat the PDMS walls to prevent adhesion. This filling step need not be done inside the heating chamber, but the PBS-BSA filled device needed to be placed into the heating chamber at least 5 minutes before loading 10 μL of diluted blood sample. During temperature calibration phase, a thermal meter was used to probe the exact temperature inside the heating chamber. When the temperature needed to be adjusted to a different value, at least 5 minutes of waiting time was required to ensure a new stable ambient temperature.

To apply a constant pressure gradient across the device, the pressure difference between inlet and outlet reservoir was generated hydrostatically by fixing the difference between inlet and outlet water column height (FIG. 55A). To ensure the pressure difference is constant throughout the experiment, a 60-ml syringe was selected to connect to the inlet reservoir such that the water column height would not vary significantly within several hours.

To capture the movement of RBCs inside the microchannels, a CCD camera (Hamamatsu Photonics, C4742-80-12AG, Japan) was connected to the inverted fluorescent microscope (Olympus IX71, Center Valley, Pa.). Images were automatically acquired by IPLab (Scanalytics, Rockville, Md.) at 100 ms time interval and the post-imaging analysis was done using imageJ. The mobility of individual RBCs was defined as the distance the cells moved divide by the time in seconds.

### Supplementary

The spleen is believed to work as a mechnical filter which removes stiffer cells from a large population. The splenic retention model has been hypothesized (Error! Reference source not found.). To quantitatively illustrate splenic clearance, data were replotted at body (37° C.) and febrile (40° C.) temperatures. A certain threshold value was assumed such that all the RBCs with mobility below that value will undergo spleen retention and vice versa.

In the experiments, the threshold mobility was set to be 34 μm/s, which is 2σ away from the average uRBC mobility measured at 37° C. This value was chosen such that too many normal RBCs were not lost (which otherwise would result in serious hemolysis), and a certain level of deformability selectivity was maintained. At 37° C., only 12 out of 25 iRBCs traverse below the threshold mobility, indicating the efficiency of splenic filtration of iRBC at 37° C. is only 48%; however, at 40° C., 24 out of 25 iRBCs has a mobility value lower than 34 um/s, suggesting 96% of iRBCs will be cleared by spleen at febrile condition.

The significant improvement in splenic filtration efficiency (from 48% to 96%) suggests an important role of fever temperature in the pathophysiology of falciparum malaria and in splenic clearance in general. On the other hand, when the splenic retention of uninfected RBCs at body and febrile temperatures was compared, it was found that while only 1.5% of uRBCs would be removed from blood stream at 37° C., 9% of uRBCs are below the threshold mobility at 40° C. While the exact mechanism for why the febrile condition would mildly reduce uRBC deformability is still unclear, the significantly increased amount of uRBC removal might be the source of malarial anemia.

### References for Example 10

1. World Health, O. World Malaria Report 2010. (2010).
2. Trampuz, A., Jereb, M., Muzlovic, I. & Prabhu, R. Clinical review: Severe malaria. Critical Care 7, 315-323 (2003).
3. Maier, A. G., Cooke, B. M., Cowman, A. F. & Tilley, L. Malaria parasite proteins that remodel the host erythrocyte. Nat. Rev. Microbiol. 7, 341-354 (2009).
4. Lamikanra, A. A., et al. Malarial anemia: of mice and men. Blood 110, 18-28 (2007).
5. Buffet, P. A., et al. The pathogenesis of Plasmodium falciparum malaria in humans: insights from splenic physiology. Blood 117, 381-392 (2011).
6. Seed, T. M. & Kreier, J. P. Erythrocyte destruction mechanisms in malaria. in Malaria, Volume 2. Pathology, vector studies, and culture (ed. Kreier, J. P.) 1-46 (1980).
7. Dondorp, A. M., Pongponratn, E. & White, N. J. Reduced microcirculatory flow in severe falciparum malaria: pathophysiology and electron-microscopic pathology. Acta Tropica 89, 309-317 (2004).
8. Suresh, S., et al. Connections between single-cell biomechanics and human disease states: gastrointestinal cancer and malaria. Acta Biomater. 1, 15-30 (2005).
9. Safeukui, I., et al. Retention of Plasmodium falciparum ring-infected erythrocytes in the slow, open microcirculation of the human spleen. Blood 112, 2520-2528 (2008).
10. Mills, J. P., et al. Effect of plasmodial RESA protein on deformability of human red blood cells harboring Plasmodium falciparum. Proceedings of the National Academy of Sciences of the United States of America 104, 9213-9217 (2007).
11. Chotivanich, K., et al. Central Role of the Spleen in Malaria Parasite Clearance. Journal of Infectious Diseases 185, 1538-1541 (2002).
12. Bessis M, M. N., Feo C. Automated ektacytometry: a new method of measuring red cell deformability and red cell indices. Blood Cells. 6, 315-327 (1980).
13. Mokken, F. C., Kedaria, M., Henny, C. P., Hardeman, M. R. & Gelb, A. W. The clinical importance of erythrocyte deformability, a hemorrheological parameter. Annals of Hematology 64, 113-122 (1992).
14. Nash, G. B., Obrien, E., Gordonsmith, E. C. & Dormandy, J. A. Abnormalities in the mechanical properties of red blood cells caused by Plasmodium falciparum. Blood 74, 855-861 (1989).
15. Chien, S., Sung, K. L., Skalak, R., Usami, S. & Toezeren, A. Theoretical and experimental studies on viscoelastic properties of erythrocyte membrane. Biophysical Journal 24, 463-487 (1978).
16. Lekka, M., et al. Elasticity of normal and cancerous human bladder cells studied by scanning force microscopy European Biophysics Journal 28, 312-316 (1999).
17. Mills, J. P., et al. Effect of plasmodial RESA protein on deformability of human red blood cells harboring Plasmodium falciparum. Proc. Natl. Acad. Sci. U.S.A. 104, 9213-9217 (2007).
18. Brody, J. P., Han, Y., Austin, R. H. & Bitensky, M. Deformation and flow of red blood cells in a synthetic lattice: evidence for an active cytoskeleton. Biophysical Journal 68, 2224-2232 (1995).
19. Bow, H., et al. A microfabricated deformability-based flow cytometer with application to malaria. Lab on a Chip.
20. Mairey, E., et al. Cerebral microcirculation shear stress levels determine Neisseria meningitidis attachment sites along the blood-brain barrier. The Journal of Experimental Medicine 203, 1939-1950 (2006).
21. MacDonald, I. C., Ragan, D. M., Schmidt, E. E. & Groom, A. C. Kinetics of red blood cell passage through interendothelial slits into venous sinuses in rat spleen, analyzed by in vivo microscopy. Microvascular Research 33, 118-134 (1987).
22. Fluxion Biosciences, I. Technical note. (2008).
23. Marinkovic, M., et al. Febrile temperature leads to significant stiffening of Plasmodium falciparum parasitized erythrocytes. Am. J. Physiol.—Cell Physiol. 296, C59-C64 (2009).
24. Layez, C., et al. Plasmodium falciparum rhoptry protein RSP2 triggers destruction of the erythroid lineage. Blood 106, 3632-3638 (2005).
25. Buffet, P. A., et al. Ex vivo perfusion of human spleens maintains clearing and processing functions. Blood 107, 3745-3752 (2006).
26. Awah, N. W., Troye-Blomberg, M., Berzins, K. & Gysin, J. Mechanisms of malarial anaemia: Potential involvement of the Plasmodium falciparum low molecular weight rhoptry-associated proteins. Acta Tropica 112, 295-302 (2009).
27. Artmann, G., et al. Hemoglobin senses body temperature. European Biophysics Journal 38, 589-600 (2009).
28. Artmann, G. M., Kelemen, C., Porst, D., Biildt, G. & Chien, S. Temperature Transitions of Protein Properties in Human Red Blood Cells. Biophysical Journal 75, 3179-3183 (1998).
29. Pei, X., et al. The ring-infected erythrocyte surface antigen (RESA) of Plasmodium falciparum stabilizes spectrin tetramers and suppresses further invasion. Blood 110, 1036-1042 (2007).
30. Manno, S., Takakuwa, Y. & Mohandas, N. Identification of a functional role for lipid asymmetry in biological membranes: Phosphatidylserine-skeletal protein interactions modulate membrane stability. Proceedings of the National Academy of Sciences of the United States of America 99, 1943-1948 (2002).
31. Tsvetkova, N. M., et al. Small heat-shock proteins regulate membrane lipid polymorphism. Proceedings of the National Academy of Sciences of the United States of America 99, 13504-13509 (2002).
32. Gershfeld, N. L. & Murayama, M. Thermal instability of red blood cell membrane bilayers: Temperature dependence of hemolysis. Journal of Membrane Biology 101, 67-72 (1988).
33. Pattanapanyasat, K., et al. Febrile temperature but not proinflammatory cytokines promotes phosphatidylserine expression on Plasmodium falciparum malaria-infected red blood cells during parasite maturation. Cytometry Part A 77A, 515-523.
34. Chien, S. Red cell deformability and its relevance to blood flow. Ann. Rev. Physiol. 49, 177-192 (1987).
35. Dondorp, A. M., et al. Prognostic significance of reduced red blood cell deformability in severe falciparum malaria. Am. J. Trop. Med. Hyg. 57, 507-511 (1997).
36. Naumann, K. M., Jones, G. L., Saul, A. & Smith, R. A Plasmodium falciparum exo-antigen alters erythrocyte membrane deformability. FEBS Letters 292, 95-97 (1991).
37. Kikuchi, Y., Horimoto, M. & Koyama, T. Reduced deformability of erythrocytes exposed to hypercapnia. Cellular and Molecular Life Sciences 35, 343-344 (1979).
38. Waugh, R. & Evans, E. A. Thermoelasticity of red blood cell membrane. Biophysical Journal 26, 115-131 (1979).
39. Yawata, Y. Red cell membrane protein band 4.2: phenotypic, genetic and electron microscopic aspects. Biochimica et Biophysica Acta (BBA)—Protein Structure and Molecular Enzymology 1204, 131-148 (1994).
40. Chien, S. Shear Dependence of Effective Cell Volume as a Determinant of Blood Viscosity. Science 168, 977-979 (1970).
41. Williamson, J. R., Shanahan, M. O. & Hochmuth, R. M. The influence of temperature on red cell deformability. Blood 46, 611-624 (1975).
42. Dao, M., Lim, C. T. & Suresh, S. Mechanics of the human red blood cell deformed by optical tweezers [Journal of the Mechanics and Physics of Solids, 51 (2003) 2259-2280]. Journal of the Mechanics and Physics of Solids 53, 493-494 (2005).
43. Hochmuth, R. M., Evans, E. A. & Colvard, D. F. Viscosity of human red cell membrane in plastic flow. Microvascular Research 11, 155-159 (1976).
44. Vyas, N., Avery, B. A., Avery, M. A. & Wyandt, C. M. Carrier-Mediated Partitioning of Artemisinin into Plasmodium falciparum-Infected Erythrocytes. Antimicrob. Agents Chemother. 46, 105-109 (2002).
45. Meshnick, S. R., et al. Iron-dependent free radical generation from the antimalarial agent artemisinin (qinghaosu). Antimicrob. Agents Chemother. 37, 1108-1114 (1993).
46. Chotivanich, K., et al. The Mechanisms of Parasite Clearance after Antimalarial Treatment of Plasmodium falciparum Malaria. Journal of Infectious Diseases 182, 629-633 (2000).

### Example 11

### Determination of the Changes in the Mechanical Properties of T Lymphocytes Due to Cell Activation and in the Absence of Wiskott-Aldrich Syndrome Protein

### Introduction

T lymphocytes, also known as T cells, recirculate in the body and selectively travel to different tissues either to become activated or to carry out effector functions, depending on the cells' activation status. During this journey, T cells are acted on by a myriad of forces and must respond with appropriate mechanical deformations. It has been recognized that T cells having insufficient deformability may fail to migrate properly, thus imposing a mechanical requisite on these cells. The selective migration of T cells to lymphoid organs is called homing (1). This phenomenon has been attributed to the interplay between homing receptors on T cells and expression patterns of chemokines and adhesion molecules at the target tissues (1, 2). However, the contribution of mechanical factors to this process was demonstrated (3). The differential migratory routes and target tissues of naïve and activated T cells likely expose these two populations to varied blood flow rates and thus dissimilar shear forces. In addition, differential cellular arrangements are observed at the sites where the cells exit the circulation (4), suggesting that the two populations may not have the same deformation requirements during the transmigration step. These observations indicate that the activation process may confer on T cells new mechanical properties that allow them to access tissues that were previously prohibited; this is further supported by the rearrangement of the cells' actin cytoskeleton that takes place during T cell activation (5, 6). In other cell types cell differentiation, which occurs during T cell activation, led to altered cell deformability. It has been shown that differentiated acute promyelocytic leukemia cells were about 45% more compliant than the control (7). It has also been discovered that a reduction in the average Young's modulus from 3.2 kPa to 1.7 kPa as human mesenchymal stem cells differentiated into osteoblasts (8).

Knowledge of the mechanical properties of immune cells and a comparison of this information to those obtained from their diseased counterparts may lead to a better understanding of the pathophysiology of the disease. For example, in leukocyte adhesion deficiency type I and II (9, 10), defective neutrophil adhesion leads to poor neutrophil chemotaxis and phagocytosis. Here, the rare genetic disease Wiskott-Aldrich syndrome (WAS) that is characterized by aczema, thrombocytopenia and immunodeficiency (11) was examined. The affected individuals have a mutated WAS gene, whose product, the WAS protein (WASp), has been shown to participate in signal transduction from cell membrane receptors to the actin cytoskeleton (12, 13). T cells from WASp knock-out (WAS−/−) mice were found to exhibit impaired CD3 conjugation-induced activation, homing in vivo, and chemotaxis in vitro (14-17). It was determined that the signaling cascade initiated by TCR ligation, including ZAP-70 and TCR tyrosine phosphorylation, as well as MAPK and SAPK/JNK activation, functioned normally in WAS−/− mice (17). Thus, the effect of WASp on T cell activation should be more downstream and was attributed to its modulation of actin polymerization and polarization (16, 17). Previous in vitro studies showed that WAS−/− T cells could undergo normal rolling and adhesion (14, 16), although the latter seemed to be ligand-dependent. The phenotypes of WAS−/− T cells, together with the actin-regulating role of WAS), suggest a defective actin arrangement of these cells compared to their WT counterparts. This leads to the hypothesis that WAS−/− and WT T cells probably possess dissimilar mechanical properties.

The mechanical behaviors of cells can be studied using a variety of methods, including micropipette aspiration, atomic force microscopy (AFM), optical tweezers, magnetic twisting cytometry, microplates, and the cell poker (18-23). Micropipette aspiration can be used to study the biomechanics of neutrophils and has been applied to adherent cell types (24, 25). This technique can be used for investigating cells that do not need adhesion for survival, such as neutrophils and erythrocytes. By aspirating a portion of the cell content and membrane into a micropipette, this technique allows the determination of a cell's elastic modulus, membrane shear modulus, and viscosity (18, 26). In contrast to the large-scale deformations that micropipette aspiration induce on a cell, AFM imposes localized perturbations. When operated in the indentation mode, AFM can be used to determine cell properties including their stiffness, viscosity, and adhesion force (19, 27, 28). Even though this method is not as frequently applied to non-adherent cells due to their tendency to slip away from the probe, this problem has been addressed by physically trapping cells using microwell arrays (28) and porous membranes (29, 30). When conducted simultaneously, these two devices allow both global and regional cellular information to be revealed.

In this example, mechanical properties of T cells were investigated. In particular, the elasticity and the viscosity, which are involved in the rolling and tethering of non-adherent cells (31-35), of T-cells were investigated. The apparent Young's modulus of WT and WAS−/− T cells before and after activation was determined using micropipette aspiration to assess elasticity. To gauge the viscous property of these cells, AFM cell indentation experiments were conducted at different indentation rates to yield the corresponding apparent Young's modulus, and the variation of this parameter with rate was examined. In one aspect of the example, both WT and WAS−/− T cells, which shown impaired chemotaxis, were induced to migrate by a chemoattractant, and micropipette aspiration testing was conducted on the migrated cells to reveal their elastic properties. Results from these experiments showed alteration of mechanical properties in WT T cells upon activation, as well as between WT and WAS−/− T cells. In another aspect of the example, chemokines were discovered to reduce the apparent Young's modulus of WT but not WAS−/− T cells.

### Results

The elastic and viscous properties of wild-type (WT) and Wiskott-Aldrich Syndrome protein-deficient (WAS−/−) T lymphocytes were probed in terms of the cells' apparent Young's modulus using micropipette aspiration and atomic force microscopy. Upon activation, WT T cells showed a 3-time reduction of their elastic modulus. Naïve WT cells were found to be 1.6 times stiffer than naïve WAS−/− cells, while activated WT and WAS−/− cells yielded comparable stiffness. When deformed at increasing rates, both naïve cell populations, as well as activated WT T cells, displayed a continuous increase of their stiffness, but this increase was significantly delayed in the case of activated WAS−/− cells. The results showed that the cell activation process led to a change in the elasticity of T cells, which may be necessary to fulfill their biological functions. The results also demonstrated that the inability of WAS−/− T cells to properly migrate might be due to a mechanical property mismatch with those of WT T cells. Chemokines were found to dramatically decrease the stiffness of WT but not WAS−/− T cells.

### Purity and Activation Status of T Cells

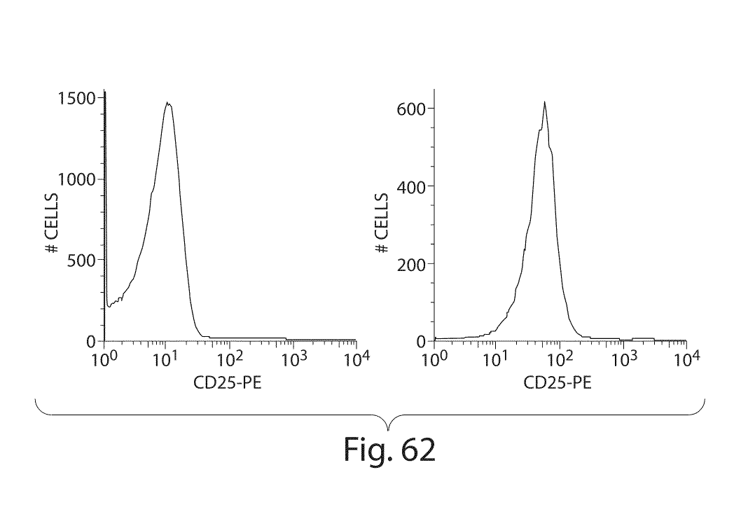

Since most splenocytes are not T cells, the cell extract from the lymph node and the spleen of mice was subjected to an enrichment procedure to separate T cells from unwanted cells. FACS evaluation of the T cell enrichment procedure showed an increase in purity from ˜10% to ˜90% for cells harvested from both WT (in this case the Balb/c strain) and WAS−/− mice. Naïve cells were tested within 24 hours of their harvest, and experiments were kept under three hours as the health of primary cells quickly deteriorates with exposure to room temperature. Similar data were obtained from the beginning and the end of a three-hour testing period. Activated cells were tested within the 24-hour period on their fourth day of activation. Day four was chosen based on the FACS data that ˜90% of the WT cells expressed a high level of CD25, a T cell activation marker (FIG. 62). In contrast to WT T cells, only about 50% of the WAS−/− T cells displayed a strong CD25 staining on day four under the same culture condition. This impaired activation through CD3 conjugation agrees with other reports (15-17). It was observed that activated WAS−/− cells were larger in size than naive cells and could be easily identified by the naked eye. For both naïve and activated cells, no difference in data was observed during the 24-hour testing period. Dead cells were labeled with trypan blue to distinguish them from live cells.

### Changes in Mechanical Properties of T Cells as a Result of Activation

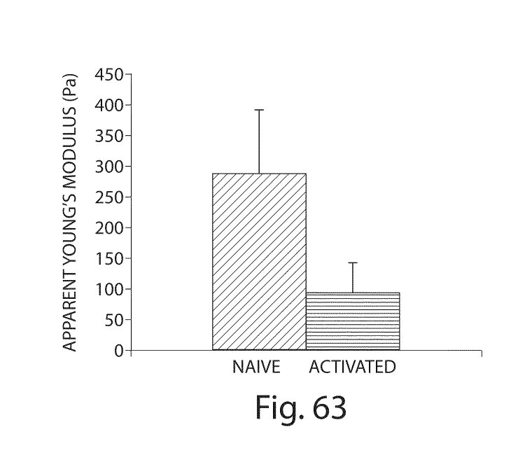

It is believed that cells possess mechanical properties that allow them to adjust to and accommodate their environments. The dissimilar homing routes of naïve and activated T cells can be a result of the activation process which alters the mechanical properties of these cells. Micropipette aspiration and AFM experiments were conducted to study their elastic and viscous responses, respectively. The change in the cell length inside a micropipette with aspiration pressure was tracked and fitted using the half-space model. Naïve WT cells yielded an apparent Young's modulus of 290+/−102 Pa (FIG. 63). Upon activation, this value decreased more than three times to 94+/−49 Pa.

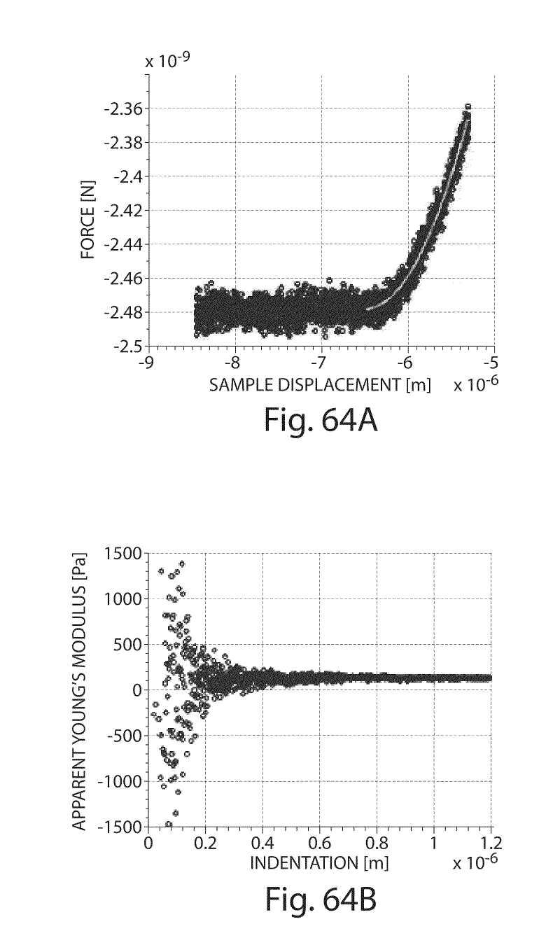

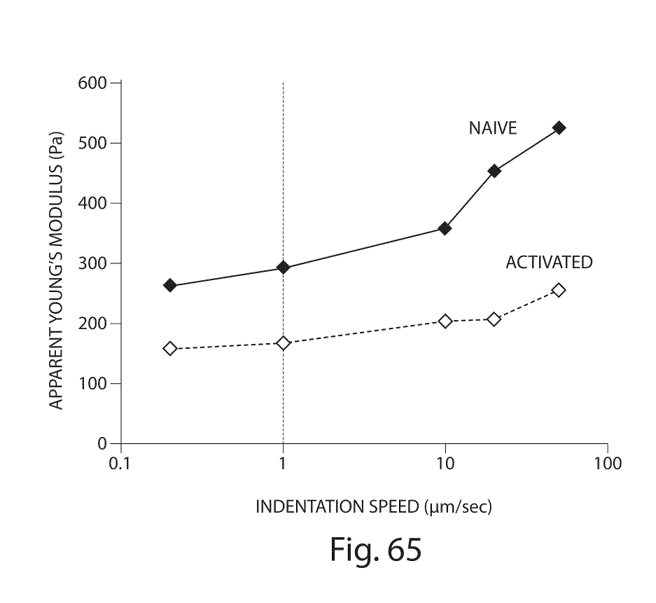

In order to investigate the viscous nature of T cells before and after activation, the variation of the cells' apparent Young's modulus with deformation rate was probed at 200 nm/sec, 1 μm/sec, 10 μm/sec, 20 μm/sec, and 50 μm/sec, using AFM. The approach curve of indentation curves from T cells both before and after activation was fitted using the linear elastic Hertz model (FIG. 64, left panel). When the modulus is plotted against indentation depth, large fluctuations are typically observed at the beginning of the plot (FIG. 64, right panel). The constant modulus at the end indicated that no substrate effect is probed. A continuous increase of the cellular stiffness for both populations was observed at a similar rate (slope) up to about 10 μm/sec, at which point this trend is interrupted by a transition in the case of naïve T cells (FIG. 65). The modulus of these cells continues to rise but does so at a higher rate. The results indicate that activated T cells do not appear to go through a transition in stiffness for the same range of indentation speeds, although such a change may occur at a higher speed, which is not achievable with this specific AFM setup.

### Changes in Mechanical Properties of T Cells as a Result of WAS

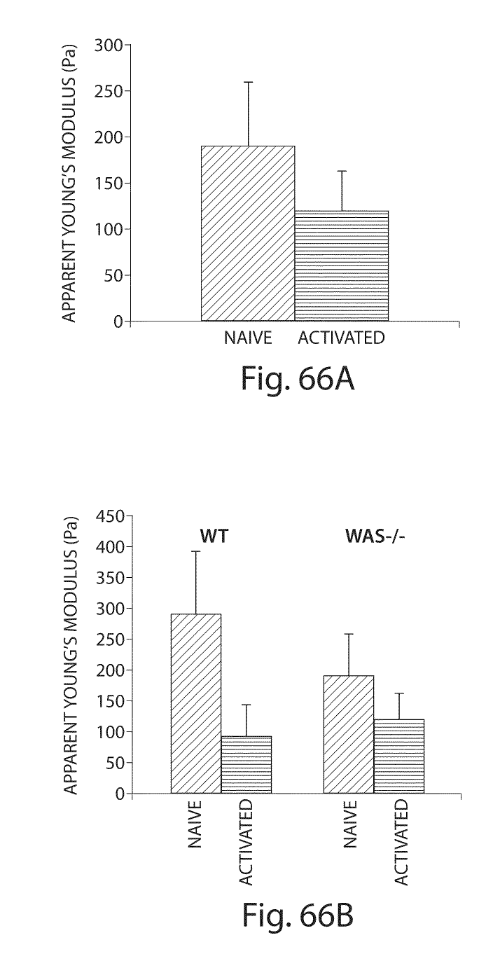

The phenotypes of WAS−/− T cells (14-17), together with the actin-regulating role of WASp (12, 13), suggest a defective actin arrangement of these cells compared to their WT counterparts. It is though that WAS−/− and WT T cells possess dissimilar mechanical properties. Micropipette aspiration experiments were conducted and revealed that naïve WAS−/− T cells had an average apparent Young's modulus of 190+/−69 Pa (FIG. 66), about 1.5 times less than the 290+/−102 Pa determined previously for naïve WT T cells. After activation, WAS−/− cells became less stiff and yielded an apparent Young's modulus of 121+/−41 Pa (FIG. 66). This 1.6-time modulus reduction is smaller than the three-time modulus reduction observed for WT T cells upon activation. Student's t tests conducted showed that the stiffness difference both between naïve WT and naïve WAS−/− T cells, and between naïve and activated WAS−/− T cells, was significant (p<0.05). In contrast, the disparity in modulus between activated WT and WAS−/− T cells did not pass the 95% significance level, indicating that the two populations exhibit similar stiffness.

### Changes in Elastic Response of T Cells as a Result of Chemokine Stimulation

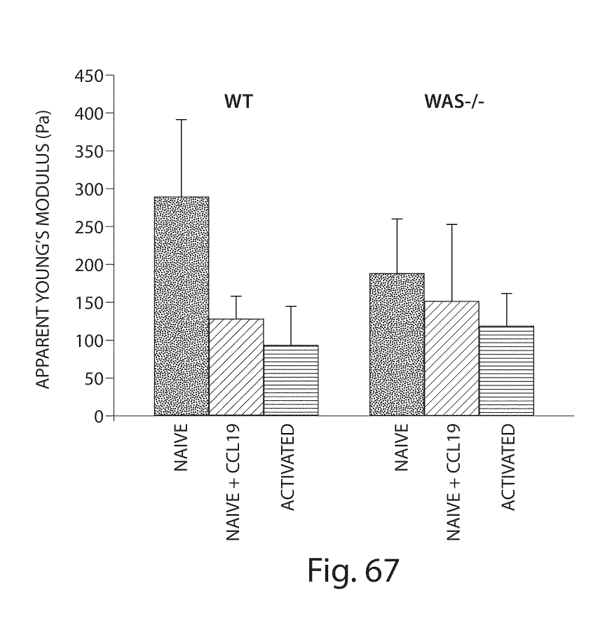

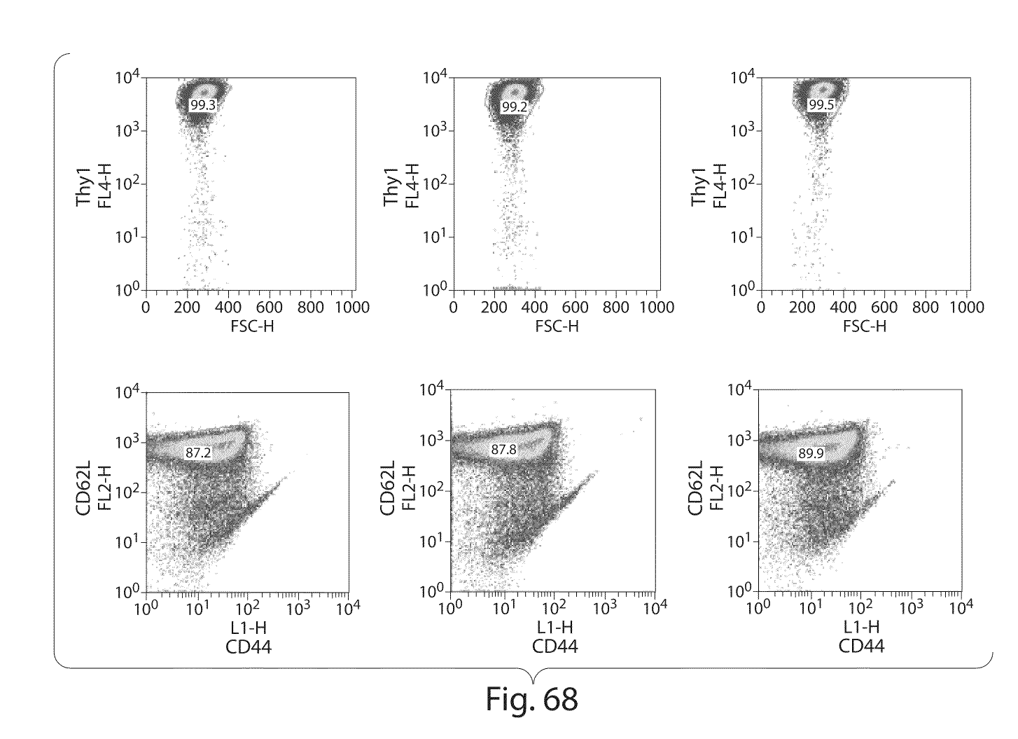

The connection of cellular elasticity to cell chemotaxis was investigated by micropipette aspiration on chemokine-stimulated T cells. About 18.5% of the WT and 6.4% of the WAS−/− cells migrated after seven hours of exposure to CCL19. Migratory WT cells had a modulus of 128+/−33 Pa, a more than 2 times reduction from the value measured in the absence of CCL19 (FIG. 67). They were about 1.4 times stiffer compared to activated cells. FACS analysis showed that ˜90% of the migrating WT cells stained CD62L high and CD44 low, indicative of their naïve phenotype (FIG. 68). The change in modulus between naïve WT T cells before and after the chemokine treatment was significantly different (p<0.05), while the modulus difference between CCL19-treated and activated WT T cells was statistically insignificant (p>0.05), as revealed by Student's t tests. In the case of naïve WAS−/− T cells, a decrease in the apparent Young's modulus from 190+/−69 Pa pre-stimulation to 152+/−102 Pa post-stimulation was observed (FIG. 67). CD62L and CD44 co-staining of these cells yielded a similar staining result as before, namely that close to 90% of the cells in all three groups had the naïve phenotype. Student's t tests were repeated and showed that the difference both between naïve untreated and naïve treated WAS−/− cells, and between naïve treated and activated WAS−/− cells, did not pass the 95% significance level.

### Materials and Methods

Naïve and Activated T Cells Preparation

Cells from the peripheral lymph nodes and the spleen of Balb/c mice and WAS−/− mice on Balb/c background were pooled and enriched using the EasySep Mouse CD8+ T Cell Enrichment Kit from STEMCELL Technologies (British Columbia, Canada). Enrichment was confirmed using fluorescence-activated cell sorting (FACS) by staining the cells with FITC/anti-Thy1.2 and PE/anti-CD8α antibodies (Abs). The memory T cell population was assessed by evaluating the expression of CD44 and CD62L via FACS. T cells in an activation medium with 100 units/mL IL-2 and 1% anti-CD28 Ab were activated by plate-bound anti-CD3 Abs for 4 day at 37° C. The activation medium included RPMI with 10% FBS, 10 mM HEPES (1M), 1% NEAA, 1% sodium pyruvate (100 mM), 50 μM β-mercaptoethanol, 4 mM L-glutamine, and 100 μg/mL Pen/Strep. Cell activation was verified via FACS measurement of CD25.

### Microwell Array Synthesis

Microwell arrays with 8- and 16-μm wells were made to confine naïve and activated T cells. The substrate of an array was a glass discs pre-treated with 3-(trimethoxysilyl) propyl methacrylate. The body of the array was made of polyethyleneglycol diacrylate (PEG DA) of MW 1000. A photoinitiator 2-hydroxy-2 methyl propiophenone was added to 20% PEG DA in PBS to an amount equivalent to 10 wt % of the polymer. A PDMS array template was coated with this solution and finger-pressed against the glass disc for 60 secs. This assembly was then UV-crosslinked for 30 mins.

### Micropipette and Glass Chamber Synthesis

Micropipettes made using a micropipette puller were trimmed to 2.5-3 or 4.5-5 μm inner diameter for testing naïve and activated T cells. MPA glass chambers consisted of a 24 mm×60 mm microscope coverslip bottom, a U-shaped parafilm spacer, and a 22 mm×22 mm microscope coverslip top. The assembly was baked for two hours at 80° C. to ensure good adhesion between the components.

### In Vitro Cell Migration

Enriched WT and WAS−/− T cells in DMEM with 1% BSA and 0.1% Pen/Strep were induced to migrate through polycarbonate Transwell inserts (Costar, Cambridge, Mass.) with 5-μm pores in a 24-well plate toward a CCL19 chemokine source (R&D Systems, Minneapolis, Minn.). Inserts contained 5×10⁵ cells in 100 μL of medium, and wells contained 1 mL of medium with either no CCL19 or 100 ng/mL of CCL19. Cells were allowed to migrate for ˜7 hours at 37° C., and those that crossed the membrane were collected and used for MPA studies.

### Micropipette Aspiration

3×10⁵ cells in 100 μL RPMI with 10% FBS and 1% HEPES was stained with 10 μL trypan blue. The mixture was added to 600 μL of either the RPMI medium or the T cell activation medium with IL-2 for naïve and activated T cell testing. Migratory T cells were tested in the medium used for migration. The MPA device was based on the design of Hochmuth et al (18). The aspiration rate and volume were 36 mL/hr and 2 mL, corresponding to a total pressure of ˜400 Pa. The cell movement in the micropipette was recorded with a CCD camera. Aspirations were performed at approximately room temperature.

### AFM Cell Indentation

A MFP-3D from Asylum Research (CA, USA) was used together with a fluid cell, which held a microwell array of the desired well size. 1×10⁶ T cells stained with trypan blue and diluted into 2.5 mL medium was prepared as above. The spring constant of the AFM probe ranged from 0.019 to 0.024 nN/nm as determined by the thermal spectrum method. The microscope stage was translated to position the T cell array directly below the AFM probe. The system was allowed to equilibrate for 30 mins before testing began, then cells were indented at speeds spanning about three orders of magnitude. For each speed, the cell displacement was tailored to be 1-1.5 μm, and 5-10 force-displacement curves were collected per cell at the center of the cell.

### Data Analysis

MPA data were fitting using the half-space model (77) described by the expressions:

$$E = ϕ ( η ) \frac{3 {r}_{i}}{2 π} ( \frac{Δ p}{L} )$$

$$η = \frac{{r}_{o} - {r}_{i}}{{r}_{i}}$$

where E is the cell modulus, L is the length from the micropipette opening to the cell leading edge, Δp is the pressure differential at a particular L, rᵢ and rₒ are the inner and outer diameter of the micropipette, and φ is the wall function that is approximately 0.2. The ImageJ software was used to manually track the change in L. By plotting Δp against ri/L and finding the best linear fit (minimal total error) a modulus could be derived from the slope of the line. The contact portion of AFM approach curves was fitted using the Hertz model (78), which states that:

$${δ}^{2} = \frac{4 F ( 1 - {v}^{2} )}{3 E tan α}$$

where E is the cell modulus, δ is the indentation depth, F is the indentation force, ν is Poisson's ratio of the cell, assumed to be 0.5 for an incompressible material, and α is the half-angle of the indenter, ˜35°. A MATLAB procedure based, at least in part, on the work of Costa (79) was written to perform least-square fitting of the AFM data, with the fitting parameters being the point of contact and the modulus. The moduli reported here are averages +/− standard deviations calculated from at least five indentation curves per cell.

### Statistics

Student's T test at 95% confidence level was conducted to determine if the difference between two data sets was significant.

### Discussion

Observing the differential migration patterns of naïve and activated T cells, it was hypothesized that the cell activation process changes these cells' mechanical properties and showed a more than three-fold reduction in these cells' modulus from 290+/−102 Pa to 94+/−49 Pa. This result likely arises from the alteration of the T cell cytoskeleton upon activation. Fluorescence staining of naïve T cells revealed a cortical actin mesh lying below the plasma membrane, an intermediate filament cage that permeates the cytoplasm, and microtubules that radiate outward from the microtubule organizing center (36). Literature demonstrates substantial changes to the T cell cytoskeleton upon activation (37-40). Actin reorganizes (37, 38) and both actin filaments and microtubule-organizing centers translocate to specific locations in these cells (39, 40). It has been observed that actin polymerization initiated immediately upon T cells contacting an activating surface, leading to T cell spreading and the formation of a ring of polymerized actin at the cell circumference over a period of 2-3 minutes (41). Macroscopically, these cells interact with the stimulatory surface by first forming small filopodial contacts that subsequently evolved into lamellipodia. In addition to the rearrangement of cytoskeletal components, engagement of TCR results in the release of ezrin-radixin-moesin (ERM) family proteins that anchor the plasma membrane to the cortical actin cytoskeleton (42). This process may relax the cortical actin layer and consequently reduce the stiffness of the cell.

Qualitative studies on the mechanical behaviors of T cells have been performed, and a few experiments have attempted to quantify the mechanical properties of T cells (43, 44). In order to assess the accuracy of the present results, the results were compared to the elastic moduli of cell types close to T cells. Both lymphocytes and neutrophils belong to the leukocyte family and follow a similar migration procedure, although the latter has a larger cytoplasm to nucleus ratio. Two other comparisons were to the Jurkat cell, an acute leukemia cell line of the lymphoid origin, and the HL-60 cell, a cell line that expresses most of the adhesion molecules found on T cells (45). The apparent Young's moduli of neutrophils, the Jurkat cell, and the HL-60 cell were determined as 156 Pa, 48 Pa, and 855 Pa in an AFM study (46). Compared to these values, the present findings of 290+/−102 Pa and 94+/−49 Pa for naïve and activated T cells are on the same order of magnitude. Activated T cells are probably more similar in nature to Jurkat cells since the patterns of cytochemical staining and membrane receptors of Jurkat cells are similar to those of lymphoblasts (47). In addition, naïve T cells are expected to be significantly stiffer than neutrophils (43), which was the trend observed.

The stiffness reduction found herein was also observed in other cell systems upon activation. Acute promyelocytic leukemia cells were induced to differentiate down the neutrophil lineage for three days using all-trans retinoic acid (48). A microfluidic optical stretcher was used to measure the creep compliance of the differentiated cells, and these cells were found to be 45% more compliant than the control and similarly compliant to neutrophils. Electron microscopy revealed an increase in the subcortical actin network size in the differentiated population compared to the undifferentiated one. Since the size of this network is inversely related to the networks' elastic shear modulus (48), this size increase explains the greater compliance of the differentiated cells. The mechanical properties of human mesenchymal stem cells were characterized as they differentiated into osteoblasts and found a reduction in their average Young's modulus from 3.2 kPa to 1.7 kPa (49). In both cases, cells became more compliant upon differentiation. More importantly, the differentiated cells displayed a higher degree of motility than their precursors. Stiffness reduction may be an universal mechanism that cells use to increase motility. A study on metastatic cancer cells showed that showed higher deformability than nonmetastatic ones (50).

The change in the elastic property of T cells due to activation may be required to prepare the cells for their new biological role, which involves traversing a different circulation path to arrive at different lymphoid tissues and subsequently extravasating into and maneuver in these tissues to reach inflammation sources. Correlation of the mechanical properties of a cell with its biological functions has been demonstrated. A higher stiffness for muscle cells than endothelial cells due to the contractile role of the former was shown by the apparent Young's modulus of endothelial, skeletal muscle, and cardiac muscle cells to be 100.3 kPa, 24.7 kPa, and 1.4−6.8 kPa, in that order (51). Cells from the different zones of the intervertebral disc were thought to have varied mechanical properties to accommodate the complex pattern of mechanical loading in this tissue (52). It was found that cells in the nucleus pulposus zone, which experience an isotropic stress-strain environment, were about three times stiffer and significantly more viscous than cells in the annulus fibrosus zone, which endure an anisotropic and heterogeneous state of tension, compression, and shear. The stiffness and collagen content of heart valve interstitial cells from the left and the right side of the heart were compared, and it was discovered that those from the left side contained a significantly higher amount of smooth muscle α-actin and collagen, correlating with the much larger transvalvular pressure that cells on the left side must endure (53).

Cells are viscoelastic materials whose apparent stiffness depends on the rate at which they are deformed. As this rate increases, the viscous nature of the cells results in their increasing resistance to deformation, causing them to appear stiffer. Biologically, the continuous rise of the T cell stiffness means that as a T cell tries to move faster, it will experience a larger resistance trying to propel its viscous content. In a study investigating the force a neutrophil generates during transmigration, it was observed that the maximum opening between two endothelial cells during neutrophil passage was about 4 μm in diameter, with the transmigration process completed in 85+/−20 sec (54). Knowing that the average diameter of a human neutrophil is approximately 8.3 μm (26) and assuming that the transmigrating neutrophil needs to squeeze through an intercellular opening 4 μm in diameter, the velocity of migration ranges from 0.23 to 0.37 μm/sec. This range is comparable to the migration velocity found for naïve T cells in an intact lymph node (55), 10 μm/min (0.17 μm/sec) on average and up to 25 μm/min (0.42 μm/sec). Inspection of the variation of the apparent Young's modulus of naïve and activated WT T cells shows that at the calculated transmigration speeds, the stiffness of naïve and activated cells is 264-293 Pa and 158-167 Pa, respectively. It should be noted that the transmigration speeds discussed here are estimations based on in vitro migration data of neutrophils. T cells in vivo may travel at different velocities depending on their stage of migration, for example, whether they are circulating in the blood and the lymph, transmigrating across a blood vessel, or maneuvering in tissues. The transition observed at 10 μm/sec for naïve T cells indicates increased cellular viscosity beyond this speed. This outcome suggests that if a naïve T cell hypothetically desires to move faster than 10 μm/sec, it will face a dramatically increased amount of resistance that may negatively impact its migration. The sources of cell viscosity are not fully understood. The contributing cellular components can be of a flow-dependent origin, such as fluid viscosity and fluid-solid interactions, and/or of a flow-independent origin, such as the viscosity of the cytoskeleton and the membrane (56-59). One or more of these factors could have contributed to the observed transition.

One of the three characterizing symptoms of the WAS disease is systemic immunodeficiency in the patients. The ability of WAS−/− T cells to perform directed cell migration (chemotaxis) is impaired (14-16), but the biomechanical origin of this behavior is unknown. This results provide evidence that the defective migration of WAS−/− T cells may be caused by a mismatch of their mechanical properties with those of WT T cells. It has been shown through micropipette aspiration studies that naïve WAS−/− T cells has an average modulus of 190+/−69 Pa, about 1.5 times smaller than the 290+/−102 Pa determined for their WT counterparts. After activation, the stiffness of WAS−/− T cells decreased to 121+/−41 Pa, roughly 1.6 times lower than before. However, this reduction is only half of the modulus reduction found previously for WT T cells upon activation, which generated a three-time modulus difference between naïve and activated WT cells.

The results confirmed the hypothesis that WT and WAS−/− T cells have dissimilar mechanical properties. WASp is a multi-modular protein that contains binding sites for both actin monomers and the Arp2/3 complex, which attaches to existing actin filaments and acts as a nucleation site for actin branching (60), thus facilitating the interaction of these molecular species. Therefore, a possible explanation for the 1.5-time modulus difference between naïve WT and naïve WAS−/− T cells is that WASp deficiency reduces and/or disrupts actin polymerization and branching, resulting in an insufficiently and/or incorrectly organized and cross-linked actin network that is less stiff. Direct evidence supporting this postulation is currently unavailable, although morphological studies demonstrate WAS−/− T cells to be severely deformed in shape (15). Since actin is critically involved in T cell activation (37, 38), the hypothesized defective actin network may also impede optimal activation of WAS−/− T cells and thus explain the smaller reduction in their apparent Young's modulus upon activation compared to their WT partners. Furthermore, prior work showed that Vav1-deficient thymocytes exhibited impaired inactivation of ERM proteins, which anchor the plasma membrane to the cortical actin cytoskeleton, upon T cell activation via CD3 conjugation (61). The same experiment also correlated ERM protein inactivation with reduced T cell rigidity, supported by a separate study showing that a smaller contact area was formed between a Vav1-deficient T cell and an APC (62). Since Vav1 is involved in modulating the reorganization of T cell actin cytoskeleton just like WASp, another possible explanation for the smaller modulus reduction in WAS−/− T cells upon activation may be reduced cytoskeletal relaxation as a result of partial inactivation of ERM proteins.

Even though chemotaxis is known to be impaired in WAS−/− T cells, it is unclear how this impairment impacts the migration speed of these cells. Assuming that in order to move normally to execute their biological functions WAS−/− T cells should travel in the same velocity range previously calculated for their WT counterparts, naïve WAS−/− T cells possess an apparent Young's modulus around 220 Pa despite the speed variation. In contrast, the stiffness of naïve WT T cells was determined to be 264-293 Pa. Examination of activated WAS−/− T cells revealed a similar pattern, that the elastic modulus of these cells remained around 130 Pa for the specific velocity range, while their WT counterparts have stiffness in the range 158-167 Pa. The minimal variation of the apparent Young's moduli of WAS−/− T cells in the biologically relevant speed range, regardless of their activation state, suggests that the chemotactic defect of these cells may stem from an inability to dynamically change their stiffness during migration. Studies have demonstrated temporal and spatial variation of cellular stiffness during cell migration (63-67). A significant stiffness decrease in the nuclear region of fibroblasts upon migration was observed, and the decrease was estimated to be from 100 kPa to several kPa (63, 67). Stiffness alteration of migrating cells is implicated in a immunocyto-chemistry study that showed that the distribution of actin in transmigrating leukocytes varied temporally, with sequential detection of actin in the cell anterior, in a podosomal structure, at early stages of dispedesis, in the caudal region where the cell is constricted by an intercellular opening, and finally in the posterior region (68).

The extravasation of T cells at their target tissues depends on chemokines (69). Chemokines promote both T cell adhesion and diapedesis during the extravasation process. Even though it is known that chemokines promote T cell transmigration by modulating the cell's actin cytostructure (69), these cytoskeletal changes have not been directly linked to any mechanical properties of T cells. T cells with deficient actin regulation (WAS−/−) have been observed to demonstrate impaired chemotaxis in transwell migration assays (14-16), and the stiffness of the cell was investigated. Micropipette aspiration experiments were conducted and determined an average apparent Young's modulus of 128+/−33 Pa for naïve WT T cells that migrated in response to the chemokine CCL19, a reduction from the 290+/−102 Pa before stimulation. The much lower modulus suggests that chemokines may promote T cell diapedesis by increasing the deformability of the cells so they can easily reshape to accommodate environmental constraints. Activated T cells have higher motility than their naïve counterparts, and the former was shown to be about three times more compliant. In this work, it was shown that transmigration corresponded to stiffness reduction. Taken together, these results suggest that modification of the elasticity of T cells may be a general requirement for T cell migration. A possible explanation for the modulus reduction of chemokine-stimulated T cells may be the release of ERM proteins that reversibly bind the plasma membrane of T cells to the actin cortical cytoskeleton (70), which may result in increased membrane fluidity that in turn increases cellular deformability.

WAS−/− T cells display impaired chemotaxis even though their expressions of adhesion and chemokine receptors were found to be normal (16), and the stiffness of WT T cells was reduced in response to chemokines. Therefore, the defective migration of WAS−/− T cells could be partially caused by insufficient and/or ineffective modulation of the cellular elasticity in the absence of WASp. The average apparent Young's modulus of naïve WAS−/− T cells that migrated in response to CCL19 was measured to be 152+/−102 Pa, an insignificant decrease (p<0.05) from the 190+/−69 Pa before the chemokine treatment based on a Student's t test. This outcome suggests that cellular elasticity is not an important regulator of the transmigration of WAS−/− T cell, in contrast with the result obtained for WT T cells. Since cell movements involve dynamic rearrangement and polymerization of actin (66, 68, 69), it is surprising that the stiffness of chemokine-stimulated WAS−/− T cells changed so little despite their successful transmigration. A possible explanation for this outcome is that the long experimental time (seven hours) compensated for the high stiffness of the cells so that some of them were still able to cross the porous insert. Other actin-regulatory molecules may be able to mobilize the migration machinery of T cells despite the absence of WASp. One such molecule is WIP, WASp interacting protein (73, 74). A study showed that T cells lacking both WASp and WIP migrated less than those without only one of the proteins, which suggests that the regulatory function of WASp and WIP is nonredundant (17). The previous section showed that even before any experimental manipulation, naïve WAS−/− T cells were already 1.5 times less stiff than their WT partners. This lower stiffness was hypothesized to originate from a defective actin cytoskeleton that arises in the absence of WASp, supported by the abnormal morphology of naïve WAS−/− T cells (16). Even though CCL19 did not appear to affect the elastic property of WAS−/− T cells, it still had an impact on the mechanical behavior of these cells, evident in the much smaller percentage of WAS−/− T cells (6.4%) recovered from the well bottom compared to WT T cells (18.5%).

T cells play a critical role in adaptive immune responses, and understanding their mechanical properties that affect their migration is important. Knowledge of T cell deformability may provide insights to controversies connected to the cells' migration process. For example, these cells are known to transmigrate into tissues via both the transcellular pathway and the paracellular pathway (75). However, it is not known whether one pathway is preferred over the other, and whether the selection could be specific to the T cell subtype (naïve, activated, memory). Quantitative measurements of the mechanical properties of T cells can be used for simulation of T cell biomechanical behaviors to reveal rare cell phenomena not easily detected, as well as allow predictions of cell responses under abnormal circumstances, such as in disease. One disease that was studied herein is WAS. The pathogenesis of the immunodeficiency phenotype of this disease has been partially attributed to impaired homing of immune cells in vivo, but why and how this defect arises are not known. Abnormal chemotaxis of T cells from WAS patients has been shown to correlate with the severity of the disease in these individuals (76). Knowledge of how the mechanical properties of WAS−/− T cells differ from those of WT T cells may lead to a better understanding of the homing defect, which in turn may help design better or new treatment regimens for the disease.

Despite the precautions taken to ensure the health of the cells, the accidental inclusion of dying T cells cannot be ruled out. Dying T cells stained faint blue by trypan blue and were difficult to identify under a microscope. This population could perhaps account for some of the data scatter. In addition, samples of activated T cells contained a heterogeneous size distribution that reflected the different phases of the cell cycle of the cells. For testing, cells of average size were selected, which were also the most abundant population in the sample. This selection could have favored cells in a particular cell cycle stage and should be kept in mind when comparing results from this study with the results of other studies. The data were fit using simple solid models based on the knowledge that naive T cells have little cytoplasm around their nuclei. Even though these models simplify the real structural complexity of a cell, they fulfill the purpose of this work, which was to assess and compare mechanical properties across different T cell populations. These models turned out to describe both the MPA and AFM data fairly well.

### References for Example 11

1. Stein J V, Nombela-Arrieta C (2005) Chemokine control of lymphocyte trafficking: a general overview. Immunology 116:1-12.
2. Mora J R, von Andrian U H (2006) T-cell homing specificity and plasticity: new concepts and future challenges. Trends Immunol 27:235-243.
3. Cinamon G, Shinder V, Alon R (2001) Shear forces promote lymphocyte migration across vascular endothelium bearing apical chemokines. Nature Immuno 2:515-522.
4. Norman M U, Hickey M J (2005) Mechanisms of lymphocyte migration in autoimmune disease. Tissue Antigens 66:163-172.
5. Burkhardt J K, Carrizosa E, Shaffer M H (2008) The actin cytoskeleton in T cell activation. Annu Rev Immunol 26:233-259.
6. Billadeau D D, Nolz J C, Gomez T S (2007) Regulation of T-cell activation by the cytoskeleton. Nature Rev Immunol 7:131-143.
7. Lautenschlager F, Paschke S, Schinkinger S, Bruel A, Beil M, Guck J (2009) The regulatory role of cell mechanics for migration of differentiating myeloid cells. PNAS 106:15696-15701.
8. Titushkin I, Cho M (2007) Modulation of cellular mechanics during osteogenic differentiation of human mesenchymal stem cells. Biophys J 93: 3693-3702.
9. Kishimoto T K, Hollander N, Roberts T M, Anderson D C, Springer T A (1987) Heterogeneous mutations in the β subunit common to the LFA-1, Mac-1, and p 150,95 glycoproteins cause leukocyte adhesion deficiency. Cell 50:193-202.
10. Etzioni A, Harlan J M, Pollack S, Phillips L M, Gershoni-Baruch R, Paulson J C (1993) Leukocyte adhesion deficiency (LAD) II: a new adhesion defect due to absence of sialyl Lewis X, the ligand for selectins. Immunodeficiency 4:307-308.
11. Thrasher A T (2002) WASp in immune-system organization and function. Nature Rev Immunol 2:635-646.
12. Symons M. et al. (1996) Wiskott-Aldrich syndrome protein, a novel effector for the GTPase Cdc42Hs, is implicated in actin polymerization. Cell 84:723-734.
13. Aspenstrom P, Lindberg U, Hall A (1996) Two GTPases, Cdc42 and Rac, bind directly to a protein implicated in the immunodeficiency disorder Wiskott-Aldrich syndrome. Curr Biol 6:70-75.
14. Snapper S B, Meelu P, Nguyen D, Stockton B M, Bozza P, Alt F W, Rosen F S, von Andrian U H, Klein C (2005) WASP deficiency leads to global defect of directed leukocyte migration in vitro and in vivo. J Leuk Bio 77:993-998.
15. Gallego M D, Santamaria M, Pena J, Molina I T (1997) Defective actin reorganization and polarization of Wiskott-Aldrich T cells in response to CD-mediated stimulation. Blood 90:3089-3097.
16. Gallego M D, de la Fuente M A, Anton I M, Snapper S, Fuhlbrigge R, Geha R S (2006) WIP and WASP play complementary roles in T cell homing and chemotaxis to SDF-1α. Int Immunol 18:221-32.
17. Zhang J et al. (1999) Antigen receptor-induced activation and cytoskeletal rearrangement are impaired in Wiskott-Aldrich syndrome protein-deficient lymphocytes. J Exp Med 190: 1329-1341.
18. Hochmuth R M (2000) Micropipette aspiration of living cells. J Biomech 33:15-22.
19. Alcaraz J, Buscemi L, Grabulosa M, Trepat X, Fabry B, Farre R, Navajas D (2003) Microrheology of human lung epithelial cells measured by atomic force microscopy. Biophys J 84:2071-2079.
20. Zhang H, Liu K-K (2008) Optical tweezers for single cells. J R Soc Interface (2008) 5:671-690.
21. Maksym G N, Fabry B, Butler J P, Navajas D, Tschumperlin D J, Laporte J D, Fredberg J J (2000) Mechanical properties of cultured human airway smooth muscle cells from 0.05 to 0.4 Hz. J App Physiol 89: 1619-1632.
22. Thoumine O, Albrecht Ott A (1997) Time scale dependent viscoelastic and contractile regimes in fibroblasts probed by microplate manipulation. J Cell Sci 110:2109-2116.
23. Zahalak G I, McConnaughey W B, Elson E L (1990) Determination of cellular mechanical properties by cell poking, with an application to leukocytes. J Biomech Eng 112:283-294.
24. Jones W R, Ting-Beall H P, Lee G M, Kelley S S, Hochmuth R M, Guilak F (1999) Alterations in the Young's modules and volumetric properties of chondrocytes isolated from normal and osteoarthritic human cartilage. J Biomechanics 32:119-127.
25. Sato, M., Theret, D. P., Wheeler, L. T., Ohshima, N., Nerem, R. M., 1990. Application of the micropipette technique to the measurement of cultured porcine aortic endothelial cell viscoelastic properties. J BiomechEng 112:263-268.
26. Evans E, Yeung A (1989) Apparent viscosity and cortical tension of blood granulocytes determined by micropipet aspiration. Biophys J 56:151-160.
27. Wu Y, Lu H, Cai J, He X, Hu Y, Zhao H, Want X (2009) Membrane surface nanostructures and adhesion property of T lymphocytes exploited by AFM. Nanoscale Res Lett 4:942-947.
28. Rosenbluth M J, Lam W A, Fletcher D A (2006) Force microscopy of nonadherent cells: a comparison of leukemia cell deformability. Biophys J 90:2994-3003.
29. Kasas S, Ikai A (1995) A method for anchoring round shaped cells for atomic force microscope imaging. Biophys J 68: 1678-1680.
30. van der Mei H C, Busscher H J, Bos R, de Vries J, Boonaert C J P, Dufrene Y F (2000) Direct probing by atomic force microscopy of the cell surface softness of a fibrillated and nonfibrillated oral streptococcal strain. Biophys J78:2668-2674.
31. Park E Y, Smith M J, Stropp E S, Snapp K R, DiVietro J A, Walker W F, Schmidtke D W, Diamond S L, Lawrence M B (2002) Comparison of PSGL-1 microbead and neutrophil rolling: microvillus elongation stabilizes P-selectin bond clusters. Biophys J 82:1835-1847.
32. McCarty 0J, Tien N, Bochner B S, Konstantopoulos K (2003) Exogenous eosinophil activation converts PSGL-1-dependent binding to CD18-dependent stable adhesion to platelets in shear flow. Am J Physiol Cell Physiol 284:C1223-C1234.
33. Jadhav S, Eggleton C D, Konstantopoulos K (2005) A 3-D Computational Model Predicts that Cell Deformation Affects Selectin-Mediated Leukocyte Rolling. Biophys J 88:96-104.
34. Dong C, Lei X X (2000) Biomechanics of cell rolling: shear flow, cell-surface adhesion, and cell deformability. J Biomechanics 33:35-43.
35. Finger E, Bruehl R E, Bainton D F, Springer T A (1996) A Differential Role for Cell Shape in Neutrophil Tethering and Rolling on Endothelial Selectins Under Flow. J Immunol 157:5085-5096.
36. Brown M J, Hallam J A, Colucci-Guyon E, Shaw S (2001) Rigidity of circulating lymphocytes is primarily conferred by vimentin intermediate filaments. J Immunol 166:6640-6646.
37. Billadeau D D, Nolz J C, Gomez T S (2007) Regulation of T-cell activation by the cytoskeleton. Nature Rev Immunol 7:131-143.
38. Burkhardt J K, Carrizosa E, Shaffer M H (2008) The actin cytoskeleton in T cell activation. Annu Rev Immunol 26:233-259.
39. Sancho D, Vicente-Manzanares M, Mittelbrunn M, Montoya M C, Gordon-Alonso M, Serrador J M, Sánchez-Madrid F (2002) Regulation of microtubule-organizing center orientation and actomyosin cytoskeleton rearrangement during immune interactions. Immunol Rev 189:84-97.
40. Geiger B, Rosen D, Berke G (1982) Spatial relationships of microtubule-organizing centers and the contact area of cytotoxic T lymphocytes and target cells. J Cell Biol 95:137-143.
41. Barda-Saad M, Braiman A, Titerence R, Bunnell S C, Barr V A, Samelson L E (2005) Dynamic molecular interactions linking the T cell antigen receptor to the actin cytoskeleton. Nature Immunol 6:80-89.
42. Faure S, Salazar-Fontana L I, Semichon M, Tybulewicz V L, Bismuth G, et al. (2004) ERM proteins regulate cytoskeleton relaxation promoting T cell-APC conjugation. Nat Immunol 5:272-79.
43. Zahalak G I, McConnaughey W B, Elson E L (1990) Determination of cellular mechanical properties by cell poking, with an application to leukocytes. J Biomech Eng 112:283-294.
44. Wu Y, Lu H, Cai J, He X, Hu Y, Zhao H, Wang X (2009) Membrane surface nanostructures and adhesion property of T lymphocytes exploited by AFM. Nanoscale Res Lett 4:942-947.
45. Collins S J, Gallo R C, Gallagher R E (1977) Continuous growth and differentiation of human myeloid leukemic-cells in suspension culture. Nature 270:347-349.
46. Rosenbluth M J, Lam W A, Fletcher D A (2006) Force microscopy of nonadherent cells: a comparison of leukemia cell deformability. Biophys J 90:2994-3003.
47. Parsey M V, Lewis G K (1993) Actin polymerization and pseudopod reorganization accompany anti-CD3-induced growth arrest in Jurkat T cells. J Immunol 151:1881-1893.
48. Lautenschlager F, Paschke S, Schinkinger S, Brueld A, Beil M, Guck J (2009) The regulatory role of cell mechanics for migration of differentiating myeloid cells. PNAS 106: 15696-15701.
49. Titushkin I, Cho M (2007) Modulation of cellular mechanics during osteogenic differentiation of human mesenchymal stem cells. Biophys J 93: 3693-3702.
50. Guck J et al. (2005) Optical deformability as an inherent cell marker for testing malignant transformation and metastatic competence. Biophys J 88:3689-98.
51. Mathur A B, Collinswortha A M, Reicherta W M, Krausb W E, Truskey G A (2001) Endothelial, cardiac muscle and skeletal muscle exhibit different viscous and elastic properties as determined by atomic force microscopy. J Biomech 34:1545-1553.
52. Guilak, Farshid et al. (1999) Viscoelastic Properties of Intervertebral Disc Cells: Identification of Two Biomechanically Distinct Cell Populations. Spine 24:2475-2483.
53. Merryman W D, Youn I, Lukoff H D, Krueger P M, Guilak F, Hopkins R A, Sacks M S (2006) Correlation between heart valve interstitial cell stiffness and transvalvular pressure: implications for collagen biosynthesis. Am J Physiol Heart Circ Physiol 290: H224-H231.
54. Rabodzey A, Alcaide P, Luscinskas F W, Ladoux B (2008) Mechanical forces induced by the transendothelial migration of human neutrophils. Biophys J 95:1428-1438.
55. Jacobelli J, Chmura S A, Buxton D B, Davis M M, Krummel M F (2004) A single class II myosin modulates T cell motility and stopping, but not synapse formation. Nat Immunol 5:531-38.
56. Koay E J, Shieh A C, Athanasiou K A (2001) Development of a novel method for creep indentation of single chondrocytes. Ann Biomed Eng 29:S22.
57. Shin D, Athanasiou K. (1999) Cytoindentation for obtaining cell biomechanical properties. J Orthop Res 17:880-90.
58. Dong C, Skalak R, Sung KL (1991) Cytoplasmic rheology of passive neutrophils. Biorheology 28:557-567.
59. Sato M, Theret D P, Wheeler L T, Ohshima N, Nerem R M (1990) Application of the micropipette technique to the measurement of cultured porcine aortic endothelial cell viscoelastic properties. J Biomech Eng 112:263-268.
60. Goley E D, Welch M D (2006) The ARP2/3 complex: an actin nucleator comes of age. Nat Rev Mol Cell Biol 7:713-26.
61. Sophie Faure et al. (2004) ERM proteins regulate cytoskeleton relaxation promoting T cell-APC conjugation. Nature Immunol 5:272-279.
62. Ardouin L (2003) Vav1 transduces TCR signals required for LFA-1 function and cell polarization at the immunological synapse. Eur J Immunol 33:790-797.
63. Nagayama M, Haga H, Kawabata K (2001) Drastic change of local stiffness distribution correlating to cell migration in living fibroblasts. Cell Motility Cytoskeleton 50:173-179.
64. Su J, Brau R R, Jiang X, Whitesides G M, Lange M J, So PT (2007) Geometric confinement influences cellular mechanical properties II—intracellular variances in polarized cells. Mol Cell Biomech 4: 105-118.
65. Yanai M, Butler J P, Suzuki T, Sasaki H, Higuchi H (2004) Regional rheological differences in locomoting neutrophils. Am J Physiol Cell Physiol 287: C603-C611.
66. Yamazaki D, Kurisu S, Takenawa T (2005) Regulation of cancer cell motility through actin reorganization. Cancer Sci 96: 379-386.
67. Haga H, Sasaki S, Kawabata K, Ito E, Ushiki T, Abe K, Sambongi T (2000). Elasticity mapping of living fibroblasts by AFM and immunofluorescence observation of cytoskeleton. Ultramicroscopy 82:253-258.
68. Wolosewick J J (1984) Distribution of actin in migrating leukocytes in vivo. Cell Tissue Res 236:517-525.
71. Vicente-Manzanares M, Sánchez-Madrid F (2004) Role of the cytoskeleton during leukocyte responses. Nature Rev Immunol 4:110-122.
72. Samstag Y, Eibert S M, Klemke M, Wabnitz G H (2003) Actin cytoskeletal dynamics in T lymphocyte activation and migration. J Leu Bio 73:30-48.
73. Ramesh N, Anton I M, Hartwig J H, Geha R S (1997) WIP, a protein associated with the Wiskott-Aldrich Syndrome Protein, induces actin polymerization and redistribution in lymphoid cells. Proc Natl Acad Sci USA 94:14671-14676.
74. Anton I M et al. (2002) WIP deficiency reveals a differential role for WIP and the actin cytoskeleton in T and B cell activation. Immunity 16:193-204.
75. Vestweber D (2007) Adhesion and signaling molecules controlling the transmigration of leukocytes through endothelium. Immunol Rev 218: 178-196.
76. Haddad E, Zugaza JL, Louache F, Debili N, Crouin C, Schwarz K, Fischer A, William Vainchenker W, Bertoglio J (2001) The interaction between Cdc42 and WASP is required for SDF-1-induced T-lymphocyte chemotaxis. Blood 97: 33-38.
77. Theret D P, Levesque M J, Sato M, Nerem R M, Wheeler L T (1988) The application of a homogeneous half-space model in the analysis of endothelial cell micropipette measurements. Trans ASME 110:190-199.
78. Costa K D, Yin F C P (1999) Analysis of indentation: implications for measuring mechanical properties with atomic force microscopy. J Biomech Eng 121:462-471.
79. Costa K D (2006) Imaging and probing cell mechanical properties with the atomic force microscope. Methods Mol Bio 319:331-361.

### SCOPE AND EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all methods, reagents, and configurations described herein are meant to be exemplary and that the actual methods, reagents, and configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the embodiments described herein are presented by way of example only and that, within the scope of the appended claims and equivalents thereto, the invention may be practiced otherwise than as specifically described and claimed. The present invention is directed to each individual feature, system, article, material, reagent, kit, and/or method described herein. In addition, any combination of two or more such features, systems, articles, materials, kits, and/or methods, if such features, systems, articles, materials, reagents, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an”, as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one act, the order of the acts of the method is not necessarily limited to the order in which the acts of the method are recited.

It should further be understood that the citation of any reference herein is not an admission that the reference is prior art.

What is claimed is:

1. A method comprising:
(a) analyzing the deformability of a T cell, and
(b) making a determination about the state of the T cell based on the analysis.

2. The method of claim 1, wherein the state of the T cells is its activation state, a function or a diseased state.

3. A method comprising:
(a) determining the deformability of a T cell,
(b) contacting the T cell with a compound,
(c) analyzing the deformability of the T cell after contact with the compound.

4. A method comprising:
contacting an isolated T cell with a compound that affects the deformability of the T cell.

5. The method of claim 4, wherein the compound is a small molecule or protein.

6. The method of claim 4, wherein small molecule is cytochalasin, latrunculin A and B, nocodazole, colchicine, vincristine, colcemid, paclitaxel, cyclosporin A or FK506.

7. The method of claim 5, wherein the protein is a cytokine, growth factor or antibody.

8. The method of claim 7, wherein the cytokine is IL-2, -4, -7, -15, or -21.

9. The method of claim 7, wherein the antibody is specific for a T-cell surface protein.

10. The method of claim 9, wherein the T-cell surface protein is CD3, CTLA4, CD28 or IL-7R.

11. A method comprising:
administering to a subject with autoimmune disease a compound that affects the deformability of the T cell.

12. The method of claim 11, wherein the compound is a small molecule or protein.

13. The method of claim 12, wherein small molecule is cytochalasin, latrunculin A and B, nocodazole, colchicine, vincristine, colcemid, paclitaxel, cyclosporin A or FK506.

14. The method of claim 12, wherein the protein is a cytokine, growth factor or antibody.

15. The method of claim 14, wherein the cytokine is IL-2, -4, -7, -15, or -21.

16. The method of claim 14, wherein the antibody is specific for a T-cell surface protein.

17. The method of claim 16, wherein the T-cell surface protein is CD3, CTLA4, CD28 or IL-7R.

18. A method comprising:
administering to a subject with an infection or an infectious disease a compound that affects the deformability of the T cell.

19. The method of claim 18, wherein the compound is a small molecule or protein.

20. The method of claim 19, wherein small molecule is cytochalasin, latrunculin A and B, nocodazole, colchicine, vincristine, colcemid, paclitaxel, cyclosporin A or FK506.

21. The method of claim 19, wherein the protein is a cytokine, growth factor or antibody.

22. The method of claim 21, wherein the cytokine is IL-2, -4, -7, -15, or -21.

23. The method of claim 21, wherein the antibody is specific for a T-cell surface protein.

24. The method of claim 23, wherein the T-cell surface protein is CD3, CTLA4, CD28 or IL-7R.
